SEQUENCE LISTING (59 sequences)

SEQ ID NO: 1 - protein, 40 aa, Drosophila melanogaster
HTVAPCPHDLVCPRLRDLADRTPCNFPISYAPLRLGSDFL

SEQ ID NO: 2 - protein, 34 aa, Homo sapiens
FVFAPCPHELPCPQLTNLACSFSQAYHPIPFSWN

SEQ ID NO: 3 - protein, 39 aa, Mus musculus
PSFVFAPCPHELPCPQLNASKSLACSFSQAYHPIPFNWN

SEQ ID NO: 4 - protein, 58 aa, Saccharomyces cerevisiae
KVIAPCPHQRKCPLQVGNPNFYTHKEGKDLKFCNFQKSIKRPKFSIELKKGKLLATSW

SEQ ID NO: 5 - protein, 33 aa, Caenorhabditis elegans
TVLAPCPPHDLGCPLGVHSSCTFNTRFQPIRAD

SEQ ID NO: 6 - protein, 33 aa, Streptomyces coelicolor
RVAAPCPHSAACPIEAGTDWCHFAARVSRSSLH

SEQ ID NO: 7 - protein, 20 aa, Drosophila melanogaster
ADRHTSLYSYAILKKGSQSD

SEQ ID NO: 8 - protein, 19 aa, Homo sapiens
KKPKEEKFSMVILARGSPE

SEQ ID NO: 9 - protein, 19 aa, Mus musculus
KKPKEEIFSMVILARGSPK

SEQ ID NO: 10 - protein, 60 aa, Saccharomyces cerevisiae
DGSQGNASRLKGTGRRNGRDYEILNYSYLIFERSHKDENTLKEIKKLRNENVNGKYDIGS

SEQ ID NO: 11 - protein, 28 aa, Caenorhabditis elegans
GRRSEKESDGTEVSKFTYMILEKSQRKV

SEQ ID NO: 12 - protein, 25 aa, Streptomyces coelicolor
RQVKSGSLAYEDEKFSYVAATRLPV

SEQ ID NO: 13 - protein, 56 aa, Drosophila melanogaster
SSSSWPRIVRPTLVRSKHAICRLCYEKGTLQEVIFTKSKHGKSAYSCAKVSRWGDR

SEQ ID NO: 14 - protein, 56 aa, Homo sapiens
EAHRWPRITQPVLKRPRHVHCHLCCPDGHMQHAVLTARRHGRDLYRCARVSSWGDL

SEQ ID NO: 15 - protein, 56 aa, Mus musculus
EANRWPRITQPVLKRPRHVHCHLCCPDGHMQHAVVTARRHGRDLYRCARVSSWGDL

SEQ ID NO: 16 - protein, 59 aa, Saccharomyces cerevisiae
LGDDTQNSWPRIINDPVKRKGHVMMDLCAPSGELEKWTVSRSFSKQIYHDARKSKKGDL

SEQ ID NO: 17 - protein, 53 aa, Caenorhabditis elegans
NNEHTERILKNRKLGGHVTCDVCTAFRGIQRITLSKKHGEMYTALRSRRDGDL

SEQ ID NO: 18 - protein, 53 aa, Streptomyces coelicolor
EPAASRVVRRPQIRKGQVLLDLCASDERLRRSTVTKRHGDLYKAARDAAWGDP

SEQ ID NO: 19 - protein, 456 aa, Homo sapiens
MAAALKCLLTLGRWCPGLGVAPQARALAALVPGVTQVDNKSGFLQKRPHRQHPGILKLPHVRLPQALANGAQLLLLGSAGPTMENQVQTLTSYLWSRHLPVEPEELQRRARHLEKKFLENPDLSQTEEKLRGAVLHALRKTTYHWQELSYTEGLSLVYMAARLDGGFAAVSRAFHEIRARNPAFQPQTLMDFGSGTGSVTWAAHSIWGQSLREYMCVDRSAAMLVLAEKLLKGGSESGEPYIPGVFFRQFLPVSPKVQFDVVVSAFSLSELPSKADRTEVVQTLWRKTGHFLVLVENGTKAGHSLLMDARDLVLKGKEKSPLDPRPGFVFAPCPHELPCPQLTNLACSFSQAYHPIPFSWNKKPKEEKFSMVILARGSPEEAHRWPRITQPVLKRPRHVHCHLCCPDGHMQHAVLTARRHGRDLYRCARVSSWGDLLPVLTPSAFPPSTAQDPSES

SEQ ID NO: 20 - protein, 478 aa, Homo sapiens
MAAALKCLLTLGRWCPGLGVAPQARALAALVPGVTQVDNKSGFLQKRPHRQHPGILKLPHVRLPQALANGAQLLLLGSAGPTMENQVQTLTSYLWSRHLPVEPEELQRRARHLEKKFLENPDLSQTEEKLRGAVLHALRKTTYHWQELSYTEGLSLVYMAARLDGGFAAVSRAFHEIRARNPAFQPQTLMDFGSGTGSVTWAAHSIWGQSLREYMCVDRSAAMLVLAEKLLKGGSESGEPYIPGVFFRQFLPVSPKVQFDVVVSAFSLSELPSKADRTEVVQTLWRKTGHFLVLVENGTKAGHSLLMDARDLVLKGKEKSPLDPRPGFVFAPCPHELPCPQLTNLACSFSQAYHPIPFSWNKKPKEEKFSMVILARGSPEEAHRWPRITQPVLKRPRHVHCHLCCPDGHMQHAVLTARRHGRYGGCDQNQWDVAGSCSPRQHLFPQGFVSLCPCQLLGRSFTCAYSVCVSSIYGSGSL

SEQ ID NO: 21 - protein, 320 aa, Homo sapiens
MAARLDGGFAAVSRAFHEIRARNPAFQPQTLMDFGSGTGSVTWAAHSIWGQSLREYMCVDRSAAMLVLAEKLLKGGSESGEPYIPGVFFRQFLPVSPKVQFDVVVSAFSLSELPSKADRTEVVQTLWRKTGHFLVLVENGTKAGHSLLMDARDLVLKGKEKSPLDPRPGFVFAPCPHELPCPQLTNLACSFSQAYHPIPFSWNKKPKEEKFSMVILARGSPEEAHRWPRITQPVLKRPRHVHCHLCCPDGHMQHAVLTARRHGRYGGCDQNQWDVAGSCSPRQHLFPQGFVSLCPCQLLGRSFTCAYSVCVSSIYGSGSL

SEQ ID NO: 22 - protein, 298 aa, Homo sapiens
MAARLDGGFAAVSRAFHEIRARNPAFQPQTLMDFGSGTGSVTWAAHSIWGQSLREYMCVDRSAAMLVLAEKLLKGGSESGEPYIPGVFFRQFLPVSPKVQFDVVVSAFSLSELPSKADRTEVVQTLWRKTGHFLVLVENGTKAGHSLLMDARDLVLKGKEKSPLDPRPGFVFAPCPHELPCPQLTNLACSFSQAYHPIPFSWNKKPKEEKFSMVILARGSPEEAHRWPRITQPVLKRPRHVHCHLCCPDGHMQHAVLTARRHGRDLYRCARVSSWGDLLPVLTPSAFPPSTAQDPSES

SEQ ID NO: 23 - protein, 461 aa, Mus sp.
MATARTPRFLLLFCRSCRSLRGPQSRAYAALVPGVSQVDNGSDFLGKKPHRKHPGILHLPHVQLPQALADAAQLMLFERPMRSVEKQVQALTNYLWSRHLPVEPEELQRRAAYLEKKFLEKQDSAPTEEKLREAVLHALRKTTYHWQELSYSEELSLIYMAARLDGGFAAVFRAFHEIQARIPEFRPQTLMDFGSGTGSVAWAAHRTWGQSLREYVCVDSSAAMLGLAEKLLKGGSESGKPCIPGVFFRQFLPVSPKVQFDVVVSAYALSELPSRADRIEVIQNLWRKTSHFLVLVENGTKAGHRLLMDARNLVLGEKEKSPLDLRPSFVFAPCPHELPCPQLNASKSLACSFSQAYHPIPFNWNKKPKEEIFSMVILARGSPKEANRWPRITQPVLKRPRHVHCHLCCPDGHMQHAVVTARRHGRDLYRCARVSSWGDLLPVIAPSEFPPSSPDEEPPEN

SEQ ID NO: 24 - protein, 457 aa, Rattus sp.
MATARASRFLLLFCRSCRSLRGPQSRAFAALVPGVSQVDNRSDFLGKKPHRKHPGILHLPHVHLPQALTDAAQLLLLERPKRSVEKQVQALTNYLWSRHLPVEPEELQRRAAYLEKKFLEKQDSTQTEEKLREAVLHALRKTTYHWQELSYNEELSLIYMAARLDGGFAAVFRAFHEIQARIPEFQPQTLMDFGSGTGSVACTWGQSLREYVCVDRSAAMLGLAEKLLKGGSESGKPCIPGVFFRQFLPVSPKVQFDVVVSAFALSELPSRADRTEVLQNLWRKTSHFLVLVENGTKAGHRLLMDARNLILGDKEKSPLDLRPSFVFAPCPHELPCPQLNAAKSLACSFSQAYQPIPFHWNKKPKEETFSMVILARGSPKEANRWPRITQPVLKRPRHVHCHLCCPDGHMQHAVVTARRHGRDLYRCARVSSWGDLLPVISPSEFPPPSPAEEPPES

SEQ ID NO: 25 - protein, 7 aa, Betapolyomavirus macacae
PKKKRKV

SEQ ID NO: 26 - protein, 10 aa, Betapolyomavirus macacae
PKKKRKVEAS

SEQ ID NO: 27 - protein, 16 aa, Betapolyomavirus macacae
KRPAATKKAGQAKKKK

SEQ ID NO: 28 - protein, 9 aa, Homo sapiens
PAAKRVKLD

SEQ ID NO: 29 - protein, 11 aa, Homo sapiens
RQRRNELKRSP

SEQ ID NO: 30 - protein, 38 aa, Homo sapiens
NQSSNFGPMKGGNFGGRSSGPYGGGGQYFAKPRNQGGY

SEQ ID NO: 31 - protein, 42 aa, Homo sapiens
RMRIZFKNKGKDTAELRRRRVEVSVELRKAKKDEQILKRRNV

SEQ ID NO: 32 - protein, 8 aa, Homo sapiens
VSRKRPRP

SEQ ID NO: 33 - protein, 8 aa, Homo sapiens
PPKKARED

SEQ ID NO: 34 - protein, 8 aa, Homo sapiens
PQPKKKPL

SEQ ID NO: 35 - protein, 12 aa, Mus sp.
SALIKKKKKMAP

SEQ ID NO: 36 - protein, 5 aa, Alphainfluenzavirus influenzae
DRLRR

SEQ ID NO: 37 - protein, 7 aa, Alphainfluenzavirus influenzae
PKQKKRK

SEQ ID NO: 38 - protein, 10 aa, Deltavirus Hepatitis delta virus
RKLKKKIKKL

SEQ ID NO: 39 - protein, 10 aa, Mus sp.
REKKKFLKRR

SEQ ID NO: 40 - protein, 20 aa, Homo sapiens
KRKGDEVDGVDEVAKKKSKK

SEQ ID NO: 41 - protein, 17 aa, Homo sapiens
RKCLQAGMNLEARKTKK

SEQ ID NO: 42 - protein, 288 aa, synthetic construct
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 43 - protein, 183 aa, synthetic construct
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 44 - protein, 401 aa, synthetic construct
MSTDATLIRTTPSHAEADATDTLVATPLMPPRRVISPWPGPGEGQSLMRIPVVDIRGMALMPCTPAKARHLLKSGNARPKRNKLGLFYVQLSYEQEPDNQSLVAGVDPGSKFEGLSVVGTKDTVLNLMVEAPDHVKGAVQTRRTMRRARRQRKWRRPKRFHNRLNRMQRIPPSTRSRWEAKARIVAHLRTILPFTDVVVEDVQAVTRKGKGGTWNGSFSPVQVGKEHLYRLLRAMGLTLHLREGWQTKELREQHGLKKTKSKSKQSFESHAVDSWVLAASISGAEHPTCTRLWYMVPAILHRRQLHRLQASKGGVRKPYGGTRSLGVKRGTLVEHKKYGRCTVGGVDRKRNTISLHEYRTNTRLTQAAKVETCRVLTWLSWRSWLLRGKRTSSKGKGSHSS

SEQ ID NO: 45 - protein, 432 aa, synthetic construct
MQPAKQQNWVFQINGDKQPLDMINPGRCRELQNRGKLASFRRFPYVVIQQQTIENPQTKEYILKIDPGSQWTGFAIQCGNDILFRAELNHRGEAIKFDLVKRAWFRRGRRSRNLRYRKKRLNRAKPEGWLAPSIRHRVLTVETWIKRFMRYCPIAWIEIEQVRFDTQKLANPEIDGVEYQQGELQGYEVREYLLQKWGRKCAYCGTENVPLEVEHIQSKSKGGSSRIGNLTLACHVCNVKKGNLDVRDFLAKSPDILNQVLENSTKPLKDAAAVNSTRYAIVKMAKSICENVKCSSGARTKMNRVRQGLEKTHSLDAACVGESGASIRVLTDRPLLITCKGHGSRQSIRVNASGFPAVKNAKTVFTHIAAGDVVRFTIGKDRKKAQAGTYTARVKTPTPKGFEVLIDGARISLSTMSNVVFVHRSDGYGYEL

SEQ ID NO: 46 - protein, 461 aa, synthetic construct
MAVFVIDKHKRPLMPCSEKRARLLLERGRAVVHRQVPFVIRLKDRTVQHSAVQPLRVALDPGSRATGMALVREKNTVDTGTGEVYRERIALNLFELVHRGHRIREQLDQRRNFRRRRRGANLRYRAPRFDNRRRPPGWLAPSLQHRVDTTMAWVRRLCRWAPASAIGIETVRFDTQRLQNPEISGVEYQQGALAGCEVREYLLEKWGRKCAYCGAENVPLEIEHIVPKSRGGSDRVSNLALACRACNQAKGNRDVRAFLADQPERLARILAQAKAPLKDAAAVNATRWALYRALVDTGLPVEAGTGGRTKWNRTRLGLPKTHALDALCVGQVDQVRHWRVPVLGIRCAGRGSYRRTRLTRHGFPRGYLTRNKSAFGFQTGDLIRAVVTKGKKAGTYLGRIAIRASGSFNIQTPMGVVQGIHHRFCTLLQRADGYGYFVQPKPTEAALSSPRLKAGVSSAGN

SEQ ID NO: 47 - protein, 439 aa, synthetic construct
MTTNVVFVIDTNQKPLQPCSAAVARKLLLRGKAAMFRRYPAVIILKKEVDSVGKPKIELRIDPGSKYTGFALVDSKDNADFIIWGTELEHRGAAICKELTKRSAIRRSRRNRKTRYRKKRFERRKPEGWLAPSLQHRVDTTLTWVKRICKFVPIMSISVEQVKFDLQKLENSDIQGIEYQQGTLAGYTLREALLEHWGRKCAYCDVENVFLEIEHIYPKSKGGSDKFSNLTLACHKCNINKGNKSIDEFLLSDHKRLEQIKLHQKKTLKDAAAVNATRKKLVTTLQEKTFLNVLVSDGASTKMTRLSSSLAKRHWIDAGCVNTTLIVILKTLQPLQVKCNGHGNKQFVTMDAYGFPRKSYEPKKVRKDWKAGDIIRVTKKDGTMLMGRVKKAAKKLVYIPFGGKEASFSSENAKAIHRSDGYRYSFAAIDSELLQKMAT

SEQ ID NO: 48 - protein, 419 aa, synthetic construct
MPNKYAFVLDSKGKLLDPTKSKKAWYLIRKGKASLVEEYPLIIKLKREVPKDQVNSDKLILGIDDGTKKVGFALVQKCQTKNKVLFKAVMEQRQDVSKKMEERRGYRRYRRSHKRYRPARFDNRSSSKRKGRIPPSILQKKQAILRVVNKLKKYIRIDKIVLEDVSIDIRKLTEGRELYNWEYQESNRLDENLRKATLYRDDCTCQLCGTTETMLHAHHIMPRRDGGADSIYNLITLCKACHKDKVDNNEYQYKDQFLAIIDSKELSDLKSASHVMQGKTWLRDKLSKIAQLEITSGGNTANKRIDYEIEKSHSNDAICTTGLLPVDNIDDIKEYYIKPLRKKSKAKIKELKCFRQRDLVKYTKRNGETYTGYITSLRIKNNKYNSKVCNFSTLKGKIFRGYGFRNLTLLNRPKGLMIV

SEQ ID NO: 49 - protein, 1409 aa, synthetic construct
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 50 - protein, 1053 aa, synthetic construct
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 51 - protein, 1365 aa, Streptococcus pyogenes
KYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 52 - DNA, 12 bp, synthetic construct
  source 1..12: mol_type = other DNA
acttgtttaagt

SEQ ID NO: 53 - protein, 9 aa, synthetic construct
IEVMYPPPY

SEQ ID NO: 54 - DNA, 10 bp, synthetic construct
  source 1..10: mol_type = other DNA
ccccctcccc

SEQ ID NO: 55 - DNA, 16 bp, synthetic construct
  source 1..16: mol_type = other DNA
ttctctgttctttcat

SEQ ID NO: 56 - DNA, 16 bp, synthetic construct
  source 1..16: mol_type = other DNA
tctctgttctttcatg

SEQ ID NO: 57 - RNA, 15 nt, synthetic construct
  source 1..15: mol_type = other RNA
tttttcttcgcagga

SEQ ID NO: 58 - RNA, 15 nt, synthetic construct
  source 1..15: mol_type = other RNA
tcgcaggatttttct

SEQ ID NO: 59 - protein, 8 aa, synthetic construct
DYKDDDDK

(19) United States
(12) Patent Application Publication
(10) Pub. No.: US 2025/0099623 A1
(43) Pub. Date: Mar. 27, 2025

(54) COMPOSITIONS AND METHODS FOR ENHANCING INTRA-MITOCHONDRIAL PROTEIN TRANSLATION AND OXIDATIVE PHOSPHORYLATION

(71) Applicants: THE BROAD INSTITUTE, INC., Cambridge, MA (US); THE GENERAL HOSPITAL CORPORATION, Boston, MA (US)

(72) Inventors: Vamsi MOOTHA, Boston, MA (US); Tslil AST, Cambridge, MA (US)

(73) Assignees: THE BROAD INSTITUTE, INC., Cambridge, MA (US); THE GENERAL HOSPITAL CORPORATION, Boston, MA (US)

(21) Appl. No.: 18/893,259

(22) Filed: Sep. 23, 2024

Related U.S. Application Data
(63) Continuation of application No. PCT/US2023/064909, filed on Mar. 24, 2023.
(60) Provisional application No. 63/323,492, filed on Mar. 24, 2022.
(60) Provisional application No. 63/427,587, filed on Nov. 23, 2022.

(51) Int. Cl. A61K 48/00; C12N 9/22; C12N 15/11; C12N 15/86

(52) U.S. Cl. CPC A61K 48/0058; C12N 9/22; C12N 15/11; C12N 15/86; C12N 2310/20; C12N 2740/16043; C12N 2750/14143

(57) ABSTRACT

The present disclosure relates to treating mitochondrial diseases, cancer and other conditions as a result of reduced oxidative phosphorylation (OXPHOS) activity by overexpressing the METTL17 gene, encoding methyltransferase-like 17. Currently, overexpression of METTL17 to increase its copy number and/or intra-mitochondrial activity has not been indicated as a possible therapeutic for treating mitochondrial disease or other diseases such as cancer or aging related to a decline in OXPHOS activity. A variety of gene therapy approaches are presented for overexpression of METTL17 including, but not limited to, AAV, adenovirus and lentiviral vector expression.

### CROSS-REFERENCE TO RELATED APPLICATIONS

This application is a continuation under 35 U.S.C. § 111(a) of International Application No. PCT/US2023/064909 filed Mar. 24, 2023, which claims the benefit of and priority to U.S. Provisional Patent Application No. 63/323,492, filed on Mar. 24, 2022, and U.S. Provisional Patent Application No. 63/427,587, filed on Nov. 23, 2022, the contents of which are incorporated by reference in their entireties herein.

### SEQUENCE LISTING

This application contains a sequence listing filed in electronic form as an xml file entitled BN00007_2423_SL.xml, created on Nov. 25, 2024, and having size of 61,609 bytes. The content of the sequence listing is incorporated herein in its entirety.

### TECHNICAL FIELD

The subject matter disclosed herein generally relates to the field of medicine, and more particularly to treating conditions as a result of reduced respiratory chain activity with agents that boost oxidative phosphorylation in mitochondria.

### BACKGROUND

A decline in the activity of the mitochondrial respiratory chain is associated with a spectrum of human conditions. For example, this decline represents one of the strongest signatures of the aging process itself. Monogenic disorders of the mitochondrial respiratory chain represent the largest class of inborn errors of metabolism. To date, lesions in over 150 genes, encoded by the nuclear (nuDNA) or mitochondrial (mtDNA) genome, have been identified as disease-causing. Mutations in these genes lead to a biochemical deficiency of one or more of the respiratory chain complexes, leading to either tissue-specific or multisystemic disease. There are no FDA approved medicines for these diseases, and current treatment consists of vitamins and antioxidants, none of which have proven benefit. Although there is great hope for emerging gene replacement or editing therapies, the allelic and locus heterogeneity make these disorders particularly challenging. Ideally, we would have mutation-agnostic, generic therapy for boosting mitochondrial respiratory chain activity.

Citation or identification of any document in this application is not an admission that such a document is available as prior art to the present invention.

### SUMMARY

Described in certain example embodiments herein are compositions for enhancing expression of intra-mitochondrial protein translation, respiratory chain activity, mitochondrial oxidative phosphorylation (OXPHOS), or any combination thereof, the composition comprising (a) one or more agents effective to increase (i) methyltransferase-like 17 (METTL17) gene expression, (ii) METTL17 protein expression and/or activity, or both (i) and (ii); (b) a polynucleotide encoding a METTL17 protein operably linked to one or more regulatory elements; (c) a recombinant METTL17 protein and/or a polynucleotide encoding the recombinant METTL17 protein; (d) a gene editing system configured to (i) insert an additional functional copy of a polynucleotide encoding METTL17; (ii) replace an existing or dysfunctional copy of DNA encoding METTL17, or (iii) modify an enhancer region of the METTL17 gene; (e) an engineered transcriptional activator system comprising a DNA-binding domain and a transcriptional activator configured to bind an enhancer of the METTL17 gene such that expression of METLL17 is increased; (f) an epigenetic modification protein comprising a DNA binding domain linked to, or otherwise engineered to associate with, a epigenetic modification domain; or any combination of (a)-(f).

In certain example embodiments, (b) is DNA incorporated into a vector, optionally a viral vector such as a lentiviral, an adenovirus or an adeno-associated (AAV) viral vector. In certain example embodiments, the vector is configured for stable integration of the DNA encoding METTL17 into a nuclear genome of target cells.

In certain example embodiments, (b) is an mRNA encoding METTL17. In certain example embodiments, the mRNA is contained in a delivery vehicle, optionally the delivery vehicle is a viral capsid, a retroelement capsid, an engineered viral like particle (eVLP), or a nanoparticle, and optionally the nanoparticle is a lipid nanoparticle.

In certain example embodiments, the gene editing system comprises a Cas polypeptide, a guide molecule capable of forming a complex with the Cas polypeptide and directing binding of the Cas polypeptide to a target insertion site, and a homology directed repair (HDR) donor template comprising a donor sequence located between a first and second homology arm.

In certain example embodiments, the gene editing system is a CRISPR-associated transposase (CAST) system comprising: i) a catalytically inactive Cas polypeptide and a transposase fused to or otherwise capable of associating with the Cas polypeptide; ii) a guide molecule capable of forming a complex with the Cas polypeptide and directing the complex to a target insertion site; and iii) a donor construct comprising the polynucleotide encoding METTL17, or a functional component thereof, and one or more transposase recognition sequences capable of facilitating recognition by the transposase, whereby the transposase facilitates insertion of the polynucleotide encoding METTL17 at the target insertion site.

In certain example embodiments, the gene editing system is a prime editing system comprising: i) a Cas polypeptide having nickase activity and a reverse transcriptase linked to the Cas polypeptide; and ii) a prime editing guide RNA (pegRNA), wherein the prime editing guide RNA is capable of forming a complex with the Cas polypeptide and direct binding of the complex to a target insertion site, and wherein the pegRNA further comprises a primer binding site configured to hybridize with a portion of a nicked strand of a target polynucleotide, such as nuclear genomic DNA, or the pegRNA further comprises a reverse transcriptase template comprising the polynucleotide encoding the METTL17 polypeptide.

In certain example embodiments, the transcriptional activator system comprises a catalytically inactive Cas polypeptide linked to a transcriptional activator and a guide sequence capable of forming a complex with the Cas polypeptide and directing binding of the dead Cas (dCas)-linked transcriptional activator to a target region such that the transcriptional activator can interact with a target enhancer region of METTL17.

In certain example embodiments, the DNA binding domain is a catalytically inactive Cas polypeptide, the composition further comprising a guide molecule capable of forming a complex with the Cas polypeptide and directing binding of the complex and the epigenetic modification domain to a target region of the genome such that the epigenetic modification domain opens modifies chromosomal architecture such that METLL17 expression is increased. In certain example embodiments, the epigenetic modification domain is a demethylation domain that demethylates one or more CpG islands responsible for silencing expression of METTL17. In certain example embodiments, the gene editing system configured to modify an enhancer region of the METTL17 gene is a base editing system comprising a catalytically inactive Cas polypeptide linked to a nucleobase deaminase and a guide molecule capable of forming a complex with the Cas polypeptide and directing the base editing system to a target modification site to introduce one or more base edits in the enhancer region of the METTL17 gene such that METTL17 expression is increased.

In certain example embodiments, the gene editing system configured to modify an enhancer region of the METTL17 gene is a prime editing system comprising: a Cas polypeptide having a nickase activity, said Cas polypeptide being linked to a reverse transcriptase; and a pegRNA, said pegRNA further comprising a primer binding site configured to hybridize with a portion of a nicked strand of a target polynucleotide, such as nuclear genomic DNA, or said pegRNA further comprises a reverse transcriptase template capable of introducing a single base edit, an insertion or a replacement of a region of the enhancer that increases METTL17 expression. In certain example embodiments, the gene editing system configured to modify an enhancer region of the METTL17 gene comprises a Cas polypeptide, a guide molecule capable of forming a complex with the Cas polypeptide and directing binding of the Cas polypeptide to an enhancer region of the METTL17 gene, and a HDR donor template comprising a donor sequence for insertion into the enhancer region such that METTL17 expression is increased.

In certain example embodiments, the gene editing system is a zinc finger nuclease, a TALEN system, or a meganuclease.

Described in certain example embodiments herein are one or more polynucleotides encoding one or more components of (a)-(f) as previously described.

Described in certain example embodiments herein are delivery systems comprising the one or more polynucleotides or compositions as previously described.

In certain example embodiments, the delivery system is a viral vector delivery system, a particle-based delivery system, or a retroelement-based delivery system.

Described in certain example embodiments herein are delivery systems comprising protein or nucleo-protein complexes of the recombinant protein, a gene editing system, or an engineered transcriptional activator system as previously described, wherein the delivery system is a viral vector, a particle-based delivery system, a retroelement-based delivery system, or an engineered virus-like particle (eVLP).

Described in certain example embodiments herein is a cell, optionally an isolated cell, or progeny thereof, comprising one or more modifications that increase methyltransferase like 17 (METTL17) gene and/or METTL17 protein expression and/or activity. In certain example embodiments, the modification results in addition of an additional copy of the polynucleotide encoding METTL17, single base pair edits, insertions, deletions, and/or substitutions to an enhancer region of an METTL17 gene, or any combination thereof. In certain example embodiments, the cell or progeny thereof is an engineered cell or progeny thereofused for adoptive cell therapy. In certain example embodiments, the cell or progeny thereof is a CAR-T cell or progeny thereof, a CAR-NK cell or progeny thereof, a TCR-T cell or progeny thereof, or a tumor infiltrating lymphocyte (TIL) or progeny thereof. In certain example embodiments, the cell or progeny thereof is a pluripotent stem cell or an induced pluripotent stem cell (iPSC). In certain example embodiments, the cell is a spermatid, spermatozoa, oogonia, or oocyte and wherein the modification does not modify the genome of a human spermatid, spermatozoa, oogonia, oocyte, or any combination thereof.

Described in certain example embodiments herein are pharmaceutical formulations comprising (a) a composition as previously described; (b) one or more polynucleotides as previously described; (c) a delivery system as previously described; (d) a cell or progeny thereof as previously described; or (e) any combination of (a)-(d); and a pharmaceutically acceptable carrier.

Described in certain example embodiments herein are methods of enhancing intra-mitochondrial protein translation and/or OXPHOS activity in a subject in need thereof or a cell population thereof comprising: administering a therapeutically effective amount of (a) a composition as previously described; (b) one or more polynucleotides as previously described, (c) a delivery system as previously described; (d) a cell or progeny thereof as previously described; and/or (e) a pharmaceutical formulation as previously described, to the subject in need thereof or a cell population thereof, thereby increasing the expression or activity of an METTL17 gene and/or a METTL17 protein.

In certain example embodiments, the subject in need thereof is affected by age-related mitochondrial dysfunction or decreased mitochondrial activity not associated with mitochondrial disease. In certain example embodiments, (a), (b), (c), (d), (e), or any combination thereof is co-administered with another therapeutic or supplement effective to counter age-related deficiencies and/or increase lifespan.

In certain example embodiments, the subject in need thereof has, or is suspected of having, a mitochondrial disease, optionally wherein a symptom of the disease is mitochondrial dysfunction or a reduced number of mitochondria. In certain example embodiments, the mitochondrial disease is caused by a mutation in either the mitochondrial DNA (mtDNA) or nuclear DNA (nucDNA). In certain example embodiments, the mitochondrial disease is a monogenic mitochondrial disease. In certain example embodiments, the mitochondrial disease is due to mutation of the frataxin (FXN) gene, optionally wherein the mitochondrial disease is Friedrich's ataxia. In certain example embodiments, the mitochondrial disease is a homoplasmic or a heteroplasmic mitochondrial DNA (mtDNA) disease.

Described in certain example embodiments herein are methods of treating cancer in a subject in need thereof, the method comprising administering an isolated cell or progeny thereof as previously described, such as an engineered cell or progeny thereof used for adoptive cell therapy and/or a pluripotent stem cells or iPSC, or a pharmaceutical formulation thereof, to the subject in need thereof. In certain example embodiments, the cell or progeny thereof is a CAR-T cell or progeny thereof, a CAR-NK cell or progeny thereof, a TCR-T cell or progeny thereof, or a tumor infiltrating lymphocyte (TIL) or progeny thereof.

Described in certain example embodiments herein are methods of increasing fertilization comprising delivering (a) a composition as previously described, (b) one or more polynucleotides as previously described, and/or (c) a delivery system as previously described, or a pharmaceutical formulation thereof, to a spermatid, spermatozoa, oogonia, or oocyte, or any combination thereof, wherein the composition increases the respiration of the spermatid, spermatozoa, oogonia, or oocyte, and wherein the composition does not modify the genome of a human spermatid, spermatozoa, oogonia, oocyte, or any combination thereof.

Described in certain example embodiments herein are methods of increasing the lifespan of a subject or cell thereof, the method comprising administering to the subject or cell thereof (a) a composition as previously described, (b) one or more polynucleotides as previously described, (c) a delivery system as previously described, (d) a cell or progeny thereof as previously described, such as an engineered cell or progeny thereof used for adoptive cell therapy and/or a pluripotent stem cells or iPSC, or any combination of (a)-(d) or a pharmaceutical formulation thereof. In certain example embodiments, the cell or progeny thereof is a CAR-T cell or progeny thereof, a CAR-NK cell or progeny thereof, a TCR-T cell or progeny thereof, or a tumor infiltrating lymphocyte (TIL) or progeny thereof.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Overexpressed METTL17 is enriched in the mitochondria. Immunoblot of GFP or Flag tagged METTL17 cells, examining both whole cell extracts as well as isolated mitochondria. The mitochondrial protein HSP60 is shown as a control.

FIG. 2—METTL17 overexpression restores viability on non-fermentable substrates in cellular models of Friedreich's ataxia. Control or FXN null cells (sgCtrl and sgFXN, respectively) were grown on the non-fermentable carbon source, galactose. These cells were either overexpressing GFP or METTL17. Cell viability was tested after 72 hours of growth on galactose. Bar plots show mean±SD.

FIG. 3—METTL17 overexpression boosts basal and maximal mitochondrial oxygen consumption in both control and FXN null cells. Whole-cell oxygen consumption was tested in control or FXN null cells, overexpressing either GFP or METTL17. Cells were treated with Oligomycin, CCCP and Antimycin. Points are mean±SD.

FIG. 4—METTL17 overexpression does not affect growth rates. Control or FXN null cells overexpressing GFP or METTL17 were grown for 72 h and their population doubling rates were calculated. Bar plots show mean±SD.

FIG. 5A-5D—Proteomic analysis of FXN null cells reveals a marked depletion of known Fe—S cluster containing proteins and reduction of small mitoribosome subunits. FIG. 5A. Quantitative whole cell proteomic analysis was carried out on K562 cells edited with control or FXN targeted guides, depleting for this allosteric regulator of Fe—S cluster biosynthesis. FIG. 5B. Waterfall plot of protein fold change in FXN/Control cells, highlighting FXN and validated human Fe—S cluster containing proteins. FIG. 5C. OXPHOS proteins are organized by complex with LogFC value in grey indicating proteins that are depleted in FXN null cells. Genes are ordered alphabetically within complex using complex-specific prefixes (NDUF, SDH, UQCR, COX, ATP5) (e.g., A2 in CI refers to NDUFA2 whereas A in CII refers to SDHA). FIG. 5D. Waterfall plot of protein fold change in FXN/Control cells, highlighting proteins in the small and large mitoribosome subunit, as well as the small subunit assembly factor, METTL17.

FIG. 6A-6D—Mitochondrial translation is attenuated in the absence of FXN. FIG. 6A. Mitochondrial translation, as assessed by autoradiography after 35S-methionine/cysteine labeling, of cells expressing sgRNAs targeting FXN, NDUFS1 or FBXL5. All cells were treated with 200 g/mL emetine, and control cells in the last lane were also treated with 50 g/L chloramphenicol. FIG. 6B. Schematic overview of the genome-wide CRISPR genetic interaction screens carried out in K562 cells. Cells were either infected with guides against FXN or a control locus before introduction of the library. Following expansion, cells were sequenced to assess the relative abundance of guides in the FXN null vs. control background. FIG. 6C. GO (top) and MitoCarta 3.0 (bottom) enrichment analysis of genetic interactors identified in for FXN null cells. FIG. 6D. Scatterplot of Z scores showing knockouts growth in sgCtrl vs. sgFXN backgrounds. The positive control (IRP2) and the mitochondrial ribosome assembly genes (METTL17 and MPV17L2) are highlighted.

FIG. 7A-7H—METTL17 is depleted in the absence of FXN and is essential for robust mitochondrial translation. The figures herein are for illustrative purposes only and are not necessarily drawn to scale. FIG. 7A. Immunoblot for FXN, METTL17 and the loading control actin in K562 cells edited with control, FXN, NDUFS1 and FBXL5 guides. FIG. 7B. qPCR for METTL17 expression levels in sgCtrl and sgFXN cells FIG. 7C. Cells edited for control, FXN, METTL17 and CDK5RAP1 genes were grown for 24 h in galactose media, and viability was assessed for each background. FIG. 7D. Immunoblot for FXN, METTL17, CDK5RAP1, select OXPHOS subunits and the loading control tubulin in cells edited with control, FXN, METTL17 and CDK5RAP1 guides. FIG. 7E. Correlation analysis of gene dependencies sourced from DepMap. Presented is the gene network that correlates with METTL17 deletion using FIREWORKS (Amici et al., 2021). Solid and dashed lines represent primary and secondary correlations, respectively. FIG. 7F. Mitochondrial translation, as assessed by autoradiography after 35S-methionine/cysteine labeling, of cells expressing sgRNAs targeting METTL17 or CDK5RAP1. All cells were treated with 200 g/mL emetine, and control cells in the last lane were also treated with 50 g/L chloramphenicol. FIG. 7G. qPCR analysis of 12S levels in cells edited with control, FXN, METTL17 or CDK5RAP1 guides. FIG. 7H. Immunoblot for METTL17 and the loading control actin in cells edited with control or FXN guides. Following editing, cells were grown in 21% or 1% oxygen. All bar plots show mean±SD. **=p<0.01, ****=p<0.0001. One-way ANOVA with Bonferroni's post-test.

FIG. 8A-8G—METTL17 has two conserved motifs linked to Fe—S binding, which are crucial for its functionality. FIG. 8A. (SEQ ID NO: 1-18) Multiple sequence alignment for METTL17 homologues showing motifs associated with Fe—S cluster binding: 4 cysteine metal binding pocket (bolded C residues, as represented in greyscale) and a LYR handoff motif (bolded Y residues, as represented in greyscale). FIG. 8B. Immunoblot from whole cell and mitoprep extracts of cells expressing GFP, METTL17-FLAG, CYSMut-FLAG or LYRMut-FLAG constructs. FIG. 8C. Control or METTL17 edited cells expressing GFP, METTL17-FLAG, CYSMut-FLAG or LYRMut-FLAG constructs were grown for 24 h in galactose, following which their viability was assessed. FIG. 8D. Immunoblots examining OXPHOS subunits or the loading control HSP60 in Control or METTL17 edited cells expressing GFP, METTL17-FLAG, CYSMut-FLAG or LYRMut-FLAG constructs. FIG. 8E. Mitochondrial translation, as assessed by autoradiography after 35S-methionine/cysteine labeling, in Control or METTL17 edited cells expressing GFP, METTL17-FLAG, CYSMut-FLAG or LYRMut-FLAG constructs. All cells were treated with 200 g/mL emetine, and control cells in the last lane were also treated with 50 g/μL chloramphenicol. FIG. 8F. qPCR analysis of 12S levels in Control or METTL17 edited cells expressing GFP, METTL17-FLAG, CYSMut-FLAG or LYRMut-FLAG constructs. FIG. 8G. Formaldehyde-linked RNA immunoprecipitation of the 12S to GFP, METTL17-FLAG, CYSMut-FLAG or LYRMut-FLAG proteins. Results were normalized to input construct and 12S levels. All bar plots show mean±SD. ****=p<0.0001. One-way ANOVA with Bonferroni's post-test.

FIG. 9A-9C—Human METTL17 expressed and purified from E. coli contains an Fe—S cluster. FIG. 9A. Gel filtration chromatography and SDS-PAGE analysis demonstrate that the purified METTL17 construct runs as a monomer near its predicted molecular weight of 50 kD. FIG. 9B. Iron content of purified METTL17 and CYSMut as determined by bicinchoninic acid assay and inductively coupled plasma mass spectrometry. The CYSMut is a METTL17 variant in which the four cysteines predicted to coordinate the cluster are mutated to serine (C333S, C339S, C347S, and C404S). FIG. 9C. The UV-Vis absorption spectra of METTL17 exhibits abroad band around 420 nm, consistent with the presence of an [Fe4S4]2+ or an [Fe3S4]+ cluster; the latter is ruled out by EPR spectroscopy as described in the text. This band is lost in the spectrum of CYSMut. For clarity, spectra were normalized to the intensity at 280 nm.

FIG. 10A-10C—Cryo-EM structure of the yeast SSU-METTL17 complex and involved elements. FIG. 10A. Overall view of METTL17 on the SSU, and close-up views. Top close up shows the position of METTL17 (C-terminal domain, CTD light grey; N-terminal domain, NTD dark grey, as represented in greyscale) between the rRNA (light grey, as represented in greyscale) of the head and body, while the C-terminal extension (CTE) occupies the mRNA path. Bottom close up shows the coordination of 4Fe-4S cluster by four cysteines, including Cys513 from the CTD, and related structural elements with their cryo-EM densities: flipped base A1100, a cis-proline, arginine that is within salt bridge distance, and a conserved histidine that can be involved in a transfer and ligation to the Fe—S unit. FIG. 10B. Conformational changes within the rRNA region h30-34 that is involved in METTL17 binding. Superimposed models of the SSU-METTL17 (light grey, as represented in greyscale) with unbound state (dark grey). Sticks represent the rRNA residues responsible for the METTL17 interaction. FIG. 10C. Superposition of SSU-METTL17 with SSU-mtIF3 showing clashes of METTL17 (dark grey, as represented in greyscale) with mtIF3 (light grey surface representation, as represented in greyscale).

FIG. 11A-11E—Overexpression of METTL17 restores the mitochondrial bioenergetics, but not growth, of FXN null human cells. FIG. 11A. Control or FXN edited cells expressing GFP, METTL17-FLAG, CYSMut-FLAG or LYRMut-FLAG constructs were grown for 24 h in glucose (left) or galactose (right), following which their viability was assessed. FIG. 11B. Immunoblots examining OXPHOS subunits or the loading control HSP60 in Control or FXN edited cells expressing GFP, METTL17-FLAG, CYSMut-FLAG or LYRMut-FLAG constructs. FIG. 11C. Oxygen consumption rate (OCR) of Control or FXN edited cells expressing GFP or METTL17-FLAG. Cells were sequentially treated with oligomycin, Bam15 and piericidin+antimycin. FIG. 11D. Population doubling over 72 h of Control or FXN edited cells expressing GFP, METTL17-FLAG, CYSMut-FLAG or LYRMut-FLAG constructs. FIG. 11E. FXN activates Fe—S cluster formation, which can be utilized to support (i) mitochondrial bioenergetics via formation of the electron transport chain (ETC) or (ii) cell growth and division. METTL17 is a key Fe—S cluster bearing modulator of mitochondrial bioenergetics, and its absence in FXN null cells accounts for much of the mito. bioenergic defects observed in these cells. All bar plots show mean±SD. **=p<0.01, ****=p<0.0001. One-way ANOVA with Bonferroni's post-test.

FIG. 12A-12E—OXPHOS, but not ISC machinery or the mito-proteome, is depleted in the absence of FXN. FIG. 12A. Waterfall plots of protein fold change in FXN/Control cells, highlighting proteins in the core ISC machinery. FIG. 12B. Analysis of MitoPathways depleted in FXN null cells. Pathways in bold are independent. FIG. 12C-12E. Waterfall plots of protein fold change in FXN/Control cells, highlighting proteins in MitoCarta 3.0 (FIG. 12C) mtDNA maintenance proteins (FIG. 12D) and mtRNA metabolism (FIG. 12E).

FIG. 13A-13G—mtDNA replication and transcription is not significantly altered in FXN null cells. FIG. 13A. Ponceau S staining of the protein membrane found in FIG. 6A. FIG. 13B. sgCtrl and sgFXN cells were grown for 72 h in escalating concentrations on chloramphenicol, and the relative growth of each strain compared to DMSO treatment was calculated. FIG. 13C. qPCR for mtDNA copy number in sgCtrl and sgFXN cells. FIG. 13D. Mitostring assay examining the levels of mtDNA encoded transcripts in sgCtrl, sgFXN and sgMTPAP cells. FIG. 13E. Histograms of the Z score of cutting controls, non-expressed genes and essential genes as defined by (Hart et al., 2015) in the genetic interaction screens preformed in sgCtrl or sgFXN cells. FIG. 13F. Scatterplot of Z scores showing knockouts growth in sgCtrl vs. sgFXN backgrounds. All the mitochondrial ribosome assembly genes, as defined by MitoCarta 3.0, are highlighted in dark grey. FIG. 13G. Relative growth rates of cells edited for control or MPV17L2 gene, on the background on sgCtrl or sgFXN. Growth rates were normalized to the unedited growth rate for each strain. All bar plots show mean±SD. *=p<0.05, **=p<0.01, ***=p<0.001, ****=p<0.0001. One-way ANOVA with Bonferroni's post-test.

FIG. 14A-14D—METTL17 is depleted in FXN null cells and is linked to mitochondrial translation. FIG. 14A. Immunoblot for FXN, METTL17 and the loading control actin in 293T and A549 cells edited with control or FXN guides. FIG. 14B. Cells edited for control, FXN, METTL17 and CDK5RAP1 genes were grown for 24 h in glucose media, and viability was assessed on each background. FIG. 14C. Protein-protein interactions identified for METTL17 in 293T cells as identified by (Huttlin et al, 2021). FIG. 14D. Ponceau S staining of the protein membrane found in FIG. 7E. All bar plots show mean±SD.

FIG. 15A-15D—METTL17 has characteristics of an Fe—S cluster binding protein. FIG. 15A. Immunoblot for METTL17 and the loading control actin in cells edited for control, ISC genes (ISCU and NFS1) or a CIA gene (CIAO3). FIG. 15B. Cells edited for control or METTL17 genes and expressing WT or mutant forms of METTL17 were grown for 24 h in glucose media, and viability was assessed on each background. FIG. 15C. Ponceau S staining of the protein membrane found in FIG. 8E. FIG. 15D. Formaldehyde-linked RNA immunoprecipitation of the 16S to GFP, METTL17-FLAG, CYSMut-FLAG or LYRMut-FLAG proteins. Results were normalized to input construct and 16S levels. All bar plots show mean±SD. *=p<0.05, One-way ANOVA with Bonferroni's post-test.

FIG. 16A-16C—Cryo-EM image processing. FIG. 16A. The data processing scheme. FIG. 16B. Overall maps, combined maps of the local-masked refinements colored by local resolution are shown for SSU-METTL17 (top), SSU-mtIF3 (middle), and SSU (bottom). FIG. 16C. Fourier Shell Correlation curves of the half maps and local-masked refinements. The 0.143 criterion is shown as dashed lines.

FIG. 17A-17G—Cryo-EM structure of the yeast SSU, METTL17 and mtIF3. FIG. 17A-17D. Improvements in the model of S. cerevisiae mitoribosome. Overview of the SSU model from the back with improved proteins identified with varying grey shades. The close-up views show modeled elements with their corresponding density map, and equivalent regions from previous studies (Desai et al., 2017) are shown for comparison. FIG. 17A. The nucleotide density for mS29 in the SSU head. FIG. 17B. The density and corresponding models of uS2m, uS3m, uS7m, mS35 that form a previously unsigned helix bundle between the head and body. FIG. 17C. Complete models for bS1m and mS26 that form contacts at the mRNA channel exit. FIG. 17D. Remodeled and reannotated mS27 interacts with h44, which was previously partially built as poly-Ala and named mS44. FIG. 17E. Comparison between the yeast cryo-EM model and human AlphaFold2 (Jumper et al., 2021) prediction of METTL17 shows that the predicted conformations of the NTD (dark grey, as represented in greyscale) and CTD (light grey, as represented in greyscale) are highly similar, including the coordination of the 4Fe-4S shown in the close-up view, and structural differences are observed only in the terminal extensions. The Fe—S cluster in the human model was placed by superposing that of the yeast cryo-EM structure. FIG. 17F. Comparison between yeast and human SSU (left) and METTL17 (right) interfaces. Phylum-specific protein extensions have been removed for clarity. The residues involved in interactions are shown in sticks for RNA and spheres for protein. FIG. 17G. Comparison between yeast and human (Khawaja et al., 2020) SSU—IF3 complex with close-up views showing that the binding of the mtIF3 CTD (dark grey, as represented in greyscale) is conserved. Thus, human mtIF3 has similar structural characteristics and would also clash with METTL17 on the SSU. The NTD of mtIF3 is not well resolved in the map, and thus hasn't been modelled. On the other hand, the C-terminal extensions (CTE) forming a helix have different orientations. The CTE in yeast keeps contacting the rRNA in the body, whereas that of human is exposed.

FIG. 18A-18D—METTL17 overexpression restores the faulty mitochondrial bioenergetics of FXN depleted cells. FIG. 18A. Oxygen consumption rate of Control (top) or FXN (bottom) edited cells expressing GFP, METTL17-FLAG, CYSMut-FLAG or LYRMut-FLAG. Cells were sequentially treated with oligomycin, Bam15 and Piericidin A+Antimycin A. FIG. 18B-18C. Basal (FIG. 18B) and maximal (FIG. 18C) OCR of Control or FXN edited cells expressing GFP or METTL17-FLAG. FIG. 18D. Immunoblots examining POLD1 or the loading control HSP60 in Control or FXN edited cells expressing GFP, METTL17-FLAG, CYSMut-FLAG or LYRMut-FLAG constructs. All bar plots show mean±SD. **=p<0.01, ****=p<0.0001. One-way ANOVA with Bonferroni's post-test.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a,” “an,” and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed compositions and methods. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid.” The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment,” “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Oxidative phosphorylation is an eukaryotic cell's main ATP producing pathway and is localized within mitochondria. OXPHOS is encoded by both mtDNA (which encodes 13 OXPHOS subunits), but also requires hundreds of nuclear encoded gene products that are imported to help express and assemble these 13 proteins along with nuclear genome encoded subunits. Mutations in mitochondrial DNA (mtDNA) or in nuclear DNA (nuDNA) can lead to monogenic mitochondrial disease. To date that are over 300 known monogenic mitochondrial diseases. OXPHOS also declines with agent and can becoming limiting in T cell activity. A common denominator in all of these conditions is low OXPHOS activity.

METTL17 is a nuclear gene product that plays a role in intra-mitochondrial protein translation. The Applicant has discovered that METTL17 is limiting for intra-mitochondrial protein translation and that over-expression of METTL17 is sufficient to boost all 13 mtDNA encoded OXPHOS subunits, which then elevate the abundance and activity of the entire OXPHOS system. Accordingly, provided herein are compositions, delivery systems, engineered cells and methods that can address conditions characterized by low OXPHOS activity in a way that is agnostic to the underlying cause, enabling a wide range of useful applications and therapeutic interventions. Overexpression of METTL17 has not heretofore been identified with boosting oxidative phosphorylation in general and has not been engineered or used to treat mitochondrial diseases, enhance T cell function, enhance oocyte fertilization, or used as an anti-aging therapy, among other potential applications.

Embodiments disclosed herein provide a gene, METTL17, which when overexpressed, increase the intra-mitochondrial translation of respiratory chain subunits in cells and tissues with a concomitant increase in respiratory chain activity, i.e., a notable increase in the oxidative phosphorylation activity of the mitochondria. The common denominator among these diseases and conditions is a decrease or decline in the oxidative phosphorylation capacity of the cells. Mitochondrial diseases tend to be functionally recessive, with a non-zero residual oxidative phosphorylation activity and so boosting METTL17 expression and activity may increase intra-mitochondrial translation leading to a concomitant increase in respiratory chain activity with positive therapeutic effects. In CAR-T cells, a decline in oxidative phosphorylation activity can contribute to poor immune function and immune exhaustion and thus may assist in rendering these cancer therapies more effective.

In one aspect, embodiments disclosed herein are directed to compositions for enhancing expression of intra-mitochondrial protein translation and/or respiratory chain activity and/or mitochondrial oxidative phosphorylation (OXPHOS) activity comprising administering one or more agents effective to increase METTL17 gene and/or a methyltransferase like 17 (METTL17) protein expression and/or activity.

In another aspect, embodiments disclosed herein are directed to compositions for enhancing expression of intra-mitochondrial protein translation and/or mitochondrial respiratory chain activity and/or mitochondrial oxidative phosphorylation (OXPHOS) activity comprising a polynucleotide encoding a methyltransferase-like 17 (METTL17) protein operably linked to one or more regulatory elements.

In another aspect, embodiments disclosed herein are directed to compositions for enhancing intra-mitochondrial protein translation and/or mitochondrial respiratory chain activity comprising a gene editing system configured to insert an additional functional copy of a polynucleotide encoding METTL17 or replace an existing or dysfunctional copy of DNA encoding METTL17.

In another aspect, embodiments disclosed herein are directed to compositions for enhancing intra-mitochondrial protein translation and/or mitochondrial respiratory chain activity comprising administering an engineered transcriptional activator system comprising a DNA-binding domain and a transcriptional activator configured to bind an enhancer of the METTL17 gene such that expression of METLL17 is increased.

In another aspect, embodiments disclosed herein are directed to compositions for enhancing intra-mitochondrial protein translation and/or mitochondrial respiratory chain activity comprising a gene editing system that modifies an enhancer region of the METTL17 gene.

In another aspect, embodiments disclosed herein are directed to methods of enhancing intra-mitochondrial protein translation and/or mitochondrial respiratory chain activity in a subject in need thereof or a cell population thereof by administering to the subject in need thereof a therapeutically effective amount of any of the disclosed compositions or pharmaceutical formulations thereof, that increases the expression or activity of a METTL17 gene and/or METTL17 protein.

In another aspect, embodiments disclosed herein are directed to methods for treating subjects affected by age-related mitochondrial dysfunction or decreased mitochondrial activity not associated with mitochondrial disease.

In another aspect, embodiments disclosed herein are directed to methods for treating subjects in need thereof who have, or is suspected of having, a mitochondrial disease, optionally wherein the mitochondrial disease is selected from the group consisting of those listed in Table 1.

In another aspect, embodiments disclosed herein are directed to methods of treating cancer.

Compositions for Enhancing METTL17 Expression and/or Activity

In one aspect, embodiments disclosed herein are directed to compositions comprising enhancing expression of intra-mitochondrial protein translation and/or respiratory activity and/or oxidative phosphorylation activity comprising administering one or more agents effective to increase METTL17 gene expression and/or METTL17 methyltransferase-like 17 protein activity.

In certain example embodiments, a method of treating subjects who are at risk for, or are suffering from a mitochondrial disease or disorder comprises administering one or more agents that increase expression of METTL17, increases an enhancer positively regulating METTL17 expression, increases METTL17 protein activity, or increases METTL17 protein stability, all of which could enhance intra-mitochondrial respiratory chain function and lead to improved oxidative phosphorylation of cells and tissues.

The term “agent” refers to biologics including biological macromolecules (e.g., proteins, peptides, polypeptides, nucleic acids, polynucleotides, etc.) that increase METTL17 expression or activity. The term “agent” may also refer to “small molecules” preferably organic compounds, with a size comparable to those organic molecules generally used in pharmaceuticals. The term “small molecules” excludes biological macromolecules (e.g., proteins, peptides, nucleic acids, etc.). Preferred small organic molecules range in size up to about 5000 Da, e.g., up to about 4000 Da, preferably up to 3000 Da, more preferably up to 2000 Da, even more preferably up to about 1000 Da, e.g., up to about 900, 800, 700, 600 or up to about 500 Da. In example embodiments, the small molecule may act as an antagonist or agonist.

In certain example embodiments herein, the compositions for enhancing expression of intra-mitochondrial protein translation, respiratory chain activity, mitochondrial oxidative phosphorylation (OXPHOS), or any combination thereof, comprise (a) one or more agents effective to increase (i) methyltransferase like 17 (METTL17) gene expression, (ii) METTL17 protein expression and/or activity, or both (i) and (ii); (b) a polynucleotide encoding a METTL17 protein operably linked to one or more regulatory elements; (c) a recombinant METTL17 protein and/or a polynucleotide encoding the recombinant METTL17 protein; (d) a gene editing system configured to (i) insert an additional functional copy of a polynucleotide encoding METTL17; (ii) replace an existing or dysfunctional copy of DNA encoding METTL17, or (iii) modify an enhancer region of the METTL17 gene; (e) an engineered transcriptional activator system comprising a DNA-binding domain and a transcriptional activator configured to bind an enhancer of the METTL17 gene such that expression of METLL17 is increased; (f) an epigenetic modification protein comprising a DNA binding domain linked to, or otherwise engineered to associate with, a epigenetic modification domain; or any combination of (a)-(f).

In certain example embodiments, (b) is DNA incorporated into a vector, optionally a viral vector such as a lentiviral, adenovirus or adeno-associated (AAV) viral vector. In certain example embodiments, the vector is configured for stable integration of the DNA encoding METTL17 into a nuclear genome of target cells.

In certain example embodiments, (b) is an mRNA encoding METTL17. In certain example embodiments, the mRNA is contained in a delivery vehicle, optionally the delivery vehicle is a viral capsid, a retroelement capsid, an engineered viral like particle, or a nanoparticle, and optionally the nanoparticle is a lipid nanoparticle.

In certain example embodiments, the gene editing system comprises a Cas polypeptide, a guide molecule capable of forming a complex with the Cas polypeptide and directing binding of the Cas polypeptide to a target insertion site, and a homology directed repair (HDR) donor template comprising a donor sequence located between a first and second homology arm.

In certain example embodiments, the gene editing system is a CRISPR-associated transposase (CAST) system comprising: i) a catalytically inactive Cas polypeptide and a transposase fused to or otherwise capable of associating with the Cas polypeptide; ii) a guide molecule capable of forming a complex with the Cas polypeptide and directing the complex to a target insertion site; and iii) a donor construct comprising the polynucleotide encoding METTL17, or a functional component thereof, and one or more transposase recognition sequences capable of facilitating recognition by the transposase, whereby the transposase facilitates insertion of the polynucleotide encoding METTL17 at the target insertion site.

In certain example embodiments, the gene editing system is a prime editing system comprising: i) a Cas polypeptide having nickase activity and a reverse transcriptase linked to the Cas polypeptide; and ii) a prime editing guide RNA (pegRNA), wherein the prime editing guide RNA is capable of forming a complex with the Cas polypeptide and direct binding of the complex to a target insertion site and wherein the pegRNA further comprises a primer binding site configured to hybridize with a portion of a nicked strand of a target polynucleotide, such as nuclear genomic DNA, or the pegRNA further comprises a reverse transcriptase template comprising the polynucleotide encoding the METTL17 polypeptide.

In certain example embodiments, the transcriptional activator system comprises a catalytically inactive Cas polypeptide linked to a transcriptional activator, and a guide sequence capable of forming a complex with the Cas polypeptide and directing binding of the dead Cas (dCas)-linked transcriptional activator to a target region, such that the transcriptional activator can interact with a target enhancer region of METTL17.

In certain example embodiments, the DNA binding domain is a catalytically inactive Cas polypeptide, and the composition further comprises a guide molecule capable of forming a complex with the Cas polypeptide and directing binding of the complex and the epigenetic modification domain to a target region of the genome, such that the epigenetic modification domain modifies chromosomal architecture, such that METLL17 expression is increased. In certain example embodiments, the epigenetic modification domain is a demethylation domain that demethylates one or more CpG islands responsible for silencing expression of METTL17. In certain example embodiments, the gene editing system configured to modify an enhancer region of the METTL17 gene is a base editing system comprising: a catalytically inactive Cas polypeptide linked to a nucleobase deaminase; and a guide molecule capable of forming a complex with the Cas polypeptide and directing the base editing system to a target modification site to introduce one or more base edits in the enhancer region of the METTL17 gene such that METTL17 expression is increased.

In certain example embodiments, the gene editing system configured to modify an enhancer region of the METTL17 gene is a prime editing system comprising a Cas polypeptide having a nickase activity and linked to a reverse transcriptase and a pegRNA further comprising a primer binding site configured to hybridize with a portion of a nicked strand of a target polynucleotide, such as nuclear genomic DNA, or the pegRNA further comprises a reverse transcriptase template capable of introducing a single base edit, insertion or replacement of a region of the enhancer that increases METTL17 expression. In certain example embodiments, the gene editing system configured to modify an enhancer region of the METTL17 gene comprises a Cas polypeptide, a guide molecule capable of forming a complex with the Cas polypeptide and directing binding of the Cas polypeptide to an enhancer region of the METTL17 gene, and a HDR donor template comprising a donor sequence for insertion into the enhancer region such that METTL17 expression is increased.

In certain example embodiments, the gene editing system is a zinc finger nuclease, a TALEN system, or a meganuclease.

These and additional embodiments are further described below and elsewhere herein.

### Gene Therapy Approaches for Increasing METTL17 Expression

In one example embodiment, subjects at risk for, or suffering from a mitochondrial disease or disorder, are treated by increasing expression of METTL17 using a gene therapy approach. As used herein, the terms “gene therapy,” “gene delivery,” “gene transfer,” and “genetic modification” are used interchangeably and refer to modifying or manipulating the expression of a gene to alter the biological properties of living cells for therapeutic use.

In one example embodiment, a vector for use in gene therapy comprises a sequence encoding METTL17 or a functional fragment thereof, and is used to deliver said sequence to cells and tissues to increase expression of METTL17 in a variety of cell types. The vector may further comprise one or more regulatory elements to control expression of METTL17. The vector may further comprise regulatory/control elements, e.g., promoters, enhancers, introns, polyadenylation signals, Kozak consensus sequences, or internal ribosome entry sites (IRES). The vector may further comprise cellular localization signals, such as a nuclear localization signal (NLS) or nuclear export signal (NES). The vector may further comprise a targeting moiety that directs the vector specifically to any cells and tissues, e.g., cardiac, lung, liver, kidney, etc. In another example embodiment, the vector may comprise a viral vector with a trophism specific for cardiac, lung, liver, kidney.

### METTL17 Sequence

METTL17, also known as methyltransferase-like 17 protein, METT11D1, is located on the human 14q11.2 locus, Accession No. NC_000014.9 from 20989980 to 20997035. In one example embodiment, the polynucleotide sequence included in the vector is a DNA sequence derived from the primary accession numbers AK024512, AL355922 and BC005053. In another example embodiment, the DNA sequence is selected from the group consisting of AK024512, AL355922 and BC005053.

In another example embodiment, the polynucleotide sequence included in the vector is a RNA sequence derived from NM_022734.3 and NM_00102999.2. In another example embodiment, the polynucleotide sequence included in the vector is an RNA sequence selected from the group consisting of NM_022734.3 and NM_00102999.2. In another example embodiment, the sequence included in the vector is derived from mRNA selected from the group consisting of AF321002.1, AK02512.1, AK303484.1, AK304180.1, AK315999.1, BC005053.1, BG437086.1, KU178747.1, KU178748.1, KU178749.1, U5643.1. In another example embodiment, the sequence included in the vector is a mRNA sequence selected from the group consisting of AF321002.1, AK02512.1, AK303484.1, AK304180.1, AK315999.1, BC005053.1, BG437086.1, KU178747.1, KU178748.1, KU178749.1, U5643.1. In another example embodiment, the amino acid sequence is derived from the primary accession numbers Q9H7H0, NP_07357.1 and NP_001025162.1. In another example embodiment, the amino acid sequence is selected from the group consisting of Q9H7H0, NP_07357.1 and NP_001025162.1. In another example embodiment, the amino acid sequence is derived from the secondary accession numbers Q9BSH1, Q9BZH2, and Q9BZH3. In another example embodiment, the amino acid sequence is selected from the group consisting of Q9BSH1, Q9BZH2, and Q9BZH3.

All gene name symbols as used throughout the specification refer to the gene as commonly known in the art. The examples described herein that refer to gene names are to be understood to encompass human genes, as well as genes in any other organism (e.g., homologous, orthologous genes). The term, homolog, may apply to the relationship between genes separated by the event of speciation (e.g., ortholog). Orthologs are genes in different species that evolved from a common ancestral gene by speciation. Normally, orthologs retain the same function in the course of evolution. Gene symbols may be those referred to by the HUGO Gene Nomenclature Committee (HGNC) or National Center for Biotechnology Information (NCBI). Any reference to the gene symbol is a reference made to the entire gene or variants of the gene. Reference to a gene encompasses the gene product (e.g., protein encoded for by the gene).

### Regulatory Elements

Recombinant expression vectors can comprise a nucleic acid of the disclosure in a form suitable for expression of the nucleic acid in a host cell, which means that the recombinant expression vectors include one or more regulatory elements, which may be selected on the basis of the host cells to be used for expression, that is operably-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). The term “operably linked” as used herein also refers to the functional relationship and position of a promoter sequence relative to a polynucleotide of interest (e.g., a promoter or enhancer is operably linked to a coding sequence if it affects the transcription of that sequence). Typically, an operably linked promoter is contiguous with the sequence of interest. However, enhancers need not be contiguous with the sequence of interest to control its expression. The term “promoter,” as used herein, refers to a nucleic acid fragment that functions to control the transcription of one or more polynucleotides, located upstream of the polynucleotide sequence(s), and which is structurally identified by the presence of a binding site for DNA-dependent RNA polymerase, transcription initiation sites, and any other DNA sequences including, but not limited to, transcription factor binding sites, repressor, and activator protein binding sites, and any other sequences of nucleotides known in the art to act directly or indirectly to regulate the amount of transcription from the promoter. A “tissue-specific” promoter is only active in specific types of differentiated cells or tissues.

In another embodiment, the vector of the disclosure further comprises expression control sequences including, but not limited to, appropriate transcription sequences (i.e., initiation, termination, promoter, and enhancer), efficient RNA processing signals (e.g., splicing and polyadenylation (polyA) signals), sequences that stabilize cytoplasmic mRNA, sequences that enhance translation efficiency (i.e., Kozak consensus sequence), and sequences that enhance protein stability. A great number of expression control sequences, including promoters which are native, constitutive, inducible, or tissue-specific are known in the art and may be utilized according to the present disclosure.

In another embodiment, the vector of the disclosure further comprises a post-transcriptional regulatory region. In a preferred embodiment, the post-transcriptional regulatory region is the Woodchuck Hepatitis Virus post-transcriptional region (WPRE) or functional variants and fragments thereof and the PPT-CTS or functional variants and fragments thereof (see, e.g., Zufferey R, et al., J. Virol. 1999; 73:2886-2892; and Kappes J, et al., WO 2001/044481). In a particular embodiment, the post-transcriptional regulatory region is WPRE. The term “Woodchuck hepatitis virus posttranscriptional regulatory element” or “WPRE,” as used herein, refers to a DNA sequence that, when transcribed, creates a tertiary structure capable of enhancing the expression of a gene (see, e.g., Lee Y, et ah, Exp. Physiol. 2005; 90(1):33-37 and Donello J, et al, J. Virol. 1998; 72(6):5085-5092).

The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990).

Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in desired cells or tissues of interest, such as cardiac tissue or particular cell types (e.g., liver, kidney). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Also encompassed by the term “regulatory element” are enhancer elements (e.g., respiratory chain-specific enhancers or Woodchuck Hepatitis Virus Posttranscriptional Regulatory Element (WPRE)). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., METTL17).

### Viral Vector Selection

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. There are no limitations regarding the type of vector that can be used. The vector can be a cloning vector, suitable for propagation and for obtaining polynucleotides, gene constructs or expression vectors incorporated to several heterologous organisms. Suitable vectors include eukaryotic expression vectors based on viral vectors (e.g., adenoviruses, adeno-associated viruses as well as retroviruses and lentiviruses), as well as non-viral vectors such as plasmids.

In one example embodiment, the vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operably-linked. Such vectors are referred to herein as “expression vectors.” Vectors for and that result in expression in a eukaryotic cell can be referred to herein as “eukaryotic expression vectors.” In another example embodiment, the vector integrates the gene into the cell genome or is maintained episomally.

In one example embodiment, METTL17 is introduced into cells and tissues by means of an AAV viral vector. The terms “adeno-associated virus,” “AAV virion,” and “AAV particle,” as used interchangeably herein, refer to a virion composed of at least one AAV capsid protein (preferably all capsid proteins of a particular AAV serotype) and an encapsidated polynucleotide AAV genome. If the particle comprises a heterologous polynucleotide flanked by AAV inverted terminal repeats (i.e., a polynucleotide that is not a wild-type AAV genome, e.g., a transgene is delivered to a mammalian cell), it is often referred to as an “AAV vector particle” or “AAV vector.” AAV refers to a virus belonging to the genus dependovirus parvoviridae. The AAV genome is approximately 4.7 kilobases long and consists of single-stranded deoxyribonucleic acid (ssDNA), which can be in either the positive or negative orientation. The genome comprises Inverted Terminal Repeats (ITRs), and two Open Reading Frames (ORFs), at both ends of the DNA strand: rep and cap. The Rep framework is formed by four overlapping genes encoding the Rep proteins required for the AAV life cycle. The cap framework contains overlapping nucleotide sequences of the capsid proteins: VP1, VP2, and VP3, which interact together to form an icosahedral symmetric capsid (see, e.g., Carter B, Adeno-assisted viruses and ado-assisted viruses vectors for genetic drive, Lassic D, et al, eds., “Gene Therapy: Therapeutic Mechanisms and Strategies” (Marcel Dekker, Inc., New York, NY, US, 2000); and Gao G, et al, J.Virol. 2004; 78(12):6381-6388). The term “adeno-associated virus ITR” or “AAV ITR” as used herein refers to inverted terminal repeats present at both ends of the DNA strand of the genome of an adeno-associated virus. The ITR sequences are required for efficient proliferation of the AAV genome. Another characteristic of these sequences is their ability to form hairpins. This property contributes to its own priming, which allows synthesis of the second DNA strand independent of the priming enzyme. It has also been shown that ITRs are essential for integration and rescue of wild-type AAV DNA into the host cell genome (i.e., chromosome 19 of humans) and for efficient encapsidation of AAV DNA that binds to the resulting fully assembled, DNase-resistant AAV particles.

The term “AAV vector” as used herein further refers to a vector comprising one or more polynucleotides of interest (or transgenes) flanked by AAV terminal repeats (ITRs). Such AAV vectors can be replicated and packaged as infectious viral particles when present in a host cell that has been transfected with a vector that can encode and express Rep and Cap gene products (i.e., AAV Rep and Cap proteins), and wherein the host cell has been transfected with a vector that encodes and expresses proteins from adenovirus open reading frame E4orf 6. When an AAV vector is incorporated into a larger polynucleotide (e.g., a chromosome or another vector, such as a plasmid for cloning or transfection), then the AAV vector is typically referred to as a “protein-vector.” This protein-vector can be “rescued” by replication and encapsidation in the presence of AAV packaging functions and the necessary helper functions provided by E4orf 6.

In one example embodiment, gene therapy uses an adeno-associated viral (AAV) vector comprising a recombinant viral genome wherein said recombinant viral genome comprises an expression cassette comprising either a general or tissue-specific transcriptional regulatory region operably linked to a polynucleotide encoding for METTL17 (AAV vectors can also be used for any compositions described herein, such as a programmable nuclease). AAV according to the present disclosure can include any serotype of the 42 serotypes of AAV known.

In particular, the AAV of the present disclosure may belong to the serotype AAV1, AAV2, AAV3 (including types 3A and 3B), AAV4, AAV5, AAV6, AAV7, AAV8, AAV9, AAV10, AAV 11 and any other AAV. In a preferred embodiment, the adeno-associated viral vector of the disclosure is of a serotype selected from the group consisting of the AAV6, AAV7, AAV8, and AAV9 serotypes. In more preferred embodiments, the adeno-associated viral vector of the disclosure is an AAV8 serotype. In more preferred embodiments, the adeno-associated viral vector of the disclosure is the engineered hybrid serotype Rec2 (see, e.g., Charbel Issa, et al., 2013, Assessment of tropism and effectiveness of new primate-derived hybrid recombinant AAV serotypes in the mouse and primate retina PLoS ONE, 8 (2013), p. e60361). In one example embodiment, Rec2 can be used for oral administration, as oral administration of Rec2 results in preferential transduction of BAT with absence of transduction in the gastrointestinal track.

The genome of the AAV according to the disclosure typically comprises the cis-acting 5′ and 3′ inverted terminal repeat sequences and an expression cassette (see, e.g., Tijsser P, Ed., “Handbook of Parvoviruses” (CRC Press, Boca Raton, FL, US, 1990, pp. 155-168)).

The polynucleotide of the disclosure can comprise ITRs derived from any one of the AAV serotypes. In a preferred embodiment, the ITRs are derived from the AAV2 serotype. The AAV of the disclosure comprises a capsid from any serotype. In particular embodiment, the capsid is derived from the AAV of the group consisting of AAV1, AAV2, AAV4, AAV5, AAV6, AAV7, AAV8 and AAV9. In a preferred embodiment, the AAV of the disclosure comprises a capsid derived from the AAV8 or AAV9 serotypes.

In another particular embodiment, the AAV vector is a pseudotyped AAV vector (i.e., the vector comprises sequences or components originating from at least two distinct AAV serotypes). In a particular embodiment, the pseudotyped AAV vector comprises an AAV genome derived from one AAV serotype (e.g., AAV2), and a capsid derived at least in part from a distinct AAV serotype. In a preferred embodiment, the adeno-associated viral vector used in the method for transducing cells in vitro or in vivo has a serotype selected from the group consisting of AAV6, AAV7, AAV8, and AAV9, and the adeno-associated virus ITRs are AAV2 ITRs.

In one example embodiment, adeno-associated viral vectors of the AAV6, AAV7, AAV8, and AAV9 serotypes are capable of transducing any tissue cells efficiently. This feature makes possible the development of methods for the treatment of diseases which require or may benefit from the expression of a polynucleotide of interest in specific tissues (e.g., METTL17). In particular, this finding facilitates the delivery of polypeptides of interest to a subject in need thereof by administering the AAV vectors of the disclosure to the patient, thus generating cells capable of expressing the polynucleotide of interest and its encoded polypeptide in vivo (e.g., METTL17).

In one embodiment the AAV vector contains one promoter with the addition of at least one target sequence of at least one miRNA.

In one example embodiment, METTL17 is introduced to cells by means of a lentiviral viral vector. Lentiviruses are enveloped, single stranded RNA viruses that belong to the family of Retroviridae. Moreover, lentiviral vectors are preferred as they are able to transduce or infect non-dividing cells and typically produce high viral titers.

In one example embodiment, the vector is a “plasmid,” which refers to a circular double stranded DNA loop into which additional DNA segments can be inserted, such as by standard molecular cloning techniques.

In one example embodiment, the vector is an mRNA vector (see, e.g., Sahin, U, Kariko, K and Tureci, O (2014). mRNA-based therapeutics—developing a new class of drugs. Nat Rev Drug Discov 13: 759-780; Weissman D, Karikó K. mRNA: Fulfilling the Promise of Gene Therapy. Mol Ther. 2015; 23(9):1416-1417. doi:10.1038/mt.2015.138; Kowalski P S, Rudra A, Miao L, Anderson D G. Delivering the Messenger: Advances in Technologies for Therapeutic mRNA Delivery. Mol Ther. 2019; 27(4):710-728. doi:10.1016/j.ymthe.2019.02.012; Magadum A, Kaur K, Zangi L. mRNA-Based Protein Replacement Therapy for the Heart. Mol Ther. 2019; 27(4):785-793. doi:10.1016/j.ymthe.2018.11.018; Reichmuth A M, Oberli M A, Jaklenec A, Langer R, Blankschtein D. mRNA vaccine delivery using lipid nanoparticles Ther Deliv. 2016; 7(5):319-334. doi:10.4155/tde-2016-0006; and Khalil A S, Yu X, Umhoefer J M, et al. Single-dose mRNA therapy via biomaterial-mediated sequestration of overexpressed proteins. Sci Adv. 2020; 6(27):eaba2422). In an exemplary embodiment, mRNA encoding for METTL17 is delivered using lipid nanoparticles (see, e.g., Reichmuth, et al., 2016) and administered directly into tissues. In an exemplary embodiment, mRNA encoding for METTL17 is delivered using biomaterial-mediated sequestration (see, e.g., Khalil, et al., 2020) and administered directly into tissues. Sequences present in mRNA molecules, as described further herein, are applicable to mRNA vectors (e.g., Kozak consensus sequence, miRNA target sites and WPRE).

In one example embodiment, the non-viral vector for use in gene transfer and/or nanoparticle formulations is a lipid. In one example embodiment the non-viral lipid vector may comprise: 1,2-Dioleoyl-sn-glycero-3-phosphatidylcholine; 1,2-Dioleoyl-sn-glycero-3-phosphatidylethanolamine; Cholesterol; N-[1-(2,3-Dioleyloxy)propyl]N,N,N-trimethylammonium chloride; 1,2-Dioleoyloxy-3-trimethylammonium-propane; Dioctadecylamidoglycylspermine; N-(3-Aminopropyl)-N,N-dimethyl-2,3-bis(dodecyloxy)-1-propanaminium bromide; Cetyltrimethylammonium bromide; 6-Lauroxyhexyl ornithinate; 1-(2,3-Dioleoyloxypropyl)-2,4,6-trimethylpyridinium; 2,3-Dioleyloxy-N-[2(sperminecarboxamido-ethyl]-N,N-dimethyl-1-propanaminium trifluoroacetate; 1,2-Dioleyl-3-trimethylammonium-propane; N-(2-Hydroxyethyl)-N,N-dimethyl-2,3-bis(tetradecyloxy)-1-propanaminium bromide; Dimyristooxypropyl dimethyl hydroxyethyl ammonium bromide; 3β-[N—(N′,N′-Dimethylaminoethane)-carbamoyl]cholesterol; Bis-guanidium-tren-cholesterol; 1,3-Diodeoxy-2-(6-carboxy-spermyl)-propylamide; Dimethyloctadecylammonium bromide; Dioctadecylamidoglicylspermidin; rac-[(2,3-Dioctadecyloxypropyl)(2-hydroxyethyl)]-dimethylammonium chloride; rac-[2(2,3-Dihexadecyloxypropyl-oxymethyloxy)ethyl]trimethylammonium bromide; Ethyldimyristoylphosphatidylcholine; 1,2-Distearyloxy-N,N-dimethyl-3-aminopropane; 1,2-Dimyristoyl-trimethylammonium propane; 0,0′-Dimyristyl-N-lysyl aspartate; 1,2-Distearoyl-sn-glycero-3-ethylphosphocholine; N-Palmitoyl D-erythro-sphingosyl carbamoyl-spermine; N-t-Butyl-N′-tetradecyl-3-tetradecylaminopropionamidine; Octadecenolyoxy[ethyl-2-heptadecenyl-3 hydroxyethyl]imidazolinium chloride; N1-Cholesteryloxycarbonyl-3,7-diazanonane-1,9-diamine; 2-(3-[Bis(3-amino-propyl)-amino]propylamino)-N-ditetradecylcarbamoylme-ethyl-acetamide; 1,2-dilinoleyloxy-3-dimethylaminopropane; 2,2-dilinoleyl-4-dimethylaminoethyl-[1,3]-dioxolane; and dilinoleyl-methyl-4-dimethylaminobutyrate.

In one example embodiment, the non-viral vector for use in gene transfer and/or nanoparticle formulations is a polymer. In one example embodiment the non-viral polymer vector may comprise: Poly(ethylene)glycol; Polyethylenimine; Dithiobis(succinimidylpropionate); Dimethyl-3,3′-dithiobispropionimidate; Poly(ethylene imine) biscarbamate; Poly(L-lysine); Histidine modified PLL; Poly(N-vinylpyrrolidone); Poly(propylenimine); Poly(amidoamine); Poly(amido ethylenimine); Triethylenetetramine; Poly(β-aminoester); Poly(4-hydroxy-L-proline ester); Poly(allylamine); Poly(α-[4-aminobutyl]-L-glycolic acid); Poly(D,L-lactic-co-glycolic acid); Poly(N-ethyl-4-vinylpyridinium bromide); Poly(phosphazene)s; Poly(phosphoester)s; Poly(phosphoramidate)s; Poly(N-2-hydroxypropylmethacrylamide); Poly (2-(dimethylamino)ethyl methacrylate); Poly(2-aminoethyl propylene phosphate); Chitosan; Galactosylated chitosan; N-Dodacylated chitosan; Histone; Collagen; and Dextran-spermine.

### Recombinant METTL17

In another example embodiment, a method for treating subjects at risk for, or suffering from, a mitochondrial disease comprises administering a METTL17 recombinant polypeptide. In certain embodiments, recombinant METTL17 protein is delivered intracellularly to a subject in need thereof and is used as a protein therapeutic. Protein therapeutics offer high specificity, and the ability to treat “undruggable” targets, in diseases associated with protein deficiencies or mutations (e.g., METTL17). As used herein METTL17 protein includes all variants and protein fragments, described further herein. Applicants have identified a factor, METTL17, that appears to be sufficient for boosting mitochondrial respiratory chain activity and can rescue some cellular models of mitochondrial disease. Thus, while not being bound by a particular scientific theory, it is expected that administration of additional copies of functional METTL17 protein may restore normal oxidative phosphorylation activity in cells. As described elsewhere herein, a polypeptide encoding the recombinant METTL17 protein can also be delivered to provide a recombinant METTL17 protein.

METTL17 has the following domains or regions (e.g., NP_073571; 456 amino acids): Transit peptide (from amino acid 1-19), AdoMet_methylatransferase (AdoMet_MTase; from amino acid 155-438), and SAM-dependent methyltransferase (SmtA; from amino acid 191-297). In certain embodiments, full length METTL17 protein is administered. In one example embodiment, a METTL17 sequence selected from Table 1 is administered. In certain embodiments, a truncated METTL17 protein is administered. For example, any domains that function only in the nucleus are not required for the recombinant protein. Various methods can be used for delivery of METTL17 to cells and tissues. In certain embodiments, METTL17 is delivered in a composition capable of delivering METTL17 intracellularly.

TABLE 1
Exemplary METTL17 polypeptides

| Locus | Sequence |
|---|---|
| NP_073571 | MAAALKCLLT LGRWCPGLGV APQARALAAL VPGVTQVDNK |
| (Human) | SGFLQKRPHR QHPGILKLPH VRLPQALANG AQLLLLGSAG |
| | PTMENQVQTL TSYLWSRHLPVEPEELQRRA RHLEKKFLEN |
| | PDLSQTEEKL RGAVLHALRK TTYHWQELSY TEGLSLVYMA |
| | ARLDGGFAAV SRAFHEIRAR NPAFQPQTLM DFGSGTGSVT |
| | WAAHSIWGQS LREYMCVDRS AAMLVLAEKL LKGGSESGEP |
| | YIPGVFFRQF LPVSPKVQFD VVVSAFSLSE LPSKADRTEV |
| | VQTLWRKTGH FLVLVENGTK AGHSLLMDAR DLVLKGKEKS |
| | PLDPRPGFVF APCPHELPCP QLTNLACSFS QAYHPIPFSW |
| | NKKPKEEKFS MVILARGSPE EAHRWPRITQ PVLKRPRHVH |
| | CHLCCPDGHM QHAVLTARRHGRDLYRCARV SSWGDLLPVL |
| | TPSAFPPSTA QDPSES (SEQ ID NO: 19) |
| NP_001025162 | MAAALKCLLT LGRWCPGLGV APQARALAALVPGVTQVDNK |
| (Human) | SGFLQKRPHR QHPGILKLPH VRLPQALANGAQLLLLGSAG |
| | PTMENQVQTL TSYLWSRHLPVEPEELQRRARHLEKKFLEN |
| | PDLSQTEEKL RGAVLHALRKTTYHWQELSYTEGLSLVYMA |
| | ARLDGGFAAV SRAFHEIRAR NPAFQPQTLM DFGSGTGSVT |
| | WAAHSIWGQS LREYMCVDRS AAMLVLAEKLLKGGSESGEP |
| | YIPGVFFRQF LPVSPKVQFD VVVSAFSLSE LPSKADRTEV |
| | VQTLWRKTGHFLVLVENGTKAGHSLLMDAR DLVLKGKEKS |
| | PLDPRPGFVFAPCPHELPCP QLTNLACSFS QAYHPIPFSW NKKPKEEKFS |
| | MVILARGSPE EAHRWPRITQ PVLKRPRHVH CHLCCPDGHM |
| | QHAVLTARRH GRYGGCDQNQ WDVAGSCSPR QHLFPQGFVS |
| | LCPCQLLGRS FTCAYSVCVS SIYGSGSL (SEQ ID NO: 20) |
| XP_006720299 | MAARLDGGFA AVSRAFHEIR ARNPAFQPQT LMDFGSGTGS |
| (Human) | VTWAAHSIWGQSLREYMCVD RSAAMLVLAE KLLKGGSESGEPYIPGVFFR |
| | QFLPVSPKVQ FDVVVSAFSL SELPSKADRT EVVQTLWRKT GHFLVLVENG |
| | TKAGHSLLMDARDLVLKGKE KSPLDPRPGF VFAPCPHELPCPQLTNLACS |
| | FSQAYHPIPF SWNKKPKEEK FSMVILARGS PEEAHRWPRI TQPVLKRPRH |
| | VHCHLCCPDG HMQHAVLTAR RHGRYGGCDQNQWDVAGSCS |
| | PRQHLFPQGF VSLCPCQLLG RSFTCAYSVCVSSIYGSGSL (SEQ ID NO: 21) |
| XP_016877090 | MAARLDGGFA AVSRAFHEIR ARNPAFQPQT LMDFGSGTGS |
| (Human) | VTWAAHSIWG QSLREYMCVD RSAAMLVLAE KLLKGGSESG |
| | EPYIPGVFFR QFLPVSPKVQ FDVVVSAFSL SELPSKADRT EVVQTLWRKT |
| | GHFLVLVENG TKAGHSLLMD ARDLVLKGKE KSPLDPRPGF VFAPCPHELP |
| | CPQLTNLACS FSQAYHPIPF SWNKKPKEEK FSMVILARGS PEEAHRWPRI |
| | TQPVLKRPRH VHCHLCCPDG HMQHAVLTAR RHGRDLYRCA |
| | RVSSWGDLLP VLTPSAFPPS TAQDPSES (SEQ ID NO: 22) |
| NP_001025161 | MATARTPRFL LLFCRSCRSL RGPQSRAYAA LVPGVSQVDN |
| (Mouse) | GSDFLGKKPH RKHPGILHLP HVQLPQALAD AAQLMLFERP |
| | MRSVEKQVQA LTNYLWSRHL PVEPEELQRR AAYLEKKFLE |
| | KQDSAPTEEK LREAVLHALR KTTYHWQELS YSEELSLIYM |
| | AARLDGGFAA VFRAFHEIQA RIPEFRPQTL MDFGSGTGSV |
| | AWAAHRTWGQ SLREYVCVDS SAAMLGLAEK LLKGGSESGK |
| | PCIPGVFFRQ FLPVSPKVQF DVVVSAYALS ELPSRADRIE |
| | VIQNLWRKTS HFLVLVENGT KAGHRLLMDA RNLVLGEKEK |
| | SPLDLRPSFV FAPCPHELPC PQLNASKSLA CSFSQAYHPI |
| | PFNWNKKPKE EIFSMVILAR GSPKEANRWP RITQPVLKRP |
| | RHVHCHLCCP DGHMQHAVVT ARRHGRDLYR CARVSSWGDL |
| | LPVIAPSEFP PSSPDEEPPE N (SEQ ID NO: 23) |
| XP_038949755 | MATARASRFL LLFCRSCRSL RGPQSRAFAA LVPGVSQVDN |
| (Rat) | RSDFLGKKPH RKHPGILHLP HVHLPQALTD AAQLLLLERP |
| | KRSVEKQVQA LTNYLWSRHL PVEPEELQRR AAYLEKKFLE |
| | KQDSTQTEEK LREAVLHALR KTTYHWQELS YNEELSLIYM |
| | AARLDGGFAA VFRAFHEIQA RIPEFQPQTL MDFGSGTGSV |
| | ACTWGQSLRE YVCVDRSAAM LGLAEKLLKG GSESGKPCIP |
| | GVFFRQFLPVSPKVQFDVVV SAFALSELPS RADRTEVLQN |
| | LWRKTSHFLVLVENGTKAGHRLLMDARNLI LGDKEKSPLD |
| | LRPSFVFAPCPHELPCPQLN AAKSLACSFS QAYQPIPFHW |
| | NKKPKEETFS MVILARGSPK EANRWPRITQ PVLKRPRHVH |
| | CHLCCPDGHMQHAVVTARRH GRDLYRCARV SSWGDLLPVI |
| | SPSEFPPPSPAEEPPES (SEQ ID NO: 24) |

mRNA-Based Therapeutics

In vitro transcribed (IVT) mRNA has recently come into focus as a potential new drug class to deliver genetic information. This synthetic mRNA can be engineered to transiently express proteins by structurally resembling natural mRNA. Advances in addressing the inherent challenges of this drug class, particularly related to controlling the translational efficacy and immunogenicity of the IVT-mRNA, provide the basis for a broad range of potential applications. mRNA-based cancer immunotherapies and infectious disease vaccines have entered clinical development or are currently commercially available in response to the SARS-Cov2 pandemic (e.g., Pfizer, Moderna). Meanwhile, emerging novel approaches include in vivo delivery of IVT-mRNA to replace or supplement proteins, IVT mRNA-based generation of pluripotent stem cells and genome engineering using IVT mRNA-encoded designer nucleases.

mRNA Polynucleotide Modifications

In some embodiments, the cargo polynucleotides include one or more modifications capable of modifying the e.g., functionality, packaging ability, stability, degradation localization, increase expression lifetime, resistance to degradation, or any combination thereof, of the at least one or more cargo polynucleotides. Modifications can be sequence modifications (e.g., mutations), chemical modifications, or other modifications, such as complexing to a lipid, polymer, etc. In some embodiments, the cargo polynucleotide is modified to protect it against degradation, by e.g., nucleases or otherwise prevent its degradation.

In some embodiments, one or more polynucleotides in the engineered polynucleotide are modified. In some embodiments, the engineered polynucleotide includes one or more non-naturally occurring nucleotides, which can be the result of modifying a naturally occurring nucleotide. In some embodiments, the modification is selected independently for each polynucleotide modified. In some embodiments, the modification(s) increase or decrease the stability of the polynucleotide, reduce the immunogenicity of the polynucleotide, increase or decrease the rate of transcription and/or translation, or any combination thereof. Non-naturally occurring nucleotides and/or nucleotide analogs may be modified at the ribose, phosphate, and/or base moiety.

Suitable modifications include, without limitation, methylpseudouridine, a phosphorothioate linkage, locked nucleic acid (LNA) nucleotides comprising a methylene bridge between the 2′ and 4′ carbons of the ribose ring, or bridged nucleic acids (BNA), 2′-O-methyl analogs, 2′-deoxy analogs, or 2′-fluoro analogs, 2-aminopurine, 5-bromo-uridine, pseudouridine, (Ψ), N1-methylpseudouridine (melΨ), 5-methoxyuridine(5moU), inosine, 7-methylguanosine, inosine, or 7-methylguanosine. Examples of RNA, including but not limited to guide RNA, chemical modifications include, without limitation, incorporation of 2′-O-methyl (M), 2′-O-methyl 3′-phosphorothioate (MS), S-constrained ethyl (cEt), or 2′-O-methyl 3′-thioPACE (MSP) at one or more terminal nucleotides.

In some embodiments, the polynucleotide (DNA and/or RNA) is modified with a 5′- and/or 3′-cap structure. In some embodiments, the 5′ cap structure is cap0, cap1, ARCA, inosine, N1-methyl-guanosine, 2′-fluoro-guanosine, 7-deaza-guanosine, 8-oxo-guanosine, 2-amino-guanosine, LNA-guanosine, or 2-azido-guanosine. In some embodiments, the 5′ terminal cap is 7mG(5′)ppp(5′)NlmpNp, m7GpppG cap, N7-methylguanine. In some embodiments, the 3′ terminal cap is a 3′-O-methyl-m7GpppG, 2′-fluoro bases, inverted dT and dTTs, phosphorylation of the 3′ end nucleotide, a C3 spacer. Exemplary 5′- and/or 3′ that protect against degradation are described in e.g., Gagliardi and Dziembowski. Philosophical transactions of the Royal Society B. 2018. 313(1762). doi.org/10.1098/rstb.2018.0160; Boo and Kim. Experimental & Molecular Medicine volume 52, pages 400-408 (2020); and Adachai et al., 2021. Biomedicines 2021, 9, 550. doi.org/10.3390/biomedicines9050550.

In some embodiments, the 5′-UTR comprises a Kozak sequence.

In some embodiments, the polynucleotide can be modified with a tailing sequence that may range from absent to 500 nucleotides in length (e.g., at least 60, 70, 80, 90, 120, 140, 160, 180, 200, 250, 300, 350, 400, 450, or 500 nucleotides). In some embodiments, the tailing region is or includes a polyA tail. Where the tailing region is a polyA tail, the length may be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional. In some embodiments, the poly-A tail is at least 160 nucleotides in length.

In some embodiments, about 10%, 15%, 20%, 24%, 30%, 35%, 40%, 45%, 50%, 55%, 60%, 65%, 70%, 75%, 80%, 85%, 90%, 95%, to/or about 100% of the uracils of a polynucleotide of the present disclosure have a chemical modification, In some embodiments, about 10%, 15%, 20%, 25%, 30%, 35%, 40%, 45%, 50%, 55%, 60%, 65%, 70%, 75%, 80%, 85%, 90%, 95%, to/or about 100% of the uracils of a polynucleotide of the present disclosure have a N1-methyl pseudouridine in the 5′-position of the uracil.

In some embodiments, the polynucleotide, optionally an RNA (e.g., an mRNA) includes a stabilization element. In some embodiments, the stabilization element is a histone stem-loop. In some embodiments, the stabilization element is a nucleic acid sequence having increased GC content relative to wild type sequence.

In some embodiments, a polynucleotide of the present disclosure includes a sequence encoding a self-cleaving peptide. The self-cleaving peptide may be, but is not limited to, a 2A peptide. In one embodiment, this sequence may be used to separate the coding regions of two or more polypeptides.

In some embodiments, the polynucleotides (e.g., mRNAs) are linear. In yet another embodiment, the polynucleotides of the present disclosure that are circular are known as “circular polynucleotides” or “circP.” As used herein, “circular polynucleotides” or “circP” means a single stranded circular polynucleotide which acts substantially like, and has the properties of, an RNA. The term “circular” is also meant to encompass any secondary or tertiary configuration of the circP.

Other RNA modifications, such as mRNA modifications, that can be incorporated into a polynucleotide of the present disclosure include, but are not limited to, any one or more of those described e.g., U.S. Pat. Nos. 8,278,036, 8,691,966, 8,748,089, 9,750,824, 10,232,055, 10,703,789, 10,702,600, 10,577,403, 10,442,756, 10,266,485, 10,064,959, 9,868,692, 10,064,959, 10,272,150; U.S. Patent Application Publication Nos US 2013/0197068, US 2017/0043037, US 2013/0261172, US 2020/0030460, US 2015/0038558, US 2019/0274968, US 2018/0303925, US 2020/0276300; and International Patent Application Publication Nos. WO 2018/081638A1, WO 2016/176330A1, which are incorporated herein by reference and can be adapted for use with the present disclosure.

### Programmable Nucleases

In certain example embodiments, a programmable nuclease may be used to edit a genomic region comprising one or more genomic variants associated with decreased expression or activity of METTL17 in cells and tissues. In certain example embodiments, a programmable nuclease may be used to edit a genomic region comprising one or more genomic variants associated with a mitochondrial disease (Tables 3-7). In example embodiments, a programmable nuclease may be used to edit a genomic region comprising one or more genomic variants associated with decreased expression or activity of METTL17. Gene editing using programmable nucleases may utilize two different cell repair pathways, non-homologous end joining (NHEJ) and homology directed repair (HDR). In certain example embodiment, HDR is used to provide a template that replaces a genomic region comprising the variant with a donor that edits the risk variant to a wild-type or non-risk variant. Example programmable nucleases for use in this manner include zinc finger nucleases (ZFN), TALE nucleases (TALENS), meganucleases, CRISPR-Cas systems, and OMEGA systems.

### CRISPR-Cas

In one example embodiment, the gene editing system is a CRISPR-Cas system. The CRISPR-Cas systems comprise a Cas polypeptide and a guide sequence, wherein the guide sequence is capable of forming a CRISPR-Cas complex with the Cas polypeptide and directing site-specific binding of the CRISPR-Cas sequence to a target sequence. The Cas polypeptide may induce a double- or single-stranded break at a designated site in the target sequence. The site of CRISPR-Cas cleavage, for most CRISPR-Cas systems, is dictated by distance from a protospacer-adjacent motif (PAM), discussed in further detail below. Accordingly, a guide sequence may be selected to direct the CRISPR-Cas system to induce cleavage at a desired target site at or near the one or more variants.

### HDR Template Based Editing

In one example embodiment, a donor template is provided to replace a genomic sequence comprising one or more variants that increase METTL17 expression. A donor template may comprise an insertion sequence flanked by two homology regions. The insertion sequence comprises an edited sequence to be inserted in place of the target sequence (e.g., a portion of genomic DNA comprising the one or more variants). The homology regions comprise sequences that are homologous to the genomic DNA strands at the site of the CRISPR-Cas induced double-strand break (DSB). Cellular HDR mechanisms then facilitate insertion of the insertion sequence at the site of the DSB.

Accordingly, in certain example embodiments, a donor template and guide sequence are selected to direct excision and replacement of a section of genomic DNA comprising a variant that increases binding to an enhancer controlling METTL17 expression with an insertion sequence that edits the one or more variants to a wild-type or non-risk variant. In one example embodiment, the insertion sequence comprises a wild-type or non-risk variant that restores or increases expression of binding to the enhancer. In one example embodiment, the insertion sequence encodes a portion of genomic DNA in which the rs6712203 variant is changed from a C to a T.

The donor template may include a sequence which results in a change in sequence of 1, 2, 3, 4, 5, 6, 7, 8, 9, 10, 11, 12 or more nucleotides of the target sequence.

A donor template may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, or 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, or 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

The homology regions of the donor template may be complementary to a portion of a polynucleotide comprising the target sequence. When optimally aligned, a donor template might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The donor template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a micrωRNA). Thus, the sequence for integration may be operably linked to an appropriate control sequence or sequences. Alternatively, the sequence to be integrated may provide a regulatory function.

Homology arms of the donor template may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000 bp.

In one example embodiment, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

The donor template may further comprise a marker. Such a marker may make it easy to screen for targeted integrations. Examples of suitable markers include restriction sites, fluorescent proteins, or selectable markers. The donor template of the disclosure can be constructed using recombinant techniques (see, for example, Sambrook et al., 2001 and Ausubel et al., 1996).

In one example embodiment, a donor template is a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length. Suzuki et al. describe in vivo genome editing via CRISPR/Cas9 mediated homology-independent targeted integration (2016, Nature 540:144-149).

### Class 1 Systems

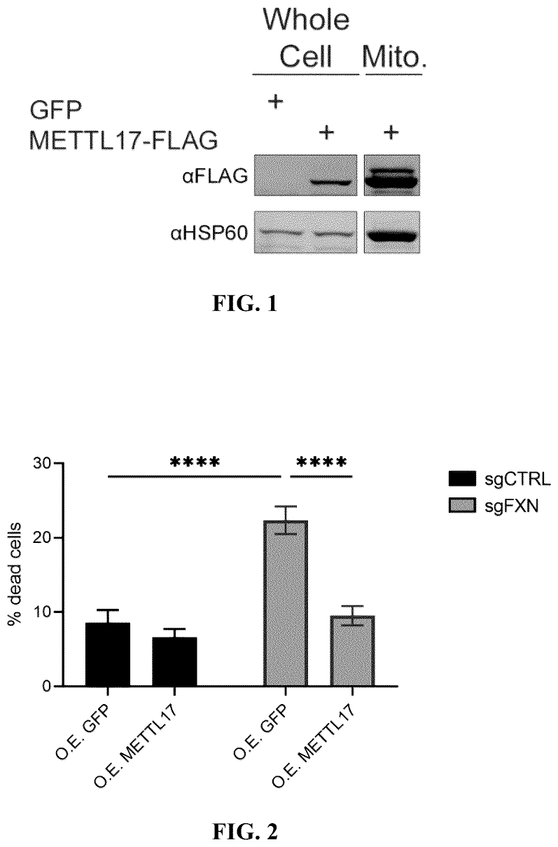

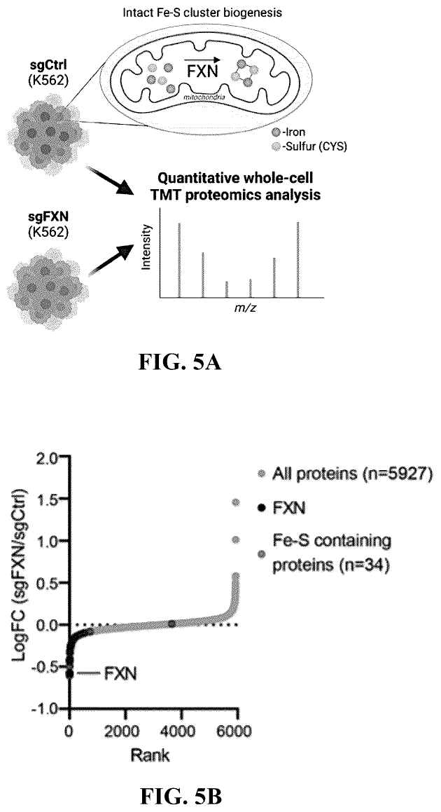

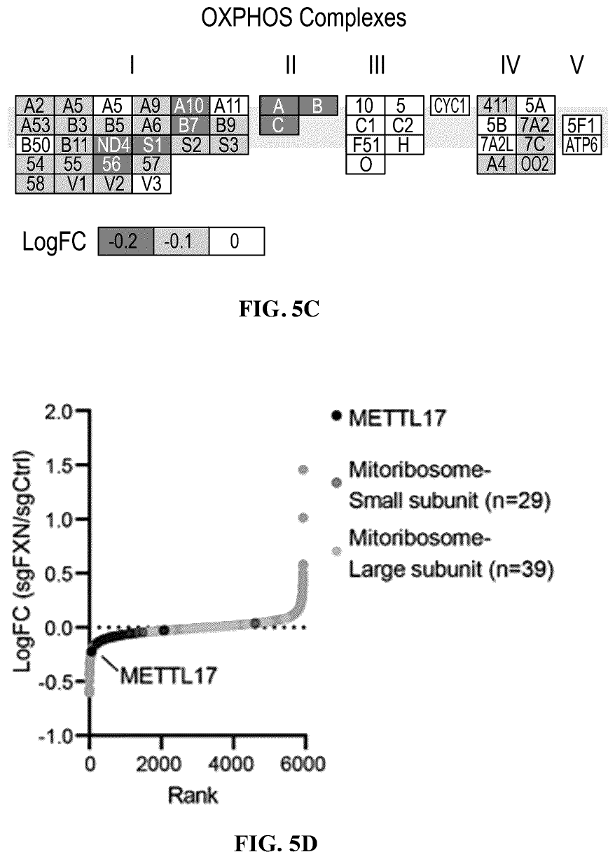

The CRISPR-Cas therapeutic methods disclosed herein may be designed for use with Class 1 CRISPR-Cas systems. In certain example embodiments, the Class 1 system may be Type I, Type III or Type IV CRISPR-Cas as described in Makarova et al. “Evolutionary classification of CRISPR-Cas systems: a burst of class 2 and derived variants” Nature Reviews Microbiology, 18:67-81 (February 2020), incorporated in its entirety herein by reference, and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g. Cas1, Cas2, RNA nuclease), and/or one or more accessory proteins (e.g., Cas 4, DNA nuclease), CRISPR-associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g., Cas 5, Cas6, Cas7. RAMP proteins are characterized by having one or more RNA recognition motif domains. Large subunits (for example cas8 or cas10) and small subunits (for example, cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374: 20180087, DOI: 10.1098/rstb.2018.0087. In one aspect, Class 1 systems are characterized by the signature protein Cas3. The Cascade in particular Class1 proteins can comprise a dedicated complex of multiple Cas proteins that binds pre-crRNA and recruits an additional Cas protein, for example Cas6 or Cas5, which is the nuclease directly responsible for processing pre-crRNA. In one aspect, the Type I CRISPR protein comprises an effector complex comprising one or more Cas5 subunits and two or more Cas7 subunits. Class 1 subtypes include Type I-A, I-B, I-C, I-U, I-D, I-E, and I-F, Type IV-A and IV-B, and Type III-A, III-D, III-C, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1, n5, FIG. 5.

### Class 2 Systems

The CRISPR-Cas therapeutic methods disclosed herein may be designed for use with Class 2 CRISPR-Cas systems. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside a split Ruv-C like nuclease domain sequence. The Type V systems (e.g., Cas12) only contain a RuvC-like nuclease domain that cleaves both strands. Some Type V systems have also been found to possess this collateral activity with two single-stranded DNA in in vitro contexts.

In one example embodiment, the Class 2 system is a Type II system. In one example embodiment, the Type II CRISPR-Cas system is a II-A CRISPR-Cas system. In one example embodiment, the Type II CRISPR-Cas system is a II-B CRISPR-Cas system. In one example embodiment, the Type II CRISPR-Cas system is a II-C1 CRISPR-Cas system. In one example embodiment, the Type II CRISPR-Cas system is a II-C2 CRISPR-Cas system. In some example embodiments, the Type II system is a Cas9 system. In some embodiments, the Type II system includes a Cas9.

In one example embodiment, the Class 2 system is a Type V system. In one example embodiment, the Type V CRISPR-Cas system is a V-A CRISPR-Cas system. In one example embodiment, the Type V CRISPR-Cas system is a V-B1 CRISPR-Cas system. In one example embodiment, the Type V CRISPR-Cas system is a V-B2 CRISPR-Cas system. In one example embodiment, the Type V CRISPR-Cas system is a V-C CRISPR-Cas system. In one example embodiment, the Type V CRISPR-Cas system is a V-D CRISPR-Cas system. In one example embodiment, the Type V CRISPR-Cas system is a V-E CRISPR-Cas system. In one example embodiment, the Type V CRISPR-Cas system is a V-F1 CRISPR-Cas system. In one example embodiment, the Type V CRISPR-Cas system is a V-F1 (V-U3) CRISPR-Cas system. In one example embodiment, the Type V CRISPR-Cas system is a V-F2 CRISPR-Cas system. In one example embodiment, the Type V CRISPR-Cas system is a V-F3 CRISPR-Cas system. In one example embodiment, the Type V CRISPR-Cas system is a V-G CRISPR-Cas system. In one example embodiment, the Type V CRISPR-Cas system is a V-H CRISPR-Cas system. In one example embodiment, the Type V CRISPR-Cas system is a V-I CRISPR-Cas system. In one example embodiment, the Type V CRISPR-Cas system is a V-K (V-U5) CRISPR-Cas system. In one example embodiment, the Type V CRISPR-Cas system is a V-U1 CRISPR-Cas system. In one example embodiment, the Type V CRISPR-Cas system is a V-U2 CRISPR-Cas system. In one example embodiment, the Type V CRISPR-Cas system is a V-U4 CRISPR-Cas system. In one example embodiment, the Type V CRISPR-Cas is a Cas12a (Cpf1), Cas12b (C2cl), Cas12c (C2c3), Cas12d (CasY), Cas12e (CasX), Cas14, and/or CasΦ.

### Guide Molecules

The following include general design principles that may be applied to the guide molecule. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In one example embodiment, a guide RNA or crRNA may comprise, consist essentially of, or consist of a direct repeat (DR) sequence and a guide sequence or spacer sequence. In another example embodiment, the guide RNA or crRNA may comprise, consist essentially of, or consist of a direct repeat sequence fused or linked to a guide sequence or spacer sequence. In another example embodiment, the direct repeat sequence may be located upstream (i.e., 5′) from the guide sequence or spacer sequence. In other embodiments, the direct repeat sequence may be located downstream (i.e., 3′) from the guide sequence or spacer sequence.

In one example embodiment, the crRNA comprises a stem loop, preferably a single stem loop. In one example embodiment, the direct repeat sequence forms a stem loop, preferably a single stem loop.

In one example embodiment, the spacer length of the guide RNA is from 15 to 35 nt. In another example embodiment, the spacer length of the guide RNA is at least 15 nucleotides. In another example embodiment, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 98.5% or 98% or 97.5% or 97% or 96.5% or 96% or 95.5% or 95% or 94.5% complementarity between the sequence and the guide.

In some embodiments according to the disclosure, the guide RNA (capable of guiding Cas to a target locus) may comprise (1) a guide sequence capable of hybridizing to a genomic target locus in the eukaryotic cell; (2) a tracr sequence; and (3) a tracr mate sequence. All of (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this disclosure. Various modifications may be used to increase the specificity of binding to the target sequence and/or increase the activity of the Cas protein and/or reduce off-target effects. Example guide sequence modifications are described in International Patent Application No. PCT/US2019/045582, specifically paragraphs [0178]-[0333]. which is incorporated herein by reference.

### Target Sequences, PAMs, and PFSs

In the context of formation of a CRISPR complex, “target sequence” refers to a sequence to which a guide sequence is designed to have complementarity, where hybridization between a target sequence and a guide sequence promotes the formation of a CRISPR complex. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

PAM elements are sequences that can be recognized and bound by Cas proteins. Cas proteins/effector complexes can then unwind the dsDNA at a position adjacent to the PAM element. It will be appreciated that Cas proteins and systems target RNA do not require PAM sequences (Marraffini et al. 2010. Nature. 463:568-571). Instead, many rely on PFSs, which are discussed elsewhere herein. In one example embodiment, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 2 (from Gleditzsch et al. 2019) below shows several Cas polypeptides and the PAM sequence they recognize.

TABLE 2
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) | TTTV |
| (including LbCpf1 | |
| and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |
| Cas1 | 5′-CTT-3′ |
| Cas8e | 5′-ATG-3′ |
| Type I-A | 5′-CCN-3′ |
| Type I-B | TTC, ACT, TAA, TAT, TAG, |
| | and CAC |
| Type I-C | NTTC |
| Type I-E | 5′-AAG-3′ |
| Type I-F | GG |

In a preferred embodiment, the CRISPR effector protein may recognize a 3′ PAM. In one example embodiment, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: 10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Sequences Related to Nucleus Targeting and Transportation

In some embodiments, one or more components (e.g., the Cas protein) in the composition for engineering cells may comprise one or more sequences related to nucleus targeting and transportation. Such sequences may facilitate the one or more components in the composition for targeting a sequence within a cell. In order to improve targeting of the CRISPR-Cas protein used in the methods of the present disclosure to the nucleus, it may be advantageous to provide one or both of these components with one or more nuclear localization sequences (NLSs).

In one example embodiment, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 25) or PKKKRKVEAS (SEQ ID NO: 26); the NLS from nucleoplasmin (e.g., the nucleoplasmin bipartite NLS with the sequence KRPAATKKAGQAKKKK (SEQ ID NO: 27)); the c-myc NLS having the amino acid sequence PAAKRVKLD (SEQ ID NO: 28) or RQRRNELKRSP (SEQ ID NO: 29); the hRNPA1 M9 NLS having the sequence NQSSNFGPMKGGNFGGRSSGPYGGGGQYFAKPRNQGGY (SEQ ID NO: 30); the sequence RMRIZFKNKGKDTAELRRRRVEVSVELRKAKKDEQILKRRNV (SEQ ID NO: 31) of the IBB domain from importin-alpha; the sequences VSRKRPRP (SEQ ID NO: 32) and PPKKARED (SEQ ID NO: 33) of the myoma T protein; the sequence PQPKKKPL (SEQ ID NO: 34) of human p53; the sequence SALIKKKKKMAP (SEQ ID NO: 35) of mouse c-abl IV; the sequences DRLRR (SEQ ID NO: 36) and PKQKKRK (SEQ ID NO: 37) of the influenza virus NS1; the sequence RKLKKKIKKL (SEQ ID NO: 38) of the Hepatitis virus delta antigen; the sequence REKKKFLKRR (SEQ ID NO: 39) of the mouse Mx1 protein; the sequence KRKGDEVDGVDEVAKKKSKK (SEQ ID NO: 40) of the human poly(ADP-ribose) polymerase; and the sequence RKCLQAGMNLEARKTKK (SEQ ID NO: 41) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the Cas protein, or exposed to a Cas protein lacking the one or more NLSs.

The Cas proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the Cas proteins, an NLS attached to the C-terminal of the protein.

### Zinc Finger Nucleases

Other preferred tools for genome editing for use in the context of this disclosure include zinc finger systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

Zinc Finger proteins can comprise a functional domain (e.g., activator domain). The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### TALENS

As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers,” “TALE monomers,” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁—(X₁₂X₁₃)—X₁₄₋₃₃ or 34 or 35, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁—(X₁₂X₁₃)—X₁₄-33 or 34 or 35)z, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011). each of which is incorporated herein by reference in its entirety.

The polypeptides used in methods of the disclosure can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the disclosure will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the disclosure may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in one example embodiment, the TALE polypeptides described herein further comprise an N-terminal capping region and/or a C-terminal capping region.

An exemplary amino acid sequence of a N-terminal capping region is:

(SEQ ID NO: 42)
MDPIRSRTPSPARELLSGPQPDGVQPTADRGVSPPAGGP
LDGLPARRTMSRTRLPSPPAPSPAFSADSFSDLLRQFDPSLENTS
LFDSLPPFGAHHTEAATGEWDEVQSGLRAADAPPPTMRVAVTA
ARPPRAKPAPRRRAAQPSDASPAAQVDLRTLGYSQQQQEKIKP
KVRSTVAQHHEALVGHGFTHAHIVALSQHPAALGTVAVKY QD
MIAALPEATHEAIVGVGKQWSGARALEALLTVAGELRGPPLQL
DTGQLLKIAKRGGVTAVEAVHAWRNALTGAPLN

An exemplary amino acid sequence of a C-terminal capping region is:

(SEQ ID NO: 43)
RPALESIVAQLSRPDPALAALTNDHLVALACLGGRPAL
DAVKKGLPHAPALIKRTNRRIPERTSHRVADHAQVVRVLGFFQ
CHSHPAQAFDDAMTQFGMSRHGLLQLFRRVGVTELEARSGTLP
PASQRWDRILQASGMKRAKPSPTSTQTPDQASLHAFADSLERD
LDAPSPMHEGDQTRAS

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the disclosure.

The entire N-terminal and/or C-terminal capping regions are not necessary to enhance the binding activity of the DNA binding region. Therefore, in one example embodiment, fragments of the N-terminal and/or C-terminal capping regions are included in the TALE polypeptides described herein.

In one example embodiment, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190,200,210,220,230,240,250, 260 or 270 amino acids of an N-terminal capping region. In another example embodiment, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In one example embodiment, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In one example embodiment, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the disclosure include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the disclosure may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the disclosure may include any combination of the activities described herein.

Other preferred tools for genome editing for use in the context of this disclosure include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

### Meganucleases

In some embodiments, a meganuclease or system thereof can be used to modify a polynucleotide of the present disclosure Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### OMEGA (Obligate Mobile Element-Guided Activity) Systems

OMEGA (Obligate Mobile Element-Guided Activity) nucleases are a class of RNA-guided nucleases encoded in a distinct family of IS200/IS605 transposons and are likely ancestors of Cas9 and Cas12 nucleases (Altae-Tran et al., The widespread IS200/IS605 transposon family encodes diverse programmable RNA-guided endonucleases. Science 374, 57-65 (2021)). These nucleases include the transposon-encoded proteins IscB (and its homologs IsrB and IshB) and TnpB, and use a non-coding RNA sequence (termed “OMEGA RNA” or “ωRNA”) as a guide to target and cleave dsDNA. Like CRISPR-Cas effector proteins, OMEGA nucleases can be reprogrammed to bind to varying target sites by using different guide RNAs specific for those sites.

In some embodiments, the programmable nuclease system is an OMEGA system. In one embodiment, the programmable nuclease is or is part of an OMEGA system. In some embodiments, the OMEGA system comprises an OMEGA protein and one or more ωRNA molecules capable of forming a complex with the OMEGA protein and directing sequence-specific binding of the complex to the target sequence within the target polynucleotide. In another embodiment, the OMEGA protein is an IscB protein, an IsrB protein, an IshB protein, a TnpB protein, or a Fanzor protein. In another embodiment, the OMEGA protein is a nickase.

OMEGA nucleases may also be mutated in one or more of their nuclease domains to generate an OMEGA nickase, which generates a single-strand nick at one or more targeted nick sites of the locus of interest. The site of the single-stranded nick at one or more targeted nick sites is determined by at least two elements, a target adjacent motif (TAM) sequence and an ωRNA.

In certain example embodiments, the programmable nickase comprises an OMEGA nickase and one or more ωRNA molecules capable of forming a complex with the OMEGA nickase and directing sequence-specific binding of the complex to the one or more targeted nick sites. In some embodiments, the OMEGA nickase may comprise an IscB nickase, an IsrB nickase, an IshB nickase, or a TnpB nickase.

### IscB Nucleases and Homologs Thereof

In certain example embodiments, the programmable nuclease protein may comprise an OMEGA nuclease from an IscB system. An IscB protein may comprise an X domain and a Y domain as described herein. The IscB system comprises an IscB protein and a nucleic acid component capable of forming a complex with the IscB protein and directing the complex to a target polynucleotide or targeted nick site. The IscB systems include the homolog IsrB and IshB systems. The nucleic acid component may also be referred to herein as a hRNA or ωRNA. In some examples, the IscB proteins may form a complex with one or more guide molecules. In some cases, the IscB proteins may form a complex with one or more hRNA molecules which serve as a scaffold molecule and comprise guide sequences. In some examples, the IscB proteins are CRISPR-associated proteins, e.g., the loci of the nucleases are associated with an CRISPR array. In some examples, the IscB proteins are not CRISPR-associated. In some examples, the IscB protein may be homolog or ortholog of IscB proteins described in Kapitonov V V et al., ISC, a Novel Group of Bacterial and Archaeal DNA Transposons That Encode Cas9 Homologs, J Bacteriol. 2015 Dec. 28; 198(5):797-807. Doi: 10.1128/JB.00783-15, which is incorporated by reference herein in its entirety.

IscB proteins, and homologs thereof, are considerably smaller than other RNA-guided nucleases. As such, IscB proteins, and homologs thereof, represent a novel class of RNA-guided nucleases that do not suffer from the delivery size limitations of other larger single-effector, RNA-guided nucleases, such as Type II and Type V CRISPR-Cas systems. Due to their smaller size, IscB proteins, and homologs thereof, may be combined with other functional domains (e.g., nucleobase deaminases, reverse transcriptases, transposases, ligases, topoisomerases, serine and threonine recombinases, etc.) and still be packaged in conventional delivery systems like certain adenovirus and lentivirus based viral vectors. Thus, among other improvements, the IscB systems and homologs thereof disclosed herein allow more flexible and effective strategies to manipulate and modify target polynucleotides. IscB nucleases and OMEGA systems are further described in Altae-Tran et al., The widespread IS200/605 transposon family encodes diverse programmable RNA-guided endonucleases, Science. 2021 October; 374(6563): 57-65, which is incorporated by reference herein in its entirety. Additional exemplary IscB proteins, systems, and examples are described in WO 2022/087494, which is incorporated by reference as if expressed in its entirety herein and can be adapted for use with the present disclosure in view of the description herein.

In certain example embodiments, the programmable DNA-binding protein may comprise an IscB nuclease or nickase. IscB proteins comprise a PLMP domain, RuvC domains, and an HNH domain. In one embodiment, the IscB is an ωRNA-guided nickase. In one embodiment, the ωRNA-guided IscB nicks a DNA target. In one embodiment, the DNA target is a dsDNA, and the nick occurs on the non-target strand of the dsDNA target. In some embodiments, the IscB nicks the dsDNA in a guide and TAM specific manner.

In certain example embodiments, the programmable DNA-binding protein may comprise an IsrB nuclease or nickase. As noted above, IsrB proteins are homologs of IscB proteins. IsrB polypeptides comprise a PLMP domain and RuvC domains but do not comprise an HNH domain. The IsrB proteins may be about 200 to about 500 amino acids in length, about 250 to about 450 amino acids in length, or about 300 to about 400 amino acids in length. In one embodiment, the IsrB is an ωRNA-guided nickase. In one embodiment, the ωRNA-guided IsrB nicks a DNA target. In one embodiment, the DNA target is a dsDNA, and the nick occurs on the non-target strand of the dsDNA target. In some embodiments, the IsrB nicks the dsDNA in a guide and TAM specific manner.

In certain example embodiments, the programmable DNA-binding protein may comprise an IshB nuclease or nickase. As noted above, IshB proteins are homologs of IscB proteins. IshB proteins are generally smaller than IscB and IsrB proteins and contain only a PLMP domain and HNH domain, but no RuvC domains. The IshB proteins may be about 150 to about 235 amino acids in length, about 160 to about 220 amino acids in length, about 170 to about 200 amino acids in length, about 170 to about 190 amino acids in length, or about 175 to 185 amino acids in length. In one embodiment, the IshB is an ωRNA-guided nickase. In one embodiment, the ωRNA-guided IshB nicks a DNA target. In one embodiment, the DNA target is a dsDNA, and the nick occurs on the non-target strand of the dsDNA target. In some embodiments, the IshB nicks the dsDNA in a guide and TAM specific manner.

In some embodiments, the IscBs may comprise one or more domains, e.g., one or more of a X domain (e.g., at N-terminus), a RuvC domain, a Bridge Helix domain, and a Y domain (e.g., at C-terminus). In some examples, the nucleic-acid guided nuclease comprises an N-terminal X domain, a RuvC domain (e.g., including a RuvC-I, RuvC-II, and RuvC-III subdomains), a Bridge Helix domain, and a C-terminal Y domain. In some examples, the nucleic-acid guided nuclease comprises In some examples, the nucleic-acid guided nuclease comprises an N-terminal X domain, a RuvC domain (e.g., Including a RuvC-I, RuvC-II, and RuvC-III subdomains), a Bridge Helix domain, an HNH domain, and a C-terminal Y domain.

In some embodiments, the nucleic acid-guided nucleases may have a small size. For example, the nucleic acid-guided nucleases may be no more than 50, no more than 100, no more than 150, no more than 200, no more than 250, no more than 300, no more than 350, no more than 400, no more than 450, no more than 500, no more than 550, no more than 600, no more than 650, no more than 700, no more than 750, no more than 800, no more than 850, no more than 900, no more than 950, or no more than 1000 amino acids in length.

In some examples, the IscB protein shares at least 8000, at least 8500, at least 900%, at least 95%, at least 99%, or 1000% sequence identity with a IscB protein selected from Table 3.

TABLE 3

| No. | Proteins | Sequences |
|---|---|---|
| 1 | IscB(−HNH) | MSTDATLIRTTPSHAEADATDTLVATPLMPPRRVISPWPGPGE |
| | EFH81386 | GQSLMRIPVVDIRGMALMPCTPAKARHLLKSGNARPKRNKL |
| | | GLFYVQLSYEQEPDNQSLVAGVDPGSKFEGLSVVGTKDTVL |
| | | NLMVEAPDHVKGAVQTRRTMRRARRQRKWRRPKRFHNRLN |
| | | RMQRIPPSTRSRWEAKARIVAHLRTILPFTDVVVEDVQAVTR |
| | | KGKGGTWNGSFSPVQVGKEHLYRLLRAMGLTLHLREGWQT |
| | | KELREQHGLKKTKSKSKQSFESHAVDSWVLAASISGAEHPTC |
| | | TRLWYMVPAILHRRQLHRLQASKGGVRKPYGGTRSLGVKRG |
| | | TLVEHKKYGRCTVGGVDRKRNTISLHEYRTNTRLTQAAKVE |
| | | TCRVLTWLSWRSWLLRGKRTSSKGKGSHSS (SEQ ID NO: 44) |
| 2 | IscB(+HNH) | MQPAKQQNWVFQINGDKQPLDMINPGRCRELQNRGKLASFR |
| | TAE54104.1 | RFPYVVIQQQTIENPQTKEYILKIDPGSQWTGFAIQCGNDILFR |
| | | AELNHRGEAIKFDLVKRAWFRRGRRSRNLRYRKKRLNRAKP |
| | | EGWLAPSIRHRVLTVETWIKRFMRYCPIAWIEIEQVRFDTQKL |
| | | ANPEIDGVEYQQGELQGYEVREYLLQKWGRKCAYCGTENVP |
| | | LEVEHIQSKSKGGSSRIGNLTLACHVCNVKKGNLDVRDFLAK |
| | | SPDILNQVLENSTKPLKDAAAVNSTRYAIVKMAKSICENVKC |
| | | SSGARTKMNRVRQGLEKTHSLDAACVGESGASIRVLTDRPLL |
| | | ITCKGHGSRQSIRVNASGFPAVKNAKTVFTHIAAGDVVRFTIG |
| | | KDRKKAQAGTYTARVKTPTPKGFEVLIDGAR |
| | | ISLSTMSNVVFVHRSDGYGYEL (SEQ ID NO: 45) |
| 3 | IscB(+HNH) | MAVFVIDKHKRPLMPCSEKRARLLLERGRAVVHRQVPFVIRL |
| | WP_038093640.1 | KDRTVQHSAVQPLRVALDPGSRATGMALVREKNTVDTGTGE |
| | | VYRERIALNLFELVHRGHRIREQLDQRRNFRRRRRGANLRYR |
| | | APRFDNRRRPPGWLAPSLQHRVDTTMAWVRRLCRWAPASAI |
| | | GIETVRFDTQRLQNPEISGVEYQQGALAGCEVREYLLEKWGR |
| | | KCAYCGAENVPLEIEHIVPKSRGGSDRVSNLALACRACNQAK |
| | | GNRDVRAFLADQPERLARILAQAKAPLKDAAAVNATRWAL |
| | | YRALVDTGLPVEAGTGGRTKWNRTRLGLPKTHALDALCVG |
| | | QVDQVRHWRVPVLGIRCAGRGSYRRTRLTRHGFPRGYLTRN |
| | | KSAFGFQTGDLIRAVVTKGKKAGTYLGRIAIRASGSFNIQTPM |
| | | GVVQGIHHRFCTLLQRADGYGYFVQP |
| | | KPTEAALSSPRLKAGVSSAGN (SEQ ID NO: 46) |
| 4 | IscB(+HNH) | MTTNVVFVIDTNQKPLQPCSAAVARKLLLRGKAAMFRRYPA |
| | WP_052490348.1 | VIILKKEVDSVGKPKIELRIDPGSKYTGFALVDSKDNADFIIWG |
| | | TELEHRGAAICKELTKRSAIRRSRRNRKTRYRKKRFERRKPEG |
| | | WLAPSLQHRVDTTLTWVKRICKFVPIMSISVEQVKFDLQKLE |
| | | NSDIQGIEYQQGTLAGYTLREALLEHWGRKCAYCDVENVFL |
| | | EIEHIYPKSKGGSDKFSNLTLACHKCNINKGNKSIDEFLLSDH |
| | | KRLEQIKLHQKKTLKDAAAVNATRKKLVTTLQEKTFLNVLV |
| | | SDGASTKMTRLSSSLAKRHWIDAGCVNTTLIVILKTLQPLQV |
| | | KCN |
| | | GHGNKQFVTMDAYGFPRKSYEPKKVRKDWKAGDIIRVTKK |
| | | DGTMLMGRVKKAAKKLVYIPFGGKEASFSS |
| | | ENAKAIHRSDGYRYSFAAIDSELLQKMAT (SEQ ID NO: 47) |
| 5 | IscB(+HNH) | MPNKYAFVLDSKGKLLDPTKSKKAWYLIRKGKASLVEEYPLI |
| | WP_015325818.1 | IKLKREVPKDQVNSDKLILGIDDGTKKVGFALVQKCQTKNKV |
| | | LFKAVMEQRQDVSKKMEERRGYRRYRRSHKRYRPARFDNR |
| | | SSSKRKGRIPPSILQKKQAILRVVNKLKKYIRIDKIVLEDVSIDI |
| | | RKLTEGRELYNWEYQESNRLDENLRKATLYRDDCTCQLCGT |
| | | TETMLHAHHIMPRRDGGADSIYNLITLCKACHKDKVDNNEY |
| | | QYKDQFLAIIDSKELSDLKSASHVMQGKTWLRDKLSKIAQLEI |
| | | TSGGNTANKRIDYEIEKSHSNDAICTTGLLPVDNIDDIKEYYIK |
| | | PLRKKSKAKIKELKCFRQRDLVKYTKRNGETYTGYITSLRIKN |
| | | NKYNSKVCNFSTLKGKIFRGYGFRNLTLLNRPKGLMIV (SEQ |
| | | ID NO: 48) |
| 6 | sp\|G3ECR1\| | MLFNKCIIISINLDFSNKEKCMTKPYSIGLDIGTNSVGWAVITD |
| | CAS9_STRTR | NYKVPSKKMKVLGNTSKKYIKKNLLGVLLFDSGITAEGRRLK |
| | | RTARRRYTRRRNRILYLQEIFSTEMATLDDAFFQRLDDSFLVP |
| | | DDKRDSKYPIFGNLVEEKVYHDEFPTIYHLRKYLADSTKKAD |
| | | LRLVYLALAHMIKYRGHFLIEGEFNSKNNDIQKNFQDFLDTY |
| | | NAIFESDLSLENSKQLEEIVKDKISKLEKKDRILKLFPGEKNSGI |
| | | FSEFLKLIVGNQADFRKCFNLDEKASLHFSKESYDEDLETLLG |
| | | YIGDDYSDVFLKAKKLYDAILLSGFLTVTDNETEAPLSSAMIK |
| | | RYNEHKEDLALLKEYIRNISLKTYNEVFKDDTKNGYAGYIDG |
| | | KTNQEDFYVYLKNLLAEFEGADYFLEKIDREDFLRKQRTFDN |
| | | GSIPYQIHLQEMRAILDKQAKFYPFLAKNKERIEKILTFRIPYY |
| | | VGPLARGNSDFAWSIRKRNEKITPWNFEDVIDKESSAEAFINR |
| | | MTSFDLYLPEEKVLPKHSLLYETFNVYNELTKVRFIAESMRD |
| | | YQFLDSKQKKDIVRLYFKDKRKVTDKDIIEYLHAIYGYDGIEL |
| | | KGIEKQFNSSLSTYHDLLNIINDKEFLDDSSNEAIIEEIIHTLTIF |
| | | EDREMIKQRLSKFENIFDKSVLKKLSRRHYTGWGKLSAKLIN |
| | | GIRDEKSGNTILDYLIDDGISNRNFMQLIHDDALSFKKKIQKA |
| | | QIIGDEDKGNIKEVVKSLPGSPAIKKGILQSIKIVDELVKVMGG |
| | | RKPESIVVEMARENQYTNQGKSNSQQRLKRLEKSLKELGSKI |
| | | LKENIPAKLSKIDNNALQNDRLYLYYLQNGKDMYTGDDLDI |
| | | DRLSNYDIDHIIPQAFLKDNSIDNKVLVSSASNRGKSDDFPSLE |
| | | VVKKRKTFWYQLLKSKLISQRKFDNLTKAERGGLLPEDKAG |
| | | FIQRQLVETRQITKHVARLLDEKFNNKKDENNRAVRTVKIITL |
| | | KSTLVSQFRKDFELYKVREINDFHHAHDAYLNAVIASALLKK |
| | | YPKLEPEFVYGDYPKYNSFRERKSATEKVYFYSNIMNIFKKSI |
| | | SLADGRVIERPLIEVNEETGESVWNKESDLATVRRVLSYPQV |
| | | NVVKKVEEQNHGLDRGKPKGLFNANLSSKPKPNSNENLVGA |
| | | KEYLDPKKYGGYAGISNSFAVLVKGTIEKGAKKKITNVLEFQ |
| | | GISILDRINYRKDKLNFLLEKGYKDIELIIELPKYSLFELSDGSR |
| | | RMLASILSTNNKRGEIHKGNQIFLSQKFVKLLYHAKRISNTINE |
| | | NHRKYVENHKKEFEELFYYILEFNENYVGAKKNGKLLNSAF |
| | | QSWQNHSIDELCSSFIGPTGSERKGLFELTSRGSAADFEFLGV |
| | | KIPRYRDYTPSSLLKDATLIHQSVTGLYETRIDLAKLGEG (SEQ |
| | | ID NO: 49) |
| 7 | sp\|J7RUA5\| | MKRNYILGLDIGITSVGYGIIDYETRDVIDAGVRLFKEANVEN |
| | CAS9_STAAU | NEGRRSKRGARRLKRRRRHRIQRVKKLLFDYNLLTDHSELSG |
| | | INPYEARVKGLSQKLSEEEFSAALLHLAKRRGVHNVNEVEED |
| | | TGNELSTKEQISRNSKALEEKYVAELQLERLKKDGEVRGSINR |
| | | FKTSDYVKEAKQLLKVQKAYHQLDQSFIDTYIDLLETRRTYY |
| | | EGPGEGSPFGWKDIKEWYEMLMGHCTYFPEELRSVKYAYNA |
| | | DLYNALNDLNNLVITRDENEKLEYYEKFQIIENVFKQKKKPT |
| | | LKQIAKEILVNEEDIKGYRVTSTGKPEFTNLKVYHDIKDITAR |
| | | KEIIENAELLDQIAKILTIYQSSEDIQEELTNLNSELTQEEIEQIS |
| | | NLKGYTGTHNLSLKAINLILDELWHTNDNQIAIFNRLKLVPKK |
| | | VDLSQQKEIPTTLVDDFILSPVVKRSFIQSIKVINAIIKKYGLPN |
| | | DIIIELAREKNSKDAQKMINEMQKRNRQTNERIEEIIRTTGKEN |
| | | AKYLIEKIKLHDMQEGKCLYSLEAIPLEDLLNNPFNYEVDHIIP |
| | | RSVSFDNSFNNKVLVKQEENSKKGNRTPFQYLSSSDSKISYET |
| | | FKKHILNLAKGKGRISKTKKEYLLEERDINRFSVQKDFINRNL |
| | | VDTRYATRGLMNLLRSYFRVNNLDVKVKSINGGFTSFLRRK |
| | | WKFKKERNKGYKHHAEDALIIANADFIFKEWKKLDKAKKV |
| | | MENQMFEEKQAESMPEIETEQEYKEIFITPHQIKHIKDFKDYK |
| | | YSHRVDKKPNRELINDTLYSTRKDDKGNTLIVNNLNGLYDK |
| | | DNDKLKKLINKSPEKLLMYHHDPQTYQKLKLIMEQYGDEKN |
| | | PLYKYYEETGNYLTKYSKKDNGPVIKKIKYYGNKLNAHLDIT |
| | | DDYPNSRNKVVKLSLKPYRFDVYLDNGVYKFVTVKNLDVIK |
| | | KENYYEVNSKCYEEAKKLKKISNQAEFIASFYNNDLIKINGEL |
| | | YRVIGVNNDLLNRIEVNMIDITYREYLENMNDKRPPRIIKTIAS |
| | | KTQSIKKYSTDILGNLYEVKSKKHPQIIKKG (SEQ ID NO: 50) |
| 8 | Streptococcus_ | KYSIGLDIGTNSVGWAVITDEYKVPSKKFKVLGNTDRHSIKK |
| | pyogenes_ | NLIGALLFDSGETAEATRLKRTARRRYTRRKNRICYLQEIFSN |
| | SF370 | EMAKVDDSFFHRLEESFLVEEDKKHERHPIFGNIVDEVAYHE |
| | | KYPTIYHLRKKLVDSTDKADLRLIYLALAHMIKFRGHFLIEGD |
| | | LNPDNSDVDKLFIQLVQTYNQLFEENPINASGVDAKAILSARL |
| | | SKSRRLENLIAQLPGEKKNGLFGNLIALSLGLTPNFKSNFDLA |
| | | EDAKLQLSKDTYDDDLDNLLAQIGDQYADLFLAAKNLSDAIL |
| | | LSDILRVNTEITKAPLSASMIKRYDEHHQDLTLLKALVRQQLP |
| | | EKYKEIFFDQSKNGYAGYIDGGASQEEFYKFIKPILEKMDGTE |
| | | ELLVKLNREDLLRKQRTFDNGSIPHQIHLGELHAILRRQEDFY |
| | | PFLKDNREKIEKILTFRIPYYVGPLARGNSRFAWMTRKSEETIT |
| | | PWNFEEVVDKGASAQSFIERMTNFDKNLPNEKVLPKHSLLYE |
| | | YFTVYNELTKVKYVTEGMRKPAFLSGEQKKAIVDLLFKTNR |
| | | KVTVKQLKEDYFKKIECFDSVEISGVEDRFNASLGTYHDLLKI |
| | | IKDKDFLDNEENEDILEDIVLTLTLFEDREMIEERLKTYAHLFD |
| | | DKVMKQLKRRRYTGWGRLSRKLINGIRDKQSGKTILDFLKSD |
| | | GFANRNFMQLIHDDSLTFKEDIQKAQVSGQGDSLHEHIANLA |
| | | GSPAIKKGILQTVKVVDELVKVMGRHKPENIVIEMARENQTT |
| | | QKGQKNSRERMKRIEEGIKELGSQILKEHPVENTQLQNEKLY |
| | | LYYLQNGRDMYVDQELDINRLSDYDVDHIVPQSFLKDDSIDN |
| | | KVLTRSDKNRGKSDNVPSEEVVKKMKNYWRQLLNAKLITQR |
| | | KFDNLTKAERGGLSELDKAGFIKRQLVETRQITKHVAQILDSR |
| | | MNTKYDENDKLIREVKVITLKSKLVSDFRKDFQFYKVREINN |
| | | YHHAHDAYLNAVVGTALIKKYPKLESEFVYGDYKVYDVRK |
| | | MIAKSEQEIGKATAKYFFYSNIMNFFKTEITLANGEIRKRPLIE |
| | | TNGETGEIVWDKGRDFATVRKVLSMPQVNIVKKTEVQTGGF |
| | | SKESILPKRNSDKLIARKKDWDPKKYGGFDSPTVAYSVLVVA |
| | | KVEKGKSKKLKSVKELLGITIMERSSFEKNPIDFLEAKGYKEV |
| | | KKDLIIKLPKYSLFELENGRKRMLASAGELQKGNELALPSKY |
| | | VNFLYLASHYEKLKGSPEDNEQKQLFVEQHKHYLDEIIEQISE |
| | | FSKRVILADANLDKVLSAYNKHRDKPIREQAENIIHLFTLTNL |
| | | GAPAAFKYFDTTIDR |
| | | KRYTSTKEVLDATLIHQSITGLYETRIDLSQLGGD (SEQ ID |
| | | NO: 51) |
| No. | Proteins | Domains and amino acid positions |
| 1 | IscB(−HNH) | X domain: 51-97 |
| | EFH81386 | RuvC-I: 104-118 |
| | | Bridge Helix: 140-160 |
| | | RuvC-II: 169-212 |
| | | RuvC-III: 226-278 |
| 2 | IscB(+HNH) | X domain: 11-56 |
| | TAE54104.1 | RuvC-I: 63-77 |
| | | Bridge Helix: 100-121 |
| | | RuvC-II: 129-172 |
| | | HNH: 211-243 |
| | | RuvC-III: 279-321 |
| 3 | IscB(+HNH) | X domain: 4-50 |
| | WP_038093640.1 | RuvC-I: 57-71 |
| | | Bridge Helix: 108-129 |
| | | RuvC-II: 138-181 |
| | | HNH: 220-252 |
| | | RuvC-III: 288-330 |
| 4 | IscB(+HNH) | X domain: 7-52 |
| | WP_052490348.1 | RuvC-I: 59-73 |
| | | Bridge Helix: 100-121 |
| | | RuvC-II: 129-172 |
| | | HNH: 211-243 |
| | | RuvC-III: 279-322 |
| 5 | IscB(+HNH) | X domain: 7-52 |
| | WP_015325818.1 | RuvC-I: 61-75 |
| | | Bridge Helix: 101-121 |
| | | RuvC-II: 132-175 |
| | | HNH: 215-247 |
| | | RuvC-III: 284-327 |
| 6 | sp\|G3ECR1\| | RuvC-I: 28-42 |
| | CAS9_STRTR | Bridge Helix: 85-108 |
| | | Rec: 118-736 |
| | | RuvC-II: 750-799 |
| | | HNH: 864-896 |
| | | RuvC-III: 957-1019 |
| | | PAM Interaction (PI): 1119-1409 |
| 7 | sp\|J7RUA5\| | RuvC-I: 7-21 |
| | CAS9_STAAU | Bridge Helix: 49-72 |
| | | Rec: 80-433 |
| | | RuvC-II: 445-493 |
| | | HNH: 553-585 |
| | | RuvC-III: 654-709 |
| | | PAM Interaction (PI): 789-1053 |
| 8 | Streptococcus_ | RuvC-I: 4-18 |
| | pyogenes_ | Bridge Helix: 61-84 |
| | SF370 | Rec: 94-718 |
| | | RuvC-II: 725-774 |
| | | HNH: 833-865 |
| | | RuvC-III: 926-988 |
| | | PAM Interaction (PI): 1099-1365 |

### X Domains

In some embodiments, the IscB proteins comprise an X domain, e.g., at its N-terminal.

In certain embodiments, the X domain include the X domains in Table 3. Examples of the X domains also include any polypeptides a structural similarity and/or sequence similarity to a X domain described in the art. In some examples, the X domain may have an amino acid sequence that share at least 5000, at least 5500, at least 60%, at least 5%, at least 70%, at least 75%, at least 80%, at least 85%, at least 90%, at least 95%, at least 99%, or 1000% sequence identity with X domains in Table 3.

In some examples, the X domain may be no more than 10, no more than 20, no more than 30, no more than 40, no more than 50, no more than 60, no more than 70, no more than 80, no more than 90, or no more than 100 amino acids in length. For example, the X domain may be no more than 50 amino acids in length, such as comprising 2 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

### Y Domain

In some embodiments, the IscB proteins comprise a Y domain, e.g., at its C-terminal.

In certain embodiments, the X domain include Y domains in Table 3. Examples of the Y domain also include any polypeptides a structural similarity and/or sequence similarity to a Y domain described in the art. In some examples, the Y domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with Y domains in Table 3.

### RuvC Domain

In some embodiments, the IscB proteins comprises at least one nuclease domain. In certain embodiments, the IscB proteins comprise at least two nuclease domains. In certain embodiments, the one or more nuclease domains are only active upon presence of a cofactor. In certain embodiments, the cofactor is Magnesium (Mg). In embodiments where more than one nuclease domain is present and the substrate is a double-strand polynucleotide, the nuclease domains each cleave a different strand of the double-strand polynucleotide. In certain embodiments, the nuclease domain is a RuvC domain.

The IscB proteins may comprise a RuvC domain. The RuvC domain may comprise multiple subdomains, e.g., RuvC-I, RuvC-II and RuvC-III. The subdomains may be separated by interval sequences on the amino acid sequence of the protein.

In certain embodiments, examples of the RuvC domain include those in Table 3. Examples of the RuvC domain also include any polypeptides a structural similarity and/or sequence similarity to a RuvC domain described in the art. For example, the RuvC domain may share a structural similarity and/or sequence similarity to a RuvC of Cas9. In some examples, the RuvC domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with RuvC domains in Table 3.

### Bridge Helix

The IscB proteins comprise a bridge helix (BH) domain. The bridge helix domain refers to a helix and arginine rich polypeptide. The bridge helix domain may be located next to anyone of the amino acid domains in the nucleic-acid guided nuclease. In some embodiments, the bridge helix domain is next to a RuvC domain, e.g., next to RuvC-I, RuvC-II, or RuvC-III subdomain. In one example, the bridge helix domain is between a RuvC-1 and RuvC2 subdomains.

The bridge helix domain may be from 10 to 100, from 20 to 60, from 30 to 50, e.g., 35, 36, 37, 38, 39, 40, 41, 42, 43, 44, 45, 46 or 47, 48, 49, or 50 amino acids in length. Examples of bridge helix includes the polypeptide of amino acids 60-93 of the sequence of S. pyogenes Cas9.

In certain embodiments, examples of the BH domain include those in Table 3. Examples of the BH domain also include any polypeptides a structural similarity and/or sequence similarity to a BH domain described in the art. For example, the BH domain may share a structural similarity and/or sequence similarity to a BH domain of Cas9. In some examples, the BH domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with BH domains in Table 3.

### HNH Domain

The IscB proteins comprise an HNH domain. In certain embodiments, at least one nuclease domain shares a substantial structural similarity or sequence similarity to a HNH domain described in the art.

In some examples, the nucleic acid-guided nuclease comprises a HNH domain and a RuvC domain. In the cases where the RuvC domain comprises RuvC-I, RuvC-II, and RuvC-III domain, the HNH domain may be located between the Ruv C II and RuvC III subdomains of the RuvC domain.

In certain embodiments, examples of the HNH domain include those in Table 3. Examples of the HNH domain also include any polypeptides a structural similarity and/or sequence similarity to a HNH domain described in the art. For example, the HNH domain may share a structural similarity and/or sequence similarity to a HNH domain of Cas9. In some examples, the HNH domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with HNH domains in Table 3.

hRNA

In some examples, the IscB proteins capable of forming a complex with one or more hRNA molecules (also referred to herein as ωRNAs). The hRNA complex can comprise a guide sequence and a scaffold that interacts with the IscB polypeptide. An hRNA molecules may form a complex with an IscB polypeptide nuclease or IscB polypeptide and direct the complex to bind with a target sequence. In certain example embodiments, the hRNA molecule is a single molecule comprising a scaffold sequence and a spacer sequence. In certain example embodiments, the spacer is 5′ of the scaffold sequence. In certain example embodiments, the hRNA molecule may further comprise a conserved nucleic acid sequence between the scaffold and spacer portions.

As used herein, a heterologous hRNA molecule is an hRNA molecule that is not derived from the same species as the IscB polypeptide nuclease, or comprises a portion of the molecule, e.g., spacer, that is not derived from the same species as the IscB polypeptide nuclease, e.g., IscB protein. For example, a heterologous hRNA molecule of a IscB polypeptide nuclease derived from species A comprises a polynucleotide derived from a species different from species A, or an artificial polynucleotide.

### TnpB Nucleases

In certain example embodiments, the programmable nuclease is or comprises a TnpB nuclease or nickase. TnpB proteins are characterized by the presence of RuvC domains and a zinc finger domain. The TnpB proteins are between 175 and 800 amino acids in size, between 200 and 790 amino acids in size, between 200 and 780 amino acids in size, between 200 and 770 amino acids in size, between 200 and 760 amino acids in size, between 200 and 750 amino acids in size, between 200 and 740 amino acids in size, between 200 and 730 amino acids in size, between 200 and 720 amino acids in size, between 200 and 720 amino acids in size, between 200 and 710 amino acids in size, between 200 and 700 amino acids in size, between 200 and 690 amino acids in size, between 200 and 680 amino acids in size, between 200 and 670 amino acids in size, between 200 and 660 amino acids in size, between 200 and 650 amino acids in size, between 200 and 640 amino acids in size, between 200 and 630 amino acids in size, between 200 and 620 amino acids in size, between 200 and 610 amino acids in size, between 200 and 600 amino acids in size, between 200 and 590 amino acids in size, between 200 and 580 amino acids in size, between 200 and 570 amino acids in size, between 200 and 560 amino acid, between 200 between 550 amino acids, between 200 and 540 amino acids, between 200 and 530 amino acids, between 200 and 520 amino acids, between 200 and 510 amino acids, between 200 and 500 amino acids, between 200 and 490 amino acids, between 200 and 480 amino acids, between 200 and 470 amino acids, between 200 and 460 amino acids, between 200 and 450 amino acids, between 200 and 440 amino acids, between 200 and 430 amino acids, between 200 and 420 amino acids, between 200 and 410 amino acids, between 210 and 500 amino acids, between 220 and 500 amino acids. between 230 and 500 amino acids, between 240 and 500 amino acids, between 250 and 500 amino acids, between 260 and 500 amino acids, between 270 and 500 amino acids, between 280 and 500 amino acids, between 290 and 500 amino acids, between 300 and 500 amino acids, between 250 and 490 amino acids, between 250 and 480 amino acids, between 250 and 490 amino acids, or between 250 and 600 amino acids. In one embodiment, the TnpB polypeptide is between 300 and 500 amino acids, or between 350 and 450 amino acids. In one embodiment, the TnpB is an ωRNA-guided nickase. In one embodiment, the ωRNA-guided TnpB nicks a DNA target. In one embodiment, the DNA target is a dsDNA, and the nicks occurs on the non-target strand of the dsDNA target. In some embodiments, the TnpB nicks the dsDNA in a guide and TAM specific manner.

The TnpB proteins also encompass homologs or orthologs of TnpB proteins. The terms “ortholog” and “homolog” are well known in the art. By means of further guidance, a “homolog” of a protein as used herein is a protein of the same species which performs the same or a similar function as the protein it is a homolog of Homologous proteins may but need not be structurally related, or are only partially structurally related. An “ortholog” of a protein as used herein is a protein of a different species which performs the same or a similar function as the protein it is an orthologue of. Orthologous proteins may but need not be structurally related or are only partially structurally related. In particular embodiments, the homolog or ortholog of a TnpB polypeptide such as referred to herein has a sequence homology or identity of at least 80%, at least 85%, at least 90%, at least 95% with a TnpB polypeptide. In further embodiments, the homolog or ortholog of a TnpB polypeptide has a sequence identity of at least 80%, at least 85%, at least 90%, or at least 95% with a wildtype TnpB polypeptide. In particular embodiments, a homolog or ortholog is identified according to its domain structure and/or function. In embodiments, the homolog or ortholog comprises catalytic residues and/or domains as defined herein, including as identified in FIG. 1. Sequence alignments conducted as described herein, as well as folding studies and domain predictions as taught herein can aid in the identification of a homolog or ortholog with the structural and functional characteristics identifying TnpB polypeptides, particularly those with conserved residues, including catalytic residues, and domains of TnpB polypeptides.

Additional exemplary TnpB proteins, systems, and examples are described in WO 2022/159892, which is incorporated by reference as if expressed in its entirety herein and can be adapted for use with the present disclosure in view of the description herein.

### Fanzor Nucleases

In certain example embodiments, the programmable nuclease is or comprises a Fanzor nuclease or nickase. TnpBs are the likely ancestor of Fanzor proteins (Altae-Tran, Science, 374 (6563), 2021). Fanzor and TnpB proteins share the same conserved amino acid motif in their C-terminal half regions: D-X(125, 275)-[TS]-[TS]-X-X-[C4 zinc finger]-X(5,50)-RD and two groups of Fanzor polypeptides have been described (Bao and Jurka. Mobile DNA (4), Article 12 (2013)).The Fanzor polypeptide described herein may comprise a Ruv-C-like domain. The RuvC domain may be a split RuvC domain comprising a RuvC-I, RuvC-II, and RuvC-III subdomains. The Fanzor polypeptide may further comprise one or more of a HTH domain, a bridge helix domain, a REC domain, a zinc finger domain, or any combination thereof. Fanzor polypeptides do not comprise an HNH domain. In one example embodiment, Fanzor proteins comprise, starting at the N-terminus a HTH domain, a RuvC-I sub-domain, a bridge helix domain, a RuvC-II sub-domain, a zinger finger domain, and a RuvC-III sub-domain. In one example embodiment, the RuvC-III sub-domain forms the C-terminus of the Fanzor polypeptide.

In certain example embodiments, the Fanzor polypeptides are or range between 125 and 850 amino acids in size. In certain example embodiments, the Fanzor polypeptides are between 175 and 800 amino acids in size, between 200 and 790 amino acids in size, between 200 and 780 amino acids in size, between 200 and 770 amino acids in size, between 200 and 760 amino acids in size, between 200 and 750 amino acids in size, between 200 and 740 amino acids in size, between 200 and 730 amino acids in size, between 200 and 720 amino acids in size, between 200 and 720 amino acids in size, between 200 and 710 amino acids in size, between 200 and 700 amino acids in size, between 200 and 690 amino acids in size, between 200 and 680 amino acids in size, between 200 and 670 amino acids in size, between 200 and 660 amino acids in size, between 200 and 650 amino acids in size, between 200 and 640 amino acids in size, between 200 and 630 amino acids in size, between 200 and 620 amino acids in size, between 200 and 610 amino acids in size, between 200 and 600 amino acids in size, between 200 and 590 amino acids in size, between 200 and 580 amino acids in size, between 200 and 570 amino acids in size, between 200 and 560 amino acid, between 200 between 550 amino acids, between 200 and 540 amino acids, between 200 and 530 amino acids, between 200 and 520 amino acids, between 200 and 510 amino acids, between 200 and 500 amino acids, between 200 and 490 amino acids, between 200 and 480 amino acids, between 200 and 470 amino acids, between 200 and 460 amino acids, between 200 and 450 amino acids, between 200 and 440 amino acids, between 200 and 430 amino acids, between 200 and 420 amino acids, between 200 and 410 amino acids, between 210 and 500 amino acids, between 220 and 500 amino acids. between 230 and 500 amino acids, between 240 and 500 amino acids, between 250 and 500 amino acids, between 260 and 500 amino acids, between 270 and 500 amino acids, between 280 and 500 amino acids, between 290 and 500 amino acids, between 300 and 500 amino acids, between 250 and 490 amino acids, between 250 and 480 amino acids, between 250 and 490 amino acids, or between 250 and 600 amino acids. In one embodiment, the Fanzor polypeptide is between 300 and 500 amino acids, or between 350 and 450 amino acids. Fanzor polypeptides may be classified as Type 1 Fanzor polypeptides, which are typically between the size of a TnpB polypeptide and Cas12a, or Type 2 Fanzor polypeptides, which are typically smaller in size than a TnpB polypeptide.

The Fanzor polypeptides also encompasses homologs or orthologs of Fanzor polypeptides whose sequences are specifically described herein. The terms “ortholog” and “homolog” are well known in the art. By means of further guidance, a “homolog” of a protein as used herein is a protein of the same species which performs the same or a similar function as the protein it is a homolog of. Homologous proteins may be, but need not be, structurally related, or are only partially structurally related. An “ortholog” of a protein as used herein is a protein of a different species which performs the same or a similar function as the protein it is an orthologue of. Orthologous proteins may but need not be structurally related or are only partially structurally related. In particular embodiments, the homolog or ortholog of a Fanzor polypeptide such as referred to herein has a sequence homology or identity of at least 80%, at least 85%, at least 90%, at least 95% with a Fanzor polypeptide. In further embodiments, the homolog or ortholog of a Fanzor polypeptide has a sequence identity of at least 80%, at least 85%, at least 90%, or at least 95% with a wildtype Fanzor polypeptide. Exemplary Fanzor polypeptides are described in e.g., Bao and Jurka. Mobile DNA (4), Article 12 (2013)), particularly at FIG. 1, FIG. 2, and Additional files 2 and 3, which are incorporated by reference as if expressed in its entirety herein, and can be adapted for use with the present disclosure in view of the description herein.

### ωRNA Molecules

The systems herein may further comprise one or more hRNA molecules, which are referred to herein interchangeably as wRNA. The hRNA complex can comprise a guide sequence and a scaffold that interacts with the IscB protein. An hRNA molecule may form a complex with IscB protein nuclease or IscB protein, or homolog thereof, and direct the complex to bind with a target sequence. In certain example embodiments, the hRNA molecule is a single molecule comprising a scaffold sequence and a spacer sequence. In certain example embodiments, the spacer is 5′ of the scaffold sequence. In certain example embodiments, the hRNA molecule may further comprise a conserved nucleic acid sequence between the scaffold and spacer portions.

In certain example embodiments, the hRNA scaffold comprises a spacer sequence and a conserved nucleotide sequence. The hRNA scaffold typically comprises conserved regions, with the scaffold comprising 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 115, 125, 135, 145, 155, 165, 175, 185, 195, 205, 215, 225, 235, 245, 255, 265, 275, 285, 295, 305, 315, 325, 335, 345, or 355 or more nt. In an aspect, the hRNA scaffold comprises one conserved nucleotide sequence. In embodiments, the conserved nucleotide sequence is on or near a 5′ end of the scaffold. In embodiments, the scaffold may comprise a short 3-4 base pairnexus, a conserved nexus hairpin and alarge ulti-stem loop region that mau consist of two intervonnected multi-stem loops. In an aspect, an IscrB associated scaffold may comprise The scaffold hRNA may further comprise a spacer, which can be re-programmed to direct site-specific binding to a target sequence of a target polynucleotide. The spacer may also be referred to herein as part of the hRNA scaffold or as gRNA, and may comprise an engineered heterologous sequence.

In certain embodiments, the spacer length of the hRNA is from 10 to 150 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer. In certain example embodiment, the guide sequence is 15, 16, 17,18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 17, 138, 19, 140, 141, 142, 143, 144, 145, 146, 147, 148, 149 or 150 nt.

In certain embodiments, the hRNA spacer length is from 15 to 50 nt. In certain embodiments, the spacer length of the hRNA is at least 15 nucleotides. In certain embodiments, the spacer length is from 15 to 50 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt, from 34 to 40 nt, e.g., 34, 35, 36, 37, 38, 39, 40, from 35 to 39, from 36 to 38 nt long, about 37 nt, or longer.

In some embodiments, the sequence of the hRNA molecule is selected to reduce the degree of secondary structure within the hRNA molecule. In some embodiments, about or less than about 75%, 50%, 40%, 30%, 25%, 20%, 15%, 10%, 5%, 1%, or fewer of the nucleotides of the nucleic acid-targeting hRNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example of a folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

As used herein, a heterologous hRNA molecule is an hRNA molecule that is not derived from the same species as the IscB protein nuclease, or comprises a portion of the molecule, e.g. spacer, that is not derived from the same species as the IscB polypeptide nuclease, e.g. IscB protein. For example, a heterologous hRNA molecule of a IscB polypeptide nuclease derived from species A comprises a polynucleotide derived from a species different from species A, or an artificial polynucleotide.

In a particular embodiment, the hRNA comprises a guide sequence linked to a conserved nucleotide sequence, wherein the conserved nucleotide sequence may comprise one or more stem loops or optimized secondary structures. In particular embodiments, the conserved nucleotide sequence has a minimum length of 16 nts and a single stem loop. In further embodiments the conserved nucleotide sequence has a length longer than 16 nts, preferably more than 17 nts, and has more than one stem loop or optimized secondary structures. In particular embodiments, the guide sequence may be linked to all or part of the natural conserved nucleotide sequence. In particular embodiments, certain aspects of the guide architecture can be modified, for example by addition, subtraction, or substitution of features, whereas certain other aspects of guide architecture are maintained. Preferred locations for engineered guide modifications, including but not limited to insertions, deletions, and substitutions include guide termini and regions of the guide that are exposed when complexed with IscB polypeptide nuclease and/or target, for example the tetraloop and/or loop2.

In some embodiments, a loop in the guide RNA is provided. This may be a stem loop or a tetra loop. The loop is preferably GAAA, but it is not limited to this sequence or indeed to being only 4 bp in length. Indeed, preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG.

In some embodiments, the hRNA forms a stem loop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the conserved nucleotide sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

The repeat:anti repeat duplex will be apparent from the secondary structure of the hRNA. It may be typically a first complimentary stretch after (in 5′ to 3′ direction) the poly U tract and before the tetraloop; and a second complimentary stretch after (in 5′ to 3′ direction) the tetraloop and before the poly A tract. The first complimentary stretch (the “repeat”) is complimentary to the second complimentary stretch (the “anti-repeat”). As such, they Watson-Crick base pair to form a duplex of dsRNA when folded back on one another. As such, the anti-repeat sequence is the complimentary sequence of the repeat and in terms to A-U or C-G base pairing, but also in terms of the fact that the anti-repeat is in the reverse orientation due to the tetraloop.

In an embodiment of the disclosure, modification of guide architecture comprises replacing bases in stem loop 2. For example, in some embodiments, “actt” (“acuu” in RNA) and “aagt” (“aagu” in RNA) bases in stemloop2 are replaced with “cgcc” and “gcgg.” In some embodiments, “actt” and “aagt” bases in stemloop2 are replaced with complimentary GC-rich regions of 4 nucleotides. In some embodiments, the complimentary GC-rich regions of 4 nucleotides are “cgcc” and “gcgg” (both in 5′ to 3′ direction). In some embodiments, the complimentary GC-rich regions of 4 nucleotides are “gcgg” and “cgcc” (both in 5′ to 3′ direction). Other combination of C and G in the complimentary GC-rich regions of 4 nucleotides will be apparent including CCCC and GGGG.

In one aspect, the stemloop 2, e.g., “ACTTgtttAAGT” (SEQ ID NO: 52) can be replaced by any “XXXXgtttYYYY,” e.g., where XXXX and YYYY represent any complementary sets of nucleotides that together will base pair to each other to create a stem.

As used herein, the term “spacer” may also be referred to as a “guide sequence.” In some embodiments, the degree of complementarity of the guide sequence to a given target sequence, when optimally aligned using a suitable alignment algorithm, is about or more than 50%, 60%, 75%, 80%, 85%, 90%, 95%, 97.5%, 99%, or more. In certain example embodiments, the hRNA molecule comprises a guide sequence that may be designed to have at least one mismatch with the target sequence, such that a RNA duplex formed between the sequence and the target sequence. Accordingly, the degree of complementarity is less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at novocraft.com), ELAND (Illumina, San Diego, CA), SOAP (available at soap.genomics.org.cn), and Maq (available at maq.sourceforge.net). The ability of a sequence (within a nucleic acid-targeting guide sequence) to direct sequence-specific binding of a nucleic acid-targeting complex to a target nucleic acid sequence may be assessed by any suitable assay. For example, the components of a hRNA system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the sequence to be tested and a control sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting hRNA may be selected to target any target nucleic acid sequence.

A hRNA sequence, and hence a nucleic acid-targeting guide, may be selected to target any target nucleic acid sequence. The target sequence may be DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In some embodiments, the hRNA molecule forms a stemloop with a separate non-covalently linked sequence, which can be DNA or RNA. In particular embodiments, the sequences forming the hRNA are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the conserved nucleotide sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain embodiments, the hRNA molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the hRNA sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the disclosure, a hRNA nucleic acid comprises ribonucleotides and non-ribonucleotides. In one such embodiment, a hRNA comprises one or more ribonucleotides and one or more deoxyribonucleotides. In an embodiment of the disclosure, the hRNA comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of hRNA chemical modifications include, without limitation, incorporation of 2′-O-methyl (M), 2′-O-methyl 3′phosphorothioate (MS), S-constrained ethyl (cEt), or 2′-O-methyl 3′-thioPACE (MSP) at one or more terminal nucleotides. Such chemically modified hRNAs can comprise increased stability and increased activity as compared to unmodified hRNAs, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a hRNA is modified by a variety of functional moieties including fluorescent dyes, polyethylene glycol, cholesterol, proteins, or detection tags. (See Kelly et al., 2016, J. Biotech. 233:74-83). In certain embodiments, a hRNA comprises ribonucleotides in a region that binds to a target sequence and one or more deoxyribonucletides and/or nucleotide analogs in a region that binds to the IscB polypeptide nuclease. In an embodiment, deoxyribonucleotides and/or nucleotide analogs are incorporated in engineered hRNA structures. In some embodiments, 3-5 nucleotides at either the 3′ or the 5′ end of a hRNA is chemically modified. In some embodiments, only minor modifications are introduced in the seed region, such as 2′-F modifications. In some embodiments, 2′-F modification is introduced at the 3′ end of a hRNA. In certain embodiments, three to five nucleotides at the 5′ and/or the 3′ end of the hRNA are chemically modified with 2′-O-methyl (M), 2′-O-methyl 3′ phosphorothioate (MS), S-constrained ethyl (cEt), or 2′-O-methyl 3′-thioPACE (MSP). Such modification can enhance genome editing efficiency (see Hendel et al., Nat. Biotechnol. (2015) 33(9): 985-989). In certain embodiments, all of the phosphodiester bonds of a hRNA are substituted with phosphorothioates (PS) for enhancing levels of gene disruption. In certain embodiments, more than five nucleotides at the 5′ and/or the 3′ end of the hRNA are chemically modified with 2′-O-Me, 2′-F or S-constrained ethyl(cEt). Such chemically modified hRNA can mediate enhanced levels of gene disruption (see Ragdarm et al., 0215, PNAS, E7110-E7111). In an embodiment of the disclosure, a hRNA is modified to comprise a chemical moiety at its 3′ and/or 5′ end. Such moieties include, but are not limited to amine, azide, alkyne, thio, dibenzocyclooctyne (DBCO), or Rhodamine. In certain embodiments, the chemical moiety is conjugated to the hRNA by a linker, such as an alkyl chain. In certain embodiments, the chemical moiety of the modified hRNA can be used to attach the hRNA to another molecule, such as DNA, RNA, protein, or nanoparticles. Such chemically modified hRNA can be used to identify or enrich cells genetically edited by a IscB polypeptide nuclease and related systems (see Lee et al., eLife, 2017, 6:e25312, DOI:10.7554).

In a particular embodiment, the conserved nucleotide sequence may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In embodiments, the IscB polypeptide utilizes the hRNA scaffold comprising a polynucleotide sequence that facilitates the interaction with the IscB protein, allowing for sequence specific binding and/or targeting of the guide sequence with the target polynucleotide. Chemical synthesis of the hRNA scaffold is contemplated, using covalent linkage using various bioconjugation reactions, loops, bridges, and non-nucleotide links via modifications of sugar, internucleotide phosphodiester bonds, purine and pyrimidine residues. Sletten et al., Angew. Chem. Int. Ed. (2009) 48:6974-6998; Manoharan, M. Curr. Opin. Chem. Biol. (2004) 8: 570-9; Behlke et al., Oligonucleotides (2008) 18: 305-19; Watts, et al., Drug. Discov. Today (2008) 13: 842-55; Shukla, et al., ChemMedChem (2010) 5: 328-49; chemical synthesis using automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain example embodiments, the scaffold and spacer may be designed as two separate molecules that can hybridize or covalently join into a single molecule. Covalent linkage can be via a linker (e.g., a non-nucleotide loop) that comprises a moiety such as spacers, attachments, bioconjugates, chromophores, reporter groups, dye labeled RNAs, and non-naturally occurring nucleotide analogues. More specifically, suitable spacers for purposes of this disclosure include, but are not limited to, polyethers (e.g., polyethylene glycols, polyalcohols, polypropylene glycol or mixtures of ethylene and propylene glycols), polyamines group (e.g., spennine, spermidine and polymeric derivatives thereof), polyesters (e.g., poly(ethyl acrylate)), polyphosphodiesters, alkylenes, and combinations thereof. Suitable attachments include any moiety that can be added to the linker to add additional properties to the linker, such as but not limited to, fluorescent labels. Suitable bioconjugates include, but are not limited to, peptides, glycosides, lipids, cholesterol, phospholipids, diacyl glycerols and dialkyl glycerols, fatty acids, hydrocarbons, enzyme substrates, steroids, biotin, digoxigenin, carbohydrates, polysaccharides. Suitable chromophores, reporter groups, and dye-labeled RNAs include, but are not limited to, fluorescent dyes such as fluorescein and rhodamine, chemiluminescent, electrochemiluminescent, and bioluminescent marker compounds. The design of example linkers conjugating two RNA components are also described in WO 2004/015075.

The linker (e.g., a non-nucleotide loop) can be of any length. In some embodiments, the linker has a length equivalent to about 0-16 nucleotides. In some embodiments, the linker has a length equivalent to about 0-8 nucleotides. In some embodiments, the linker has a length equivalent to about 0-4 nucleotides. In some embodiments, the linker has a length equivalent to about 2 nucleotides. Example linker design is also described in International Patent Publication No. WO 2011/008730.

### Engineered Transcriptional Activators (CRISPRa)

In one example embodiment, a programmable nuclease system is used to recruit an activator protein to the METTL17 gene in order to enhance expression. In one example embodiment, the activator protein is recruited to the enhancer region of the METTL17 gene. In another example embodiment, the nuclease system is programmed to bind a sequence variant responsible for decreased METTL17 expression. In another example embodiment, the nuclease system is recruited to a binding site comprising a mutation that decreases or eliminates binding of a positive regulator of METTL17 expression. In another example embodiment, the nuclease system is recruited to an enhancer possessing the variant. For example, if a subject comprises a variant that prevents binding of a transcription factor to an enhancer controlling expression of METTL17, a catalytically inactive Cas protein (“dCas”) fused to an activator can be used to recruit that activator protein to the mutated sequence. Accordingly, a guide sequence is designed to direct binding of the dCas-activator fusion such that the activator can interact with the target genomic region and induce METTL17 expression. In one example embodiment, the guide is designed to bind within 10, 20, 30, 40, 50, 60, 70, 80, 90, 100 or up to 500 base pairs of the variant nucleotide. In one example embodiment, a CRISPR guide sequence includes the specific variant nucleotide. The Cas protein used may be any of the Cas proteins disclosed above. In one example protein, the Cas protein is a dCas9.

In one embodiment, the programmable nuclease system is a CRISPRa system (see, e.g., US 2018/0057810A1; and Konermann et al. “Genome-scale transcriptional activation by an engineered CRISPR-Cas9 complex” Nature. 2014 Dec. 10. doi: 10.1038/nature14136). Numerous genetic variants associated with disease phenotypes are found to be in non-coding region of the genome, and frequently coincide with transcription factor (TF) binding sites and non-coding RNA genes. In one embodiment, a CRISPR system may be used to activate gene transcription. A nuclease-dead RNA-guided DNA binding domain, dCas9, tethered to transcriptional activator domains that promote gene activation (e.g., p65) may be used for “CRISPRa” that activates transcription. In one example embodiment, for use of dCas9 as an activator (CRISPRa), a guide RNA is engineered to carry RNA binding motifs (e.g., MS2) that recruit effector domains fused to RNA-motif binding proteins, increasing transcription. A key dendritic cell molecule, p65, may be used as a signal amplifier, but is not required.

In certain embodiments, one or more activator domains are recruited. In one example embodiment, the activation domain is linked to the CRISPR enzyme. In another example embodiment, the guide sequence includes aptamer sequences that bind to adaptor proteins fused to an activation domain. In general, the positioning of the one or more activator domains on the inactivated CRISPR enzyme or CRISPR complex is one which allows for correct spatial orientation for the activator domain to affect the target with the attributed functional effect. For example, the transcription activator is placed in a spatial orientation which allows it to affect the transcription of the target. This may include positions other than the N—/C-terminus of the CRISPR enzyme.

In another example embodiment, a zinc finger system is used to recruit an activation domain to the METTL17 gene. In one example embodiment, the activation domain is linked to the zinc finger system. In general, the positioning of the one or more activator domains on the zinc finger system is one which allows for correct spatial orientation for the activator domain to affect the target with the attributed functional effect.

In another example embodiment, a TALE system is used to recruit an activation domain to the METTL17 gene. In one example embodiment, the activation domain is linked to the TALE system. In general, the positioning of the one or more activator domains on the TALE system is one which allows for correct spatial orientation for the activator domain to affect the target with the attributed functional effect. For example, the transcription activator is placed in a spatial orientation which allows it to affect the transcription of the target.

In another example embodiment, a meganuclease system is used to recruit an activation domain to the METTL17 gene. In one example embodiment, the activation domain is linked to the meganuclease system. In general, the positioning of the one or more activator domains on the inactivated meganuclease system is one which allows for correct spatial orientation for the activator domain to affect the target with the attributed functional effect. For example, the transcription activator is placed in a spatial orientation which allows it to affect the transcription of the target.

### Epigenetic Editing

In one aspect of the disclosure, is provided a fusion protein comprising from N-terminus to C-terminus, a demethylation domain, an XTEN linker, and a nuclease-deficient RNA-guided DNA endonuclease enzyme or a nuclease-deficient endonuclease enzyme. In aspects, the fusion protein further comprises a transcriptional activator. In aspects, the transcriptional activator is VP64, p65, Rta, or a combination of two or more thereof. In another aspect, the fusion protein further comprises a nuclear localization sequence. In embodiments, the fusion protein comprises the nuclease-deficient RNA-guided DNA endonuclease enzyme. In embodiments, the fusion protein comprises the nuclease-deficient DNA endonuclease enzyme.

In certain embodiments, the present disclosure provides a fusion protein comprising from N-terminus to C-terminus, an RNA-binding sequence, an XTEN linker, and a transcriptional activator. In aspects, the transcriptional activator is VP64, p65, Rta, or a combination of two or more thereof. In aspects, the fusion protein further comprises a demethylation domain, a nuclease-deficient RNA-guided DNA endonuclease enzyme or a nuclease-deficient endonuclease enzyme, a nuclear localization sequence, or a combination of two or more thereof. In embodiments, the fusion protein comprises the nuclease-deficient RNA-guided DNA endonuclease enzyme. In embodiments, the fusion protein comprises the nuclease-deficient DNA endonuclease enzyme.

In certain embodiments, the present disclosure provides a method of activating a target nucleic acid sequence in a cell, the method comprising: (i) delivering a first polynucleotide encoding a fusion protein described herein including embodiments thereof to a cell containing the silenced target nucleic acid; and (ii) delivering to the cell a second polynucleotide comprising: (a) a sgRNA or (b) a cr:tracrRNA; thereby reactivating the silenced target nucleic acid sequence in the cell. In aspects, the sgRNA comprises at least one MS2 stem loop. In aspects, the second polynucleotide comprises a transcriptional activator. In aspects, the second polynucleotide comprises two or more sgRNA.

In certain embodiments, the present disclosure provides a method of screening for one or more genetic elements that modulate expression of the METTL17 gene, the method comprising: contacting a plurality of cells with a library of structurally distinct small guide RNAs (sgRNAs) that target a plurality of genetic elements, thereby generating a plurality of test cells, the plurality of test cells each comprising: a small guide RNA (sgRNA); and a nuclease deficient sgRNA-mediated nuclease (dCas9), wherein the dCas9 comprises a dCas9 domain fused to a transcriptional modulator; or a dCas9 domain fused to an epitope fusion domain, selecting the test cells on the basis of the phenotype; and quantitating the frequency of the structurally distinct sgRNAs within the population of selected cells, wherein the sgRNAs that target genetic elements that modulate the phenotype are overrepresented or underrepresented in the selected cells.

In certain embodiments, the dCas9 comprises a dCas9 domain and a transcriptional activator. In some cases, the library of sgRNAs is targeted to a region between 0-750 bp upstream of the transcription start site of the METTL17 gene. In some cases, the dCas9 comprises a dCas9 domain and a transcriptional repressor. In some cases, the library of sgRNAs is targeted to a region between 0-1000 bp downstream of the transcription start site of the METTL17 gene. In some cases, wherein the dCas9 comprises: a first dCas9 fused to a transcriptional repressor; and a second dCas9 fused to a transcriptional activator; or a second dCas9 fused to an epitope fusion domain. In some cases, at least a portion of the plurality of test cells comprise a Cas9 nuclease.

### Base Editing

In one example embodiment, a method of treating subjects suffering from, or at risk of developing, a mitochondrial disease comprises administering a base editing system that corrects one or more variants associated with decreased expression or activity of METTL17 in cells and tissues. A base-editing system may comprise a Cas polypeptide linked to a nucleobase deaminase (“base editing system”) and a guide molecule capable of forming a complex with the Cas polypeptide and directing sequence-specific binding of the base editing system at a target sequence. In one example embodiment, the Cas polypeptide is catalytically inactive. In another example embodiment, the Cas polypeptide is a nickase. The Cas polypeptide may be any of the Cas polypeptides disclosed above. In one example embodiment, the Cas polypeptide is a Type II Cas polypeptide. In one example embodiment, the Cas polypeptide is a Cas9 polypeptide. In another example embodiment, the Cas polypeptide is a Type V Cas polypeptide. In one example embodiment, the Cas polypeptide is a Cas12a or Cas12b polypeptide. The nucleobase deaminase may be cytosine base editor (CBE) or adenosine base editors (ABEs). CBEs convert C•G base pairs into a T•A base pair (Komor et al. 2016. Nature. 533:420-424; Nishida et al. 2016. Science. 353; and Li et al. Nat. Biotech. 36:324-327) and ABEs convert an A•T base pair to a G•C base pair. Collectively, CBEs and ABEs can mediate all four possible transition mutations (C to T, A to G, T to C, and G to A). Example base editing systems are disclosed in Rees and Liu. 2018. Nat. Rev. Genet. 19(12): 770-788, particularly at FIGS. 1b, 2a-2c, 3a-3f, and Table 1, which is specifically incorporated herein by reference. In certain example embodiments, the base editing system may further comprise a DNA glycosylase inhibitor.

The editing window of a base editing system may range over a 5-8 nucleotide window, depending on the base editing system used. Id. Accordingly, given the base editing system used, a guide sequence may be selected to direct the base editing system to convert a base or base pair of one or more variants resulting in reduced regulatory element binding to an enhancer controlling METTL17 expression to a wild-type or non-risk variant.

### ARCUS Based Editing

In one example embodiment, a method of treating subjects suffering from, or at risk of developing, a mitochondrial disease comprises administering an ARCUS base editing system. Exemplary methods for using ARCUS can be found in U.S. Pat. No. 10,851,358, US Publication No. 2020-0239544, and WIPO Publication No. 2020/206231 which are incorporated herein by reference.

### Prime Editing

In one example embodiment, a method of treating subjects suffering from, or at risk of developing, a mitochondrial disease comprises administering a prime editing system that corrects one or more variants associated with decreased expression or activity of METTL17 in cells and tissues. In one example embodiment, a method of treating subjects suffering from, or at risk of developing, a mitochondrial disease comprises administering a prime editing system that corrects one or more variants associated with decreased expression or activity of METTL17 in cells or tissues. In one example embodiment, a prime editing system comprises a Cas polypeptide having nickase activity, a reverse transcriptase, and a prime editing guide RNA (pegRNA). Cas polypeptide, and/or reverse transcriptase can be coupled together or otherwise associate with each other to form a prime editing complex and edit a target sequence. The Cas polypeptide may be any of the Cas polypeptides disclosed above. In one example embodiment, the Cas polypeptide is a Type II Cas polypeptide. In another example embodiment, the Cas polypeptide is a Cas9 nickase. In one example embodiment, the Cas polypeptide is a Type V Cas polypeptide. In another example embodiment, the Cas polypeptide is a Cas12a or Cas12b.

The prime editing guide molecule (pegRNA) comprises a primer binding site (PBS) configured to hybridize with a portion of a nicked strand on a target polynucleotide (e.g., genomic DNA) a reverse transcriptase (RT) template comprising the edit to be inserted in the genomic DNA and a spacer sequence designed to hybridize to a target sequence at the site of the desired edit. The nicking site is dependent on the Cas polypeptide used and standard cutting preference for that Cas polypeptide relative to the PAM. Thus, based on the Cas polypeptide used, a pegRNA can be designed to direct the prime editing system to introduce a nick where the desired edit should take place. In one example embodiment, a pegRNA is configured to direct the prime editing system to convert a single base or base pair of the one or more variants associated with reduced METTL17 expression to a wild-type or non-risk variant. In one example embodiment, a pegRNA is configured to direct the prime editing system to convert a single base or base pair of one or more variants associated with reduced positive regulator binding to an enhancer controlling METTL17 expression such that the positive regulator binding affinity to the enhancer is increased. In another example embodiment, a pegRNA is configured to direct the prime editing system to convert to C of rs6712203 to a T. In another example embodiment, a pegRNA is configured to direct the prime editing system to excise a portion of genomic DNA comprising one or more variants associated with reduced expression of METTL17 with a sequence that replaces the one or more variants with a wild-type or non-risk variant. In another example embodiment, a pegRNA is configured to direct the prime editing system to excise a portion of genomic DNA comprising one or more variants that reduce a positive regulator binding to an enhancer controlling METTL17 expression such that the binding affinity of the positive regulator is restored.

The pegRNA can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c. CRISPR Associated Transposases (CAST)

In one example embodiment, a method of treating subject suffering from, or at risk of developing, a mitochondrial disease comprises administering a CAST system that replaces a genomic region comprising one or more variants associated with decreased expression or activity of METTL17 in cells or tissues with a polynucleotide sequence comprising a wild type sequence or non-risk variant. In one example embodiment, a CAST system is used to replace all or a portion of an enhancer controlling METTL17 expression and comprising one or more variants that reduce positive regulator binding to the enhancer.

In one example embodiment, a method of treating subject suffering from, or at risk of developing, a mitochondrial disease comprises administering a CAST system that replaces a genomic region comprising one or more variants associated with decreased expression or activity of METTL17 with a polynucleotide sequence comprising a wild type sequence or non-risk variant. In one example embodiment, a method of treating subject suffering from, or at risk of developing, a mitochondrial disease comprises administering a CAST system that replaces a genomic region comprising one or more variants associated with decreased expression or activity of METTL17.

CAST systems comprise a Cas polypeptide, a guide sequence, a transposase, and a donor construct. The transposase is linked to or otherwise capable of forming a complex with the Cas polypeptide. The donor construct comprises a donor sequence to be inserted into a target polynucleotide and one or more transposase recognition elements. The transposase is capable of binding the donor construct and excising the donor template and directing insertion of the donor template into a target site on a target polynucleotide (e.g., genomic DNA). The guide molecule is capable of forming a CRISPR-Cas complex with the Cas polypeptide, and can be programmed to direct the entire CAST complex such that the transposase is positioned to insert the donor sequence at the target site on the target polynucleotide. For multimeric transposase, only those transposases needed for recognition of the donor construct and transposition of the donor sequence into the target polypeptide may be required. The Cas may be naturally catalytically inactive or engineered to be catalytically inactive.

In one example embodiment, the CAST system is a Tn7-like CAST system, wherein the transposase comprises one or more polypeptides from a Tn7 or Tn7-like transposase. The Cas polypeptide of the Tn7-like transposase may be a Class 1 (multimeric effector complex) or Class 2 (single protein effector) Cas polypeptide.

In one example embodiments, the Cas polypeptide is a Class 1 Type-if Cas polypeptide. In one example embodiment, the Cas polypeptide may comprise a cas6, a cas7, and a cas8-cas5 fusion. In one example embodiments, the Tn7 transposase may comprise TnsB, TnsC, and TniQ. In another example embodiment, the Tn7 transposase may comprise TnsB, TnsC, and TnsD. In certain example embodiments, the Tn7 transposase may comprise TnsD, TnsE, or both. As used herein, the terms “TnsAB,” “TnsAC,” “TnsBC,” or “TnsABC” refer to a transposon complex comprising TnsA and TnsB, TnsA and TnsC, TnsB and TnsC, TnsA and TnsB and TnsC, respectively. In these combinations, the transposases (TnsA, TnsB, TnsC) may form complexes or fusion proteins with each other. Similarly, the term TnsABC-TniQ refer to a transposon comprising TnsA, TnsB, TnsC, and TniQ, in a form of complex or fusion protein. An example Type 1f-Tn7 CAST system is described in Klompe et al. Nature, 2019, 571:219-224 and Vo et al. bioRxiv, 2021, doi.org/10.1101/2021.02.11.430876, which are incorporated herein by reference.

In one example embodiment, the Cas polypeptide is a Class 1 Type-1b Cas polypeptide. In one example embodiment, the Cas polypeptide may comprise a cas6, a cas7, and a cas8b (e.g., a ca8b3). In one example embodiments, the Tn7 transposase may comprise TnsB, TnsC, and TniQ. In another example embodiment, the Tn7 transposase may comprise TnsB, TnsC, and TnsD. In certain example embodiments, the Tn7 transposase may comprise TnsD, TnsE, or both. As used herein, the terms “TnsAB,” “TnsAC,” “TnsBC,” or “TnsABC” refer to a transposon complex comprising TnsA and TnsB, TnsA and TnsC, TnsB and TnsC, TnsA and TnsB and TnsC, respectively. In these combinations, the transposases (TnsA, TnsB, TnsC) may form complexes or fusion proteins with each other. Similarly, the term TnsABC-TniQ refer to a transposon comprising TnsA, TnsB, TnsC, and TniQ, in a form of complex or fusion protein.

In one example embodiment, the Cas polypeptide is Class 2, Type V Cas polypeptide. In one example embodiment, the Type V Cas polypeptide is a Cas12k. In one example embodiments, the Tn7 transposase may comprise TnsB, TnsC, and TniQ. In another example embodiment, the Tn7 transposase may comprise TnsB, TnsC, and TnsD. In certain example embodiments, the Tn7 transposase may comprise TnsD, TnsE, or both. As used herein, the terms “TnsAB,” “TnsAC,” “TnsBC,” or “TnsABC” refer to a transposon complex comprising TnsA and TnsB, TnsA and TnsC, TnsB and TnsC, TnsA and TnsB and TnsC, respectively. In these combinations, the transposases (TnsA, TnsB, TnsC) may form complexes or fusion proteins with each other. Similarly, the term TnsABC-TniQ refer to a transposon comprising TnsA, TnsB, TnsC, and TniQ, in a form of complex or fusion protein. An example Cas12k-Tn7 CAST system is described in Strecker et al. Science, 2019 365:48-53, which is incorporated herein by reference.

In one example embodiment, the CAST system is a Mu CAST system, wherein the transposase comprises one or more polypeptides of a Mu transposase. An example Mu CAST system is disclosed in WO 2021/041922 which is incorporated herein by reference.

In one example embodiment, the CAST comprise a catalytically inactive Type II Cas polypeptide (e.g., dCas9) fused to one or more polypeptides of a Tn5 transposase. In another example embodiment, the CAST system comprises a catalytically inactive Type II Cas polypeptide (e.g. dCas9) fused to a piggyback transposase.

### Donor Polynucleotides

The system may further comprise one or more donor polynucleotides (e.g., for insertion into the target polynucleotide). A donor polynucleotide may be an equivalent of a transposable element that can be inserted or integrated to a target site. The donor polynucleotide may be or comprise one or more components of a transposon. A donor polynucleotide may be any type of polynucleotides, including, but not limited to, a gene, a gene fragment, a non-coding polynucleotide, a regulatory polynucleotide, a synthetic polynucleotide, etc. The donor polynucleotide may include a transposon left end (LE) and transposon right end (RE). The LE and RE sequences may be endogenous sequences for the CAST used or may be heterologous sequences recognizable by the CAST used, or the LE or RE may be synthetic sequences that comprise a sequence or structure feature recognized by the CAST and sufficient to allow insertion of the donor polynucleotide into the target polynucleotides. In certain example embodiments, the LE and RE sequences are truncated. In certain example embodiments may be between 100-200 bps, between 100-190 base pairs, 100-180 base pairs, 100-170 base pairs, 100-160 base pairs, 100-150 base pairs, 100-140 base pairs, 100-130 base pairs, 100-120 base pairs, 100-110 base pairs, 20-100 base pairs, 20-90 base pairs, 20-80 base pairs, 20-70 base pairs, 20-60 base pairs, 20-50 base pairs, 20-40 base pairs, 20-30 base pairs, 50 to 100 base pairs, 60-100 base pairs, 70-100 base pairs, 80-100 base pairs, or 90-100 base pairs in length.

The donor polynucleotide may be inserted at a position upstream or downstream of a PAM on a target polynucleotide. In some embodiments, a donor polynucleotide comprises a PAM sequence. Examples of PAM sequences include TTTN, ATTN, NGTN, RGTR, VGTD, or VGTR.

The donor polynucleotide may be inserted at a position between 10 bases and 200 bases, e.g., between 20 bases and 150 bases, between 30 bases and 100 bases, between 45 bases and 70 bases, between 45 bases and 60 bases, between 55 bases and 70 bases, between 49 bases and 56 bases or between 60 bases and 66 bases, from a PAM sequence on the target polynucleotide. In some cases, the insertion is at a position upstream of the PAM sequence. In some cases, the insertion is at a position downstream of the PAM sequence. In some cases, the insertion is at a position from 49 to 56 bases or base pairs downstream from a PAM sequence. In some cases, the insertion is at a position from 60 to 66 bases or base pairs downstream from a PAM sequence.

The donor polynucleotide may be used for editing the target polynucleotide. In some cases, the donor polynucleotide comprises one or more mutations to be introduced into the target polynucleotide. Examples of such mutations include substitutions, deletions, insertions, or a combination thereof. The mutations may cause a shift in an open reading frame on the target polynucleotide. In some cases, the donor polynucleotide alters a stop codon in the target polynucleotide. For example, the donor polynucleotide may correct a premature stop codon. The correction may be achieved by deleting the stop codon or introduces one or more mutations to the stop codon. In other example embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild type gene or non-coding regulatory sequence (e.g., sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of a corresponding wild-type gene. In certain example embodiments, these defective genes may be associated with one or more disease phenotypes. In certain example embodiments, the defective gene or gene fragment is not replaced but the systems described herein are used to insert donor polynucleotides that encode gene or gene fragments that compensate for or override defective gene expression such that cell phenotypes associated with defective gene expression are eliminated or changed to a different or desired cellular phenotype.

In certain embodiments of the disclosure, the donor may include, but not be limited to, genes or gene fragments, encoding proteins or RNA transcripts to be expressed, regulatory elements, repair templates, and the like. According to the disclosure, the donor polynucleotides may comprise left end and right end sequence elements that function with transposition components that mediate insertion.

In certain cases, the donor polynucleotide manipulates a splicing site on the target polynucleotide. In some examples, the donor polynucleotide disrupts a splicing site. The disruption may be achieved by inserting the polynucleotide to a splicing site and/or introducing one or more mutations to the splicing site. In certain examples, the donor polynucleotide may restore a splicing site. For example, the polynucleotide may comprise a splicing site sequence.

The donor polynucleotide to be inserted may have a size from 10 bases to 50 kb in length, e.g., from 50 to 40 kb, from 100 to 30 kb, from 100 bases to 300 bases, from 200 bases to 400 bases, from 300 bases to 500 bases, from 400 bases to 600 bases, from 500 bases to 700 bases, from 600 bases to 800 bases, from 700 bases to 900 bases, from 800 bases to 1000 bases, from 900 bases to from 1100 bases, from 1000 bases to 1200 bases, from 1100 bases to 1300 bases, from 1200 bases to 1400 bases, from 1300 bases to 1500 bases, from 1400 bases to 1600 bases, from 1500 bases to 1700 bases, from 600 bases to 1800 bases, from 1700 bases to 1900 bases, from 1800 bases to 2000 bases, from 1900 bases to 2100 bases, from 2000 bases to 2200 bases, from 2100 bases to 2300 bases, from 2200 bases to 2400 bases, from 2300 bases to 2500 bases, from 2400 bases to 2600 bases, from 2500 bases to 2700 bases, from 2600 bases to 2800 bases, from 2700 bases to 2900 bases, or from 2800 bases to 3000 bases in length.

The components in the systems herein may comprise one or more mutations that alter their (e.g., the transposase(s)) binding affinity to the donor polynucleotide. In some examples, the mutations increase the binding affinity between the transposase(s) and the donor polynucleotide. In certain examples, the mutations decrease the binding affinity between the transposase(s) and the donor polynucleotide. The mutations may alter the activity of the Cas and/or transposase(s).

In certain embodiments, the systems disclosed herein are capable of unidirectional insertion, that is the system inserts the donor polynucleotide in only one orientation.

Delivery mechanisms for CAST systems includes those discussed above for CRISPR-Cas systems.

### Pharmaceutical Formulations and Administration

Also described herein are pharmaceutical formulations that can contain an amount, effective amount, and/or least effective amount, and/or therapeutically effective amount of one or more compounds, molecules, compositions, vectors, vector systems, cells as described above, or a combination thereof (which are also referred to as the primary active agent or ingredient elsewhere herein) described in greater detail elsewhere herein a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In some embodiments, the pharmaceutical formulation can include, such as an active ingredient, a CRISPR-Cas system or component thereof described in greater detail elsewhere herein. In some embodiments, the pharmaceutical formulation can include, such as an active ingredient, a CRISPR-Cas polynucleotide described in greater detail elsewhere herein. In some embodiments, the pharmaceutical formulation can include, such as an active ingredient one or more modified cells, such as one or more modified cells described in greater detail elsewhere herein.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a Type-2 Diabetes or a symptom thereof. In some embodiments, the subject in need thereof has or is suspected of having, a metabolic disease or disorder, insulin resistance, or glucose intolerance, or a combination thereof. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g., polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects. In some embodiments, the one or more therapeutic effects are promoting actin cytoskeleton remodeling processes, promoting accumulation of lipids in targeted cells, and promoting insulin-sensitivity.

The effective amount, least effective amount, and/or therapeutically effective amount of the primary and optional secondary active agent described elsewhere herein contained in the pharmaceutical formulation can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, g, mg, or g or be any numerical value with any of these ranges.

In some embodiments, the effective amount, least effective amount, and/or therapeutically effective amount can be an effective concentration, least effective concentration, and/or therapeutically effective concentration, which can each range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, M, mM, or M or be any numerical value with any of these ranges.

In other embodiments, the effective amount, least effective amount, and/or therapeutically effective amount of the primary and optional secondary active agent can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, the primary and/or the optional secondary active agent present in the pharmaceutical formulation can range from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In some embodiments where a cell population is present in the pharmaceutical formulation (e.g., as a primary and/or or secondary active agent), the effective amount of cells can range from about 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL.

In some embodiments, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In some embodiments, the effective amount can be 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more.

In some embodiments, the amount or effective amount of the one or more of the active agent(s) described herein contained in the pharmaceutical formulation can range from about 1 μg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total secondary active agent in the pharmaceutical formulation. In additional embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy,” 20th ed., Lippincott Williams & Wilkins, Baltimore, MD, 2000, and “Pharmaceutical dosage forms and drug delivery systems,” 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D5o value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, manitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein can be part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

In some embodiments, the composition and formulations of the present disclosure can be a co-therapy to an adoptive cell therapy described elsewhere herein, including but not limited to an engineered T cell therapy.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Viral Vector Formulation, Dosage, and Delivery

Compositions of the disclosure may be formulated for delivery to human subjects, as well as to animals for veterinary purposes (e.g., livestock (cattle, pigs, others)), and other non-human mammalian subjects. The dosage of the formulation can be measured or calculated as viral particles or as genome copies (“GC”)/viral genomes (“vg”). Any method known in the art can be used to determine the genome copy (GC) number of the viral compositions of the disclosure. In one example embodiment, the viral compositions can be formulated in dosage units to contain an amount of viral vectors that is in the range of about 1.0×10⁹ GC to about 1.0×10¹⁵ C (to treat an average subject of 70 kg in body weight), and preferably 1.0×10¹² C to 1.0×10¹⁴ C for a human patient. Preferably, the dose of virus in the formulation is 1.0×10⁹ C, 5.0×10⁹ C, 1.0×10¹⁰ C, 5.0×10¹⁰ C, 1.0×10¹¹ C, 5.0×10¹¹ C, 1.0×10¹² C, 5.0×10¹² C, or 1.0×10¹³ C, 5.0×10 C, 1.0×10¹⁴ C, 5.0×10¹⁴ C, or 1 0.0×10¹⁵C.

The viral vectors can be formulated in a conventional manner using one or more physiologically acceptable carriers or excipients. The viral vectors may be formulated for parenteral administration by injection (e.g., by bolus injection or continuous infusion). Formulations for injection may be presented in unit dosage form (e.g., in ampoules or in multi-dose containers) with an added preservative. The viral compositions may take such forms as suspensions, solutions, or emulsions in oily or aqueous vehicles, and may contain formulatory agents such as suspending, stabilizing, or dispersing agents. Liquid preparations of the viral vector formulations may be prepared by conventional means with pharmaceutically acceptable additives such as suspending agents (e.g., sorbitol syrup, cellulose derivatives or hydrogenated edible fats), emulsifying agents (e.g., lecithin or acacia), non-aqueous vehicles (e.g., almond oil, oily esters, ethyl alcohol or fractionated vegetable oils), and preservatives (e.g., methyl or propyl-p-hydroxybenzoates or sorbic acid). The preparations may also contain buffer salts. Alternatively, the compositions may be in powder form for constitution with a suitable vehicle (e.g., sterile pyrogen-free water) before use.%

### Recombinant Protein Formulation, Dosage, and Delivery

In one example embodiment, virus like particles (VLPs) are used to facilitate intracellular recombinant protein therapy (see, e.g., WO 2020/252455A1, U.S. Ser. No. 10/577,397B2). In certain embodiments, VLPs include a Gag-METTL17 fusion protein. The Gag-METTL17 fusion protein may include a matrix protein, a capsid protein, and/or a nucleocapsid protein covalently linked to METTL17. In certain embodiments, the VLPs include a membrane comprising a phospholipid bilayer with one or more human endogenous retrovirus (HERV) derived ENV/glycoprotein(s) on the external side; a HERV-derived GAG protein in the VLP core, and a METTL17 fusion protein on the inside of the membrane, wherein METTL17 is fused to a human-endogenous GAG or other plasma membrane recruitment domain (see, e.g., WO 2020/252455A1). Fusion proteins can be obtained using standard recombinant protein technology.

In one example embodiment, cell-penetrating peptides (CPPs) are used to facilitate intracellular recombinant protein therapy (see, e.g., Dinca A, Chien W-M, Chin M T. Intracellular Delivery of Proteins with Cell-Penetrating Peptides for Therapeutic Uses in Human Disease. International Journal of Molecular Sciences. 2016; 17(2):263). In certain embodiments, cell-penetrating peptides can be conjugated to METTL17, for example, using standard recombinant protein technology. In certain embodiments, cell-penetrating peptides can be concurrently delivered with a recombinant METTL17.

In one example embodiment, nanocarriers are used to facilitate intracellular recombinant protein therapy (see, e.g., Lee Y W, Luther D C, Kretzmann J A, Burden A, Jeon T, Zhai S, Rotello V M. Protein Delivery into the Cell Cytosol using Non-Viral Nanocarriers. Theranostics 2019; 9(11):3280-3292). Non-limiting nanocarriers include, but are not limited to nanoparticles (e.g., silica, gold), polymers, lipid based (e.g., cationic lipid within a polymer shell, lipid-like nanoparticles).

The pharmaceutical composition of the disclosure may be administered locally or systemically. In a preferred embodiment, the pharmaceutical composition is administered near the tissue whose cells are to be transduced. In a particular embodiment, the pharmaceutical composition of the disclosure is administered locally to the subcutaneous tissue. In another preferred embodiment, the pharmaceutical composition of the disclosure is administered systemically.

The “adeno-associated virus” (AAV) can be formulated with a physiologically acceptable carrier for use in gene transfer and gene therapy applications. The dosage of the formulation can be measured or calculated as viral particles or as genome copies (“GC”)/viral genomes (“vg”). Any method known in the art can be used to determine the genome copy (GC) number of the viral compositions of the disclosure. One method for performing AAV GC number titration is as follows: purified AAV vector samples are first treated with DNase to eliminate un-encapsulated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome.

In any of the described methods the one or more vectors may be comprised in a delivery system. In any of the described methods the vectors may be delivered via liposomes, particles (e.g., nanoparticles), exosomes, microvesicles, a gene-gun. In any of the described methods viral vectors may be delivered by transduction of viral particles. The delivery systems may be administered systemically or by localized administration (e.g., direct injection). The term “systemically administered” and “systemic administration,” as used herein, means that the polynucleotides, vectors, polypeptides, or pharmaceutical compositions of the disclosure are administered to a subject in a non-localized manner. The systemic administration of the polynucleotides, vectors, polypeptides, or pharmaceutical compositions of the disclosure may reach several organs or tissues throughout the body of the subject or may reach specific organs or tissues of the subject. For example, the intravenous administration of a pharmaceutical composition of the disclosure may result in the transduction of more than one tissue or organ in a subject. The term “transduce” or “transduction,” as used herein, refers to the process whereby a foreign nucleotide sequence is introduced into a cell via a viral vector. The term “transfection,” as used herein, refers to the introduction of DNA into a recipient eukaryotic cell.

Recombinant protein compositions described herein may be administered systemically (e.g., intravenously) or administered locally to a tissue (e.g., injection). In preferred embodiments, the recombinant protein compositions are administered with an appropriate carrier to be administered to a mammal, especially a human, preferably a pharmaceutically acceptable composition. A “pharmaceutically acceptable composition” refers to a non-toxic semisolid, liquid, or aerosolized filler, diluent, encapsulating material, colloidal suspension or formulation auxiliary of any type. Preferably, this composition is suitable for injection. These may be in particular isotonic, sterile, saline solutions (monosodium or disodium phosphate, sodium, potassium, calcium or magnesium chloride and similar solutions or mixtures of such salts), or dry, especially freeze-dried compositions which upon addition, depending on the case, of sterilized water or physiological saline, permit the constitution of injectable solutions.

### CRISPR-Cas Delivery

The CRISPR-Cas systems disclosed herein may be delivered using vectors comprising polynucleotides encoding the Cas polypeptide and the guide molecule. For HDR based embodiments, the donor template may also be encoded on a vector. Vectors, dosages, and tissue-specific configurations suitable for delivery of these components include those discussed above.

The vector(s) can include regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well-established cloning protocols and the teachings in this disclosure one skilled in the art can readily practice the compositions and methods of the present disclosure as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector is to express an array of promoter-RNAs separated by cleavable sequences in the intron of a coding sequence or gene; and, in this instance, it is advantageous to use a polymerase II promoter, which can have increased expression and enable the transcription of long RNA in a tissue specific manner. (see, e.g., Chung K H, Hart C C, Al-Bassam S, et al. Polycistronic RNA polymerase II expression vectors for RNA interference based on BIC/miR-155. Nucleic Acids Res. 2006; 34(7):e53). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters, especially as to the numbers of RNAs or guides discussed herein, without any undue experimentation.

The Cas polypeptide and guide molecule (and donor) may also be delivered as a preformed ribonucleoprotein complex (RNP). Delivery methods for delivery RNPs include virus like particles, cell-penetrating peptides, and nanocarriers discussed above.

Delivery mechanisms for CRISPRa systems include virus like particles, cell-penetrating peptides, and nanocarriers discussed above for CRISPR-Cas systems.

### Base Editing Delivery

Base editing systems may deliver on one or more vectors encoding the Cas-nucleobase deaminase and guide sequence. Vector systems suitable for this purpose includes those discussed above. Alternatively, base editing systems may be delivered as pre-complex Ribonucleoprotein complex (RNP). Systems for delving RNPs include the protein delivery systems: virus like particles; cell-penetrating peptides; and nanocarriers, discuss above.

A further example method for delivery of base-editing systems may include use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Engineered Cells

In another aspect, example embodiments are directed to isolated and modified cells comprising one or more modifications that increase methyltransferase like 17 (METTL17) gene and/or METTL17 protein expression and/or activity. In one example embodiment, the modified cell may be obtained by modified an isolated cell using any of the compositions disclosed above. Accordingly, the modified cell may comprise one or more modification that result in addition provision of an additional copy of a polynucleotide encoding METTL17 protein, single base pair edits, insertions or substitutions to an enhancer region of METT117 gene, or a combination thereof.

Further intended are isolated human cells or tissues, plants or non-human animals comprising one or more of the polynucleotide molecules, vectors, vector systems, or cells described in any of the embodiments herein. In an aspect, host cells and cell lines modified by or comprising the compositions, systems or modified enzymes of present disclosure are provided, including (isolated) stem cells, and progeny thereof.

In one embodiment, the plants or non-human animals comprise at least one of the system components, polynucleotide molecules, vectors, vector systems, or cells described in any of the embodiments herein at least one tissue type of the plant or non-human animal. In one embodiment, non-human animals comprise at least one of the system components, polynucleotide molecules, vectors, vector systems, or cells described in any of the embodiments herein in at least one tissue type. In one embodiment, the presence of the system components is transient, in that they are degraded overtime. In one embodiment, expression of the components of the systems and compositions described in any of the embodiments comprised in polynucleotide molecules, vectors, vector systems, or cells is limited to certain tissue types or regions in the plant or non-human animal. In one embodiment, the expression of the components of the systems and compositions described in any of the embodiments comprised in polynucleotide molecules, vectors, vector systems, or cells is dependent of a physiological cue. In one embodiment, expression of the components of the systems and compositions described in any of the embodiments comprised in polynucleotide molecules, vectors, vector systems, or cells may be triggered by an exogenous molecule. In one embodiment, expression of the components of the systems and compositions described in any of the embodiments comprised in polynucleotide molecules, vectors, vector systems, or cells is dependent on the expression of a non-METTL17 molecule in the plant or non-human animal.

### Engineered Cells for Adoptive Cell Therapy

The compositions, systems, and components thereof described herein can be used to modify cells for an adoptive cell therapy. In an aspect of the disclosure, methods and compositions which involve editing a target nucleic acid sequence, or modulating expression of a target nucleic acid sequence, and applications thereof in connection with cancer immunotherapy are comprehended by adapting the composition, system, of the present disclosure. In some examples, the compositions, systems, and methods may be used to modify a stem cell (e.g., induced pluripotent cell) to derive modified natural killer cells, gamma delta T cells, and alpha beta T cells, which can be used for the adoptive cell therapy. In certain examples, the compositions, systems, and methods may be used to modify modified natural killer cells, gamma delta T cells, and alpha beta T cells. In one example embodiment is an engineered CAR-T cell, a CAR-NK cell, a TCR-T cell, or a tumor infiltrating lymphocyte (TIL).

As used herein, “ACT,” “adoptive cell therapy,” and “adoptive cell transfer” may be used interchangeably. In one embodiment, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 Jun. 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In one embodiment, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the disclosure involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In one embodiment, an antigen (such as a tumor antigen) to be targeted in adoptive cell therapy (such as particularly CAR or TCR T-cell therapy) of a disease (such as particularly of tumor or cancer) may be selected from a group consisting of: MR1 (see, e.g., Crowther, et al., 2020, Genome-wide CRISPR-Cas9 screening reveals ubiquitous T cell cancer targeting via the monomorphic MHC class I-related protein MR1, Nature Immunology volume 21, pages 178-185), B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); x-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD138; CD44v6; B7H3 (CD276); KIT (CD 117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY—BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In one embodiment, an antigen to be targeted in adoptive cell therapy (such as particularly CAR-T, CAR-NK or TCR T-cell therapy) of a disease (such as particularly of tumor or cancer) is a tumor-specific antigen (TSA).

In one embodiment, an antigen to be targeted in adoptive cell therapy (such as particularly CAR-T, CAR-NK or TCR T-cell therapy) of a disease (such as particularly of tumor or cancer) is a neoantigen.

In one embodiment, an antigen to be targeted in adoptive cell therapy (such as particularly CAR-T, CAR-NK or TCR T-cell therapy) of a disease (such as particularly of tumor or cancer) is a tumor-associated antigen (TAA).

In one embodiment, an antigen to be targeted in adoptive cell therapy (such as particularly CAR-T, CAR-NK or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (Dl), and any combinations thereof.

In one embodiment, an antigen (such as a tumor antigen) to be targeted in adoptive cell therapy (such as particularly CAR-T, CAR-NK or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR a and R chains with selected peptide specificity (see U.S. Pat. No. 8,697,854; PCT Patent Publications: WO 2003/020763, WO 2004/033685, WO 2004/044004, WO 2005/114215, WO 2006/000830, WO 2008/038002, WO 2008/039818, WO 2004/074322, WO 2005/113595, WO 2006/125962, WO 2013/166321, WO 2013/039889, WO 2014/018863, WO 2014/083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO 92/15322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, In one embodiment, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD3ζ or FcRγ (scFv-CD3ζ or scFv-FcRγ; see U.S. Pat. Nos. 7,741,465; 5,912,172; U.S. Pat. No. 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI 1a-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO 2014/134165; PCT Publication No. WO 2012/079000). In one embodiment, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon Rib), CD79a, CD79b, Fc gamma RIIa, DAP10, and DAP12. In certain preferred embodiments, the primary signaling domain comprises a functional signaling domain of CD3ζ or FcRγ. In one embodiment, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In one embodiment, the one or more costimulatory signaling domains comprise a functional signaling domain of a protein selected, each independently, from the group consisting of: 4-1BB, CD27, and CD28. In one embodiment, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139. Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, co-stimulation may be orchestrated by expressing CARs in antigen-specific T cells, chosen so as to be activated and expanded following engagement of their native αβTCR, for example by antigen on professional antigen-presenting cells, with attendant co-stimulation. In addition, additional engineered receptors may be provided on the immunoresponsive cells, for example to improve targeting of a T-cell attack and/or minimize side effects.

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-(molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ. I.D. No. 53) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in one embodiment, cells intended for adoptive cell therapies, more particularly immunoresponsive cells such as T cells, may express the FMC63-28Z CAR as described by Kochenderfer et al. (supra). Hence, in one embodiment, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY (SEQ ID NO: 53) and continuing all the way to the carboxy-terminus of the protein. Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in International Patent Publication No. WO 2015/187528. More particularly Example 1 and Table 1 of WO 2015/187528, incorporated by reference herein, demonstrate the generation of anti-CD19 CARs based on a fully human anti-CD19 monoclonal antibody (47G4, as described in US 2010/0104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signaling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcεRI gamma chain; or CD28-FcεRI gamma chain) were disclosed. Hence, in one embodiment, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO 2015/187528 and an intracellular T-cell signaling domain as set forth in Table 1 of International Application No. WO 2015/187528. Preferably, the antigen is CD19, more preferably the antigen-binding element is an anti-CD19 scFv, even more preferably the mouse or human anti-CD19 scFv as described in Example 1 of WO 2015/187528. In one embodiment, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO 2015/187528.

By means of an example and without limitation, chimeric antigen receptor that recognizes the CD70 antigen is described in WO 2012/058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J. Pathol. 1995; 147: 1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US 2016/0046724A1; WO 2016/014789A2; WO 2017/211900A1; WO 2015/158671A1; US 2018/0085444A1; WO 2018/028647A1; US 2017/0283504A1; and WO 2013/154760A1).

In one embodiment, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In one embodiment, the chimeric inhibitory receptor comprises an extracellular antigen-binding element (or portion or domain) configured to specifically bind to a target antigen, a transmembrane domain, and an intracellular immunosuppressive or repressive signaling domain. In one embodiment, the second target antigen is an antigen that is not expressed on the surface of a cancer cell or infected cell or the expression of which is downregulated on a cancer cell or an infected cell. In one embodiment, the second target antigen is an MHC-class I molecule. In one embodiment, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in one embodiment, TCR expression may eliminated using RNA interference (e.g., nucleic acid component, siRNA, miRNA, etc.), METTL17 polypeptide, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US Patent Publication Nos. US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (International Patent Publication No. WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In one embodiment, ACT includes co-transferring CD4+ Th1 cells and CD8+ CTLs to induce a synergistic antitumor response (see, e.g., Li et al., Adoptive cell therapy with CD4+ T helper 1 cells and CD8+ cytotoxic T cells enhances complete rejection of an established tumor, leading to generation of endogenous memory responses to non-targeted tumor epitopes. Clin Transl Immunology. 2017 Oct. 6(10): e160).

In one embodiment, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4+ T cells that express a TCR recognizing tyrosinase tumor antigen. Exploitation of the TCR leads to rapid expansion of Th17 populations to large numbers ex vivo for reinfusion into the autologous tumor-bearing hosts.

In one embodiment, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In one embodiment, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In one embodiment, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells, or population of cells, may be made resistant to at least one immunosuppressive agent due to the inactivation of a gene encoding a receptor for such immunosuppressive agent. In one embodiment, the immunosuppressive treatment provides for the selection and expansion of the immunoresponsive T cells within the patient.

In one embodiment, the treatment can be administered before primary treatment (e.g., surgery or radiation therapy) to shrink a tumor before the primary treatment. In another embodiment, the treatment can be administered after primary treatment to remove any remaining cancer cells.

In one embodiment, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In one embodiment, the disclosed CARs may be delivered or administered into a cavity formed by the resection of tumor tissue (i.e., intracavity delivery) or directly into a tumor prior to resection (i.e., intratumoral delivery). In one embodiment, the cell compositions of the present disclosure are preferably administered by intravenous injection.

The administration of the cells or population of cells can consist of the administration of 104-109 cells per kg body weight, preferably 105 to 106 cells/kg body weight including all integer values of cell numbers within those ranges. Dosing in CAR-T, CAR-NK, TCR-T cell therapies may for example involve administration of from 106 to 109 cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; International Patent Publication WO 2011/146862; International Patent Publication WO 2014/011987; International Patent Publication WO 2013/040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25; 9(374); Legut, et al., 2018, CRISPR-mediated TCR replacement generates superior anticancer transgenic T cells. Blood, 131(3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. The composition and systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g., TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see International Patent Publication Nos. WO 2013/176915, WO 2014/059173, WO 2014/172606, WO 2014/184744, and WO 2014/191128).

In one embodiment, editing may result in inactivation of a gene. By inactivating a gene, it is intended that the gene of interest is not expressed in a functional protein form. In a particular embodiment, the system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In one embodiment, homology directed repair (HDR) is used to concurrently inactivate a gene (e.g., TRAC) and insert an endogenous TCR or CAR into the inactivated locus.

Hence, in one embodiment, editing of cells, particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable “safe harbor” loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in one embodiment, editing of cells, particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as METTL17 overexpression system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present disclosure further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present disclosure allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In one embodiment, editing of cells, particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In one embodiment, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy?Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

International Patent Publication No. WO 2014/172606 relates to the use of MT 1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In one embodiment, metallothioneins are targeted by gene editing in adoptively transferred T cells.

In one embodiment, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, International Patent Publication No. WO 2016/196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO 2015/142675 relates to immune effector cells comprising a CAR in combination with an agent (such as the composition or system herein) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, 3-2 microglobulin (B2M) and PDi simultaneously, to generate gene-disrupted allogeneic CAR T cells deficient of TCR, HLA class I molecule and PD1.

In one embodiment, cells may be engineered to express a CAR, wherein expression and/or function of methylcytosine dioxygenase genes (TET1, TET2 and/or TET3) in the cells has been reduced or eliminated, (such as the composition or system herein) (for example, as described in WO 2017/04916).

In one embodiment, editing of cells, particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In one embodiment, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WTi), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in International Patent Publication Nos. WO 2016/011210 and WO 2017/011804).

In one embodiment, editing of cells, particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas mRNA and gRNAs targeting endogenous TCR, 3-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In one embodiment, a cell may be multiplied edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment, T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In one embodiment, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In one embodiment of the present disclosure, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the disclosure, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed, and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (e.g., CD14+ cells) may be depleted from blood preparations by a variety of methodologies, including anti-CD14 coated beads or columns, or utilization of the phagocytotic activity of these cells to facilitate removal. Accordingly, in one embodiment, the disclosure uses paramagnetic particles of a size sufficient to be engulfed by phagocytotic monocytes. In one embodiment, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In one embodiment, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In one embodiment, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells are minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×106/ml. In other embodiments, the concentration used can be from about 1x 105/ml to 1x 106/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present disclosure may also be antigen-specific T cells. For example, tumor-specific T cells can be used. In one embodiment, antigen-specific T cells can be isolated from a patient of interest, such as a patient afflicted with a cancer or an infectious disease. In one embodiment, neoepitopes are determined for a subject, and T cells specific to these antigens are isolated. Antigen-specific cells for use in expansion may also be generated in vitro using any number of methods known in the art, for example, as described in U.S. Patent Publication No. US 2004/0224402 entitled, Generation and Isolation of Antigen-Specific T Cells, or in U.S. Pat. No. 6,040,177. Antigen-specific cells for use in the present disclosure may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g., via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of 1251 labeled p2-microglobulin (02m) into MHC class I/02m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present disclosure, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO 2014/133567 and WO 2014/133568, herein incorporated by reference in their entirety. Additionally, activated T cells can be selected for based on surface expression of CD107a.

In one embodiment of the disclosure, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003/057171, U.S. Pat. No. 8,034,334, and U.S. Patent Publication No. 2012/0244133, each of which is incorporated herein by reference.

In one embodiment, ex vivo T cell expansion can be performed by isolation of T cells and subsequent stimulation or activation followed by further expansion. In one embodiment of the disclosure, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In one embodiment, T cells comprising a CAR or an exogenous TCR, may be manufactured as described in International Patent Publication No. WO 2015/120096, by a method comprising enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In one embodiment, T cells comprising a CAR or an exogenous TCR, may be manufactured as described in WO 2015/120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In one embodiment, T cell maturation or differentiation in vitro may be delayed or inhibited by the method as described in International Patent Publication No. WO 2017/070395, comprising contacting one or more T cells from a subject in need of a T cell therapy with an AKT inhibitor (such as, e.g., one or a combination of two or more AKT inhibitors disclosed in claim 8 of WO 2017/070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In one embodiment, a patient in need of a T cell therapy may be conditioned by a method as described in International Patent Publication No. WO 2016/191756 comprising administering to the patient a dose of cyclophosphamide between 200 mg/m2/day and 2000 mg/m2/day and a dose of fludarabine between 20 mg/m2/day and 900 mg/m2/day.

### Engineered Stem Cells

In another example embodiment, the isolated cell may be pluripotent stem cell or induced pluripotent stem cells (iPSC). Pluripotent stem cells have a number of potential uses including deriving cells for adoptive cell therapy as described above. In addition, recent research has shown that inducing cell to express the Yamanaka factors can rewind many of the molecular hallmarks of aging and render such treated cells nearly indistinguishable from younger pluripotent cells. Such reprogrammed cells have a number of therapeutic uses from reversing the effects of aging to treating other diseases. For example, osteoarthritic cells isolated from subjects with osteoarthritis have been reprogrammed using Yamanaka factors and resulted in cells with reduced secretion of inflammatory molecules and an improved ability to divide and function. Sarkar et al. “Transient non-integrative expression of nuclear reprogramming factors promotes multifaceted amelioration of aging in human cells” 11 Nature Communications 1545 (2020). Accordingly, modified pluripotent stems possessing modifications to increase METTL17 expression or activity may be used as a stand-alone treatment in a similar fashion, or in combination with other modifications to render new and useful therapeutics.

### Engineered Gametes for Enhanced Fertilization

The respiration demands on spermatozoa and oocytes in the immediate events leading up to fertilization, during, and through early embryonic development are dependent at least in part on adequate cellular respiration. For example, sperm cells have increased demand to propel themselves through the female reproductive track to reach the oocyte for fertilization. The oocyte must undergo many cellular processes upon contact with a sperm to facilitate entry of the sperm into the oocyte and zygote formation. Accordingly, in one example embodiment, the engineered cells are an engineered gamete. In one example embodiment, the one or more modifications do not modify the genome of a human gamete. In some of these embodiments the cells are spermatids, oogonia, oocytes, or spermatozoa. In some embodiments, non-human animal spermatids, oogonia, oocytes, or spermatozoa comprise one or more compositions of the present disclosure. Such cells can have improved respiration and thus the inventive compositions herein can be useful for improving fertilization during natural or in vitro fertilization. In some embodiments, the compositions delivered to spermatozoa are mRNA or protein compositions as spermatozoa generally do not carry out transcription. In some embodiments, the inventive compositions can be included in a formulation adapted for culturing, storing, extending, diluting, or otherwise containing spermatozoa or oocytes. Such formulations, in some embodiments, can also include one or more spermatozoa or oocytes.

Methods for Enhancing Intra-Mitochondrial Protein Translation and/or OXPHOS Activity

In another aspects, embodiments disclosed herein are directed to methods of enhancing intra-mitochondrial protein translation and/or OXPHOS activity. In one example embodiment, the method comprises administration of one or more of the compositions disclosed herein, a polynucleotide disclosed herein, a delivery system disclosed herein, or an engineered cell disclosed herein. The potential applications include treatment of age-related mitochondrial dysfunction or decreased activity not associated with mitochondrial diseases, as well as use in treating mitochondrial diseases in a manner that is agnostic to the underlying genetic cause of the mitochondrial disease.

In general, the method can include delivering a composition, system, and/or component thereof to a subject or cell thereof, or to an infectious or symbiotic organism by a suitable delivery technique and/or composition. Once administered the components can operate as described elsewhere herein to elicit a nucleic acid modification event. In some aspects, the nucleic acid modification event can occur at the genomic, epigenomic, and/or transcriptomic level. DNA and/or RNA cleavage, gene activation, and/or gene deactivation can occur. Additional features, uses, and advantages are described in greater detail below. On the basis of this concept, several variations are appropriate to elicit a genomic locus event, including DNA cleavage, gene activation, or gene deactivation. Using the provided compositions, the person skilled in the art can advantageously and specifically target single or multiple loci with the same or different functional domains to elicit one or more genomic locus events. In addition to treating and/or preventing a disease in a subject, the compositions may be applied in a wide variety of methods for screening in libraries in cells and functional modeling in vivo (e.g., gene activation of lincRNA and identification of function; gain-of-function modeling; loss-of-function modeling; the use the compositions of the disclosure to establish cell lines and transgenic animals for optimization and screening purposes).

The composition, system, and components thereof described elsewhere herein can be used to treat and/or prevent a disease, such as a genetic and/or epigenetic disease, in a subject. The composition, system, and components thereof described elsewhere herein can be used to treat and/or prevent genetic infectious diseases in a subject, such as bacterial infections, viral infections, fungal infections, parasite infections, and combinations thereof. The composition, system, and components thereof described elsewhere herein can be used to modify the composition or profile of a microbiome in a subject, which can in turn modify the health status of the subject. The composition, system, described herein can be used to modify cells ex vivo, which can then be administered to the subject whereby the modified cells can treat or prevent a disease or symptom thereof. This is also referred to in some contexts as adoptive therapy. The composition, system, and components thereof described herein can be used to treat mitochondrial diseases, where the mitochondrial disease etiology involves a mutation in the mitochondrial DNA.

Also provided is a method of treating a subject, e.g., a subject in need thereof, comprising inducing gene editing by transforming the subject with the polynucleotide encoding one or more components of the composition, system, or complex or any of polynucleotides or vectors described herein, and administering them to the subject. A suitable repair template may also be provided, for example delivered by a vector comprising said repair template. The repair template may be a recombination template herein. Also provided is a method of treating a subject, e.g., a subject in need thereof, comprising inducing transcriptional activation or repression of multiple target gene loci by transforming the subject with the polynucleotides or vectors described herein, wherein said polynucleotide or vector encodes or comprises one or more components of composition, system, complex or component thereof comprising multiple METTL17 polypeptides. Where any treatment is occurring ex vivo, for example in a cell culture, then it will be appreciated that the term “subject” may be replaced by the phrase “cell or cell culture.”

Also provided is a method of treating a subject, e.g., a subject in need thereof, comprising inducing gene editing by transforming the subject with the METTL17 polypeptide(s), advantageously encoding and expressing in vivo the remaining portions of the composition, system, (e.g., RNA), complex or component thereof. A suitable repair template may also be provided, for example delivered by a vector comprising said repair template. Also provided is a method of treating a subject, e.g., a subject in need thereof, comprising inducing transcriptional activation or repression by transforming the subject with the METTL17 polypeptide(s) advantageously encoding and expressing in vivo the remaining portions of the composition, system, (e.g., nucleic acid component molecule), complex or component thereof. In one embodiment the METTL17 polypeptide is a catalytically inactive METTL17 polypeptide and includes one or more associated functional domains. Where any treatment is occurring ex vivo, for example in a cell culture, then it will be appreciated that the term “subject” may be replaced by the phrase “cell or cell culture.”

One or more components of the composition and system described herein can be included in a composition, such as a pharmaceutical composition, and administered to a host individually or collectively. Alternatively, these components may be provided in a single composition for administration to a host. Administration to a host may be performed via viral vectors known to the skilled person or described herein for delivery to a host (e.g., lentiviral vector, adenoviral vector, AAV vector). As explained herein, use of different selection markers (e.g., for lentiviral nucleic acid component selection) and concentration of nucleic acid component (e.g., dependent on whether multiple nucleic acid components are used) may be advantageous for eliciting an improved effect.

Thus, also described herein are methods of inducing one or more polynucleotide modifications in a eukaryotic or prokaryotic cell or component thereof (e.g., a mitochondria) of a subject, infectious organism, and/or organism of the microbiome of the subject. The modification can include the introduction, deletion, or substitution of one or more nucleotides at a target sequence of a polynucleotide of one or more cell(s). The modification can occur in vitro, ex vivo, in situ, or in vivo.

In one embodiment, the method of treating or inhibiting a condition or a disease caused by one or more mutations in a genomic locus in a eukaryotic organism or a non-human organism can include manipulation of a target sequence within a coding, non-coding or regulatory element of said genomic locus in a target sequence in a subject or a non-human subject in need thereof comprising modifying the subject or a non-human subject by manipulation of the target sequence and wherein the condition or disease is susceptible to treatment or inhibition by manipulation of the target sequence including providing treatment comprising delivering a composition comprising the particle delivery system or the delivery system or the virus particle of any one of the above embodiments or the cell of any one of the above embodiments.

Also provided herein is the use of the particle delivery system or the delivery system or the virus particle of any one of the above embodiments or the cell of any one of the above embodiments in ex vivo or in vivo gene or genome editing; or for use in in vitro, ex vivo or in vivo gene therapy. Also provided herein are particle delivery systems, non-viral delivery systems, and/or the virus particle of any one of the above embodiments or the cell of any one of the above embodiments used in the manufacture of a medicament for in vitro, ex vivo or in vivo gene or genome editing or for use in in vitro, ex vivo or in vivo gene therapy or for use in a method of modifying an organism or a non-human organism by manipulation of a target sequence in a genomic locus associated with a disease or in a method of treating or inhibiting a condition or disease caused by one or more mutations in a genomic locus in a eukaryotic organism or a non-human organism.

In one embodiment, polynucleotide modification can include the introduction, deletion, or substitution of 1-75 nucleotides at each target sequence of said polynucleotide of said cell(s). The modification can include the introduction, deletion, or substitution of at least 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence. The modification can include the introduction, deletion, or substitution of at least 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of at least 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of at least 20, 21, 22, 23, 24, 25, 26, 27, 28, 29, 30, 35, 40, 45, 50, or 75 nucleotides at each target sequence of said cell(s). The modification can include the introduction, deletion, or substitution of at least 40, 45, 50, 75, 100, 200, 300, 400 or 500 nucleotides at each target sequence of said cell(s). The modification can include the introduction, deletion, or substitution of at least 500, 600, 700, 800, 900, 1000, 1100, 1200, 1300, 1400, 1500, 1600, 1700, 1800, 1900, 2000, 2100, 2200, 2300, 2400, 2500, 2600, 2700,2800,2900,3000,3100,3200,3300,3400,3500,3600,3700,3800, 3900, 4000, 4100,4200, 4300, 4400, 4500, 4600, 4700,4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, or 9900 to 10000 nucleotides at each target sequence of said cell(s).

In one embodiment, the modifications can include the introduction, deletion, or substitution of nucleotides at each target sequence of said cell(s) via nucleic acid components (e.g., nucleic acid component molecule(s) RNA(s) or nucleic acid component(s)), such as those mediated by a composition, system, or a component thereof described elsewhere herein. In one embodiment, the modifications can include the introduction, deletion, or substitution of nucleotides at a target or random sequence of said cell(s) via a composition, system, or technique.

In one embodiment, the composition, system, or component thereof can promote Non-Homologous End-Joining (NHEJ). In one embodiment, modification of a polynucleotide by a composition, system, or a component thereof, such as a diseased polynucleotide, can include NHEJ. In one embodiment, promotion of this repair pathway by the composition, system, or a component thereof can be used to target gene or polynucleotide specific knock-outs and/or knock-ins. In one embodiment, promotion of this repair pathway by the composition, system, or a component thereof can be used to generate NHEJ-mediated indels. Nuclease-induced NHEJ can also be used to remove (e.g., delete) a sequence in a gene of interest. Generally, NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. The indel can range in size from 1-50 or more base pairs. In one embodiment the indel can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 base pairs or more. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences.

In one embodiment, composition, system, mediated NHEJ can be used in the method to delete small sequence motifs. In one embodiment, composition, system, mediated NHEJ can be used in the method to generate NHEJ-mediate indels that can be targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp). In an embodiment, in which a nucleic acid component and METTL17 polypeptide generate a double strand break for the purpose of inducing NHEJ-mediated indels, a nucleic acid component may be configured to position one double-strand break in close proximity to a nucleotide of the target position. In an embodiment, the cleavage site may be between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position). In an embodiment, in which two component RNAs complexing with one or more nickases induce two single strand breaks for the purpose of inducing NHEJ-mediated indels, two component RNAs may be configured to position two single-strand breaks to provide for NHEJ repair a nucleotide of the target position.

For minimization of toxicity and off-target effect, it may be important to control the concentration of METTL17 polypeptide mRNA and component RNA delivered. Optimal concentrations of METTL17 polypeptide mRNA and component RNA can be determined by testing different concentrations in a cellular or non-human eukaryote animal model and using deep sequencing the analyze the extent of modification at potential off-target genomic loci. Alternatively, to minimize the level of toxicity and off-target effect, nickase mRNA (for example a mutated METTL17) can be delivered with a pair of nucleic acid components targeting a site of interest.

Typically, in the context of an endogenous METTL17 polypeptide, formation of a METTL17 polypeptide or complex (comprising a polynucleotide component sequence hybridized to a target sequence and complexed with one or more METTL17 polypeptides) results in cleavage, nicking, and/or another modification of one or both strands in or near (e.g., within 1, 2, 3, 4, 5, 6, 7, 8, 9, 10, 20, 50, or more base pairs from) the target sequence.

In one embodiment, a method of modifying a target polynucleotide in a cell to treat or prevent a disease can include allowing a composition, system, or component thereof to bind to the target polynucleotide, e.g., to effect cleavage, nicking, or other modification as the composition, system, is capable of said target polynucleotide, thereby modifying the target polynucleotide, wherein the composition, system, or component thereof, complex with a nucleic acid component molecule sequence, and hybridize said nucleic acid component molecule sequence to a target sequence within the target polynucleotide, wherein said nucleic acid component molecule sequence is optionally linked to a nucleic acid component scaffold sequence. In some of these embodiments, the composition, system, or component thereof can be or include a METTL17 polypeptide complexed with a nucleic acid component molecule sequence. In one embodiment, modification can include cleaving or nicking one or two strands at the location of the target sequence by one or more components of the composition, system, or component thereof.

The cleavage, nicking, or other modification capable of being performed by the composition, system, can modify transcription of a target polynucleotide. In one embodiment, modification of transcription can include decreasing transcription of a target polynucleotide. In one embodiment, modification can include increasing transcription of a target polynucleotide. In one embodiment, the method includes repairing said cleaved target polynucleotide by homologous recombination with a recombination template polynucleotide, wherein said repair results in a modification such as, but not limited to, an insertion, deletion, or substitution of one or more nucleotides of said target polynucleotide. In one embodiment, said modification results in one or more amino acid changes in a protein expressed from a gene comprising the target sequence. In one embodiment, the modification imparted by the composition, system, or component thereof provides a transcript and/or protein that can correct a disease or a symptom thereof, including but not limited to, any of those described in greater detail elsewhere herein.

In one embodiment, the method of treating or preventing a disease can include delivering one or more vectors or vector systems to a cell, such as a eukaryotic or prokaryotic cell, wherein one or more vectors or vector systems include the composition, system, or component thereof. In one embodiment, the vector(s) or vector system(s) can be a viral vector or vector system, such as an AAV or lentiviral vector system, which are described in greater detail elsewhere herein. In one embodiment, the method of treating or preventing a disease can include delivering one or more viral particles, such as an AAV or lentiviral particle, containing the composition, system, or component thereof. In one embodiment, the viral particle has a tissue specific tropism. In one embodiment, the viral particle has a liver, muscle, eye, heart, pancreas, kidney, neuron, epithelial cell, endothelial cell, astrocyte, glial cell, immune cell, or red blood cell specific tropism.

It will be understood that the composition and system, according to the disclosure as described herein, such as the composition and system, for use in the methods according to the disclosure as described herein, may be suitably used for any type of application known for the composition or system, preferably in eukaryotes. In certain aspects, the application is therapeutic, preferably therapeutic in a eukaryote organism, such as including but not limited to animals (including human), plants, algae, fungi (including yeasts), etc. Alternatively, or in addition, in certain aspects, the application may involve accomplishing or inducing one or more particular traits or characteristics, such as genotypic and/or phenotypic traits or characteristics, as also described elsewhere herein.

### Methods of Treating Mitochondrial Diseases

In one example embodiment, a method of treating a mitochondrial disease comprises administering one or more of the compositions, polynucleotides, delivery systems, or engineered cells disclosed herein to a subject in need thereof. Mitochondrial dysfunctions are known to be responsible for a number of heterogenous clinical presentations with multi-systemic involvement. Impaired oxidative phosphorylation leading to a decrease in cellular energy (ATP) production is the most important cause underlying these diseases and disorders. Mitochondrial dysfunctions are associated with a large number of human diseases such as neurodegenerative disorders, cardiovascular disorder, neurometabolic diseases, cancer, and obesity. The mitochondrial disease may be a monogenic disease characterized by a defect in oxidative phosphorylation caused by pathogenic variants of over 300 known genes. These pathogenic variants may occur in nuclear DNA (nuDNA), mitochondrial (DNA) or a combination thereof. Mitochondrial disorders can also arise from secondary influences such as viral infections and off-target drug effects. Mitochondrial disease and disorder may also be heteroplasmic, that is a subject suffering from a mitochondrial disease may comprise a mixture of both wildtype and mutant mtDNA resulting in marked clinical heterogeneity across subjects. Given this disease complexity, a particular advantage of the present disclosure is the fact that the Applicant has demonstrated that METTL17 is limiting for intra-mitochondrial protein expression, and over-expressing METTL17 is sufficient to boost all 13 mtDNA encoded OXPHOS subunits, which then elevates the abundance of the entire OXPHOS systems and activity. Accordingly, the embodiments disclosed herein provide a method for treating mitochondrial diseases and disorders in a way that is agnostic to the underlying genetic cause of the disease or disorder.

In some embodiments, the mitochondrial disease that is treated is MELAS (mitochondrial myopathy encephalopathy, and lactic acidosis and stroke-like episodes), CPEO/PEO (chronic progressive external ophthalmoplegia syndrome/progressive external ophthalmoplegia), KSS (Kearns-Sayre syndrome), MIDD (maternally inherited diabetes and deafness), MERRF (myoclonic epilepsy associated with ragged red fibers), NIDDM (noninsulin-dependent diabetes mellitus), LHON (Leber hereditary optic neuropathy), LS (Leigh Syndrome) an aminoglycoside induced hearing disorder, NARP (neuropathy, ataxia, and pigmentary retinopathy), Extrapyramidal disorder with akinesia-rigidity, psychosis and SNHL, Nonsyndromic hearing loss, a cardiomyopathy, an encephalomyopathy, Pearson's syndrome, a disease identified as being caused or attributed to a mtDNA mutation set forth at mitomap.org, or a combination thereof.

In one example embodiment, the mitochondrial disease or disorder is characterized by mtDNA mutations is selected from the group consisting of: A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-tandem (SEQ ID NO: 54) repeats at positions 305-314 and/or 956-965, deletion at positions from 8,469-13,447, 4,308-14,874, and/or 4,398-14,822, 961ins/delC, the mitochondrial common deletion (e.g. mtDNA 4,977 bp deletion), and combinations thereof.

In another example embodiment, the mitochondrial disease or disorder is caused by one or more of the mutations shown in FIG. 3 of Frazer et al. “Mitochondrial energy generation disorders: genes, mechanisms, and clues to pathology” 294 Journal of Biological Chemistry Reviews, 5386-5395 (2019), which is incorporated herein by reference.

In another example embodiment, the mitochondrial mutation can be any mutation as set forth in or as identified by use of one or more bioinformatic tools available at Mitomap available at mitomap.org. Such tools include, but are not limited to, “Variant Search, aka Market Finder,” Find Sequences for Any Haplogroup, aka “Sequence Finder,” “Variant Info,” “POLG Pathogenicity Prediction Server,” “MITOMASTER,” “Allele Search,” “Sequence and Variant Downloads,” or “Data Downloads.” MitoMap contains reports of mutations in mtDNA that can be associated with disease and maintains a database of reported mitochondrial DNA Base Substitution Diseases: rRNA/tRNA mutations. In another example embodiment, the mutation can be a mutation shown in any of Tables 4-8 or a combination thereof.

TABLE 4
Exemplary mtDNA mutations.

| A | B | C | D | E | F | G | H | I | J | K | L |
|---|---|---|---|---|---|---|---|---|---|---|---|
| 582 | MT-TF | Mitochondrial myopathy | T582C | tRNA Phe | − | + | Reported | 72.90% | 0% | 0 | 2 |
| | | | | | | | | | (0%) | (0) | |
| 583 | MT-TF | MELAS/MM & EXIT | G583A | tRNA Phe | − | + | Cfrm | Pathogenic | 0% | 0 | 3 |
| | | | | | | | | | (0%) | (0) | |
| 586 | MT-TF | Extrapyramidal disorder | G586A | tRNA Phe | − | + | Reported | 89.70% | 0% | 0 | 2 |
| | | with akinesia-rigidity, | | | | | | | (0%) | (0) | |
| | | psychosis and SNHL | | | | | | | | | |
| 593 | MT-TF | Nonsyndromic hearing | T593C | tRNA Phe | + | − | Reported | 0.80% | 0.4% | 205 | 1 |
| | | loss | | | | | | | (0%) | (0) | |
| 602 | MT-TF | Axial myopathy with | C602T | tRNA Phe | − | + | Reported | 85.90% | 0% | 0 | 2 |
| | | encephalopathy | | | | | | | (0%) | (0) | |
| 606 | MT-TF | Myoglobinuria | A606G | tRNA Phe | + | + | Unclear | 64.90% | 0% | 15 | 3 |
| | | | | | | | | | (0%) | (0) | |
| 608 | MT-TF | Tubulo-interstitial | A608G | tRNA Phe | + | − | Reported | 65.00% | 0% | 0 | 2 |
| | | nephritis | | | | | | | (0%) | (0) | |
| 611 | MT-TF | MERRF | G611A | tRNA Phe | ′ | + | Reported | 51.20% | 0% | 0 | 3 |
| | | | | | | | | | (0%) | (0) | |
| 616 | MT-TF | Maternally inherited | T616C | tRNA Phe | + | + | Cfrm | Pathogenic | 0% | 1 | 2 |
| | | epilepsy/kidney disease | | | | | | | (0%) | (0) | |
| 616 | MT-TF | Maternally inherited | T616G | tRNA Phe | + | + | Reported | 95.60% | 0% | 1 | 1 |
| | | epilepsy | | | | | | | (0%) | (0) | |
| 617 | MT-TF | Carotid artery | G617A | tRNA Phe | − | + | Reported | 81.70% | 0% | 0 | 1 |
| | | stenosis | | | | | | | (0%) | (0) | |
| 618 | MT-TF | MM | T618C | tRNA Phe | − | + | Reported | 65.80% | 0% | 0 | 1 |
| | | | | | | | | | (0%) | (0) | |
| 618 | MT-TF | Ptosis CPEO | T618G | tRNA Phe | − | + | Reported | 77.50% | 0% | 0 | 1 |
| | | MM & EXIT | | | | | | | (0%) | (0) | |
| 622 | MT-TF | EXIT & Deafness | G622A | tRNA Phe | − | + | Reported | 41.50% | 0% | 0 | 2 |
| | | | | | | | | | (0%) | (0) | |
| 625 | MT-TF | SNHL & Epilepsy | G625A | tRNA Phe | − | + | Reported | 81.30% | 0% | 0 | 1 |
| | | | | | | | | | (0%) | (0) | |
| 628 | MT-TF | DEAF | C628T | tRNA Phe | − | + | Reported | 34.80% | 0% | 3 | 1 |
| | | | | | | | | | (0%) | (0) | |
| 636 | MT-TF | DEAF | A636G | tRNA Phe | + | − | Reported | 1.30% | 0% | 18 | 3 |
| | | | | | | | | | (0%) | (0) | |
| 641 | MT-TF | Epileptic Encephalopathy | A641T | tRNA Phe | − | + | Reported | 69.00% | 0% | 0 | 1 |
| | | | | | | | | | (0%) | (0) | |
| 642 | MT-TF | Ataxia, PEO, deafness | T642C | tRNA Phe | − | + | Reported | 67.60% | 0% | 0 | 1 |
| | | | | | | | | | (0%) | (0) | |
| 652 | MT- | Atherosclerosis risk | G652del | 12S rRNA | − | + | Reported | N/A | 0% | 0 | 2 |
| | RNR1 | | | | | | | | (0%) | (0) | |
| 652 | MT- | Atherosclerosis study | G652GG | 12S rRNA | . | . | Reported | N/A | 0% | 0 | 1 |
| | RNR1 | | | | | | | | (0%) | (0) | |
| 663 | MT- | Coronary atherosclerosis | A663G | 12S rRNA | + | − | Reported | N/A | 2.8% | 1404 | 1 |
| | RNR1 | risk | | | | | | | (0%) | (0) | |
| 669 | MT- | DEAF | T669C | 12S rRNA | + | − | Reported | N/A | 0.2% | 87 | 4 |
| | RNR1 | | | | | | | | (0%) | (0) | |
| 721 | MT- | Possibly LVNC- | T721C | 12S rRNA | + | − | Reported | N/A | 0.2% | 125 | 1 |
| | RNR1 | associated | | | | | | | (0%) | (0) | |
| 735 | MT- | DEAF | A735G | 12S rRNA | . | . | Reported | N/A | 0.1% | 73 | 1 |
| | RNR1 | | | | | | | | (0%) | (0) | |
| 745 | MT- | DEAF-associated | A745G | 12S rRNA | + | − | Reported | N/A | 0.1% | 32 | 1 |
| | RNR1 | | | | | | | | (0%) | (0) | |
| 750 | MT- | SZ-associated | A750A | 12S rRNA | + | − | Reported | N/A | 1.7% | 864 | 3 |
| | RNR1 | | | | | | | | (0%) | (0) | |
| 792 | MT- | Increased risk of | C792T | 12S rRNA | + | − | Reported | N/A | 0% | 4 | 1 |
| | RNR1 | nonsyndromic deafness | | | | | | | (0%) | (0) | |
| 801 | MT- | DEAF-associated | A801G | 12S rRNA | + | − | Reported | N/A | 0% | 6 | 1 |
| | RNR1 | | | | | | | | (0%) | (0) | |
| 827 | MT- | DEAF | A827G | 12S rRNA | + | − | Conflicting | N/A | 2.5% | 1276 | 16 |
| | RNR1 | | | | | | reports | | (0%) | (0) | |
| 839 | MT- | DEAF-associated | A839G | 12S rRNA | + | − | Reported | N/A | 0% | 6 | 1 |
| | RNR1 | | | | | | | | (0%) | (0) | |
| 850 | MT- | Possibly LVNC- | T850C | 12S rRNA | + | − | Reported | N/A | 0.2% | 123 | 1 |
| | RNR1 | associated | | | | | | | (0%) | (0) | |
| 856 | MT- | LHON helper/AD/ | A856G | 12S rRNA | + | − | Reported | N/A | 0% | 19 | 3 |
| | RNR1 | DEAF-associated | | | | | | | (0%) | (0) | |
| 869 | MT- | found in 1 HCM | C869T | 12S rRNA | + | − | Reported | N/A | 0.1% | 70 | 1 |
| | RNR1 | patient | | | | | | | (0%) | (0) | |
| 921 | MT- | Possibly LVNC- | T921C | 12S rRNA | + | − | Reported | N/A | 0.8% | 397 | 2 |
| | RNR1 | associated | | | | | | | (0%) | (0) | |
| 960 | MT- | Possibly DEAF- | C960del | 12S rRNA | + | − | Reported | N/A | 0% | 0 | 1 |
| | RNR1 | associated | | | | | | | (0%) | (0) | |
| 960 | MT- | Possibly DEAF- | C960CC | 12S rRNA | + | − | Reported | N/A | 0.6% | 282 | 4 |
| | RNR1 | associated | | | | | | | (0%) | (0) | |
| 961 | MT- | DEAF, possibly | T961C | 12S rRNA | + | − | Unclear | N/A | 0.9% | 442 | 7 |
| | RNR1 | LVNC-associated | | | | | | | (0%) | (0) | |
| 961 | MT- | DEAF/AD-associated/ | T961delT+/−C(n)ins | 12S rRNA | + | + | Unclear | N/A | 0% | 0 | 21 |
| | RNR1 | intellectual | | | | | | | (0%) | (0) | |
| | | disability | | | | | | | | | |
| 961 | MT- | Possibly DEAF- | T961G | 12S rRNA | + | − | Unclear | N/A | 0.4% | 189 | 6 |
| | RNR1 | associated | | | | | | | (0%) | (0) | |
| 961 | MT- | DEAF | T961TC | 12S rRNA | + | − | Unclear | N/A | 0% | 0 | 13 |
| | RNR1 | | | | | | | | (0%) | (0) | |
| 988 | MT- | Possible DEAF | G988A | 12S rRNA | . | . | Reported | N/A | 0.1% | 39 | 1 |
| | RNR1 | risk factor | | | | | | | (0%) | (0) | |
| 990 | MT- | DEAF | T990C | 12S rRNA | + | − | Reported | N/A | 0.1% | 33 | 1 |
| | RNR1 | | | | | | | | (0%) | (0) | |
| 1005 | MT- | DEAF | T1005C | 12S rRNA | + | − | Unclear | N/A | 0.5% | 230 | 5 |
| | RNR1 | | | | | | | | (0%) | (0) | |
| 1027 | MT- | DEAF-associated | A1027G | 12S rRNA | + | − | Reported | N/A | 0% | 14 | 1 |
| | RNR1 | | | | | | | | (0%) | (0) | |
| 1095 | MT- | SNHL | T1095C | 12S rRNA | + | + | Unclear | N/A | 0.1% | 62 | 15 |
| | RNR1 | | | | | | | | (0%) | (0) | |
| 1116 | MT- | DEAF | A1116G | 12S rRNA | + | − | Reported | N/A | 0% | 10 | 2 |
| | RNR1 | | | | | | | | (0%) | (0) | |
| 1180 | MT- | Possibly DEAF- | T1180G | 12S rRNA | + | − | Reported | N/A | 0% | 0 | 2 |
| | RNR1 | associated | | | | | | | (0%) | (0) | |
| 1192 | MT- | DEAF-associated | C1192A | 12S rRNA | + | − | Reported | N/A | 0% | 8 | 2 |
| | RNR1 | | | | | | | | (0%) | (0) | |
| 1192 | MT- | DEAF-associated | C1192T | 12S rRNA | + | − | Reported | N/A | 0% | 12 | 1 |
| | RNR1 | | | | | | | | (0%) | (0) | |
| 1226 | MT- | Possibly DEAF- | C1226G | 12S rRNA | + | − | Reported | N/A | 0% | 0 | 2 |
| | RNR1 | associated | | | | | | | (0%) | (0) | |
| 1291 | MT- | DEAF | T1291C | 12S rRNA | + | − | Unclear | N/A | 0.1% | 53 | 3 |
| | RNR1 | | | | | | | | (0%) | (0) | |
| 1310 | MT- | DEAF-associated | C1310T | 12S rRNA | + | − | Reported | N/A | 0.1% | 37 | 1 |
| | RNR1 | | | | | | | | (0%) | (0) | |
| 1331 | MT- | DEAF-associated | A1331G | 12S rRNA | + | − | Reported | N/A | 0% | 10 | 1 |
| | RNR1 | | | | | | | | (0%) | (0) | |
| 1349 | MT- | DEAF | T1349G | 12S rRNA | − | + | Reported | N/A | 0% | 0 | 1 |
| | RNR1 | | | | | | | | (0%) | (0) | |
| 1374 | MT- | DEAF-associated | A1374G | 12S rRNA | + | − | Reported | N/A | 0% | 1 | 2 |
| | RNR1 | | | | | | | | (0%) | (0) | |
| 1391 | MT- | found in 1 HCM | T1391C | 12S rRNA | + | − | Reported | N/A | 0.3% | 132 | 1 |
| | RNR1 | patient | | | | | | | (0%) | (0) | |
| 1420 | MT- | DEAF | T1420G | 12S rRNA | + | + | Reported | N/A | 0% | 0 | 1 |
| | RNR1 | | | | | | | | (0%) | (0) | |
| 1438 | MT- | SZ-associated | A1438A | 12S rRNA | + | − | Reported | N/A | 5.2% | 2602 | 3 |
| | RNR1 | | | | | | | | (0%) | (0) | |
| 1452 | MT- | DEAF-associated | T1452C | 12S rRNA | + | − | Reported | N/A | 0.1% | 48 | 1 |
| | RNR1 | | | | | | | | (0%) | (0) | |
| 1453 | MT- | Possible DEAF | A1453G | 12S rRNA | . | . | Reported | N/A | 0.2% | 107 | 1 |
| | RNR1 | risk factor | | | | | | | (0%) | (0) | |
| 1492 | MT- | DEAF | A1492C | 12S rRNA | − | + | Reported | N/A | 0% | 0 | 1 |
| | RNR1 | | | | | | | | (0%) | (0) | |
| 1494 | MT- | DEAF | C1494T | 12S rRNA | + | − | Cfrm | N/A | 0% | 4 | 29 |
| | RNR1 | | | | | | | | (0%) | (0) | |
| 1517 | MT- | DEAF | A1517C | 12S rRNA | − | + | Reported | N/A | 0% | 0 | 1 |
| | RNR1 | | | | | | | | (0%) | (0) | |
| 1537 | MT- | DEAF; intellectual | C1537T | 12S rRNA | + | − | Reported | N/A | 0% | 6 | 1 |
| | RNR1 | disability | | | | | | | (0%) | (0) | |
| 1544 | MT- | DEAF | A1544T | 12S rRNA | + | − | Reported | N/A | 0% | 0 | 2 |
| | RNR1 | | | | | | | | (0%) | (0) | |
| 1546 | MT- | DEAF | A1546T | 12S rRNA | + | − | Reported | N/A | 0% | 0 | 1 |
| | RNR1 | | | | | | | | (0%) | (0) | |
| 1554 | MT- | DEAF | G1554A | 12S rRNA | + | − | Reported | N/A | 0% | 0 | 1 |
| | RNR1 | | | | | | | | (0%) | (0) | |
| 1555 | MT- | DEAF; autism spectrum | A1555G | 12S rRNA | + | − | Cfrm | N/A | 0.1% | 74 | 138 |
| | RNR1 | intellectual | | | | | | | (0%) | (0) | |
| | | disability; possibly | | | | | | | | | |
| | | antiatherosclerotic | | | | | | | | | |
| 1556 | MT- | found in 1 HCM | C1556T | 12S rRNA | + | − | Reported | N/A | 0% | 4 | 1 |
| | RNR1 | patient | | | | | | | (0%) | (0) | |
| 1575 | MT- | DEAF | T1575G | 12S rRNA | + | − | Reported | N/A | 0% | 0 | 1 |
| | RNR1 | | | | | | | | (0%) | (0) | |
| 1577 | MT- | DEAF | T1577G | 12S rRNA | − | + | Reported | N/A | 0% | 0 | 1 |
| | RNR1 | | | | | | | | (0%) | (0) | |
| 1606 | MT-TV | AMDF | G1606A | tRNA Val | − | + | Cfrm | Pathogenic | 0% | 0 | 4 |
| | | | | | | | | | (0%) | (0) | |
| 1607 | MT-TV | Suspected mito | T1607C | tRNA Val | + | + | Reported | 18.10% | 0% | 10 | 1 |
| | | disease | | | | | | | (0%) | (0) | |
| 1616 | MT-TV | MELAS | A1616G | tRNA Val | . | . | Reported | 36.70% | 0% | 0 | 1 |
| | | | | | | | | | (0%) | (0) | |
| 1624 | MT-TV | Leigh Syndrome | C1624T | tRNA Val | + | − | Reported | 68.70% | 0% | 0 | 4 |
| | | | | | | | | | (0%) | (0) | |
| 1630 | MT-TV | MNGIE-like | A1630G | tRNA Val | − | + | Cfrm | Pathogenic | 0% | 0 | 3 |
| | | disease/MELAS | | | | | | | (0%) | (0) | |
| 1642 | MT-TV | MELAS | G1642A | tRNA Val | − | + | Reported | 74.30% | 0% | 0 | 2 |
| | | | | | | | | | (0%) | (0) | |
| 1643 | MT-TV | Late infantile onset | A1643G | tRNA Val | + | + | Reported | 42.00% | 0% | 1 | 1 |
| | | fatal mito disease | | | | | | | (0%) | (0) | |
| 1644 | MT-TV | LS/HCM/MELAS | G1644A | tRNA Val | − | + | Cfrm | Pathogenic | 0% | 0 | 4 |
| | | | | | | | | | (0%) | (0) | |
| 1644 | MT-TV | Adult Leigh | G1644T | tRNA Val | − | + | Reported | 48.40% | 0% | 0 | 1 |
| | | Syndrome | | | | | | | (0%) | (0) | |
| 1659 | MT-TV | Movement | T1659C | tRNA Val | − | + | Reported | 69.60% | 0% | 0 | 1 |
| | | Disorder | | | | | | | (0%) | (0) | |
| 2158 | MT- | Reduced | T2158C | 16S rRNA | . | . | Reported | N/A | 0.4% | 200 | 2 |
| | RNR2 | risk PD | | | | | | | (0%) | (0) | |
| 2336 | MT- | Hypertrophic | T2336C | 16S rRNA | + | − | Reported | N/A | 0% | 0 | 2 |
| | RNR2 | cardiomyopathy | | | | | | | (0%) | (0) | |
| 2352 | MT- | Possibly LVNC- | T2352C | 16S rRNA | + | − | Reported | N/A | 2.6% | 1281 | 3 |
| | RNR2 | associated | | | | | | | (0%) | (0) | |
| 2361 | MT- | Possibly LVNC- | G2361A | 16S rRNA | + | − | Reported | N/A | 0.3% | 135 | 1 |
| | RNR2 | associated | | | | | | | (0%) | (0) | |
| 2639 | MT- | Rare mutation in a | C2639A | 16S rRNA | + | − | Reported | N/A | 0% | 1 | 1 |
| | RNR2 | single POAG patient | | | | | | | (0%) | (0) | |
| 2706 | MT- | Increased risk of | A2706A | 16S rRNA | + | − | Reported | N/A | 21% | 10515 | 1 |
| | RNR2 | T2DM in haplogroup H | | | | | | | (0%) | (0) | |
| 2755 | MT- | Possibly LVNC- | A2755G | 16S rRNA | + | − | Reported | N/A | 0.5% | 262 | 2 |
| | RNR2 | associated | | | | | | | (0%) | (0) | |
| 2835 | MT- | Rett Syndrome | C2835T | 16S rRNA | − | + | Reported | N/A | 0.1% | 58 | 2 |
| | RNR2 | | | | | | | | (0%) | (0) | |
| 3010 | MT- | Cyclic Vomiting | G3010A | 16S rRNA | + | − | Reported | N/A | 14.4% | 7223 | 6 |
| | RNR2 | Syndrome with | | | | | | | (0%) | (0) | |
| | | Migraine | | | | | | | | | |
| 3090 | MT- | Myopathy | G3090A | 16S rRNA | − | + | Reported | N/A | 0% | 2 | 1 |
| | RNR2 | | | | | | | | (0%) | (0) | |
| 3093 | MT- | MELAS | C3093G | 16S rRNA | − | + | Reported | N/A | 0% | 0 | 2 |
| | RNR2 | | | | | | | | (0%) | (0) | |
| 3111 | MT- | Migraine | A3111T | 16S rRNA | + | − | Reported | N/A | 0% | 6 | 1 |
| | RNR2 | | | | | | | | (0%) | (0) | |
| 3196 | MT- | ADPD | G3196A | 16S rRNA | + | + | Reported | N/A | 0% | 13 | 3 |
| | RNR2 | | | | | | | | (0%) | (0) | |
| 3236 | MT-TL1 | Sporadic bilateral | A3236G | tRNA Leu | . | . | Reported | 37.80% | 0% | 2 | 2 |
| | | optic neuropathy | | (UUR) | | | | | (0%) | (0) | |
| 3242 | MT-TL1 | MM/HCM + renal | G3242A | tRNA Leu | + | + | Reported | 18.50% | 0% | 0 | 5 |
| | | tubular dysfunction | | (UUR) | | | | | (0%) | (0) | |
| 3243 | MT-TL1 | MELAS/LS/DMDF/ | A3243G | tRNA Leu | − | + | Cfrm | Pathogenic | 0% | 9 | 392 |
| | | MIDD/SNHL/CPEO/ | | (UUR) | | | | | (0%) | (0) | |
| | | MM/FSGS/ASD/ | | | | | | | | | |
| | | Cardiac + multi- | | | | | | | | | |
| | | organ dysfunction | | | | | | | | | |
| 3243 | MT-TL1 | MM/MELAS/ | A3243T | tRNA Leu | − | + | Cfrm | Pathogenic | 0% | 0 | 6 |
| | | SNHL/CPEO | | (UUR) | | | | | (0%) | (0) | |
| 3244 | MT-TL1 | MELAS | G3244A | tRNA Leu | − | + | Reported | 41.60% | 0% | 6 | 4 |
| | | | | (UUR) | | | | | (0%) | (0) | |
| 3249 | MT-TL1 | KSS | G3249A | tRNA | − | + | Reported | 39.30% | 0% | 0 | 3 |
| | | | | Leu(UUR) | | | | | (0%) | (0) | |
| 3250 | MT-TL1 | MM/CPEO | T3250C | tRNA Leu | − | + | Reported | 33.40% | 0% | 0 | 11 |
| | | | | (UUR) | | | | | (0%) | (0) | |
| 3251 | MT-TL1 | MM/MELAS with | A3251G | tRNA Leu | − | + | Reported | 43.50% | 0% | 0 | 4 |
| | | chorea-ballism | | (UUR) | | | | | (0%) | (0) | |
| 3252 | MT-TL1 | MELAS | A3252G | tRNA Leu | − | + | Reported | 39.40% | 0% | 0 | 4 |
| | | | | (UUR) | | | | | (0%) | (0) | |
| 3252 | MT-TL1 | EXIT | A3252T | tRNA Leu | − | + | Reported | 39.40% | 0% | 0 | 1 |
| | | | | (UUR) | | | | | (0%) | (0) | |
| 3253 | MT-TL1 | Maternally inherited | T3253C | tRNA Leu | + | − | Reported | 0.40% | 0% | 6 | 3 |
| | | hypertension | | (UUR) | | | | | (0%) | (0) | |
| 3254 | MT-TL1 | Gestational | C3254A | tRNA Leu | − | + | Reported | 60.80% | 0.1% | 26 | 1 |
| | | Diabetes (GDM) | | (UUR) | | | | | (0%) | (0) | |
| 3254 | MT-TL1 | MM | C3254G | tRNA Leu | − | + | Reported | 60.80% | 0% | 0 | 3 |
| | | | | (UUR) | | | | | (0%) | (0) | |
| 3254 | MT-TL1 | CPEO/poss. hypertension | C3254T | tRNA Leu | + | − | Reported | 25.30% | 0% | 17 | 5 |
| | | factor | | (UUR) | | | | | (0%) | (0) | |
| 3255 | MT-TL1 | MERRF/KSS overlap | G3255A | tRNA Leu | − | + | Reported | 75.80% | 0% | 0 | 3 |
| | | | | (UUR) | | | | | (0%) | (0) | |
| 3256 | MT-TL1 | MELAS; possible | C3256T | tRNA Leu | − | + | Cfrm | Pathogenic | 0% | 0 | 18 |
| | | atherosclerosis risk | | (UUR) | | | | | (0%) | (0) | |
| 3258 | MT-TL1 | MELAS/Myopathy | T3258C | tRNA Leu | − | + | Cfrm | Pathogenic | 0% | 1 | 5 |
| | | | | (UUR) | | | | | (0%) | (0) | |
| 3260 | MT-TL1 | MMC/MELAS | A3260G | tRNA Leu | − | + | Cfrm | Pathogenic | 0% | 0 | 10 |
| | | | | (UUR) | | | | | (0%) | (0) | |
| 3264 | MT-TL1 | DM | T3264C | tRNA Leu | − | + | Reported | 47.30% | 0% | 0 | 3 |
| | | | | (UUR) | | | | | (0%) | (0) | |
| 3271 | MT-TL1 | PEM/retinal dystrophy | T3271del | tRNA Leu | − | + | Cfrm | Pathogenic | 0% | 0 | 3 |
| | | in MELAS | | (UUR) | | | | | (0%) | (0) | |
| 3271 | MT-TL1 | MELAS/DM | T3271C | tRNA Leu | − | + | Cfrm | Pathogenic | 0% | 0 | 25 |
| | | | | (UUR) | | | | | (0%) | (0) | |
| 3273 | MT-TL1 | Ocular myopathy | T3273C | tRNA Leu | − | + | Reported | 71.20% | 0% | 0 | 3 |
| | | | | (UUR) | | | | | (0%) | (0) | |
| 3274 | MT-TL1 | Neuropsychiatric | A3274G | tRNA Leu | − | + | Reported | 77.10% | 0% | 0 | 2 |
| | | syndrome + cataract | | (UUR) | | | | | (0%) | (0) | |
| 3275 | MT-TL1 | LHON | C3275A | tRNA Leu | + | − | Reported | 2.20% | 0% | 1 | 3 |
| | | | | (UUR) | | | | | (0%) | (0) | |
| 3275 | MT-TL1 | Metabolic syndrome | C3275T | tRNA Leu | + | − | Reported | 2.20% | 0% | 2 | 2 |
| | | and polycystic | | (UUR) | | | | | (0%) | (0) | |
| | | ovary syndrome | | | | | | | | | |
| 3277 | MT-TL1 | Poss. hypertension | G3277A | tRNA Leu | + | − | Reported | 2.90% | 0.1% | 32 | 1 |
| | | factor | | (UUR) | | | | | (0%) | (0) | |
| 3278 | MT-TL1 | Poss. hypertension | T3278C | tRNA Leu | + | − | Reported | 13.10% | 0% | 14 | 1 |
| | | factor | | (UUR) | | | | | (0%) | (0) | |
| 3280 | MT-TL1 | Myopathy | A3280G | tRNA Leu | − | + | Cfrm | Pathogenic | 0% | 0 | 6 |
| | | | | (UUR) | | | | | (0%) | (0) | |
| 3283 | MT-TL1 | Late onset ocular | G3283A | tRNA Leu | − | + | Reported | 58.70% | 0% | 0 | 1 |
| | | myopathy | | (UUR) | | | | | (0%) | (0) | |
| 3287 | MT-TL1 | Encephalomyopathy | C3287A | tRNA Leu | − | + | Reported | 38.30% | 0% | 0 | 2 |
| | | | | (UUR) | | | | | (0%) | (0) | |
| 3288 | MT-TL1 | Myopathy | A3288G | tRNA Leu | − | + | Reported | 36.10% | 0% | 0 | 3 |
| | | | | (UUR) | | | | | (0%) | (0) | |
| 3290 | MT-TL1 | Poss. hypertension | T3290C | tRNA Leu | + | − | Reported | 1.40% | 0.2% | 121 | 2 |
| | | factor | | (UUR) | | | | | (0%) | (0) | |
| 3291 | MT-TL1 | MELAS/Myopathy/ | T3291C | tRNA Leu | − | + | Cfrm | Pathogenic | 0% | 0 | 14 |
| | | Deafness + Cognitive | | (UUR) | | | | | (0%) | (0) | |
| | | Impairment | | | | | | | | | |
| 3302 | MT-TL1 | MM | A3302G | tRNA Leu | − | + | Cfrm | Pathogenic | 0% | 0 | 10 |
| | | | | (UUR) | | | | | (0%) | (0) | |
| 3303 | MT-TL1 | MMC | C3303T | RNA Leu | + | + | Cfrm | Pathogenic | 0% | 0 | 12 |
| | | | | (UUR) | | | | | (0%) | (0) | |
| 4263 | MT-TI | Maternally inherited | A4263G | tRNA Ile | + | − | Reported | 67.80% | 0% | 4 | 4 |
| | | essential hypertension | | | | | | | (0%) | (0) | |
| 4267 | MT-TI | MM/CPEO | A4267G | tRNA Ile | − | + | Reported | 71.10% | 0% | 0 | 4 |
| | | | | | | | | | (0%) | (0) | |
| 4269 | MT-TI | FICP | A4269G | tRNA Ile | − | + | Reported | 82.80% | 0% | 0 | 9 |
| | | | | | | | | | (0%) | (0) | |
| 4274 | MT-TI | CPEO/Motor Neuron | T4274C | tRNA Ile | − | + | Reported | 85.50% | 0% | 0 | 5 |
| | | Disease | | | | | | | (0%) | (0) | |
| 4277 | MT-TI | HCM/Poss. hypertension | T4277C | tRNA Ile | + | − | Reported | 8.90% | 0% | 18 | 2 |
| | | factor | | | | | | | (0%) | (0) | |
| 4279 | MT-TI | Myoclonic epilepsy | A4279G | tRNA Ile | − | + | Reported | 54.90% | 0% | 0 | 1 |
| | | | | | | | | | (0%) | (0) | |
| 4281 | MT-TI | Recurrent | A4281G | tRNA Ile | − | + | Reported | 87.90% | 0% | 1 | 1 |
| | | Myoglobinuria | | | | | | | (0%) | (0) | |
| 4282 | MT-TI | CPEO Plus | G4282A | tRNA Ile | − | + | Reported | 82.30% | 0% | 0 | 1 |
| | | | | | | | | | (0%) | (0) | |
| 4284 | MT-TI | Varied familial | G4284A | tRNA Ile | − | + | Reported | 35.30% | 0% | 2 | 6 |
| | | presentation/spastic | | | | | | | (0%) | (0) | |
| | | paraparesis | | | | | | | | | |
| 4285 | MT-TI | CPEO | T4285C | tRNA Ile | − | + | Reported | 84.80% | 0% | 0 | 5 |
| | | | | | | | | | (0%) | (0) | |
| 4289 | MT-TI | Retinopathy + | T4289C | tRNA Ile | − | + | Reported | 84.30% | 0% | 0 | 1 |
| | | diabetes + | | | | | | | (0%) | (0) | |
| | | dysphagia + | | | | | | | | | |
| | | cerebral atrophy | | | | | | | | | |
| 4290 | MT-TI | Progressive | T4290C | tRNA Ile | + | + | Reported | 47.70% | 0% | 0 | 4 |
| | | Encephalopathy/PEO, | | | | | | | (0%) | (0) | |
| | | myopathy | | | | | | | | | |
| 4291 | MT-TI | Hypomagnesemic | T4291C | tRNA Ile | + | − | Reported | 31.80% | 0% | 0 | 1 |
| | | Metabolic Syndrome | | | | | | | (0%) | (0) | |
| 4295 | MT-TI | MHCM/Maternally | A4295G | tRNA Ile | + | + | Reported | 44.00% | 0.2% | 95 | 11 |
| | | inherited | | | | | | | (0%) | (0) | |
| | | hypertension/ | | | | | | | | | |
| | | Maternally | | | | | | | | | |
| | | inherited deafness | | | | | | | | | |
| 4296 | MT-TI | Leigh Syndrome | G4296A | tRNA Ile | − | + | Reported | 46.60% | 0% | 0 | 3 |
| | | | | | | | | | (0%) | (0) | |
| 4298 | MT-TI | CPEO/MS | G4298A | tRNA Ile | − | + | Cfrm | Pathogenic | 0% | 0 | 9 |
| | | | | | | | | | (0%) | (0) | |
| 4300 | MT-TI | MICM | A4300G | tRNA Ile | + | + | Cfrm | Pathogenic | 0% | 0 | 9 |
| | | | | | | | | | (0%) | (0) | |
| 4302 | MT-TI | CPEO | A4302G | tRNA Ile | − | + | Reported | 42.00% | 0% | 0 | 1 |
| | | | | | | | | | (0%) | (0) | |
| 4308 | MT-TI | CPEO | G4308A | tRNA Ile | − | + | Cfrm | Pathogenic | 0% | 0 | 2 |
| | | | | | | | | | (0%) | (0) | |
| 4309 | MT-TI | CPEO | G4309A | tRNA Ile | − | + | Reported | 64.10% | 0% | 1 | 3 |
| | | | | | | | | | (0%) | (0) | |
| 4314 | MT-TI | Poss. hypertension | T4314C | tRNA Ile | + | − | Reported | 1.70% | 0.1% | 42 | 1 |
| | | factor | | | | | | | (0%) | (0) | |
| 4316 | MT-TI | HCM with hearing loss/ | A4316G | tRNA Ile | + | + | Reported | 37.10% | 0.1% | 35 | 2 |
| | | poss. hypertension factor | | | | | | | (0%) | (0) | |
| 4317 | MT-TI | FICP/poss. Hypertension/ | A4317G | tRNA Ile | + | − | Reported | 2.10% | 0.1% | 38 | 11 |
| | | DEAF factor | | | | | | | (0%) | (0) | |
| 4317 | MT-TI | Ptosis, deafness, | A4317del | tRNA Ile | . | . | Reported | 2.10% | 0% | 0 | 1 |
| | | stroke-like | | | | | | | (0%) | (0) | |
| | | episodes | | | | | | | | | |
| 4320 | MT-TI | Mitochondrial | C4320T | tRNA Ile | − | + | Reported | 25.60% | 0% | 4 | 4 |
| | | Encephalocardio- | | | | | | | (0%) | (0) | |
| | | myopathy | | | | | | | | | |
| 4322 | MT-TI | Idiopathic | C4322CC | tRNA Ile | − | + | Reported | — | 0% | 3 | 1 |
| | | Dilated | | | | | | | (0%) | (0) | |
| | | Cardiomopathy | | | | | | | | | |
| 4322 | MT-TI | mtDNA deletion and | C4322del | tRNA Ile | + | − | Reported | 88.10% | 0% | 0 | 1 |
| | | depletion with dilated | | | | | | | (0%) | (0) | |
| | | cardiomyopathy | | | | | | | | | |
| 4332 | MT-TQ | Encephalopathy/MELAS | G4332A | tRNA Gln | − | + | Cfrm | Pathogenic | 0% | 0 | 4 |
| | | | | | | | | | (0%) | (0) | |
| 4336 | MT-TQ | ADPD/Hearing Loss & | T4336C | tRNA Gln | + | + | Unclear | 37.30% | 0.8% | 416 | 26 |
| | | Migraine/autism | | | | | | | (0%) | (0) | |
| | | spectrum/ | | | | | | | | | |
| | | intellectual | | | | | | | | | |
| | | disability | | | | | | | | | |
| 4343 | MT-TQ | Poss. hypertension | A4343G | tRNA Gln | + | − | Reported | 5.10% | 0.1% | 53 | 1 |
| | | factor | | | | | | | (0%) | (0) | |
| 4345 | MT-TQ | Poss. hypertension | C4345T | tRNA Gln | + | − | Reported | 13.20% | 0% | 2 | 1 |
| | | factor | | | | | | | (0%) | (0) | |
| 4353 | MT-TQ | Poss. hypertension | T4353C | tRNA Gln | + | − | Reported | 31.60% | 0% | 23 | 1 |
| | | factor | | | | | | | (0%) | (0) | |
| 4363 | MT-TQ | Metabolic syndrome and | T4363C | tRNA Gln | + | − | Reported | 9.50% | 0.1% | 45 | 5 |
| | | polycystic ovary | | | | | | | (0%) | (0) | |
| | | syndrome/possibly | | | | | | | | | |
| | | associated w | | | | | | | | | |
| | | DEAF + RP + dev | | | | | | | | | |
| | | delay/hypertension | | | | | | | | | |
| 4369 | MT-TQ | Myopathy | A4369AA | tRNA Gln | − | + | Reported | — | 0% | 0 | 2 |
| | | | | | | | | | (0%) | (0) | |
| 4372 | MT-TQ | Suspected mito | C4372T | tRNA Gln | − | + | Reported | 71.30% | 0% | 0 | 1 |
| | | disease | | | | | | | (0%) | (0) | |
| 4373 | MT-TQ | Possibly LVNC- | T4373C | tRNA Gln | + | − | Reported | 29.10% | 0% | 8 | 1 |
| | | associated | | | | | | | (0%) | (0) | |
| 4381 | MT-TQ | LHON | A4381G | tRNA Gln | + | − | Reported | 15.30% | 0% | 4 | 1 |
| | | | | | | | | | (0%) | (0) | |
| 4386 | MT-TQ | Heart disease/myopathy/ | T4386C | tRNA Gln | + | − | Conflicting | 6.90% | 0.3% | 167 | 3 |
| | | hypertension | | | | | reports | | (0%) | (0) | |
| 4387 | MT-TQ | Poss. hypertension | C4387A | tRNA Gln | + | − | Reported | 12.80% | 0% | 0 | 1 |
| | | factor | | | | | | | (0%) | (0) | |
| 4388 | MT-TQ | Poss. hypertension | A4388G | tRNA Gln | + | − | Reported | 0.10% | 0.1% | 64 | 2 |
| | | factor; intellectual | | | | | | | (0%) | (0) | |
| | | disability | | | | | | | | | |
| 4392 | MT-TQ | Poss. hypertension | C4392T | tRNA Gln | + | − | Reported | 15.70% | 0% | 18 | 1 |
| | | factor | | | | | | | (0%) | (0) | |
| 4395 | MT-TQ | Poss. hypertension | A4395G | tRNA Gln | + | − | Reported | 0.20% | 0% | 24 | 1 |
| | | factor | | | | | | | (0%) | (0) | |
| 4401 | MT- | Hypertension + | A4401G | NC2 | + | − | Reported | N/A | 0% | 3 | 3 |
| | NC2 | Ventricular | | Gln-Met | | | | | (0%) | (0) | |
| | | Hypertrophy | | spacer | | | | | | | |
| 4403 | MT-TM | Mitochondrial | G4403A | tRNA Met | − | + | Reported | 84.80% | 0% | 0 | 1 |
| | | myopathy | | | | | | | (0%) | (0) | |
| 4409 | MT-TM | Mitochondrial | T4409C | tRNA Met | − | + | Reported | 46.50% | 0% | 0 | 5 |
| | | myopathy | | | | | | | (0%) | (0) | |
| 4410 | MT-TM | Poss. hypertension | C4410A | tRNA Met | + | − | Reported | 32.90% | 0% | 0 | 1 |
| | | factor | | | | | | | (0%) | (0) | |
| 4412 | MT-TM | Seizures with | G4412A | tRNA Met | − | + | Reported | 76.50% | 0% | 0 | 1 |
| | | myopathy & | | | | | | | (0%) | (0) | |
| | | retinopathy | | | | | | | | | |
| 4415 | MT-TM | EXIT & APS2 | A4415G | tRNA Met | − | + | Reported | 44.10% | 0% | 0 | 1 |
| | | | | | | | | | (0%) | (0) | |
| 4435 | MT-TM | LHON modulator/ | A4435G | tRNA Met | + | − | Reported | 13.80% | 0.1% | 52 | 9 |
| | | hypertension; autism | | | | | | | (0%) | (0) | |
| | | spectrum; intellectual | | | | | | | | | |
| | | disability | | | | | | | | | |
| 4437 | MT-TM | Hypotonia, seizure, | C4437T | tRNA Met | + | − | Reported | 67.20% | 0% | 1 | 2 |
| | | muscle weakness, | | | | | | | (0%) | (0) | |
| | | lactic acidosis, | | | | | | | | | |
| | | hearing loss | | | | | | | | | |
| 4440 | MT-TM | Mitochondrial | G4440A | tRNA Met | − | + | Reported | 58.20% | 0% | 0 | 3 |
| | | myopathy | | | | | | | (0%) | (0) | |
| 4450 | MT-TM | Myopathy/MELAS/ | G4450A | tRNA Met | − | + | Cfrm | Pathogenic | 0% | 0 | 4 |
| | | Leigh Syndrome | | | | | | | (0%) | (0) | |
| 4456 | MT-TM | Poss. hypertension | C4456T | tRNA Met | − | + | Reported | 32.00% | 0% | 7 | 1 |
| | | factor | | | | | | | (0%) | (0) | |
| 4467 | MT-TM | Maternally inherited | C4467A | tRNA Met | − | + | Reported | 75.60% | 0% | 0 | 1 |
| | | hypertension | | | | | | | (0%) | (0) | |
| 5512 | MT-TW | Maternally inherited | A5512G | tRNA Trp | + | − | Reported | 38.60% | 0% | 5 | 1 |
| | | hypertension | | | | | | | (0%) | (0) | |
| 5513 | MT-TW | Mitochondrial | G5513A | tRNA Trp | − | + | Reported | 32.60% | 0% | 1 | 1 |
| | | encephalomyopathy | | | | | | | (0%) | (0) | |
| | | with RP | | | | | | | | | |
| 5514 | MT-TW | Neonatal onset | A5514G | tRNA Trp | + | − | Reported | 19.70% | 0.1% | 42 | 1 |
| | | mito disease | | | | | | | (0%) | (0) | |
| 5521 | MT-TW | Mitochondrial | G5521A | tRNA Trp | − | + | Cfrm | Pathogenic | 0% | 0 | 5 |
| | | myopathy | | | | | | | (0%) | (0) | |
| 5522 | MT-TW | Mitochondrial | G5522A | tRNA Trp | − | + | Reported | 83.00% | 0% | 0 | 2 |
| | | myopathy | | | | | | | (0%) | (0) | |
| 5523 | MT-TW | Leigh Syndrome | T5523G | tRNA Trp | − | + | Reported | 80.90% | 0% | 0 | 1 |
| | | | | | | | | | (0%) | (0) | |
| 5532 | MT-TW | Gastrointestinal | G5532A | tRNA Trp | − | + | Reported | 19.40% | 0% | 1 | 3 |
| | | Syndrome | | | | | | | (0%) | (0) | |
| 5537 | MT-TW | Leigh Syndrome | A5537insT | tRNA Trp | − | + | Cfrm | — | 0% | 0 | 5 |
| | | | | | | | | | (0%) | (0) | |
| 5538 | MT-TW | Encephalomyopathy | G5538A | tRNA Trp | − | + | Reported | 76.70% | 0% | 0 | 1 |
| | | | | | | | | | (0%) | (0) | |
| 5540 | MT-TW | Encephalomyopathy/DEAF | G5540A | tRNA Trp | − | + | Reported | 73.70% | 0% | 0 | 3 |
| | | | | | | | | | (0%) | (0) | |
| 5541 | MT-TW | MELAS + stroke- | C5541T | tRNA Trp | − | + | Reported | 84.30% | 0% | 0 | 1 |
| | | like episodes | | | | | | | (0%) | (0) | |
| | | and cortical | | | | | | | | | |
| | | blindness + | | | | | | | | | |
| | | MRI shows occipital | | | | | | | | | |
| | | lobe infarct | | | | | | | | | |
| 5543 | MT-TW | Mitochondrial myopathy | T5543C | tRNA Trp | − | + | Reported | 47.30% | 0% | 0 | 5 |
| | | | | | | | | | (0%) | (0) | |
| 5545 | MT-TW | HCM severe multisystem | C5545T | tRNA Trp | − | + | Reported | 53.00% | 0% | 0 | 1 |
| | | disorder | | | | | | | (0%) | (0) | |
| 5549 | MT-TW | DEMCHO | G5549A | tRNA Trp | − | + | Reported | 83.30% | 0% | 0 | 1 |
| | | | | | | | | | (0%) | (0) | |
| 5556 | MT-TW | Mito encephalomyopathy | G5556C | tRNA Trp | − | + | Reported | 44.50% | 0% | 0 | 1 |
| | | | | | | | | | (0%) | (0) | |
| 5556 | MT-TW | Combined OXPHOS | G5556A | tRNA Trp | − | + | Reported | 44.50% | 0% | 0 | 2 |
| | | defects | | | | | | | (0%) | (0) | |
| 5559 | MT-TW | Leigh Syndrome | A5559G | tRNA Trp | − | + | Reported | 70.10% | 0% | 0 | 1 |
| | | | | | | | | | (0%) | (0) | |
| 5567 | MT-TW | Myopathy | T5567C | tRNA Trp | − | + | Reported | 32.70% | 0.1% | 50 | 2 |
| | | | | | | | | | (0%) | (0) | |
| 5568 | MT-TW | DEAF | A5568G | tRNA Trp | + | − | Reported | 9.70% | 0% | 9 | 1 |
| | | | | | | | | | (0%) | (0) | |
| 5587 | MT-TA | LHON/possible DEAF | T5587C | tRNA Ala | + | + | Reported | 12.10% | 0.1% | 34 | 4 |
| | | modifier/dilated | | | | | | | (0%) | (0) | |
| | | cardiomyopathy/ | | | | | | | | | |
| | | hypertension | | | | | | | | | |
| 5591 | MT-TA | Myopathy | G5591A | tRNA Ala | − | + | Reported | 68.40% | 0% | 0 | 3 |
| | | | | | | | | | (0%) | (0) | |
| 5592 | MT-TA | Coronary Heart | A5592G | tRNA Ala | + | − | Reported | 0.40% | 0.1% | 27 | 2 |
| | | Disease | | | | | | | (0%) | (0) | |
| 5610 | MT-TA | Myopathy | G5610A | tRNA Ala | − | + | Reported | 38.70% | 0% | 0 | 1 |
| | | | | | | | | | (0%) | (0) | |
| 5613 | MT-TA | CPEO | T5613C | tRNA Ala | − | + | Reported | 59.30% | 0% | 0 | 1 |
| | | | | | | | | | (0%) | (0) | |
| 5628 | MT-TA | CPEO/DEAF | T5628C | tRNA Ala | − | + | Reported | 78.90% | 0.2% | 97 | 4 |
| | | enhancer/gout | | | | | | | (0%) | (0) | |
| 5631 | MT-TA | Myopathy | G5631A | tRNA Ala | − | + | Reported | 43.40% | 0% | 1 | 2 |
| | | | | | | | | | (0%) | (0) | |
| 5636 | MT-TA | PEO | T5636C | tRNA Ala | − | + | Reported | 73.50% | 0% | 0 | 1 |
| | | | | | | | | | (0%) | (0) | |
| 5650 | MT-TA | Myopathy | G5650A | tRNA Ala | − | + | Cfrm | Pathogenic | 0% | 1 | 7 |
| | | | | | | | | | (0%) | (0) | |
| 5652 | MT-TA | Dilated | C5652G | tRNA Ala | + | − | Reported | 69.90% | 0% | 0 | 1 |
| | | Cardiomyopathy | | | | | | | (0%) | (0) | |
| 5655 | MT-TA | DEAF enhancer/ | T5655C | tRNA Ala | + | − | Reported | 26.70% | 0.6% | 324 | 3 |
| | | Hypertension | | | | | | | (0%) | (0) | |
| | | risk | | | | | | | | | |
| 5658 | MT-TN | Mitochondrial | T5658C | tRNA Asn | − | + | Reported | 94.30% | 0% | 0 | 1 |
| | | myopathy | | | | | | | (0%) | (0) | |
| 5667 | MT-TN | Ptosis | G5667A | tRNA Asn | . | . | Reported | 44.60% | 0% | 0 | 1 |
| | | | | | | | | | (0%) | (0) | |
| 5690 | MT-TN | CPEO + ptosis + | A5690G | tRNA Asn | − | + | Cfrm | Pathogenic | 0% | 0 | 2 |
| | | proximal myopathy | | | | | | | (0%) | (0) | |
| 5692 | MT-TN | CPEO/MM | T5692C | tRNA Asn | − | + | Reported | 46.60% | 0% | 0 | 4 |
| | | | | | | | | | (0%) | (0) | |
| 5693 | MT-TN | Encephalomyopathy | T5693C | tRNA Asn | + | − | Reported | 31.20% | 0% | 0 | 1 |
| | | | | | | | | | (0%) | (0) | |
| 5698 | MT-TN | CPEO/MM | G5698A | tRNA Asn | − | + | Reported | 47.70% | 0% | 1 | 4 |
| | | | | | | | | | (0%) | (0) | |
| 5703 | MT-TN | CPEO/MM | G5703A | tRNA Asn | − | + | Cfrm | Pathogenic | 0% | 0 | 7 |
| | | | | | | | | | (0%) | (0) | |
| 5709 | MT-TN | Ophthalmoparesis + | T5709C | tRNA Asn | − | + | Reported | 49.80% | 0% | 0 | 1 |
| | | respiratory | | | | | | | (0%) | (0) | |
| | | impairment | | | | | | | | | |
| 5728 | MT-TN | Multiorgan | T5728C | tRNA Asn | − | + | Cfrm | Pathogenic | 0% | 1 | 3 |
| | | failure/myopathy | | | | | | | (0%) | (0) | |
| 5780 | MT-TC | SNHL | G5780A | tRNA Cys | − | + | Reported | 35.50% | 0% | 15 | 1 |
| | | | | | | | | | (0%) | (0) | |
| 5783 | MT-TC | Myopathy/deafness/gout | G5783A | tRNA Cys | − | + | Reported | 66.90% | 0.1% | 43 | 2 |
| | | | | | | | | | (0%) | (0) | |
| 5802 | MT-TC | DEAF1555 increased | T5802C | tRNA Cys | + | − | Reported | 58.90% | 0% | 1 | 2 |
| | | penetrance | | | | | | | (0%) | (0) | |
| 5814 | MT-TC | Encephalopathy/gout | T5814C | tRNA Cys | − | + | L2b marker | 38.80% | 0.3% | 146 | 10 |
| | | | | | | | | | (0%) | (0) | |
| 5816 | MT-TC | Progressive Dystonia | A5816G | tRNA Cys | + | − | Reported | 59.90% | 0% | 0 | 3 |
| | | | | | | | | | (0%) | (0) | |
| 5821 | MT-TC | DEAF helper mut. | G5821A | tRNA Cys | + | − | Reported | 20.90% | 0.7% | 341 | 4 |
| | | | | | | | | | (0%) | (0) | |
| 5843 | MT-TY | FSGS/Mitochondrial | A5843G | tRNA Tyr | + | − | Reported | 8.40% | 0.4% | 207 | 1 |
| | | Cytopathy | | | | | | | (0%) | (0) | |
| 5874 | MT-TY | EXIT | T5874G | tRNA Tyr | − | + | Reported | 38.90% | 0% | 0 | 1 |
| | | | | | | | | | (0%) | (0) | |
| 7445 | MT-TS1 | DEAF | A7445C | tRNA Ser | + | − | Reported | — | 0% | 13 | 5 |
| | precursor | | | (UCN) | | | | | (0%) | (0) | |
| | | | | precursor | | | | | | | |
| 7445 | MT-TS1 | SNHL | A7445G | tRNA Ser | + | + | Cfrm | — | 0% | 1 | 32 |
| | precursor | | | (UCN) | | | | | (0%) | (0) | |
| | | | | precursor | | | | | | | |
| 7445 | MT-TS1 | SNHL | A7445T | tRNA Ser | + | − | Reported | — | 0% | 3 | 1 |
| | precursor | | | (UCN) | | | | | (0%) | (0) | |
| | | | | precursor | | | | | | | |
| 7451 | MT-TS1 | CPEO + ptosis | A7451T | tRNA Ser | − | + | Reported | 80.70% | 0% | 0 | 1 |
| | | | | (UCN) | | | | | (0%) | (0) | |
| | | | | precursor | | | | | | | |
| 7453 | MT-TS1 | Fatal neonatal | G7453A | tRNA Ser | + | − | Reported | 68.00% | 0% | 0 | 2 |
| | | lactic acidosis | | (UCN) | | | | | (0%) | (0) | |
| 7456 | MT-TS1 | DEAF | A7456G | tRNA Ser | + | − | Unclear | 16.00% | 0% | 1 | 1 |
| | | | | (UCN) | | | | | (0%) | (0) | |
| 7458 | MT-TS1 | PEO | G7458A | tRNA Ser | − | + | Reported | 86.00% | 0% | 0 | 1 |
| | | | | (UCN) | | | | | (0%) | (0) | |
| 7462 | MT-TS1 | DEAF | C7462T | tRNA Ser | + | − | Reported | 11.20% | 0% | 6 | 1 |
| | | | | (UCN) | | | | | (0%) | (0) | |
| 7471 | MT-TS1 | PEM/AMDF/Motor neuron | C7471CC | tRNA Ser | + | + | Cfrm | — | 0% | 7 | 28 |
| | | disease-like | | (UCN) | | | | | (0%) | (0) | |
| 7472 | MT-TS1 | PEM/AMDF/Motor neuron | A7472CA | tRNA Ser | + | + | See | — | 0% | 0 | 1 |
| | | disease-like | | (UCN) | | | 7471insC | | (0%) | (0) | |
| 7472 | MT-TS1 | MM/DMDF modulator | A7472C | tRNA Ser | + | − | Reported | 3.20% | 0% | 9 | 3 |
| | | | | (UCN) | | | | | (0%) | (0) | |
| 7480 | MT-TS1 | MM | T7480G | tRNA Ser | − | + | Reported | 46.60% | 0% | 0 | 3 |
| | | | | (UCN) | | | | | (0%) | (0) | |
| 7486 | MT-TS1 | CPEO | G7486A | tRNA Ser | − | + | Reported | 50.50% | 0% | 0 | 1 |
| | | | | (UCN) | | | | | (0%) | (0) | |
| 7492 | MT-TS1 | Hypertension | C7492T | tRNA Ser | + | − | Reported | 0.10% | 0% | 8 | 1 |
| | | | | (UCN) | | | | | (0%) | (0) | |
| 7497 | MT-TS1 | MM/EXIT | G7497A | tRNA Ser | + | + | Cfrm | Pathogenic | 0% | 1 | 7 |
| | | | | (UCN) | | | | | (0%) | (0) | |
| 7501 | MT-TS1 | Cardiovascular disease; | T7501A | tRNA Ser | . | . | Reported | 1.90% | 0% | 1 | 3 |
| | | renal disease patient | | (UCN) | | | | | (0%) | (0) | |
| 7505 | MT-TS1 | Maternally inherited | T7505C | tRNA Ser | + | − | Reported | 58.60% | 0% | 0 | 2 |
| | | hearing loss | | (UCN) | | | | | (0%) | (0) | |
| 7506 | MT-TS1 | PEO with hearing loss | G7506A | tRNA Ser | − | + | Reported | 81.40% | 0% | 0 | 1 |
| | | | | (UCN) | | | | | (0%) | (0) | |
| 7510 | MT-TS1 | SNHL | T7510C | tRNA Ser | − | + | Cfrm | Pathogenic | 0% | 1 | 13 |
| | | | | (UCN) | | | | | (0%) | (0) | |
| 7511 | MT-TS1 | SNHL/Deafness | T7511C | tRNA Ser | + | + | Cfrm | Pathogenic | 0% | 2 | 20 |
| | | | | (UCN) | | | | | (0%) | (0) | |
| 7512 | MT-TS1 | PEM/MERME | T7512C | tRNA Ser | + | + | Reported | 64.20% | 0% | 0 | 10 |
| | | | | (UCN) | | | | | (0%) | (0) | |
| 7520 | MT-TD | Sporadic bilateral | G7520A | tRNA Asp | . | . | Reported | 54.90% | 0% | 0 | 1 |
| | | optic neuropathy | | | | | | | (0%) | (0) | |
| 7526 | MT-TD | Mitochondrial | A7526G | tRNA Asp | − | + | Reported | 50.40% | 0% | 0 | 1 |
| | | myopathy | | | | | | | (0%) | (0) | |
| 7539 | MT-TD | Multisystemic | C7539T | tRNA Asp | − | + | Reported | 93.70% | 0% | 0 | 1 |
| | | mitochondrial disorder | | | | | | | (0%) | (0) | |
| 7543 | MT-TD | MEPR | A7543G | tRNA Asp | − | + | Reported | 67.30% | 0.1% | 47 | 1 |
| | | | | | | | | | (0%) | (0) | |
| 7551 | MT-TD | DEAF increased | A7551G | tRNA Asp | + | − | Reported | 28.90% | 0% | 2 | 2 |
| | | penetrance | | | | | | | (0%) | (0) | |
| | | (1555G helper) | | | | | | | | | |
| 7554 | MT-TD | Myopathy + | G7554A | tRNA Asp | − | + | Reported | 71.20% | 0% | 1 | 1 |
| | | ataxia + | | | | | | | (0%) | (0) | |
| | | nystagmus + | | | | | | | | | |
| | | migraines + | | | | | | | | | |
| | | lactic acidosis | | | | | | | | | |
| 8296 | MT-TK | DMDF/MERRF/HCM/ | A8296G | tRNA Lys | + | + | Reported | 72.30% | 0.1% | 37 | 17 |
| | | epilepsy | | | | | | | (0%) | (0) | |
| 8299 | MT-TK | PEO + respiratory | G8299A | tRNA Lys | − | + | Reported | 63.80% | 0% | 0 | 1 |
| | | impairment | | | | | | | (0%) | (0) | |
| 8302 | MT-TK | Encephalopathy | A8302T | tRNA Lys | + | − | Unclear | 15.20% | 0% | 0 | 1 |
| | | | | | | | | | (0%) | (0) | |
| 8304 | MT-TK | Epilepsy + | G8304A | tRNA Lys | − | + | Reported | 89.70% | 0% | 0 | 1 |
| | | ataxia + visual | | | | | | | (0%) | (0) | |
| | | disturbance + | | | | | | | | | |
| | | deafness | | | | | | | | | |
| 8305 | MT-TK | Mitochondrial | C8305T | tRNA Lys | − | + | Reported | 74.50% | 0% | 0 | 3 |
| | | myopathy | | | | | | | (0%) | (0) | |
| 8306 | MT-TK | Severe adult-onset | T8306C | tRNA Lys | − | + | Cfrm | Pathogenic | 0% | 0 | 3 |
| | | multisymptom | | | | | | | (0%) | (0) | |
| | | myopathy/ | | | | | | | | | |
| | | Myoclonic | | | | | | | | | |
| | | epilepsy | | | | | | | | | |
| 8311 | MT-TK | Poss. hypertension | T8311C | tRNA Lys | + | − | Reported | 6.80% | 0.1% | 56 | 1 |
| | | factor | | | | | | | (0%) | (0) | |
| 8313 | MT-TK | MNGIE/Progressive | G8313A | tRNA Lys | − | + | Cfrm | Pathogenic | 0% | 1 | 6 |
| | | mito cytopathy | | | | | | | (0%) | (0) | |
| 8316 | MT-TK | MELAS | T8316C | tRNA Lys | − | + | Reported | 80.20% | 0% | 0 | 3 |
| | | | | | | | | | (0%) | (0) | |
| 8319 | MT-TK | Kearns-Sayre | A8319G | tRNA Lys | − | + | Reported | 69.60% | 0% | 0 | 1 |
| | | syndrome | | | | | | | (0%) | (0) | |
| 8326 | MT-TK | Mitochondrial | A8326G | tRNA Lys | − | + | Reported | 46.20% | 0% | 0 | 3 |
| | | Cytopathy | | | | | | | (0%) | (0) | |
| 8328 | MT-TK | Mito Encephalopathy/ | G8328A | tRNA Lys | − | + | Reported | 83.30% | 0% | 0 | 5 |
| | | EXIT with myopathy | | | | | | | (0%) | (0) | |
| | | and ptosis | | | | | | | | | |
| 8332 | MT-TK | Dystonia and stroke- | A8332G | tRNA Lys | + | − | Reported | 62.80% | 0% | 0 | 1 |
| | | like episodes | | | | | | | (0%) | (0) | |
| 8337 | MT-TK | Poss. hypertension | T8337C | tRNA Lys | + | − | Reported | 6.80% | 0.3% | 175 | 1 |
| | | factor | | | | | | | (0%) | (0) | |
| 8340 | MT-TK | Myopathy/Exercise | G8340A | tRNA Lys | − | + | Cfrm | Pathogenic | 0% | 0 | 7 |
| | | Intolerance/Eye | | | | | | | (0%) | (0) | |
| | | disease + SNHL | | | | | | | | | |
| 8342 | MT-TK | PEO and Myoclonus | G8342A | tRNA Lys | − | + | Reported | 77.20% | 0% | 0 | 4 |
| | | | | | | | | | (0%) | (0) | |
| 8343 | MT-TK | Metabolic syndrome | A8343G | tRNA Lys | + | − | Reported | 4.70% | 0.1% | 53 | 3 |
| | | and polycystic ovary | | | | | | | (0%) | (0) | |
| | | syndrome/possible | | | | | | | | | |
| | | PD risk factor | | | | | | | | | |
| 8344 | MT-TK | MERRF; Other - LD/ | A8344G | tRNA Lys | − | + | Cfrm | Pathogenic | 0% | 4 | 124 |
| | | Depressive mood disorder/ | | | | | | | (0%) | (0) | |
| | | leukoencephalopathy/HiCM | | | | | | | | | |
| 8347 | MT-TK | Poss. hypertension | A8347G | tRNA Lys | + | − | Reported | 2.60% | 0% | 19 | 2 |
| | | factor | | | | | | | (0%) | (0) | |
| 8348 | MT-TK | Cardiomyopathy/SNHL/ | A8348G | tRNA Lys | + | + | Reported | 33.80% | 0.2% | 118 | 8 |
| | | poss. hypertension | | | | | | | (0%) | (0) | |
| | | factor | | | | | | | | | |
| 8355 | MT-TK | Myopathy | T8355C | tRNA Lys | − | + | Reported | 67.20% | 0% | 0 | 2 |
| | | | | | | | | | (0%) | (0) | |
| 8356 | MT-TK | MERRF | T8356C | tRNA Lys | − | + | Cfrm | Pathogenic | 0% | 0 | 10 |
| | | | | | | | | | (0%) | (0) | |
| 8357 | MT-TK | Multiple symmetric | T8357C | tRNA Lys | − | + | Reported | 59.10% | 0% | 0 | 1 |
| | | lipomatosis | | | | | | | (0%) | (0) | |
| 8361 | MT-TK | MERRF | G8361A | tRNA Lys | − | + | Reported | 64.80% | 0% | 0 | 3 |
| | | | | | | | | | (0%) | (0) | |
| 8362 | MT-TK | Myopathy | T8362G | tRNA Lys | − | + | Reported | 93.00% | 0% | 0 | 5 |
| | | | | | | | | | (0%) | (0) | |
| 8363 | MT-TK | MICM + DEAF/MERRF/ | G8363A | tRNA Lys | − | + | Cfrm | Pathogenic | 0% | 0 | 20 |
| | | Autism/LS/ | | | | | | | (0%) | (0) | |
| | | Ataxia + Lipomas | | | | | | | | | |
| 9997 | MT-TG | MHCM | T9997C | tRNA Gly | − | + | Reported | 80.30% | 0% | 1 | 5 |
| | | | | | | | | | (0%) | (0) | |
| 10003 | MT-TG | Hypertension | T10003C | tRNA Gly | . | . | Reported | 0.40% | 0% | 8 | 1 |
| | | | | | | | | | (0%) | (0) | |
| 10006 | MT-TG | CIPO/Encephalopathy | A10006G | tRNA Gly | + | − | Unclear | 19.30% | 0% | 9 | 4 |
| | | | | | | | | | (0%) | (0) | |
| 10010 | MT-TG | PEM | T10010C | tRNA Gly | − | + | Cfrm | Pathogenic | 0% | 0 | 9 |
| | | | | | | | | | (0%) | (0) | |
| 10014 | MT-TG | Myopathy | G10014A | tRNA Gly | + | − | Unclear | 60.90% | 0% | 1 | 1 |
| | | | | | | | | | (0%) | (0) | |
| 10044 | MT-TG | SIDS | A10044G | tRNA Gly | − | + | Unclear | 34.70% | 0.3% | 135 | 8 |
| | | | | | | | | | (0%) | (0) | |
| 10406 | MT-TR | Mitochondrial | G10406A | tRNA Arg | − | + | Reported | 72.30% | 0% | 0 | 2 |
| | | myopathy | | | | | | | (0%) | (0) | |
| 10411 | MT-TR | Dilated | A10411T | tRNA Arg | + | − | Reported | 26.40% | 0% | 0 | 1 |
| | | Cardiomyopathy | | | | | | | (0%) | (0) | |
| 10415 | MT-TR | Dilated | T10415C | tRNA Arg | + | − | Reported | 76.50% | 0% | 0 | 1 |
| | | Cardiomyopathy | | | | | | | (0%) | (0) | |
| 10437 | MT-TR | Mitochondrial | G10437A | tRNA Arg | − | + | Reported | 51.70% | 0% | 0 | 1 |
| | | myopathy | | | | | | | (0%) | (0) | |
| 10438 | MT-TR | Progressive | A10438G | tRNA Arg | − | + | Reported | 46.20% | 0% | 0 | 1 |
| | | Encephalopathy | | | | | | | (0%) | (0) | |
| 10450 | MT-TR | Combined OXPHOS | A10450G | tRNA Arg | − | + | Reported | 69.60% | 0% | 0 | 1 |
| | | defects & severe | | | | | | | (0%) | (0) | |
| | | multisystem disorder | | | | | | | | | |
| 10454 | MT-TR | DEAF helper mut. | T10454C | tRNA Arg | + | − | Reported | 4.80% | 0.4% | 181 | 3 |
| | | | | | | | | | (0%) | (0) | |
| 12146 | MT-TH | MELAS | A12146G | tRNA His | + | + | Reported | 61.60% | 0% | 0 | 1 |
| | | | | | | | | | (0%) | (0) | |
| 12147 | MT-TH | MERRF-MELAS/ | G12147A | tRNA His | − | + | Cfrm | Pathogenic | 0% | 0 | 5 |
| | | Encephalopathy | | | | | | | (0%) | (0) | |
| 12148 | MT-TH | Developmental delay, | T12148C | tRNA His | − | + | Reported | 74.70% | 0% | 1 | 1 |
| | | optic atrophy, | | | | | | | (0%) | (0) | |
| | | cataract, hearing | | | | | | | | | |
| | | loss, myopathy | | | | | | | | | |
| 12183 | MT-TH | RP + DEAF | G12183A | tRNA His | − | + | Reported | 70.30% | 0% | 1 | 3 |
| | | | | | | | | | (0%) | (0) | |
| 12187 | MT-TH | Asthenozoospermia | C12187A | tRNA His | + | − | Reported | 15.40% | 0% | 0 | 1 |
| | | | | | | | | | (0%) | (0) | |
| 12192 | MT-TH | MICM | G12192A | tRNA His | + | − | Reported | 4.50% | 0.2% | 112 | 2 |
| | | | | | | | | | (0%) | (0) | |
| 12201 | MT-TH | Maternally inherited non- | T12201C | tRNA His | − | + | Reported | 66.70% | 0% | 1 | 1 |
| | | syndromic deafness | | | | | | | (0%) | (0) | |
| 12206 | MT-TH | MELAS-like | C12206T | tRNA His | − | + | Reported | 44.20% | 0% | 0 | 1 |
| | | encephalopathy + bilateral | | | | | | | (0%) | (0) | |
| | | optic atrophy | | | | | | | | | |
| 12207 | MT-TS2 | Myopathy/Encephalopathy | G12207A | tRNA Ser | − | + | Reported | 76.40% | 0% | 0 | 3 |
| | | | | (AGY) | | | | | (0%) | (0) | |
| 12224 | MT-TS2 | DEAF helper mut. | C12224T | tRNA Ser | + | − | Reported | 30.40% | 0% | 4 | 1 |
| | | | | (AGY) | | | | | (0%) | (0) | |
| 12236 | MT-TS2 | DEAF | G12236A | tRNA Ser | + | − | Reported | 2.20% | 0.7% | 373 | 4 |
| | | | | (AGY) | | | | | (0%) | (0) | |
| 12246 | MT-TS2 | CIPO | C12246A | tRNA Ser | . | . | Reported | 3.20% | 0% | 3 | 2 |
| | | | | (AGY) | | | | | (0%) | (0) | |
| 12258 | MT-TS2 | DMDF/RP + SNHL | C12258A | tRNA Ser | − | + | Cfrm | Pathogenic | 0% | 1 | 7 |
| | | | | (AGY) | | | | | (0%) | (0) | |
| 12261 | MT-TS2 | Myopathy + | T12261C | tRNA Ser | − | + | Reported | 65.30% | 0% | 0 | 1 |
| | | epilepsy + retinal | | (AGY) | | | | | (0%) | (0) | |
| | | degeneration + DEAF | | | | | | | | | |
| 12262 | MT-TS2 | Progressive MM + | C12262A | tRNA Ser | − | + | Reported | 84.50% | 0% | 0 | 1 |
| | | Deafness + Seizures | | (AGY) | | | | | (0%) | (0) | |
| 12264 | MT-TS2 | Multisystem Disease | C12264T | tRNA Ser | + | + | Reported | 79.30% | 0% | 0 | 2 |
| | | with Cataracts/ | | (AGY) | | | | | (0% | (0) | |
| | | Myopathy + epilepsy + | | | | | | | | | |
| | | DEAF + atypical | | | | | | | | | |
| | | autism | | | | | | | | | |
| 12276 | MT-TL2 | CPEO | G12276A | tRNA Leu | − | + | Cfrm | Pathogenic | 0% | 1 | 3 |
| | | | | (CUN) | | | | | (0%) | (0) | |
| 12280 | MT-TL2 | Hypertension | A12280G | tRNA Leu | + | − | Reported | 6.50% | 0.1% | 72 | 1 |
| | | | | (CUN) | | | | | (0%) | (0) | |
| 12283 | MT-TL2 | CPEO | G12283A | tRNA Leu | − | + | Reported | 43.20% | 0% | 1 | 2 |
| | | | | (CUN) | | | | | (0%) | (0) | |
| 12293 | MT-TL2 | Axial mitochondrial | G12293A | tRNA Leu | − | + | Reported | 66.90% | 0% | 0 | 1 |
| | | myopathy | | (CUN) | | | | | (0%) | (0) | |
| 12294 | MT-TL2 | CPEO/EXIT + | G12294A | tRNA Leu | − | + | Cfrm | Pathogenic | 0% | 0 | 2 |
| | | Ophthalmoplegia | | (CUN) | | | | | (0%) | (0) | |
| 12297 | MT-TL2 | Dilated | T12297C | tRNA Leu | + | + | Reported | 47.30% | 0.1% | 39 | 5 |
| | | Cardiomyopathy/LS/ | | (CUN) | | | | | (0%) | (0) | |
| | | Failure to | | | | | | | | | |
| | | Thrive & LA | | | | | | | | | |
| 12299 | MT-TL2 | MELAS | A12299C | tRNA Leu | − | + | Reported | 53.00% | 0% | 0 | 1 |
| | | | | (CUN) | | | | | (0%) | (0) | |
| 12300 | MT-TL2 | 3243 suppressor mutant | G12300A | tRNA Leu | − | + | Reported | 51.70% | 0% | 0 | 4 |
| | | | | (CUN) | | | | | (0%) | (0) | |
| 12308 | MT-TL2 | CPEO/Stroke/CM/Breast & | A12308G | tRNA Leu | + | + | Reported | 42.00% | 12.4% | 6215 | 19 |
| | | Renal & Prostate Cancer | | (CUN) | | | | | (0%) | (0) | |
| | | Risk/Altered brain pH/sCJD | | | | | | | | | |
| 12311 | MT-TL2 | CPEO | T12311C | tRNA Leu | + | + | Reported | 34.40% | 0.1% | 57 | 3 |
| | | | | (CUN) | | | | | (0%) | (0) | |
| 12313 | MT-TL2 | FSHD | T12313C | tRNA Leu | − | + | Reported | 73.20% | 0% | 0 | 1 |
| | | | | (CUN) | | | | | (0%) | (0) | |
| 12315 | MT-TL2 | CPEO/KSS/possible | G12315A | tRNA Leu | − | + | Cfrm | Pathogenic | 0% | 0 | 13 |
| | | carotid atherosclerosis risk, | | (CUN) | | | | | (0%) | (0) | |
| | | trend toward myocardial | | | | | | | | | |
| | | infarction risk | | | | | | | | | |
| 12316 | MT-TL2 | CPEO | G12316A | tRNA Leu | − | + | Cfrm | Pathogenic | 0% | 0 | 2 |
| | | | | (CUN) | | | | | (0%) | (0) | |
| 12317 | MT-TL2 | CPEO + ptosis + | T12317C | tRNA Leu | − | + | Reported | 41.30% | 0% | 1 | 1 |
| | | myopathy + exereise | | (CUN) | | | | | (0%) | (0) | |
| | | intolerance + diabetes | | | | | | | | | |
| 12320 | MT-TL2 | MM | A12320G | tRNA Leu | − | + | Reported | 37.30% | 0% | 0 | 7 |
| | | | | (CUN) | | | | | (0%) | (0) | |
| 14674 | MT-TE | Reversible COX | T14674C | tRNA Glu | + | − | Cfrm | Pathogenic | 0% | 7 | 6 |
| | | deficiency myopathy | | | | | | | (0%) | (0) | |
| 14674 | MT-TE | Reversible COX | T14674G | tRNA Glu | + | − | Reported | 29.40% | 0% | 0 | 1 |
| | | deficiency myopathy | | | | | | | (0%) | (0) | |
| 14680 | MT-TE | Mitochondrial | C14680A | tRNA Glu | − | + | Reported | 35.50% | 0% | 0 | 1 |
| | | encephalomyopathy | | | | | | | (0%) | (0) | |
| 14685 | MT-TE | Cataracts w spastic | G14685A | tRNA Glu | − | + | Reported | 77.40% | 0% | 0 | 1 |
| | | paraparesis & ataxia | | | | | | | (0%) | (0) | |
| 14687 | MT-TE | Mito myopathy | A14687G | tRNA Glu | + | − | Reported | 7.00% | 0.6% | 299 | 3 |
| | | w respiratory | | | | | | | (0%) | (0) | |
| | | failure; intellectual | | | | | | | | | |
| | | disability | | | | | | | | | |
| 14692 | MT-TE | LHON helper/Maternally | A14692G | tRNA Glu | + | − | Reported | 2.40% | 0% | 19 | 3 |
| | | inherited diabetes & | | | | | | | (0%) | (0) | |
| | | deafness | | | | | | | | | |
| 14693 | MT-TE | MELAS/LHON/DEAF/ | A14693G | tRNA Glu | + | + | Reported | 39.50% | 0.5% | 262 | 12 |
| | | hypertension helper | | | | | | | (0%) | (0) | |
| 14696 | MT-TE | Progressive | A14696G | tRNA Glu | − | + | Reported | 22.00% | 0.1% | 46 | 1 |
| | | Encephalopathy | | | | | | | (0%) | (0) | |
| 14709 | MT-TE | MM + DMDF/ | T14709C | tRNA Glu | + | + | Cfrm | Pathogenic | 0% | 1 | 22 |
| | | Encephalomyopathy/ | | | | | | | (0%) | (0) | |
| | | Dementia + diabetes + | | | | | | | | | |
| | | ophthalmoplegia | | | | | | | | | |
| 14710 | MT-TE | Encephalomyopathy + | G14710A | tRNA Glu | − | + | Cfrm | Pathogenic | 0% | 0 | 5 |
| | | Retinopathy | | | | | | | (0%) | (0) | |
| 14721 | MT-TE | Isolated complex | G14721A | tRNA Glu | − | + | Reported | 82.90% | 0% | 0 | 1 |
| | | I deficiency | | | | | | | (0%) | (0) | |
| 14723 | MT-TE | CPEO + | T14723C | tRNA Glu | − | + | Reported | 73.50% | 0% | 0 | 2 |
| | | Myopathy | | | | | | | (0%) | (0) | |
| 14724 | MT-TE | Mito | G14724A | tRNA Glu | − | + | Reported | 88.80% | 0% | 0 | 3 |
| | | Leukoencephalopathy | | | | | | | (0%) | (0) | |
| 14728 | MT-TE | Late-onset | T14728C | tRNA Glu | − | + | Reported | 48.50% | 0% | 0 | 1 |
| | | mitochondrial | | | | | | | (0%) | (0) | |
| | | encephalomyopathy | | | | | | | | | |
| 14739 | MT-TE | EXIT | G14739A | tRNA Glu | − | + | Reported | 62.10% | 0% | 0 | 2 |
| | | | | | | | | | (0%) | (0) | |
| 15894 | MT-TT | Gout | G15894A | tRNA Thr | + | − | Reported | 28.20% | 0.1% | 29 | 1 |
| | | | | | | | | | (0%) | (0) | |
| 15908 | MT-TT | DEAF helper mut. | T15908C | tRNA Thr | + | − | Reported | 28.00% | 0.3% | 127 | 2 |
| | | | | | | | | | (0%) | (0) | |
| 15909 | MT-TT | Hypertension | A15909G | tRNA Thr | + | − | Reported | 25.90% | 0% | 7 | 2 |
| | | | | | | | | | (0%) | (0) | |
| 15915 | MT-TT | Encephalomyopathy | G15915A | tRNA Thr | − | + | Reported | 73.70% | 0% | 1 | 2 |
| | | | | | | | | | (0%) | (0) | |
| 15923 | MT-TT | LIMM/MERRF/mito | A15923G | tRNA Thr | − | + | Reported | 46.60% | 0% | 0 | 5 |
| | | disease | | | | | | | (0%) | (0) | |
| 15924 | MT-TT | LIMM | A15924G | tRNA Thr | . | . | Reported | 22.70% | 3.5% | 1764 | 6 |
| | | | | | | | | | (0%) | (0) | |
| 15927 | MT-TT | LHON/Multiple Sclerosis/ | G15927A | tRNA Thr | + | − | Reported | 16.20% | 0.9% | 430 | 12 |
| | | DEAF1555 increased | | | | | | | (0%) | (0) | |
| | | penetrance/CHD | | | | | | | | | |
| 15928 | MT-TT | Multiple Sclerosis/ | G15928A | tRNA Thr | + | − | Reported | 20.20% | 4.9% | 2447 | 7 |
| | | idiopathic repeat | | | | | | | (0%) | (0) | |
| | | miscarriage/ | | | | | | | | | |
| | | AD protection | | | | | | | | | |
| 15933 | MT-TT | Suspected mito | G15933A | tRNA Thr | + | − | Reported | 66.80% | 0% | 0 | 1 |
| | | disease | | | | | | | (0%) | (0) | |
| 15942 | MT-TT | Possibly LVNC- | T15942C | tRNA Thr | + | − | Reported | 28.60% | 0.8% | 408 | 1 |
| | | associated | | | | | | | (0%) | (0) | |
| 15944 | MT-TT | MM | T15944del | tRNA Thr | + | − | Conflicting | 19.90% | 1.5% | 754 | 2 |
| | | | | | | | reports | | (0%) | (0) | |
| 15950 | MT-TT | Dopaminergic nerve | G15950A | tRNA Thr | + | − | Reported | 54.50% | 0% | 1 | 1 |
| | | cell death (PD) | | | | | | | (0%) | (0) | |
| 15951 | MT-TT | LHON/LHON modulator | A15951G | tRNA Thr | + | − | Conflicting | 23.70% | 0.8% | 381 | 6 |
| | | | | | | | reports | | (0%) | (0) | |
| 15965 | MT-TP | Dopaminergic nerve | A15965G | tRNA Pro | + | − | Reported | 2.10% | 0% | 9 | 1 |
| | | cell death (PD) | | | | | | | (0%) | (0) | |
| 15967 | MT-TP | MERRF-like disease | G15967A | tRNA Pro | − | + | Reported | 78.90% | 0% | 0 | 2 |
| | | | | | | | | | (0%) | (0) | |
| 15975 | MT-TP | Ataxia + RP + | C15975T | tRNA Pro | − | + | Reported | 78.30% | 0% | 0 | 1 |
| | | deafness | | | | | | | (0%) | (0) | |
| 15990 | MT-TP | MM | C15990T | tRNA Pro | − | + | Reported | 51.70% | 0% | 0 | 4 |
| | | | | | | | | | (0%) | (0) | |
| 15995 | MT-TP | Mitochondrial | G15995A | tRNA Pro | − | + | Reported | 80.00% | 0% | 0 | 2 |
| | | cytopathy | | | | | | | (0%) | (0) | |
| 15998 | MT-TP | Mitochondrial | A15998T | tRNA Pro | − | + | Reported | 57.50% | 0% | 0 | 1 |
| | | myopathy | | | | | | | (0%) | (0) | |
| 16002 | MT-TP | Mitochondrial | T16002C | tRNA Pro | − | + | Reported | 75.80% | 0% | 0 | 1 |
| | | cytopathy | | | | | | | (0%) | (0) | |
| 16015 | MT-TP | Mitochondrial | T16015C | tRNA Pro | − | + | Reported | 50.40% | 0% | 0 | 1 |
| | | myopathy | | | | | | | (0%) | (0) | |
| 16018 | MT-TP | Dilated | T16018TTCT | tRNA Pro | − | + | Reported | — | 0% | 0 | 1 |
| | | cardiomyopathy | CTGTTCTT | | | | | | (0%) | (0) | |
| | | (15 bp dup), | TCAT(SEQ | | | | | | | | |
| | | alternate notation | ID NO: 55) | | | | | | | | |
| 16021 | MT-TP | Mitochondrial | 16021_16022 | tRNA Pro | − | + | Reported | — | 0% | 0 | 1 |
| | | myopathy | delCT | | | | | | (0% | (0) | |
| 16023 | MT-TP | Migraine + pigmentary | G16023A | tRNA Pro | − | + | Reported | 83.70% | 0% | 0 | 1 |
| | | retinopathy + | | | | | | | (0%) | (0) | |
| | | deafness + leukariosis | | | | | | | | | |
| 16032 | MT-TP | Dilated | T16032TTCT | tRNA Pro | − | + | Reported | — | 0% | 1 | 1 |
| | | cardiomyopathy | CTGTTCTT | | | | | | (0%) | (0) | |
| | | (15 bp dup) | TCAT (SEQ | | | | | | | | |
| | | | ID NO: 55) | | | | | | | | |
| 16033 | MT-TP | Dilated | G16033TCT | tRNA Pro | − | + | Reported | — | 0% | 0 | 1 |
| | | cardiomyopathy | CTGTTCTT | | | | | | (0% | (0) | |
| | | (15 bp dup), | TCATG(SEQ | | | | | | | | |
| | | alternate notation | ID NO: 56) | | | | | | | | |

Column Heading Key: A: Position; B: Locus; C: Disease; D: Allele; E: RNA; F: Homoplasmy; G: Heteroplasmy; H: Status; I: MitoTip; J: GB Freq FL (CR); K: GB Seqs FL (CR); L: Reference

TABLE 5
MITOMAP: Mitochondrial DNA Base Substitution Diseases: rRNA/tRNA Mutations with Cfrm Status

| A | B | C | D | E | F | G | H | I | J | K | L |
|---|---|---|---|---|---|---|---|---|---|---|---|
| 583 | MT-TF | MELAS/MM & | G583A | tRNA | − | + | Cfrm | Pathogenic | 0.0% | 0 (0) | 3 |
| | | EXIT | | Phe | | | | | (0.0%) | | |
| 616 | MT-TF | Maternally inherited | T616C | tRNA | + | + | Cfrm | Pathogenic | 0.0% | 1 (0) | 2 |
| | | epilepsy/kidney | | Phe | | | | | (0.0%) | | |
| | | disease | | | | | | | | | |
| 1494 | MT- | DEAF | C1494T | 12S | + | − | Cfrm | N/A | 0.0% | 4 (0) | 29 |
| | RNR1 | | | rRNA | | | | | (0.0%) | | |
| 1555 | MT- | DEAF; autism | A1555G | 12S | + | − | Cfrm | N/A | 0.1% | 74 (0) | 138 |
| | RNR1 | spectrum intellectual | | rRNA | | | | | (0.0%) | | |
| | | disability; possibly | | | | | | | | | |
| | | antiatherosclerotic | | | | | | | | | |
| 1606 | MT-TV | AMDF | G1606A | tRNA | − | + | Cfrm | Pathogenic | 0.0% | 0 (0) | 4 |
| | | | | Val | | | | | (0.0%) | | |
| 1630 | MT-TV | MNGIE-like disease/ | A1630G | tRNA | − | + | Cfrm | Pathogenic | 0.0% | 0 (0) | 3 |
| | | MELAS | | Val | | | | | (0.0%) | | |
| 1644 | MT-TV | LS/HCM/MELAS | G1644A | tRNA | − | + | Cfrm | Pathogenic | 0.0% | 0 (0) | 4 |
| | | | | Val | | | | | (0.0%) | | |
| 3243 | MT-TL1 | MELAS/LS/ | A3243G | tRNA | − | + | Cfrm | Pathogenic | 0.0% | 9 (0) | 392 |
| | | DMDF/MIDD/ | | Leu | | | | | (0.0%) | | |
| | | SNHL/CPEO/MM/ | | (UUR) | | | | | | | |
| | | FSGS/ASD/ | | | | | | | | | |
| | | Cardiac + multi-organ | | | | | | | | | |
| | | dysfunction | | | | | | | | | |
| 3243 | MT-TL1 | MM/MELAS/ | A3243T | tRNA | − | + | Cfrm | Pathogenic | 0.0% | 0 (0) | 6 |
| | | SNHL/CPEO | | Leu | | | | | (0.0%) | | |
| | | | | (UUR) | | | | | | | |
| 3256 | MT-TL1 | MELAS; possible | C3256T | tRNA | − | + | Cfrm | Pathogenic | 0.0% | 0 (0) | 18 |
| | | atherosclerosis risk | | Leu | | | | | (0.0%) | | |
| | | | | (UUR) | | | | | | | |
| 3258 | MT-TL1 | MELAS/Myopathy | T3258C | tRNA | − | + | Cfrm | Pathogenic | 0.0% | 1 (0) | 5 |
| | | | | Leu | | | | | (0.0%) | | |
| | | | | (UUR) | | | | | | | |
| 3260 | MT-TL1 | MMC/MELAS | A3260G | tRNA | − | + | Cfrm | Pathogenic | 0.0% | 0 (0) | 10 |
| | | | | Leu | | | | | (0.0%) | | |
| | | | | (UUR) | | | | | | | |
| 3271 | MT-TL1 | PEM/retinal | T3271del | tRNA | − | + | Cfrm | Pathogenic | 0.0% | 0 (0) | 3 |
| | | dystrophy in | | Leu | | | | | (0.0%) | | |
| | | MELAS | | (UUR) | | | | | | | |
| 3271 | MT-TL1 | MELAS/DM | T3271C | tRNA | − | + | Cfrm | Pathogenic | 0.0% | 0 (0) | 25 |
| | | | | Leu | | | | | (0.0%) | | |
| | | | | (UUR) | | | | | | | |
| 3280 | MT-TL1 | Myopathy | A3280G | tRNA | − | + | Cfrm | Pathogenic | 0.0% | 0 (0) | 6 |
| | | | | Leu | | | | | (0.0%) | | |
| | | | | (UUR) | | | | | | | |
| 3291 | MT-TL1 | MELAS/Myopathy/ | T3291C | tRNA | − | + | Cfrm | Pathogenic | 0.0% | 0 (0) | 14 |
| | | Deafness + | | Leu | | | | | (0.0%) | | |
| | | Cognitive | | (UUR) | | | | | | | |
| | | Impairment | | | | | | | | | |
| 3302 | MT-TL1 | MM | A3302G | tRNA | − | + | Cfrm | Pathogenic | 0.0% | 0 (0) | 10 |
| | | | | Leu | | | | | (0.0%) | | |
| | | | | (UUR) | | | | | | | |
| 3303 | MT-TL1 | MMC | C3303T | tRNA | + | + | Cfrm | Pathogenic | 0.0% | 0 (0) | 12 |
| | | | | Leu | | | | | (0.0%) | | |
| | | | | (UUR) | | | | | | | |
| 4298 | MT-TI | CPEO/MS | G4298A | tRNA | − | + | Cfrm | Pathogenic | 0.0% | 0 (0) | 9 |
| | | | | Ile | | | | | (0.0%) | | |
| 4300 | MT-TI | MICM | A4300G | tRNA | + | + | Cfrm | Pathogenic | 0.0% | 0 (0) | 9 |
| | | | | Ile | | | | | (0.0%) | | |
| 4308 | MT-TI | CPEO | G4308A | tRNA | − | + | Cfrm | Pathogenic | 0.0% | 0 (0) | 2 |
| | | | | Ile | | | | | (0.0%) | | |
| 4332 | MT-TQ | Encephalopathy/ | G4332A | tRNA | − | + | Cfrm | Pathogenic | 0.0% | 0 (0) | 4 |
| | | MELAS | | Gln | | | | | (0.0%) | | |
| 4450 | MT-TM | Myopathy/MELAS/ | G4450A | tRNA | − | + | Cfrm | Pathogenic | 0.0% | 0 (0) | 4 |
| | | Leigh Syndrome | | Met | | | | | (0.0%) | | |
| 5521 | MT-TW | Mitochondrial | G5521A | tRNA | − | + | Cfrm | Pathogenic | 0.0% | 0 (0) | 5 |
| | | myopathy | | Trp | | | | | (0.0%) | | |
| 5537 | MT-TW | Leigh Syndrome | A5537insT | tRNA | − | + | Cfrm | — | 0.0% | 0 (0) | 5 |
| | | | | Trp | | | | | (0.0%) | | |
| 5650 | MT-TA | Myopathy | G5650A | tRNA | − | + | Cfrm | Pathogenic | 0.0% | 1 (0) | 7 |
| | | | | Ala | | | | | (0.0%) | | |
| 5690 | MT-TN | CPEO + ptosis + | A5690G | tRNA | − | + | Cfrm | Pathogenic | 0.0% | 0 (0) | 2 |
| | | proximal myopathy | | Asn | | | | | (0.0%) | | |
| 5703 | MT-TN | CPEO/MM | G5703A | tRNA | − | + | Cfrm | Pathogenic | 0.0% | 0 (0) | 7 |
| | | | | Asn | | | | | (0.0%) | | |
| 5728 | MT-TN | Multiorgan failure/ | T5728C | tRNA | − | + | Cfrm | Pathogenic | 0.0% | 1 (0) | 3 |
| | | myopathy | | Asn | | | | | (0.0%) | | |
| 7445 | MT-TS1 | SNHL | A7445G | tRNA | + | + | Cfrm | — | 0.0% | 1 (0) | 32 |
| | precursor | | | Ser | | | | | (0.0%) | | |
| | | | | (UCN) | | | | | | | |
| | | | | precursor | | | | | | | |
| 7471 | MT-TS1 | PEM/AMDF/ | C7471CC | tRNA | + | + | Cfrm | — | 0.0% | 7 (0) | 28 |
| | | Motor neuron | | Ser | | | | | (0.0%) | | |
| | | disease-like | | (UCN) | | | | | | | |
| 7497 | MT-TS1 | MM/EXIT | G7497A | tRNA | + | + | Cfrm | Pathogenic | 0.0% | 1 (0) | 7 |
| | | | | Ser | | | | | (0.0%) | | |
| | | | | (UCN) | | | | | | | |
| 7510 | MT-TS1 | SNHL | T7510C | tRNA | − | + | Cfrm | Pathogenic | 0.0% | 1 (0) | 13 |
| | | | | Ser | | | | | (0.0%) | | |
| | | | | (UCN) | | | | | | | |
| 7511 | MT-TS1 | SNHL/Deafness | T7511C | tRNA | + | + | Cfrm | Pathogenic | 0.0% | 2 (0) | 20 |
| | | | | Ser | | | | | (0.0%) | | |
| | | | | (UCN) | | | | | | | |
| 8306 | MT-TK | Severe adult-onset | T8306C | tRNA | − | + | Cfrm | Pathogenic | 0.0% | 0 (0) | 3 |
| | | multisymptom | | Lys | | | | | (0.0%) | | |
| | | myopathy/ | | | | | | | | | |
| | | Myoclonic epilepsy | | | | | | | | | |
| 8313 | MT-TK | MNGIE/ | G8313A | tRNA | − | + | Cfrm | Pathogenic | 0.0% | 1 (0) | 6 |
| | | Progressive mito | | Lys | | | | | (0.0%) | | |
| | | cytopathy | | | | | | | | | |
| 8340 | MT-TK | Myopathy/Exercise | G8340A | tRNA | − | + | Cfrm | Pathogenic | 0.0% | 0 (0) | 7 |
| | | Intolerance/Eye | | Lys | | | | | (0.0%) | | |
| | | disease + SNHL | | | | | | | | | |
| 8344 | MT-TK | MERRF; Other - LD/ | A8344G | tRNA | − | + | Cfrm | Pathogenic | 0.0% | 4 (0) | 124 |
| | | Depressive mood | | Lys | | | | | (0.0%) | | |
| | | disorder/ | | | | | | | | | |
| | | leukoencephalopathy/ | | | | | | | | | |
| | | HiCM | | | | | | | | | |
| 8356 | MT-TK | MERRF | T8356C | tRNA | − | + | Cfrm | Pathogenic | 0.0% | 0 (0) | 10 |
| | | | | Lys | | | | | (0.0%) | | |
| 8363 | MT-TK | MICM + DEAF/ | G8363A | tRNA | − | + | Cfrm | Pathogenic | 0.0% | 0 (0) | 20 |
| | | MERRF/Autism/ | | Lys | | | | | (0.0%) | | |
| | | LS/Ataxia + | | | | | | | | | |
| | | Lipomas | | | | | | | | | |
| 10010 | MT-TG | PEM | T10010C | tRNA | − | + | Cfrm | Pathogenic | 0.0% | 0 (0) | 9 |
| | | | | Gly | | | | | (0.0%) | | |
| 12147 | MT-TH | MERRF-MELAS/ | G12147A | tRNA | − | + | Cfrm | Pathogenic | 0.0% | 0 (0) | 5 |
| | | Encephalopathy | | His | | | | | (0.0%) | | |
| 12258 | MT-TS2 | DMDF/RP + | C12258A | tRNA | − | + | Cfrm | Pathogenic | 0.0% | 1 (0) | 7 |
| | | SNHL | | Ser | | | | | (0.0%) | | |
| | | | | (AGY) | | | | | | | |
| 12276 | MT-TL2 | CPEO | G12276A | tRNA | − | + | Cfrm | Pathogenic | 0.0% | 1 (0) | 3 |
| | | | | Leu | | | | | (0.0%) | | |
| | | | | (CUN) | | | | | | | |
| 12294 | MT-TL2 | CPEO/EXIT + | G12294A | tRNA | − | + | Cfrm | Pathogenic | 0.0% | 0 (0) | 2 |
| | | Ophthalmoplegia | | Leu | | | | | (0.0%) | | |
| | | | | (CUN) | | | | | | | |
| 12315 | MT-TL2 | CPEO/KSS/ | G12315A | tRNA | − | + | Cfrm | Pathogenic | 0.0% | 0 (0) | 13 |
| | | possible carotid | | Leu | | | | | (0.0%) | | |
| | | atherosclerosis risk, | | (CUN) | | | | | | | |
| | | trend toward | | | | | | | | | |
| | | myocardial | | | | | | | | | |
| | | infarction risk | | | | | | | | | |
| 12316 | MT-TL2 | CPEO | G12316A | tRNA | − | + | Cfrm | Pathogenic | 0.0% | 0 (0) | 2 |
| | | | | Leu | | | | | (0.0%) | | |
| | | | | (CUN) | | | | | | | |
| 14674 | MT-TE | Reversible COX | T14674C | tRNA | + | − | Cfrm | Pathogenic | 0.0% | 7 (0) | 6 |
| | | deficiency myopathy | | Glu | | | | | (0.0%) | | |
| 14709 | MT-TE | MM + DMDF/ | T14709C | tRNA | + | + | Cfrm | Pathogenic | 0.0% | 1 (0) | 22 |
| | | Encephalomyopathy/ | | Glu | | | | | (0.0%) | | |
| | | Dementia + | | | | | | | | | |
| | | diabetes + | | | | | | | | | |
| | | ophthalmoplegia | | | | | | | | | |
| 14710 | MT-TE | Encephalomyopathy + | G14710A | tRNA | − | + | Cfrm | Pathogenic | 0.0% | 0 (0) | 5 |
| | | Retinopathy | | Glu | | | | | (0.0%) | | |

Column Heading Key:
A: Position;
B: Locus;
C: Disease;
D: Allele;
E: RNA;
F: Homoplasmy;
G: Heteroplasmy;
H: Status;
I: MitoTip;
J: GB Freq FL (CR);
K: GB Seqs FL (CR);
L: Reference

TABLE 6
MITOMAP: Reported Mitochondrial DNA Base Substitution
Diseases: Coding and Region Point Mutations

| A | B | C | D | E |
|---|---|---|---|---|
| 114 | MT- | BD-associated | C114T | C-T |
| | CR | | | |
| 146 | MT- | Absence of | T146C | T-C |
| | CR | Endometriosis | | |
| 150 | MT- | Longevity/Cervical | C150T | C-T |
| | CR | Carcinoma/HPV | | |
| | | infection risk | | |
| 195 | MT- | BD-associated/ | T195C | T-C |
| | CR | melanoma pts | | |
| 302 | MT- | Higher in melanoma | A302ACC | A-ACC |
| | CR | patient group | | |
| 309 | MT- | AD-weakly associated | C309CC | C-CC |
| | CR | | | |
| 310 | MT- | Melanoma patients | T310TC | T-TC |
| | CR | | | |
| 499 | MT- | Endometriosis | G499A | G-A |
| | CR | | | |
| 547 | MT- | Tubulointerstitial | A547T | A-T |
| | CR | kidney disease | | |
| 573 | MT- | Absence of | C573CCC | C-CCC |
| | CR | Endometriosis | | |
| 3308 | MT- | MELAS/DEAF enhancer/ | T3308C | T-C |
| | ND1 | hypertension/LVNC/ | | |
| | | putative LHON | | |
| 3308 | MT- | Sudden Infant Death | T3308G | T-G |
| | ND1 | | | |
| 3310 | MT- | Diabetes/HCM | C3310T | C-T |
| | ND1 | | | |
| 3316 | MT- | Diabetes/LHON/ | G3316A | G-A |
| | ND1 | PEO | | |
| 3335 | MT- | LHON | T3335C | T-C |
| | ND1 | | | |
| 3336 | MT- | Carotid | T3336C | T-C |
| | ND1 | atherosclerosis risk | | |
| 3337 | MT- | Cardiomyopathy | G3337A | G-A |
| | ND1 | | | |
| 3340 | MT- | Encephaloneuromyopathy | C3340T | C-T |
| | ND1 | | | |
| 3376 | MT- | LHON MELAS overlap | G3376A | G-A |
| | ND1 | | | |
| 3380 | MT- | MELAS | G3380A | G-A |
| | ND1 | | | |
| 3388 | MT- | Materally Inherited | C3388A | C-A |
| | ND1 | Nonsyndromic Deafness | | |
| 3391 | MT- | LHON | G3391A | G-A |
| | ND1 | | | |
| 3394 | MT- | LHON/Diabetes/ | T3394C | T-C |
| | ND1 | CPTdeficiency/high | | |
| | | altitude adaptation | | |
| 3395 | MT- | LHON/HCM with | A3395G | A-G |
| | ND1 | hearing loss | | |
| 3396 | MT- | NSHL/MIDD | T3396C | T-C |
| | ND1 | | | |
| 3397 | MT- | ADPD/Possibly | A3397G | A-G |
| | ND1 | LVNC-cardiomyopathy | | |
| | | associated | | |
| 3398 | MT- | DMDF + HCM/GDM/ | T3398C | T-C |
| | ND1 | possibly LVNC | | |
| | | cardiomyopathy- | | |
| | | associated | | |
| 3399 | MT- | Gestational Diabetes | A3399T | A-T |
| | ND1 | (GDM) | | |
| 3407 | MT- | HCM/Muscle | G3407A | G-A |
| | ND1 | involvement | | |
| 3418 | MT- | AMegL | A3418G | A-G |
| | ND1 | | | |
| 3421 | MT- | MIDD | G3421A | G-A |
| | ND1 | | | |
| 3460 | MT- | LHON | G3460A | G-A |
| | ND1 | | | |
| 3472 | MT- | LHON | T3472C | T-C |
| | ND1 | | | |
| 3481 | MT- | MELAS/Progressive | G3481A | G-A |
| | ND1 | Encephalomyopathy | | |
| 3488 | MT- | LHON | T3488C | T-C |
| | ND1 | | | |
| 3496 | MT- | LHON | G3496T | G-T |
| | ND1 | | | |
| 3497 | MT- | LHON | C3497T | C-T |
| | ND1 | | | |
| 3551 | MT- | LHON | C3551T | C-T |
| | ND1 | | | |
| 3571 | MT- | Possible LHON helper | C3571T | C-T |
| | ND1 | mut. | | |
| 3632 | MT- | LHON | C3632T | C-T |
| | ND1 | | | |
| 3634 | MT- | LHON | A3634G | A-G |
| | ND1 | | | |
| 3635 | MT- | LHON | G3635A | G-A |
| | ND1 | | | |
| 3644 | MT- | BD-associated | T3644C | T-C |
| | ND1 | | | |
| 3667 | MT- | Peripheral neuropathy | T3667G | T-G |
| | ND1 | of T2 diabetes | | |
| 3688 | MT- | Leigh Syndrome | G3688A | G-A |
| | ND1 | | | |
| 3697 | MT- | MELAS/LS/LDYT/ | G3697A | G-A |
| | ND1 | BSN | | |
| 3700 | MT- | LHON | G3700A | G-A |
| | ND1 | | | |
| 3713 | MT- | LHON | T3713C | T-C |
| | ND1 | | | |
| 3733 | MT- | LHON | G3733A | G-A |
| | ND1 | | | |
| 3733 | MT- | LHON | G3733C | G-C |
| | ND1 | | | |
| 3736 | MT- | LHON | G3736A | G-A |
| | ND1 | | | |
| 3745 | MT- | LHON/high altitude | G3745A | G-A |
| | ND1 | variant | | |
| 3769 | MT- | LHON | C3769G | C-G |
| | ND1 | | | |
| 3781 | MT- | LHON | T3781C | T-C |
| | ND1 | | | |
| 3796 | MT- | Adult-Onset Dystonia | A3796G | A-G |
| | ND1 | | | |
| 3833 | MT- | PEG | T3833A | T-A |
| | ND1 | | | |
| 3866 | MT- | LHON + limb | T3866C | T-C |
| | ND1 | claudication | | |
| 3890 | MT- | Progressive | G3890A | G-A |
| | ND1 | Encephalomyopathy/ | | |
| | | LS/Optic Atrophy | | |
| 3902 | MT- | EXIT + myalgia/severe | 3902_{—} | ACCTTGC- |
| | ND1 | LA + cardiac/3-MGA | 3908inv | GCAAGGT |
| | | aciduria | | |
| 3919 | MT- | LHON | T3919C | T-C |
| | ND1 | | | |
| 3945 | MT- | Leigh-like phenotype | C3945A | C-A |
| | ND1 | | | |
| 3946 | MT- | MELAS | G3946A | G-A |
| | ND1 | | | |
| 3949 | MT- | MELAS | T3949C | T-C |
| | ND1 | | | |
| 3958 | MT- | LHON | G3958A | G-A |
| | ND1 | | | |
| 3959 | MT- | MELAS | G3959A | G-A |
| | ND1 | | | |
| 3995 | MT- | MELAS | A3995G | A-G |
| | ND1 | | | |
| 4081 | MT- | LHON | T4081C | T-C |
| | ND1 | | | |
| 4123 | MT- | LHON | A4123T | A-T |
| | ND1 | | | |
| 4132 | MT- | NAION-associated | G4132A | G-A |
| | ND1 | | | |
| 4136 | MT- | LHON | A4136G | A-G |
| | ND1 | | | |
| 4142 | MT- | Developmental delay, | G4142A | G-A |
| | ND1 | seizure, hypotonia | | |
| 4160 | MT- | LHON/LHON plus | T4160C | T-C |
| | ND1 | | | |
| 4163 | MT- | LHON | T4163C | T-C |
| | ND1 | | | |
| 4171 | MT- | LHON/Leigh-like | C4171A | C-A |
| | ND1 | phenotype | | |
| 4216 | MT- | LHON/Insulin | T4216C | T-C |
| | ND1 | Resistance/possible | | |
| | | adaptive high altitude | | |
| | | variant | | |
| 4633 | MT- | LHON candidate | C4633G | C-G |
| | ND2 | | | |
| 4640 | MT- | LHON/Epilepsy | C4640A | C-A |
| | ND2 | | | |
| 4648 | MT- | PEG | T4648C | T-C |
| | ND2 | | | |
| 4659 | MT- | possible PD risk | G4659A | G-A |
| | ND2 | factor | | |
| 4681 | MT- | Leigh Syndrome | T4681C | T-C |
| | ND2 | | | |
| 4769 | MT- | SZ-associated | A4769A | A-A |
| | ND2 | | | |
| 4833 | MT- | Diabetes helper | A4833G | A-G |
| | ND2 | mutation AD, PD | | |
| 4852 | MT- | LHON | T4852A | T-A |
| | ND2 | | | |
| 4883 | MT- | Glaucoma | C4883T | C-T |
| | ND2 | | | |
| 4917 | MT- | LHON/Insulin | A4917G | A-G |
| | ND2 | Resistance/AMD/ | | |
| | | NRTI-PN | | |
| 5001 | MT- | Developmental delay, | A5001AA | A-AA |
| | ND2 | seizure, cardiomyopathy, | | |
| | | lactic acidosis | | |
| 5095 | MT- | Proximal muscle | T5095C | T-C |
| | ND2 | weakness and atrophy | | |
| 5133 | MT- | Exercise intolerance | 5133_{—} | AA-del |
| | ND2 | (EXIT) | 5134delAA | |
| 5178 | MT- | Longevity/ | C5178A | C-A |
| | ND2 | Extraversion/diabetes/ | | |
| | | AMS protection/ | | |
| | | blood iron metabolism/ | | |
| | | correlation with | | |
| | | myocardial infarction/ | | |
| | | atherosclerosis | | |
| 5244 | MT- | LHON | G5244A | G-A |
| | ND2 | | | |
| 5452 | MT- | Progressive | C5452T | C-T |
| | ND2 | Encephalomyopathy | | |
| 5460 | MT- | AD/PD | G5460A | G-A |
| | ND2 | | | |
| 5460 | MT- | AD | G5460T | G-T |
| | ND2 | | | |
| 5911 | MT- | Prostate Cancer | C5911T | C-T |
| | CO1 | | | |
| 5913 | MT- | Prostate Cancer/ | G5913A | G-A |
| | CO1 | hypertension | | |
| 5920 | MT- | Myoglobinuria/EXIT | G5920A | G-A |
| | CO1 | | | |
| 5935 | MT- | Prostate Cancer | A5935G | A-G |
| | CO1 | | | |
| 5973 | MT- | Prostate Cancer | G5973A | G-A |
| | CO1 | | | |
| 6020 | MT- | Motor Neuron Disease | 6020_{—} | CGAGC- |
| | CO1 | | 6024delCGAGC | del |
| 6081 | MT- | Prostate Cancer | G6081A | G-A |
| | CO1 | | | |
| 6150 | MT- | Prostate Cancer/ | G6150A | G-A |
| | CO1 | enriched in POAG | | |
| | | cohort | | |
| 6253 | MT- | Prostate Cancer/ | T6253C | T-C |
| | CO1 | enriched in POAG | | |
| | | cohort | | |
| 6261 | MT- | Prostate Cancer/ | G6261A | G-A |
| | CO1 | LHON | | |
| 6267 | MT- | Prostate Cancer | G6267A | G-A |
| | CO1 | | | |
| 6285 | MT- | Prostate Cancer | G6285A | G-A |
| | CO1 | | | |
| 6307 | MT- | Asthenozoospermic | A6307G | A-G |
| | CO1 | infertility | | |
| 6328 | MT- | EXIT (Exercise | C6328T | C-T |
| | CO1 | Intolerance) | | |
| 6340 | MT- | Prostate Cancer | C6340T | C-T |
| | CO1 | | | |
| 6459 | MT- | Sepsis susceptibility | T6459C | T-C |
| | CO1 | | | |
| 6480 | MT- | Prostate Cancer/ | G6480A | G-A |
| | CO1 | enriched in POAG | | |
| | | cohort | | |
| 6489 | MT- | CO1 deficiency with | C6489A | C-A |
| | CO1 | epilepsia partialis | | |
| | | continua | | |
| 6597 | MT- | MELAS-like syndrome | C6597A | C-A |
| | CO1 | | | |
| 6663 | MT- | Prostate Cancer | A6663G | A-G |
| | CO1 | | | |
| 6698 | MT- | Myopathy | A6698del | A-del |
| | CO1 | | | |
| 6708 | MT- | MM & | G6708A | G-A |
| | CO1 | Rhabdomyolysis | | |
| 6721 | MT- | Acquired Idiopathic | T6721C | T-C |
| | CO1 | Sideroblastic Anemia | | |
| 6742 | MT- | Acquired Idiopathic | T6742C | T-C |
| | CO1 | Sideroblastic Anemia | | |
| 6860 | MT- | Dilated | A6860C | A-C |
| | CO1 | Cardiomyopathy | | |
| 6930 | MT- | Multisystem Disorder | G6930A | G-A |
| | CO1 | | | |
| 6955 | MT- | Mild EXIT and MR | G6955A | G-A |
| | CO1 | | | |
| 6962 | MT- | Possible helper | G6962A | G-A |
| | CO1 | variant for 15927A | | |
| 7023 | MT- | MELAS-like syndrome | G7023A | G-A |
| | CO1 | | | |
| 7041 | MT- | Prostate Cancer | G7041A | G-A |
| | CO1 | | | |
| 7080 | MT- | Prostate Cancer | T7080C | T-C |
| | CO1 | | | |
| 7083 | MT- | Prostate Cancer | A7083G | A-G |
| | CO1 | | | |
| 7158 | MT- | Prostate Cancer | A7158G | A-G |
| | CO1 | | | |
| 7305 | MT- | Prostate Cancer | A7305C | A-C |
| | CO1 | | | |
| 7402 | MT- | Isolated complex IV | C7402del | C-del |
| | CO1 | deficiency | | |
| 7443 | MT- | DEAF | A7443G | A-G |
| | CO1 | | | |
| 7444 | MT- | LHON/SNHL/ | G7444A | G-A |
| | CO1 | DEAF | | |
| 7445 | MT- | DEAF | A7445C | A-C |
| | CO1 | | | |
| 7445 | MT- | SNHL | A7445G | A-G |
| | CO1 | | | |
| 7587 | MT- | Mitochondrial | T7587C | T-C |
| | CO2 | Encephalomyopathy | | |
| 7598 | MT- | Possible LHON helper | G7598A | G-A |
| | CO2 | variant | | |
| 7623 | MT- | LHON | C7623T | C-T |
| | CO2 | | | |
| 7630 | MT- | MELAS | T7630del | T-del |
| | CO2 | | | |
| 7637 | MT- | PD risk factor | G7637A | G-A |
| | CO2 | | | |
| 7671 | MT- | MM | T7671A | T-A |
| | CO2 | | | |
| 7697 | MT- | Possible HCM | G7697A | G-A |
| | CO2 | susceptibility | | |
| 7706 | MT- | Alpers-Huttenlocher- | G7706A | G-A |
| | CO2 | like | | |
| 7859 | MT- | Progressive | G7859A | G-A |
| | CO2 | Encephalomyopathy | | |
| 7868 | MT- | LHON | C7868T | C-T |
| | CO2 | | | |
| 7877 | MT- | PEG glaucoma | A7877C | A-C |
| | CO2 | | | |
| 7896 | MT- | Multisystem Disorder | G7896A | G-A |
| | CO2 | | | |
| 7965 | MT- | Hepatic failure/COX | T7965C | T-C |
| | CO2 | deficiency | | |
| 7970 | MT- | Encephalopathy | G7970T | G-T |
| | CO2 | | | |
| 7989 | MT- | Rhabdomyolysis | T7989C | T-C |
| | CO2 | | | |
| 8010 | MT- | Developmental delay, | T8010C | T-C |
| | CO2 | ataxia, seizure, | | |
| | | hypotonia, lactic | | |
| | | acidosis | | |
| 8021 | MT- | Asthenozoospermia | A8021G | A-G |
| | CO2 | | | |
| 8042 | MT- | Lactic Acidosis | 8042_{—} | AT-del |
| | CO2 | | 8403delAT | |
| 8078 | MT- | DEAF | G8078A | G-A |
| | CO2 | | | |
| 8088 | MT- | Mitochondrial myopathy with | T8088del | T-del |
| | CO2 | complex IV deficiency | | |
| 8108 | MT- | SNHL | A8108G | A-G |
| | CO2 | | | |
| 8119 | MT- | Biliary atresia | T8119del | T-del |
| | CO2 | | | |
| 8156 | MT- | Multi-system | G8156del | G-del |
| | CO2 | mitochondrial disorder | | |
| 8241 | MT- | MIDD + retinopathy | T8241G | T-G |
| | CO2 | | | |
| 8249 | MT- | Mitochondrial myopathy | G8249A | G-A |
| | CO2 | | | |
| 8381 | MT- | MIDD/LVNC | A8381G | A-G |
| | ATP8 | cardiomyopathy-assoc. | | |
| 8393 | MT- | Reversible brain | C8393T | C-T |
| | ATP8 | pseudoatrophy | | |
| 8403 | MT- | Episodic weakness and | T8403C | T-C |
| | ATP8 | progressive neuropathy | | |
| 8411 | MT- | Severe mitochondrial | A8411G | A-G |
| | ATP8 | disorder | | |
| 8414 | MT- | Increased risk of | C8414T | C-T |
| | ATP8 | T2DM in haplogroup | | |
| | | D4/Longevity | | |
| 8481 | MT- | Tetralogy of Fallot | C8481T | C-T |
| | ATP8 | patient | | |
| 8490 | MT- | Peripheral neuropathy | T8490C | T-C |
| | ATP8 | of T2DM | | |
| 8519 | MT- | Susceptibility to | G8519A | G-A |
| | ATP8 | bullous pemphigoid | | |
| 8527 | MT- | Neuromuscular disorder, | A8527G | A-G |
| | ATP8/6 | possible helper mutation | | |
| 8528 | MT- | Infantile | T8528C | T-C |
| | ATP8/6 | cardiomyopathy | | |
| 8529 | MT- | Apical HCM | G8529A | G-A |
| | ATP8/6 | | | |
| 8558 | MT- | Possibly LVNC | C8558T | C-T |
| | ATP8/6 | cardiomyopathy-associated | | |
| 8561 | MT- | Ataxia w neuropathy, DM, | C8561G | C-G |
| | ATP8/6 | SNHL, and hypogonadism | | |
| 8561 | MT- | Ataxia w psychomotor | C8561T | C-T |
| | ATP8/6 | delay | | |
| 8611 | MT- | Ataxia, microcephaly, | C8611CC | C-CC |
| | ATP6 | developmental delay, | | |
| | | intellectual disability | | |
| 8618 | MT- | NARP | T8618TT | T-TT |
| | ATP6 | | | |
| 8668 | MT- | LHON | T8668C | T-C |
| | ATP6 | | | |
| 8719 | MT- | Suspected mito disease | G8719A | G-A |
| | ATP6 | | | |
| 8741 | MT- | MILS protective factor | T8741G | T-G |
| | ATP6 | | | |
| 8794 | MT- | Exercise Endurance/Coronary | C8794T | C-T |
| | ATP6 | Atherosclerosis risk | | |
| 8795 | MT- | MILS protective factor | A8795G | A-G |
| | ATP6 | | | |
| 8821 | MT- | Possible LHON helper | T8821G | T-G |
| | ATP6 | variant | | |
| 8836 | MT- | LHON | A8836G | A-G |
| | ATP6 | | | |
| 8851 | MT- | BSN/Leigh syndrome | T8851C | T-C |
| | ATP6 | | | |
| 8890 | MT- | Juvenile-onset | A8890G | A-G |
| | ATP6 | metabolic syndrome | | |
| 8932 | MT- | Prostate Cancer/ | C8932T | C-T |
| | ATP6 | Neuromuscular disorder | | |
| 8950 | MT- | LDYT | G8950A | G-A |
| | ATP6 | | | |
| 8959 | MT- | Developmental delay, | G8959A | G-A |
| | ATP6 | intellectual disability, | | |
| | | low citrilline | | |
| 8969 | MT- | Mitochondrial myopathy, | G8969A | G-A |
| | ATP6 | lactic acidosis and | | |
| | | sideroblastic anemia | | |
| | | (MLASA)/IgG nephropathy | | |
| 8993 | MT- | NARP/Leigh Disease/ | T8993C | T-C |
| | ATP6 | MILS/other | | |
| 8993 | MT- | NARP/Leigh Disease/ | T8993G | T-G |
| | ATP6 | MILS/other | | |
| 9010 | MT- | Unspecified | G9010A | G-A |
| | ATP6 | neurological disorder | | |
| 9016 | MT- | LHON | A9016G | A-G |
| | ATP6 | | | |
| 9017 | MT- | Unspecified | T9017C | T-C |
| | ATP6 | neurological disorder | | |
| 9025 | MT- | Motor neuropathy, LS- | G9025A | G-A |
| | ATP6 | like, colon cancer | | |
| 9029 | MT- | LHON-like | A9029G | A-G |
| | ATP6 | | | |
| 9032 | MT- | NARP | T9032C | T-C |
| | ATP6 | | | |
| 9035 | MT- | Ataxia syndromes | T9035C | T-C |
| | ATP6 | | | |
| 9055 | MT- | PD protective factor | G9055A | G-A |
| | ATP6 | | | |
| 9058 | MT- | Possibly LVNC | A9058G | A-G |
| | ATP6 | cardiomyopathy-associated | | |
| 9071 | MT- | Potentially functional | C9071T | C-T |
| | ATP6 | variant cosegregating | | |
| | | with LHON3635A | | |
| 9098 | MT- | Predisposition to anti- | T9098C | T-C |
| | ATP6 | retroviral mito disease | | |
| 9101 | MT- | LHON | T9101C | T-C |
| | ATP6 | | | |
| 9127 | MT- | NARP | 9127_{—} | AT- |
| | ATP6 | | 9128delAT | del |
| 9134 | MT- | Hypotonia, lactic | A9134G | A-G |
| | ATP6 | acidosis, HCM, IUGR | | |
| 9139 | MT- | LHON | G9139A | G-A |
| | ATP6 | | | |
| 9155 | MT- | MIDD, renal | A9155G | A-G |
| | ATP6 | insufficiency | | |
| 9155 | MT- | Developmental delay, | A9155T | A-T |
| | ATP6 | intellectual disability, | | |
| | | low citrilline | | |
| 9176 | MT- | FBSN/Leigh Disease | T9176C | T-C |
| | ATP6 | | | |
| 9176 | MT- | Leigh Disease/Spastic | T9176G | T-G |
| | ATP6 | Paraplegia | | |
| 9185 | MT- | Leigh Disease/Ataxia | T9185C | T-C |
| | ATP6 | syndromes/NARP- | | |
| | | like disease | | |
| 9191 | MT- | Leigh Disease | T9191C | T-C |
| | ATP6 | | | |
| 9205 | MT- | Encephalopathy/ | 9205_{—} | TA-del |
| | ATP6 | Seizures/Lacticacidemia | 9206delTA | |
| 9267 | MT- | MIDD | G9267C | G-C |
| | CO3 | | | |
| 9379 | MT- | MM w lactic acidosis | G9379A | G-A |
| | CO3 | | | |
| 9387 | MT- | Asthenozoospermia | G9387A | G-A |
| | CO3 | | | |
| 9438 | MT- | LHON/gout | G9438A | G-A |
| | CO3 | | | |
| 9478 | MT- | Leigh Disease | T9478C | T-C |
| | CO3 | | | |
| 9480 | MT- | Myoglobinuria | 9480_{—} | TTTTTCTTCGCAGGA-del |
| | CO3 | | 9494del15 | (SEQ ID NO: 57) |
| 9487 | MT- | Myoglobinuria | 9487_{—} | TCGCAGGATTTTTCT-del |
| | CO3 | | 9501del15 | (SEQ ID NO: 58) |
| 9490 | MT- | Gout | C9490T | C-T |
| | CO3 | | | |
| 9537 | MT- | Leigh Disease | C9537CC | C-CC |
| | CO3 | | | |
| 9544 | MT- | Sporadic bilateral optic | G9544A | G-A |
| | CO3 | neuropathy | | |
| 9559 | MT- | Rhabdomyolysis | C9559del | C-del |
| | CO3 | | | |
| 9660 | MT- | LHON | A9660C | A-C |
| | CO3 | | | |
| 9738 | MT- | LHON | G9738T | G-T |
| | CO3 | | | |
| 9789 | MT- | Myopathy | T9789C | T-C |
| | CO3 | | | |
| 9804 | MT- | LHON | G9804A | G-A |
| | CO3 | | | |
| 9856 | MT- | LVNC cardiomyopathy/ | T9856C | T-C |
| | CO3 | gout | | |
| 9861 | MT- | AD | T9861C | T-C |
| | CO3 | | | |
| 9952 | MT- | Mitochondrial | G9952A | G-A |
| | CO3 | Encephalopathy | | |
| 9957 | MT- | PEM/MELAS/ | T9957C | T-C |
| | CO3 | NAION/HCM/gout | | |
| 9966 | MT- | LHON possible helper | G9966A | G-A |
| | CO3 | variant | | |
| 9972 | MT- | EXIT & APS2 - | A9972C | A-C |
| | CO3 | possible link | | |
| 10086 | MT- | Hypertensive end-stage | A10086G | A-G |
| | ND3 | renal disease | | |
| 10158 | MT- | Leigh Disease/ | T10158C | T-C |
| | ND3 | MELAS | | |
| 10191 | MT- | Leigh Disease/Leigh- | T10191C | T-C |
| | ND3 | like Disease/ESOC | | |
| 10197 | MT- | Leigh Disease/ | G10197A | G-A |
| | ND3 | Dystonia/Stroke/LDYT | | |
| 10237 | MT- | LHON | T10237C | T-C |
| | ND3 | | | |
| 10254 | MT- | Leigh Disease | G10254A | G-A |
| | ND3 | | | |
| 10398 | MT- | Invasive Breast Cancer risk | A10398A | A-A |
| | ND3 | factor AD PD BD | | |
| | | lithium response | | |
| | | Type 2 DM | | |
| 10398 | MT- | PD protective factor/ | A10398G | A-G |
| | ND3 | longevity/altered cell | | |
| | | pH/metabolic syndrome/ | | |
| | | breast cancer risk/LS | | |
| | | risk/ADHD/cognitive | | |
| | | decline/SCA2 age of onset | | |
| 10543 | MT- | LHON | A10543G | A-G |
| | ND4L | | | |
| 10591 | MT- | LHON | T10591G | T-G |
| | ND4L | | | |
| 10652 | MT- | BD/MDD-associated | T10652C | T-C |
| | ND4L | | | |
| 10663 | MT- | LHON | T10663C | T-C |
| | ND4L | | | |
| 10680 | MT- | LHON/synergistic | G10680A | G-A |
| | ND4L | combo 10680A + | | |
| | | 12033G + 14258A | | |
| 11042 | MT- | Biliary atresia | T11042C | T-C |
| | ND4 | | | |
| 11048 | MT- | Biliary atresia | T11048del | T-del |
| | ND4 | | | |
| 11084 | MT- | AD, PD MELAS | A11084G | A-G |
| | ND4 | | | |
| 11232 | MT- | CPEO | T11232C | T-C |
| | ND4 | | | |
| 11240 | MT- | Leigh Syndrome | C11240T | C-T |
| | ND4 | | | |
| 11251 | MT- | Reduced risk of PD | A11251G | A-G |
| | ND4 | | | |
| 11253 | MT- | LHON PD | T11253C | T-C |
| | ND4 | | | |
| 11365 | MT- | found in 1 HCM | T11365C | T-C |
| | ND4 | patient | | |
| 11375 | MT- | found in 1 sCJD patient | A11375C | A-C |
| | ND4 | | | |
| 11467 | MT- | Altered brain pH/ | A11467G | A-G |
| | ND4 | sCJD patients | | |
| 11470 | MT- | MELAS | A11470C | A-C |
| | ND4 | | | |
| 11621 | MT- | CPEO, exercise | 11621_{—} | TA-del |
| | ND4 | intolerance | 11622delTA | |
| 11696 | MT- | LHON/LDYT/DEAF/ | G11696A | G-A |
| | ND4 | hypertension helper mut. | | |
| 11777 | MT- | Leigh Disease | C11777A | C-A |
| | ND4 | | | |
| 11778 | MT- | LHON/Progressive | G11778A | G-A |
| | ND4 | Dystonia | | |
| 11832 | MT- | EXIT/oncocytoma | G11832A | G-A |
| | ND4 | | | |
| 11874 | MT- | LHON | C11874A | C-A |
| | ND4 | | | |
| 11919 | MT- | Thyroid Cancer Cell | C11919T | C-T |
| | ND4 | Line | | |
| 11984 | MT- | Leigh Syndrome | T11984C | T-C |
| | ND4 | | | |
| 11994 | MT- | Oligoasthenoteratozoos | C11994T | C-T |
| | ND4 | permia (OAT) | | |
| 12015 | MT- | Atypical MELAS | T12015C | T-C |
| | ND4 | | | |
| 12026 | MT- | DM | A12026G | A-G |
| | ND4 | | | |
| 12027 | MT- | SZ-associated | T12027C | T-C |
| | ND4 | | | |
| 12033 | MT- | LHON synergistic | A12033G | A-G |
| | ND4 | combo 10680A + | | |
| | | 12033G + 14258A | | |
| 12338 | MT- | DEAF1555 increased | T12338C | T-C |
| | ND5 | penetrance/LHON | | |
| 12361 | MT- | Non-alcoholic fatty | A12361G | A-G |
| | ND5 | liver disease | | |
| 12372 | MT- | Altered brain pH/ | G12372A | G-A |
| | ND5 | sCJD patients | | |
| 12397 | MT- | PD, early onset | A12397G | A-G |
| | ND5 | | | |
| 12414 | MT- | EXIT | T12414del | T-del |
| | ND5 | | | |
| 12425 | MT- | Mitochondrial Myopathy | A12425del | A-del |
| | ND5 | & Renal Failure | | |
| 12477 | MT- | possible HCM | T12477C | T-C |
| | ND5 | susceptibility | | |
| 12622 | MT- | Leigh Disease | G12622A | G-A |
| | ND5 | | | |
| 12631 | MT- | found in 2 sCJD | T12631A | T-A |
| | ND5 | patients | | |
| 12634 | MT- | Thyroid Cancer Cell | A12634G | A-G |
| | ND5 | Line | | |
| 12686 | MT- | Dilated | T12686A | T-A |
| | ND5 | Cardiomyopathy | | |
| 12706 | MT- | Leigh Disease | T12706C | T-C |
| | ND5 | | | |
| 12770 | MT- | MELAS | A12770G | A-G |
| | ND5 | | | |
| 12778 | MT- | Dilated | G12778C | G-C |
| | ND5 | Cardiomyopathy | | |
| 12782 | MT- | LHON | T12782G | T-G |
| | ND5 | | | |
| 12811 | MT- | Possible LHON factor | T12811C | T-C |
| | ND5 | | | |
| 12848 | MT- | LHON | C12848T | C-T |
| | ND5 | | | |
| 13042 | MT- | Optic neuropathy/ | G13042A | G-A |
| | ND5 | retinopathy/LD | | |
| 13045 | MT- | MELAS/LHON/Leigh | A13045C | A-C |
| | ND5 | overlap syndrome | | |
| 13046 | MT- | LHON/MELAS | T13046C | T-C |
| | ND5 | overlap syndrome | | |
| 13051 | MT- | LHON | G13051A | G-A |
| | ND5 | | | |
| 13063 | MT- | Adult-onset | G13063A | G-A |
| | ND5 | Encephalopathy/Ataxia | | |
| 13084 | MT- | MELAS/Leigh | A13084T | A-T |
| | ND5 | Disease | | |
| 13094 | MT- | Ataxia + PEO/MELAS, | T13094C | T-C |
| | ND5 | LD, LHON, myoclonus, fatigue | | |
| 13135 | MT- | possible HCM | G13135A | G-A |
| | ND5 | susceptibility | | |
| 13204 | MT- | Peripheral neuropathy | G13204A | G-A |
| | ND5 | of T2 diabetes | | |
| 13271 | MT- | Exercise intolerance | T13271C | T-C |
| | ND5 | (EXIT) | | |
| 13276 | MT- | MIDD + retinopathy | A13276G | A-G |
| | ND5 | | | |
| 13379 | MT- | LHON | A13379C | A-C |
| | ND5 | | | |
| 13511 | MT- | Leigh-like syndrome | A13511T | A-T |
| | ND5 | | | |
| 13513 | MT- | Leigh Disease/MELAS/ | G13513A | G-A |
| | ND5 | LHON-MELAS Overlap | | |
| | | Syndrome/negative | | |
| | | association w Carotid | | |
| | | Atherosclerosis | | |
| 13514 | MT- | Leigh Disease/MELAS/ | A13514G | A-G |
| | ND5 | Ca2+ downregulation | | |
| 13528 | MT- | LHON-like, LHON, | A13528G | A-G |
| | ND5 | MELAS | | |
| 13580 | MT- | Thyroid Cancer | C13580G | C-G |
| | ND5 | | | |
| 13637 | MT- | Possible LHON factor | A13637G | A-G |
| | ND5 | | | |
| 13708 | MT- | LHON/Increased MS risk/ | G13708A | G-A |
| | ND5 | higher freq in PD-ADS | | |
| 13730 | MT- | LHON | G13730A | G-A |
| | ND5 | | | |
| 13831 | MT- | Thyroid Cancer Cell | C13831A | C-A |
| | ND5 | Line | | |
| 13849 | MT- | MELAS | A13849C | A-C |
| | ND5 | | | |
| 13967 | MT- | Possible LHON factor | C13967T | C-T |
| | ND5 | | | |
| 14063 | MT- | Potentially functional | T14063C | T-C |
| | ND5 | variant cosegregating | | |
| | | with LHON3635A | | |
| 14091 | MT- | Developmental delay, | A14091T | A-T |
| | ND5 | seizure, hearing loss, | | |
| | | diabetes | | |
| 14163 | MT- | Possible deafness | C14163T | C-T |
| | ND6 | factor | | |
| 14258 | MT- | LHON synergistic | G14258A | G-A |
| | ND6 | combo 10680A + | | |
| | | 12033G + 14258A also | | |
| | | combo 14258A + | | |
| | | 14582G | | |
| 14279 | MT- | LHON | G14279A | G-A |
| | ND6 | | | |
| 14319 | MT- | PD, early onset | T14319C | T-C |
| | ND6 | | | |
| 14325 | MT- | LHON | T14325C | T-C |
| | ND6 | | | |
| 14340 | MT- | SNHL | C14340T | C-T |
| | ND6 | | | |
| 14430 | MT- | Thyroid Cancer | A14430G | A-G |
| | ND6 | | | |
| 14439 | MT- | Mitochondrial Respiratory | G14439A | G-A |
| | ND6 | Chain Disorder | | |
| 14441 | MT- | Leigh-like phenotype | T14441C | T-C |
| | ND6 | | | |
| 14453 | MT- | MELAS/Leigh | G14453A | G-A |
| | ND6 | Disease | | |
| 14459 | MT- | LDYT/Leigh Disease/ | G14459A | G-A |
| | ND6 | dystonia/carotid | | |
| | | atherosclerosis risk | | |
| 14482 | MT- | LHON | C14482A | C-A |
| | ND6 | | | |
| 14482 | MT- | LHON | C14482G | C-G |
| | ND6 | | | |
| 14484 | MT- | LHON | T14484C | T-C |
| | ND6 | | | |
| 14487 | MT- | Dystonia/Leigh Disease/ | T14487C | T-C |
| | ND6 | ataxia/ptosis/epilepsy | | |
| 14495 | MT- | LHON | A14495G | A-G |
| | ND6 | | | |
| 14498 | MT- | LHON | T14498C | T-C |
| | ND6 | | | |
| 14502 | MT- | LHON | T14502C | T-C |
| | ND6 | | | |
| 14536 | MT- | DMDF | C14535CC | C-CC |
| | ND6 | | | |
| 14568 | MT- | LHON | C14568T | C-T |
| | ND6 | | | |
| 14577 | MT- | MIDM | T14577C | T-C |
| | ND6 | | | |
| 14582 | MT- | LHON synergistic | A14582G | A-G |
| | ND6 | combo 14258A + | | |
| | | 14582G | | |
| 14596 | MT- | LHON | A14596T | A-T |
| | ND6 | | | |
| 14600 | MT- | Leigh Disease w/optic | G14600A | G-A |
| | ND6 | atrophy | | |
| 14668 | MT- | Depressive Disorder | C14668T | C-T |
| | ND6 | associated | | |
| 14787 | MT- | PD/MELAS | 14787_{—} | TTAA-del |
| | CYB | | 14790del TTAA | |
| 14831 | MT- | LHON | G14831A | G-A |
| | CYB | | | |
| 14841 | MT- | LHON helper mut. | A14841G | A-G |
| | CYB | | | |
| 14846 | MT- | EXIT/possibly | G14846A | G-A |
| | CYB | antiatherogenic, poss. | | |
| | | myocardial infarction | | |
| | | association | | |
| 14849 | MT- | EXIT/Septo-Optic | T14849C | T-C |
| | CYB | Dysplasia | | |
| 14864 | MT- | MELAS | T14864C | T-C |
| | CYB | | | |
| 14894 | MT- | LHON | T14894C | T-C |
| | CYB | | | |
| 15024 | MT- | Possible DEAF | G15024A | G-A |
| | CYB | modifier | | |
| 15043 | MT- | MDD-associated | G15043A | G-A |
| | CYB | | | |
| 15059 | MT- | MM/carotid | G15059A | G-A |
| | CYB | atherosclerosis risk/ | | |
| | | essential hypertension | | |
| 15077 | MT- | DEAF | G15077A | G-A |
| | CYB | | | |
| 15084 | MT- | EXIT | G15084A | G-A |
| | CYB | | | |
| 15092 | MT- | MELAS | G15092A | G-A |
| | CYB | | | |
| 15150 | MT- | EXIT | G15150A | G-A |
| | CYB | | | |
| 15153 | MT- | Suspected mito | G15153A | G-A |
| | CYB | disease | | |
| 15158 | MT- | Suspected mito | A15158G | A-G |
| | CYB | disease | | |
| 15168 | MT- | EXIT | G15168A | G-A |
| | CYB | | | |
| 15170 | MT- | EXIT | G15170A | G-A |
| | CYB | | | |
| 15197 | MT- | EXIT | T15197C | T-C |
| | CYB | | | |
| 15209 | MT- | Prader-Willi syndrome | T15209C | T-C |
| | CYB | | | |
| 15234 | MT- | Leigh stroke-like | G15234A | G-A |
| | CYB | leukodystrophy | | |
| 15237 | MT- | Potentially functional | T15237C | T-C |
| | CYB | variant cosegregating | | |
| | | with LHON3635A | | |
| 15242 | MT- | Mitochondrial | G15242A | G-A |
| | CYB | Encephalomyopathy | | |
| 15243 | MT- | HCM | G15243A | G-A |
| | CYB | | | |
| 15256 | MT- | Peripheral neuropathy | A15256G | A-G |
| | CYB | of T2 diabetes | | |
| 15257 | MT- | LHON | G15257A | G-A |
| | CYB | | | |
| 15287 | MT- | Possible DEAF helper | T15287C | T-C |
| | CYB | mut. | | |
| 15395 | MT- | Possible LHON factor | A15395G | A-G |
| | CYB | | | |
| 15453 | MT- | Isolated complex III | T15453C | T-C |
| | CYB | deficiency | | |
| 15497 | MT- | EXIT/Obesity | G15497A | G-A |
| | CYB | | | |
| 15498 | MT- | EXIT | 15498_{—} | 24bp_deletion |
| | CYB | | 15521de124 | |
| 15498 | MT- | DEAF/Infantile | G15498A | G-A |
| | CYB | histiocytoid | | |
| | | cardiomyopathy | | |
| 15579 | MT- | Multisystem Disorder, | A15579G | A-G |
| | CYB | EXIT | | |
| 15615 | MT- | EXIT/Antimycin | G15615A | G-A |
| | CYB | resistance | | |
| 15620 | MT- | Leigh Syndrome helper | C15620A | C-A |
| | CYB | mut | | |
| 15635 | MT- | Polyvisceral failure | T15635C | T-C |
| | CYB | | | |
| 15649 | MT- | Multisystem Disorder, | 15649_{—} | 18bp_deletion |
| | CYB | EXIT | 15666del1 | |
| 15662 | MT- | Complex | A15662G | A-G |
| | CYB | mitochondriopathy-associated | | |
| 15674 | MT- | LHON | T15674C | T-C |
| | CYB | | | |
| 15693 | MT- | Possibly LVNC | T15693C | T-C |
| | CYB | cardiomyopathy-associated | | |
| 15699 | MT- | Muscle Weakness | G15699C | G-C |
| | CYB | SNHL and Migraine | | |
| 15723 | MT- | EXIT | G15723A | G-A |
| | CYB | | | |
| 15761 | MT- | MM | G15761A | G-A |
| | CYB | | | |
| 15762 | MT- | MM | G15762A | G-A |
| | CYB | | | |
| 15773 | MT- | LHON | G15773A | G-A |
| | CYB | | | |
| 15784 | MT- | POAG - potential | T15784C | T-C |
| | CYB | for association | | |
| 15800 | MT- | EXIT/Myopathy | C15800T | C-T |
| | CYB | | | |
| 15804 | MT- | Fibromyalgia | T15804C | T-C |
| | CYB | | | |
| 15812 | MT- | LHON | G15812A | G-A |
| | CYB | | | |
| 16081 | MT- | Cyclic Vomiting | A16081G | A-G |
| | CR | Syndrome | | |
| 16093 | MT- | Cyclic Vomiting | T16093C | T-C |
| | CR | Syndrome | | |
| 16129 | MT- | Cyclic Vomiting | G16129A | G-A |
| | CR | Syndrome with Migraine | | |
| 16176 | MT- | Cyclic Vomiting | C16176T | C-T |
| | CR | Syndrome with Migraine | | |
| 16183 | MT- | Melanoma patients | A16183C | A-C |
| | CR | | | |
| 16189 | MT- | Diabetes/Cardiomyopathy/ | T16189C | T-C |
| | CR | cancer risk/mtDNA copy nbr/ | | |
| | | Metabolic Syndrome/ | | |
| | | Melanoma patients | | |
| 16192 | MT- | Melanoma patients | C16192T | C-T |
| | CR | | | |
| 16217 | MT- | Endometriosis | T16217C | T-C |
| | CR | | | |
| 16270 | MT- | Melanoma patients | C16270T | C-T |
| | CR | | | |
| 16300 | MT- | BD-associated | A16300G | A-G |
| | CR | | | |
| 16318 | MT- | Non-alcoholic | A16318C | A-C |
| | CR | steatohepatitis - | | |
| | | potential for | | |
| | | association | | |
| 16390 | MT- | POAG - potential for | G16390A | G-A |
| | CR | association | | |
| 16519 | MT- | Cyclic Vomiting Syndrome | T16519T | T-T |
| | CR | with Migraine/metastasis | | |

| A | F | G | H | I | J | K | L |
|---|---|---|---|---|---|---|---|
| 114 | noncoding | + | − | Reported | 0.4% | 216 (108) | 1 |
| | | | | | (0.1%) | | |
| 146 | noncoding | + | − | Reported | 19.5% | 9763 (8481) | 1 |
| | | | | | (11.6%) | | |
| 150 | noncoding | + | + | Conflicting | 13.4% | 6722 (7062) | 8 |
| | | | | reports | (9.6%) | | |
| 195 | noncoding | + | − | Reported | 19.6% | 9817 (8590) | 3 |
| | | | | | (11.7%) | | |
| 302 | noncoding | . | . | Reported | 0.3% | 167 (14) | 1 |
| | | | | | (0.0%) | | |
| 309 | noncoding | . | . | Reported | 1.1% | 531 (952) | 1 |
| | | | | | (1.3%) | | |
| 310 | noncoding | . | . | Reported | 0.0% | 0 (0) | 1 |
| | | | | | (0.0%) | | |
| 499 | noncoding | + | − | Reported | 3.7% | 1832 (1356) | 1 |
| | | | | | (1.9%) | | |
| 547 | noncoding | + | − | Reported | 0.0% | 0 (0) | 1 |
| | | | | | (0.0%) | | |
| 573 | noncoding | + | − | Reported | 0.0% | 0 (20) | 1 |
| | | | | | (0.0%) | | |
| 3308 | M-T | − | + | P.M.- | 0.7% | 352 (0) | 15 |
| | | | | possibly | (0.0%) | | |
| | | | | synergistic | | | |
| 3308 | M-Term | + | + | Reported | 0.0% | 6 (0) | 1 |
| | | | | | (0.0%) | | |
| 3310 | P-S | + | + | Reported | 0.0% | 12 (0) | 4 |
| | | | | | (0.0%) | | |
| 3316 | A-T | + | − | Unclear | 1.0% | 513 (0) | 21 |
| | | | | | (0.0%) | | |
| 3335 | I-T | + | − | Reported | 0.1% | 54 (0) | 1 |
| | | | | | (0.0%) | | |
| 3336 | I-I | − | + | Reported | 0.4% | 194 (0) | 3 |
| | | | | | (0.0%) | | |
| 3337 | V-M | + | − | Possibly | 0.2% | 79 (0) | 2 |
| | | | | synergistic | (0.0%) | | |
| 3340 | P-S | + | + | Reported | 0.0% | 3 (0) | 2 |
| | | | | | (0.0%) | | |
| 3376 | E-K | + | + | Cfrm | 0.0% | 0 (0) | 3 |
| | | | | | (0.0%) | | |
| 3380 | R-Q | − | + | Reported | 0.0% | 3 (0) | 1 |
| | | | | | (0.0%) | | |
| 3388 | L-M | . | . | Reported | 0.0% | 17 (0) | 1 |
| | | | | | (0.0%) | | |
| 3391 | G-S | + | − | Reported | 0.1% | 52 (0) | 1 |
| | | | | | (0.0%) | | |
| 3394 | Y-H | + | − | Reported/ | 1.3% | 633 (0) | 32 |
| | | | | Population- | (0.0%) | | |
| | | | | dependent | | | |
| 3395 | Y-C | + | + | Reported | 0.0% | 23 (0) | 3 |
| | | | | | (0.0%) | | |
| 3396 | Y-Y | + | − | Reported/ | 0.8% | 402 (0) | 2 |
| | | | | Unclear | (0.0%) | | |
| 3397 | M-V | + | − | Reported | 0.3% | 150 (0) | 11 |
| | | | | | (0.0%) | | |
| 3398 | M-T | + | − | Reported | 0.4% | 196 (0) | 5 |
| | | | | | (0.0%) | | |
| 3399 | M-I | + | − | Reported | 0.0% | 25 (0) | 1 |
| | | | | | (0.0%) | | |
| 3407 | R-H | + | − | Conflicting | 0.0% | 1 (0) | 3 |
| | | | | reports | (0.0%) | | |
| 3418 | N-D | + | − | Reported | 0.0% | 1 (0) | 1 |
| | | | | | (0.0%) | | |
| 3421 | V-I | + | − | Reported | 0.1% | 70 (0) | 2 |
| | | | | | (0.0%) | | |
| 3460 | A-T | + | + | Cfrm | 0.0% | 23 (0) | 160 |
| | | | | | (0.0%) | | |
| 3472 | F-L | + | + | Reported | 0.0% | 5 (0) | 7 |
| | | | | | (0.0%) | | |
| 3481 | E-K | − | + | Reported | 0.0% | 0 (0) | 3 |
| | | | | | (0.0%) | | |
| 3488 | L-P | + | − | Reported | 0.0% | 1 (0) | 1 |
| | | | | | (0.0%) | | |
| 3496 | A-S | + | − | Reported/ | 0.0% | 11 (0) | 3 |
| | | | | Secondary | (0.0%) | | |
| 3497 | A-V | + | − | Reported/ | 0.4% | 184 (0) | 5 |
| | | | | Secondary | (0.0%) | | |
| 3551 | A-V | + | − | Reported | 0.0% | 0 (0) | 1 |
| | | | | | (0.0%) | | |
| 3571 | L-F | . | . | Reported | 0.2% | 122 (0) | 3 |
| | | | | | (0.0%) | | |
| 3632 | S-F | + | − | Reported | 0.0% | 0 (0) | 1 |
| | | | | | (0.0%) | | |
| 3634 | S-G | + | − | Reported | 0.0% | 0 (0) | 1 |
| | | | | | (0.0%) | | |
| 3635 | S-N | + | − | Cfrm | 0.0% | 9 (0) | 11 |
| | | | | | (0.0%) | | |
| 3644 | V-A | . | . | Reported | 0.4% | 207 (0) | 4 |
| | | | | | (0.0%) | | |
| 3667 | W-G | + | − | Reported | 0.0% | 1 (0) | 1 |
| | | | | | (0.0%) | | |
| 3688 | A-T | + | − | Reported | 0.0% | 0 (0) | 2 |
| | | | | | (0.0%) | | |
| 3697 | G-S | + | + | Cfrm | 0.0% | 0 (0) | 13 |
| | | | | | (0.0%) | | |
| 3700 | A-T | + | − | Cfrm | 0.0% | 3 (0) | 5 |
| | | | | | (0.0%) | | |
| 3713 | V-A | + | − | Reported | 0.0% | 0 (0) | 1 |
| | | | | | (0.0%) | | |
| 3733 | E-K | + | + | Cfrm | 0.0% | 2 (0) | 8 |
| | | | | | (0.0%) | | |
| 3733 | E-Q | − | + | Reported | 0.0% | 0 (0) | 1 |
| | | | | | (0.0%) | | |
| 3736 | V-I | . | . | Reported | 0.2% | 82 (0) | 2 |
| | | | | | (0.0%) | | |
| 3745 | A-T | . | . | Reported/ | 0.2% | 102 (0) | 3 |
| | | | | Population- | (0.0%) | | |
| | | | | dependent | | | |
| 3769 | L-V | + | − | Reported | 0.0% | 0 (0) | 1 |
| | | | | | (0.0%) | | |
| 3781 | S-P | + | − | Reported | 0.0% | 0 (0) | 1 |
| | | | | | (0.0%) | | |
| 3796 | T-A | − | + | Reported | 0.5% | 236 (0) | 4 |
| | | | | | (0.0%) | | |
| 3833 | L-Q | + | − | Reported | 0.0% | 0 (0) | 2 |
| | | | | | (0.0%) | | |
| 3866 | I-T | . | . | Reported | 0.3% | 143 (0) | 5 |
| | | | | | (0.0%) | | |
| 3890 | R-Q | − | + | Cfrm | 0.0% | 1 (0) | 7 |
| | | | | | (0.0%) | | |
| 3902 | DLA- | − | + | Cfrm | 0.0% | 0 (0) | 3 |
| | GKV | | | | (0.0%) | | |
| 3919 | S-P | + | − | Reported | 0.0% | 0 (0) | 1 |
| | | | | | (0.0%) | | |
| 3945 | I-M | . | . | Reported | 0.0% | 0 (0) | 1 |
| | | | | | (0.0%) | | |
| 3946 | E-K | + | + | Reported | 0.0% | 2 (0) | 7 |
| | | | | | (0.0%) | | |
| 3949 | Y-H | − | + | Reported | 0.0% | 1 (0) | 7 |
| | | | | | (0.0%) | | |
| 3958 | G-S | + | − | Reported | 0.0% | 0 (0) | 1 |
| | | | | | (0.0%) | | |
| 3959 | G-D | . | . | Reported | 0.0% | 0 (0) | 1 |
| | | | | | (0.0%) | | |
| 3995 | N-S | . | . | Reported | 0.0% | 18 (0) | 2 |
| | | | | | (0.0%) | | |
| 4081 | F-L | + | − | Reported | 0.0% | 0 (0) | 1 |
| | | | | | (0.0%) | | |
| 4123 | I-F | + | − | Reported | 0.0% | 0 (0) | 1 |
| | | | | | (0.0%) | | |
| 4132 | A-T | + | − | Reported | 0.0% | 7 (0) | 2 |
| | | | | | (0.0%) | | |
| 4136 | Y-C | + | − | Possibly | 0.1% | 66 (0) | 11 |
| | | | | synergistic | (0.0%) | | |
| 4142 | R-Q | − | + | Reported | 0.0% | 0 (0) | 2 |
| | | | | | (0.0%) | | |
| 4160 | L-P | + | − | Reported | 0.0% | 1 (0) | 12 |
| | | | | | (0.0%) | | |
| 4163 | M-T | + | − | Reported | 0.0% | 0 (0) | 1 |
| | | | | | (0.0%) | | |
| 4171 | L-M | + | + | Cfrm | 0.0% | 2 (0) | 10 |
| | | | | | (0.0%) | | |
| 4216 | Y-H | + | − | Reported | 9.9% | 4952 (0) | 42 |
| | | | | | (0.0%) | | |
| 4633 | A-G | + | − | Reported | 0.0% | 0 (0) | 1 |
| | | | | | (0.0%) | | |
| 4640 | I-M | + | − | Reported | 0.4% | 177 (0) | 8 |
| | | | | | (0.0%) | | |
| 4648 | F-S | + | − | Reported | 0.0% | 1 (0) | 2 |
| | | | | | (0.0%) | | |
| 4659 | A-T | + | − | Reported | 0.1% | 65 (0) | 1 |
| | | | | | (0.0%) | | |
| 4681 | L-P | − | + | Reported | 0.0% | 1 (0) | 3 |
| | | | | | (0.0%) | | |
| 4769 | M-M | + | − | Reported | 2.3% | 1167 (0) | 2 |
| | | | | | (0.0%) | | |
| 4833 | T-A | + | − | Reported | 0.9% | 452 (0) | 3 |
| | | | | | (0.0%) | | |
| 4852 | L-Q | + | − | Reported | 0.0% | 0 (0) | 1 |
| | | | | | (0.0%) | | |
| 4883 | P-P | + | − | Conflicting | 4.8% | 2393 (0) | 2 |
| | | | | reports | (0.0%) | | |
| 4917 | N-D | + | − | Reported | 4.8% | 2390 (0) | 28 |
| | | | | | (0.0%) | | |
| 5001 | frameshift | − | + | Reported | 0.0% | 0 (0) | 2 |
| | | | | | (0.0%) | | |
| 5095 | I-T | . | . | Reported | 0.0% | 20 (0) | 1 |
| | | | | | (0.0%) | | |
| 5133 | frameshift | . | . | Reported | 0.0% | 0 (0) | 5 |
| | | | | | (0.0%) | | |
| 5178 | L-M | + | − | Reported | 4.7% | 2370 (0) | 23 |
| | | | | | (0.0%) | | |
| 5244 | G-S | − | + | Reported | 0.0% | 0 (0) | 7 |
| | | | | | (0.0%) | | |
| 5452 | T-M | + | − | Reported | 0.0% | 15 (0) | 2 |
| | | | | | (0.0%) | | |
| 5460 | A-T | + | + | Conflicting | 6.5% | 3272 (0) | 9 |
| | | | | reports | (0.0%) | | |
| 5460 | A-S | + | + | Reported | 0.0% | 0 (0) | 5 |
| | | | | | (0.0%) | | |
| 5911 | A-V | + | − | Reported | 0.5% | 248 (0) | 1 |
| | | | | | (0.0%) | | |
| 5913 | D-N | + | − | Reported | 1.0% | 482 (0) | 3 |
| | | | | | (0.0%) | | |
| 5920 | W-Ter | − | + | Reported | 0.0% | 0 (0) | 4 |
| | | | | | (0.0%) | | |
| 5935 | N-S | + | − | Reported | 0.0% | 1 (0) | 1 |
| | | | | | (0.0%) | | |
| 5973 | A-T | + | − | Reported | 0.0% | 11 (0) | 1 |
| | | | | | (0.0%) | | |
| 6020 | AELGQ- | − | + | Reported | 0.0% | 0 (0) | 1 |
| | AGPATer | | | | (0.0%) | | |
| 6081 | A-T | + | − | Reported | 0.0% | 1 (0) | 1 |
| | | | | | (0.0%) | | |
| 6150 | V-I | + | − | Reported | 0.5% | 233 (0) | 2 |
| | | | | | (0.0%) | | |
| 6253 | M-T | + | − | Reported | 1.0% | 524 (0) | 3 |
| | | | | | (0.0%) | | |
| 6261 | A-T | + | − | Reported | 0.7% | 361 (0) | 3 |
| | | | | | (0.0%) | | |
| 6267 | A-T | + | − | Reported | 0.2% | 77 (0) | 1 |
| | | | | | (0.0%) | | |
| 6285 | V-I | + | − | Reported | 0.2% | 121 (0) | 1 |
| | | | | | (0.0%) | | |
| 6307 | N-S | . | + | Reported | 0.0% | 2 (0) | 1 |
| | | | | | (0.0%) | | |
| 6328 | S-F | + | − | Reported | 0.0% | 0 (0) | 2 |
| | | | | | (0.0%) | | |
| 6340 | T-I | + | − | Reported | 0.2% | 82 (0) | 2 |
| | | | | | (0.0%) | | |
| 6459 | W-R | + | − | Reported | 0.0% | 0 (0) | 1 |
| | | | | | (0.0%) | | |
| 6480 | V-I | + | − | Reported | 0.3% | 146 (0) | 4 |
| | | | | | (0.0%) | | |
| 6489 | L-I | − | + | Reported | 0.2% | 86 (0) | 3 |
| | | | | | (0.0%) | | |
| 6597 | Q-K | − | + | Reported | 0.0% | 0 (0) | 1 |
| | | | | | (0.0%) | | |
| 6663 | I-V | + | − | Reported | 0.3% | 151 (0) | 3 |
| | | | | | (0.0%) | | |
| 6698 | K-K_{—} | − | + | Reported | 0.0% | 0 (0) | 1 |
| | frameshift | | | | (0.0%) | | |
| 6708 | G-Ter | − | + | Reported | 0.0% | 0 (0) | 1 |
| | | | | | (0.0%) | | |
| 6721 | M-T | − | + | Reported | 0.0% | 0 (0) | 2 |
| | | | | | (0.0%) | | |
| 6742 | I-T | − | + | Reported | 0.0% | 0 (0) | 2 |
| | | | | | (0.0%) | | |
| 6860 | K-N | + | − | Reported | 0.0% | 0 (0) | 1 |
| | | | | | (0.0%) | | |
| 6930 | G-Ter | − | + | Reported | 0.0% | 0 (0) | 3 |
| | | | | | (0.0%) | | |
| 6955 | G-D | + | + | Reported | 0.0% | 1 (0) | 1 |
| | | | | | (0.0%) | | |
| 6962 | L-L | + | − | Reported | 2.4% | 1206 (0) | 1 |
| | | | | | (0.0%) | | |
| 7023 | V-M | − | + | Reported | 0.0% | 1 (0) | 1 |
| | | | | | (0.0%) | | |
| 7041 | V-I | + | − | Reported | 0.0% | 6 (0) | 1 |
| | | | | | (0.0%) | | |
| 7080 | F-L | + | − | Reported | 0.1% | 55 (0) | 1 |
| | | | | | (0.0%) | | |
| 7083 | I-V | + | − | Reported | 0.0% | 15 (0) | 1 |
| | | | | | (0.0%) | | |
| 7158 | I-V | + | − | Reported | 0.1% | 36 (0) | 1 |
| | | | | | (0.0%) | | |
| 7305 | M-L | + | − | Reported | 0.0% | 0 (0) | 1 |
| | | | | | (0.0%) | | |
| 7402 | frameshift | − | + | Reported | 0.0% | 0 (0) | 1 |
| | | | | | (0.0%) | | |
| 7443 | Ter-G | + | − | Reported | 0.0% | 1 (0) | 4 |
| | | | | | (0.0%) | | |
| 7444 | Ter-K | + | − | Reported | 0.4% | 183 (0) | 26 |
| | | | | | (0.0%) | | |
| 7445 | Ter-S | + | − | Reported | 0.0% | 13 (0) | 7 |
| | | | | | (0.0%) | | |
| 7445 | Ter-Ter | + | + | Cfrm | 0.0% | 1 (0) | 32 |
| | | | | | (0.0%) | | |
| 7587 | M-T | − | + | Reported | 0.0% | 0 (0) | 2 |
| | | | | | (0.0%) | | |
| 7598 | A-T | − | + | Reported | 1.2% | 608 (0) | 2 |
| | | | | | (0.0%) | | |
| 7623 | T-I | + | − | Reported | 0.0% | 0 (0) | 1 |
| | | | | | (0.0%) | | |
| 7630 | frameshift | − | + | Reported | 0.0% | 0 (0) | 1 |
| | | | | | (0.0%) | | |
| 7637 | E-K | − | + | Reported | 0.0% | 2 (0) | 1 |
| | | | | | (0.0%) | | |
| 7671 | M-K | − | + | Reported | 0.0% | 0 (0) | 2 |
| | | | | | (0.0%) | | |
| 7697 | V-I | + | − | Reported | 0.5% | 253 (0) | 3 |
| | | | | | (0.0%) | | |
| 7706 | A-T | | + | Reported | 0.0% | 9 (0) | 1 |
| | | | | | (0.0%) | | |
| 7859 | D-N | + | − | Reported | 0.3% | 150 (0) | 1 |
| | | | | | (0.0%) | | |
| 7868 | L-F | + | − | Possibly | 0.0% | 17 (0) | 1 |
| | | | | synergistic | (0.0%) | | |
| 7877 | K-Q | + | − | Reported | 0.0% | 0 (0) | 1 |
| | | | | | (0.0%) | | |
| 7896 | W-Ter | − | + | Reported | 0.0% | 0 (0) | 1 |
| | | | | | (0.0%) | | |
| 7965 | F-S | . | + | Reported | 0.0% | 1 (0) | 3 |
| | | | | | (0.0%) | | |
| 7970 | E-Ter | − | + | Reported | 0.0% | 0 (0) | 1 |
| | | | | | (0.0%) | | |
| 7989 | L-P | − | + | Reported | 0.0% | 0 (0) | 2 |
| | | | | | (0.0%) | | |
| 8010 | V-A | − | + | Reported | 0.0% | 2 (0) | 1 |
| | | | | | (0.0%) | | |
| 8021 | I-V | + | − | Reported | 0.0% | 4 (0) | 1 |
| | | | | | (0.0%) | | |
| 8042 | frameshift | − | + | Reported | 0.0% | 0 (0) | 1 |
| | | | | | (0.0%) | | |
| 8078 | V-I | + | − | Reported | 0.1% | 27 (0) | 2 |
| | | | | | (0.0%) | | |
| 8088 | frameshift | − | + | Reported | 0.0% | 0 (0) | 1 |
| | | | | | (0.0%) | | |
| 8108 | I-V | + | − | Reported | 0.1% | 71 (0) | 1 |
| | | | | | (0.0%) | | |
| 8119 | frameshift | − | + | Reported | 0.0% | 0 (0) | 1 |
| | | | | | (0.0%) | | |
| 8156 | frameshift | − | + | Reported | 0.0% | 0 (0) | 1 |
| | | | | | (0.0%) | | |
| 8241 | F-C | − | + | Conflicting | 0.0% | 0 (0) | 2 |
| | | | | reports | (0.0%) | | |
| 8249 | G-Ter | + | − | Reported | 0.0% | 1 (0) | 2 |
| | | | | | (0.0%) | | |
| 8381 | T-A | + | − | Reported | 0.0% | 13 (0) | 2 |
| | | | | | (0.0%) | | |
| 8393 | P-S | − | + | Reported | 0.3% | 174 (0) | 2 |
| | | | | | (0.0%) | | |
| 8403 | I-T | + | − | Reported | 0.0% | 3 (0) | 1 |
| | | | | | (0.0%) | | |
| 8411 | M-V | + | − | Reported | 0.0% | 2 (0) | 1 |
| | | | | | (0.0%) | | |
| 8414 | L-F | + | − | Reported | 3.9% | 1963 (0) | 1 |
| | | | | | (0.0%) | | |
| 8481 | P-L | + | − | Reported | 0.0% | 8 (0) | 1 |
| | | | | | (0.0%) | | |
| 8490 | M-T | + | − | Reported | 0.1% | 27 (0) | 4 |
| | | | | | (0.0%) | | |
| 8519 | E-K | + | − | Reported | 0.2% | 117 (0) | 1 |
| | | | | | (0.0%) | | |
| 8527 | ATP8: K-K | + | − | Reported | 0.4% | 212 (0) | 1 |
| | ATP6: M-V | | | | (0.0%) | | |
| 8528 | ATP8: W-R | + | + | Cfrm | 0.0% | 0 (0) | 3 |
| | ATP6: M-T | | | | (0.0%) | | |
| 8529 | ATP8: W-Term | + | − | Reported | 0.0% | 0 (0) | 1 |
| | ATP6: M-M | | | | (0.0%) | | |
| 8558 | ATP8: P-S | + | − | Reported | 0.0% | 12 (0) | 1 |
| | ATP6: A-V | | | | (0.0%) | | |
| 8561 | ATP8: P-A | + | + | Reported | 0.0% | 0 (0) | 1 |
| | ATP6: P-R | | | | (0.0%) | | |
| 8561 | ATP8: P-S | − | + | Reported | 0.0% | 0 (0) | 1 |
| | ATP6: P-L | | | | (0.0%) | | |
| 8611 | frameshift | − | + | Reported | 0.0% | 0 (0) | 2 |
| | | | | | (0.0%) | | |
| 8618 | frameshift | − | + | Reported | 0.0% | 0 (0) | 1 |
| | | | | | (0.0%) | | |
| 8668 | W-R | + | − | Reported | 0.1% | 34 (0) | 1 |
| | | | | | (0.0%) | | |
| 8719 | G-Ter | − | + | Reported | 0.0% | 0 (0) | 1 |
| | | | | | (0.0%) | | |
| 8741 | L-R | − | + | Reported | 0.0% | 0 (0) | 1 |
| | | | | | (0.0%) | | |
| 8794 | H-Y | + | − | Reported | 2.8% | 1399 (0) | 2 |
| | | | | | (0.0%) | | |
| 8795 | H-R | − | + | Reported | 0.0% | 0 (0) | 1 |
| | | | | | (0.0%) | | |
| 8821 | S-A | . | . | Reported | 0.0% | 0 (0) | 1 |
| | | | | | (0.0%) | | |
| 8836 | M-V | + | − | Reported | 0.3% | 152 (0) | 2 |
| | | | | | (0.0%) | | |
| 8851 | W-R | + | + | Cfrm | 0.0% | 3 (0) | 6 |
| | | | | | (0.0%) | | |
| 8890 | K-E | − | + | Reported | 0.0% | 0 (0) | 1 |
| | | | | | (0.0%) | | |
| 8932 | P-S | + | − | Reported | 0.4% | 212 (0) | 3 |
| | | | | | (0.0%) | | |
| 8950 | V-I | + | − | Reported | 0.1% | 74 (0) | 2 |
| | | | | | (0.0%) | | |
| 8959 | E-K | + | + | Reported | 0.0% | 4 (0) | 2 |
| | | | | | (0.0%) | | |
| 8969 | S-N | − | + | Cfrm | 0.0% | 0 (0) | 4 |
| | | | | | (0.0%) | | |
| 8993 | L-P | − | + | Cfrm | 0.0% | 2 (0) | 36 |
| | | | | | (0.0%) | | |
| 8993 | L-R | + | + | Cfrm | 0.0% | 6 (0) | 114 |
| | | | | | (0.0%) | | |
| 9010 | A-T | − | + | Reported | 0.1% | 27 (0) | 1 |
| | | | | | (0.0%) | | |
| 9016 | I-V | − | + | Reported | 0.0% | 13 (0) | 2 |
| | | | | | (0.0%) | | |
| 9017 | I-T | − | + | Reported | 0.0% | 11 (0) | 1 |
| | | | | | (0.0%) | | |
| 9025 | G-S | + | − | Reported | 0.1% | 29 (0) | 1 |
| | | | | | (0.0%) | | |
| 9029 | H-R | + | + | Reported | 0.0% | 1 (0) | 1 |
| | | | | | (0.0%) | | |
| 9032 | L-P | − | + | Reported | 0.0% | 0 (0) | 1 |
| | | | | | (0.0%) | | |
| 9035 | L-P | + | + | Cfrm | 0.0% | 0 (0) | 2 |
| | | | | | (0.0%) | | |
| 9055 | A-T | + | − | Reported | 4.2% | 2107 (0) | 2 |
| | | | | | (0.0%) | | |
| 9058 | T-A | + | − | Reported | 0.1% | 28 (0) | 1 |
| | | | | | (0.0%) | | |
| 9071 | S-L | + | − | Reported | 0.0% | 14 (0) | 1 |
| | | | | | (0.0%) | | |
| 9098 | I-T | + | − | Reported | 0.1% | 52 (0) | 1 |
| | | | | | (0.0%) | | |
| 9101 | I-T | + | − | Reported | 0.1% | 37 (0) | 5 |
| | | | | | (0.0%) | | |
| 9127 | IL- | − | + | Reported | 0.0% | 0 (0) | 1 |
| | PTer | | | | (0.0%) | | |
| 9134 | E-G | . | . | Reported | 0.0% | 0 (0) | 1 |
| | | | | | (0.0%) | | |
| 9139 | A-T | + | − | Reported - | 0.1% | 40 (0) | 1 |
| | | | | possibly | (0.0%) | | |
| | | | | synergistic | | | |
| 9155 | Q-R | − | + | Cfrm | 0.0% | 0 (0) | 3 |
| | | | | | (0.0%) | | |
| 9155 | Q-L | + | + | Reported | 0.0% | 0 (0) | 1 |
| | | | | | (0.0%) | | |
| 9176 | L-P | + | + | Cfrm | 0.0% | 3 (0) | 21 |
| | | | | | (0.0%) | | |
| 9176 | L-R | + | + | Cfrm | 0.0% | 1 (0) | 9 |
| | | | | | (0.0%) | | |
| 9185 | L-P | + | + | Cfrm | 0.0% | 3 (0) | 16 |
| | | | | | (0.0%) | | |
| 9191 | L-P | − | + | Reported | 0.0% | 0 (0) | 1 |
| | | | | | (0.0%) | | |
| 9205 | Ter-M | + | − | Cfrm | 0.0% | 0 (0) | 7 |
| | | | | | (0.0%) | | |
| 9267 | A-P | − | + | Reported | 0.0% | 0 (0) | 1 |
| | | | | | (0.0%) | | |
| 9379 | W-Ter | − | + | Reported | 0.0% | 0 (0) | 1 |
| | | | | | (0.0%) | | |
| 9387 | V-M | − | + | Reported | 0.0% | 0 (0) | 1 |
| | | | | | (0.0%) | | |
| 9438 | G-S | + | − | Conflicting | 1.1% | 559 (0) | 14 |
| | | | | reports | (0.0%) | | |
| 9478 | V-A | − | + | Reported | 0.0% | 18 (0) | 2 |
| | | | | | (0.0%) | | |
| 9480 | FFFAG- | − | + | Reported | 0.0% | 0 (0) | 5 |
| | del | | | | (0.0%) | | |
| 9487 | FFAGFF- | − | + | Reported | 0.0% | 0 (0) | 1 |
| | del | | | (alt loc) | (0.0%) | | |
| 9490 | A-V | + | − | Reported | 0.0% | 22 (0) | 1 |
| | | | | | (0.0%) | | |
| 9537 | frameshift | + | − | Reported | 0.0% | 0 (0) | 2 |
| | | | | | (0.0%) | | |
| 9544 | G-E | . | . | Reported | 0.0% | 0 (0) | 1 |
| | | | | | (0.0%) | | |
| 9559 | frameshift | − | + | Reported | 0.0% | 0 (0) | 1 |
| | | | | | (0.0%) | | |
| 9660 | M-L | + | − | Reported | 0.0% | 0 (0) | 1 |
| | | | | | (0.0%) | | |
| 9738 | A-S | + | − | Reported | 0.0% | 0 (0) | 1 |
| | | | | | (0.0%) | | |
| 9789 | S-P | − | + | Reported | 0.0% | 0 (0) | 1 |
| | | | | | (0.0%) | | |
| 9804 | A-T | + | − | Reported | 0.3% | 149 (0) | 10 |
| | | | | | (0.0%) | | |
| 9856 | I-T | + | − | Reported | 0.0% | 17 (0) | 2 |
| | | | | | (0.0%) | | |
| 9861 | F-L | + | − | Reported | 0.2% | 101 (0) | 1 |
| | | | | | (0.0%) | | |
| 9952 | W-Ter | − | + | Reported | 0.0% | 0 (0) | 1 |
| | | | | | (0.0%) | | |
| 9957 | F-L | − | + | Reported | 0.1% | 41 (0) | 8 |
| | | | | | (0.0%) | | |
| 9966 | V-I | . | . | Reported | 0.7% | 346 (0) | 1 |
| | | | | | (0.0%) | | |
| 9972 | I-L | − | + | Reported | 0.0% | 1 (0) | 1 |
| | | | | | (0.0%) | | |
| 10086 | N-D | + | − | Reported | 0.8% | 422 (0) | 4 |
| | | | | | (0.0%) | | |
| 10158 | S-P | + | + | Cfrm | 0.0% | 0 (0) | 27 |
| | | | | | (0.0%) | | |
| 10191 | S-P | − | + | Cfrm | 0.0% | 0 (0) | 25 |
| | | | | | (0.0%) | | |
| 10197 | A-T | + | + | Cfrm | 0.0% | 4 (0) | 20 |
| | | | | | (0.0%) | | |
| 10237 | I-T | + | − | Reported | 0.2% | 82 (0) | 3 |
| | | | | | (0.0%) | | |
| 10254 | D-N | − | + | Reported | 0.0% | 0 (0) | 1 |
| | | | | | (0.0%) | | |
| 10398 | T-T | + | − | Reported; | 55.7% | 27929 (0) | 19 |
| | | | | lineage N | (0.0%) | | |
| | | | | marker | | | |
| | | | | except hg IJK | | | |
| 10398 | T-A | + | − | Reported; | 44.3% | 22239 (0) | 34 |
| | | | | lineage L & M | (0.0%) | | |
| | | | | marker, also | | | |
| | | | | hg IJK | | | |
| 10543 | H-R | − | + | Reported | 0.0% | 0 (0) | 1 |
| | | | | | (0.0%) | | |
| 10591 | F-C | − | + | Reported | 0.0% | 0 (0) | 1 |
| | | | | | (0.0%) | | |
| 10652 | I-I | − | + | Reported | 0.1% | 53 (0) | 1 |
| | | | | | (0.0%) | | |
| 10663 | V-A | + | − | Cfrm | 0.0% | 1 (0) | 13 |
| | | | | | (0.0%) | | |
| 10680 | A-T | + | − | Reported/ | 0.0% | 18 (0) | 4 |
| | | | | possibly | (0.0%) | | |
| | | | | synergistic | | | |
| 11042 | Y-H | − | + | Reported | 0.0% | 0 (0) | 1 |
| | | | | | (0.0%) | | |
| 11048 | frameshift | − | + | Reported | 0.0% | 0 (0) | 1 |
| | | | | | (0.0%) | | |
| 11084 | T-A | + | + | Conflicting | 0.4% | 202 (0) | 7 |
| | | | | reports | (0.0%) | | |
| 11232 | L-P | − | + | Reported | 0.0% | 0 (0) | 4 |
| | | | | | (0.0%) | | |
| 11240 | L-F | − | + | Reported | 0.0% | 0 (0) | 2 |
| | | | | | (0.0%) | | |
| 11251 | L-L | . | . | Reported | 9.3% | 4660 (0) | 2 |
| | | | | | (0.0%) | | |
| 11253 | I-T | + | − | Reported | 0.5% | 257 (0) | 7 |
| | | | | | (0.0%) | | |
| 11365 | A-A | + | − | Reported | 0.2% | 110 (0) | 1 |
| | | | | | (0.0%) | | |
| 11375 | K-Q | + | − | Reported | 0.0% | 0 (0) | 1 |
| | | | | | (0.0%) | | |
| 11467 | L-L | + | − | Reported | 12.4% | 6234 (0) | 3 |
| | | | | | (0.0%) | | |
| 11470 | K-N | − | + | Reported | 0.0% | 0 (0) | 1 |
| | | | | | (0.0%) | | |
| 11621 | frameshift | − | + | Reported | 0.0% | 0 (0) | 1 |
| | | | | | (0.0%) | | |
| 11696 | V-I | + | + | Reported - | 0.6% | 299 (0) | 16 |
| | | | | possibly | (0.0%) | | |
| | | | | synergistic | | | |
| 11777 | R-S | − | + | Cfrm | 0.0% | 0 (0) | 12 |
| | | | | | (0.0%) | | |
| 11778 | R-H | + | + | Cfrm | 0.2% | 120 (0) | 301 |
| | | | | | (0.0%) | | |
| 11832 | W-Ter | − | + | Reported | 0.0% | 0 (0) | 6 |
| | | | | | (0.0%) | | |
| 11874 | T-N | + | − | Reported | 0.0% | 0 (0) | 2 |
| | | | | | (0.0%) | | |
| 11919 | S-F | + | − | Reported | 0.0% | 0 (0) | 2 |
| | | | | | (0.0%) | | |
| 11984 | Y-H | + | − | Reported | 0.1% | 51 (0) | 1 |
| | | | | | (0.0%) | | |
| 11994 | T-I | + | − | Conflicting | 0.0% | 0 (0) | 3 |
| | | | | reports | (0.0%) | | |
| 12015 | L-P | − | + | Reported | 0.0% | 2 (0) | 2 |
| | | | | | (0.0%) | | |
| 12026 | I-V | + | − | Reported | 0.5% | 245 (0) | 4 |
| | | | | | (0.0%) | | |
| 12027 | I-T | . | . | Reported | 0.0% | 2 (0) | 2 |
| | | | | | (0.0%) | | |
| 12033 | N-S | + | − | Reported: | 0.0% | 21 (0) | 1 |
| | | | | individually | (0.0%) | | |
| | | | | neutral | | | |
| | | | | variants | | | |
| | | | | causing | | | |
| | | | | LHON in | | | |
| | | | | combination | | | |
| 12338 | M-T | + | − | Conflicting | 0.3% | 174 (0) | 11 |
| | | | | reports | (0.0%) | | |
| 12361 | T-A | + | − | Reported | 0.5% | 275 (0) | 2 |
| | | | | | (0.0%) | | |
| 12372 | L-L | + | − | Reported | 13.4% | 6742 (0) | 3 |
| | | | | | (0.0%) | | |
| 12397 | T-A | + | − | Reported | 0.7% | 335 (0) | 3 |
| | | | | | (0.0%) | | |
| 12414 | frameshift | . | . | Reported | 0.0% | 0 (0) | 1 |
| | | | | | (0.0%) | | |
| 12425 | frameshift | − | + | Reported | 0.0% | 2 (0) | 1 |
| | | | | | (0.0%) | | |
| 12477 | S-S | + | − | Reported | 0.5% | 263 (0) | 1 |
| | | | | | (0.0%) | | |
| 12622 | V-I | + | + | Significance | 0.0% | 10 (0) | 2 |
| | | | | unclear | (0.0%) | | |
| 12631 | S-T | + | − | Reported | 0.0% | 0 (0) | 2 |
| | | | | | (0.0%) | | |
| 12634 | I-V | + | − | Reported | 0.3% | 141 (0) | 3 |
| | | | | | (0.0%) | | |
| 12686 | F-Y | + | − | Reported | 0.0% | 0 (0) | 1 |
| | | | | | (0.0%) | | |
| 12706 | F-L | − | + | Cfrm | 0.0% | 0 (0) | 10 |
| | | | | | (0.0%) | | |
| 12770 | E-G | − | + | Reported | 0.0% | 1 (0) | 5 |
| | | | | | (0.0%) | | |
| 12778 | G-R | + | − | Reported | 0.0% | 0 (0) | 1 |
| | | | | | (0.0%) | | |
| 12782 | I-S | − | + | Reported | 0.0% | 0 (0) | 1 |
| | | | | | (0.0%) | | |
| 12811 | Y-H | + | − | Reported | 1.3% | 633 (0) | 9 |
| | | | | | (0.0%) | | |
| 12848 | A-V | − | + | Reported | 0.0% | 0 (0) | 3 |
| | | | | | (0.0%) | | |
| 13042 | A-T | − | + | Cfrm | 0.0% | 1 (0) | 7 |
| | | | | | (0.0%) | | |
| 13045 | M-L | − | + | Reported | 0.0% | 0 (0) | 4 |
| | | | | | (0.0%) | | |
| 13046 | M-T | − | + | Reported | 0.0% | 0 (0) | 1 |
| | | | | | (0.0%) | | |
| 13051 | G-S | + | − | Cfrm | 0.0% | 0 (0) | 2 |
| | | | | | (0.0%) | | |
| 13063 | V-I | − | + | Reported | 0.0% | 2 (0) | 3 |
| | | | | | (0.0%) | | |
| 13084 | S-C | − | + | Reported | 0.0% | 0 (0) | 4 |
| | | | | | (0.0%) | | |
| 13094 | V-A | + | + | Cfrm | 0.0% | 1 (0) | 7 |
| | | | | | (0.0%) | | |
| 13135 | A-T | + | − | Reported | 0.9% | 463 (0) | 2 |
| | | | | | (0.0%) | | |
| 13204 | V-I | + | − | Reported | 0.1% | 40 (0) | 4 |
| | | | | | (0.0%) | | |
| 13271 | L-P | − | + | Reported | 0.0% | 1 (0) | 2 |
| | | | | | (0.0%) | | |
| 13276 | M-V | + | − | Conflicting | 3.3% | 1673 (0) | 2 |
| | | | | Reports | (0.0%) | | |
| 13379 | H-P | + | − | Reported | 0.0% | 0 (0) | 1 |
| | | | | | (0.0%) | | |
| 13511 | K-M | − | + | Reported | 0.0% | 0 (0) | 3 |
| | | | | | (0.0%) | | |
| 13513 | D-N | − | + | Cfrm | 0.0% | 1 (0) | 41 |
| | | | | | (0.0%) | | |
| 13514 | D-G | − | + | Cfrm | 0.0% | 0 (0) | 15 |
| | | | | | (0.0%) | | |
| 13528 | T-A | + | − | Reported | 0.1% | 49 (0) | 5 |
| | | | | | (0.0%) | | |
| 13580 | A-G | − | + | Reported | 0.0% | 0 (0) | 1 |
| | | | | | (0.0%) | | |
| 13637 | Q-R | + | − | Reported | 0.8% | 382 (0) | 4 |
| | | | | | (0.0%) | | |
| 13708 | A-T | + | − | Conflicting | 7.1% | 3563 (0) | 49 |
| | | | | reports | (0.0%) | | |
| 13730 | G-E | − | + | Reported | 0.0% | 0 (0) | 7 |
| | | | | | (0.0%) | | |
| 13831 | L-M | − | + | Reported | 0.0% | 3 (0) | 2 |
| | | | | | (0.0%) | | |
| 13849 | N-H | + | − | Reported - | 0.0% | 1 (0) | 2 |
| | | | | possible | (0.0%) | | |
| | | | | secondary | | | |
| 13967 | T-M | + | − | Reported | 0.3% | 163 (0) | 4 |
| | | | | | (0.0%) | | |
| 14063 | I-T | + | − | Reported | 0.1% | 27 (0) | 2 |
| | | | | | (0.0%) | | |
| 14091 | K-N | − | + | Reported | 0.0% | 0 (0) | 2 |
| | | | | | (0.0%) | | |
| 14163 | A-T | + | − | Conflicting | 0.0% | 13 (0) | 3 |
| | | | | reports | (0.0%) | | |
| 14258 | P-L | + | − | Reported: | 0.0% | 25 (0) | 1 |
| | | | | individually | (0.0%) | | |
| | | | | neutral | | | |
| | | | | variants | | | |
| | | | | causing | | | |
| | | | | LHON in | | | |
| | | | | combination | | | |
| 14279 | S-L | + | − | Reported | 0.0% | 6 (0) | 3 |
| | | | | | (0.0%) | | |
| 14319 | N-D | + | − | Reported | 0.1% | 65 (0) | 3 |
| | | | | | (0.0%) | | |
| 14325 | N-D | + | − | Reported | 0.1% | 52 (0) | 3 |
| | | | | | (0.0%) | | |
| 14340 | V-M | + | − | Reported | 0.0% | 23 (0) | 2 |
| | | | | | (0.0%) | | |
| 14430 | W-R | + | − | Reported | 0.0% | 0 (0) | 1 |
| | | | | | (0.0%) | | |
| 14439 | P-S | + | − | Reported | 0.0% | 0 (0) | 2 |
| | | | | | (0.0%) | | |
| 14441 | Y-C | . | . | Reported | 0.0% | 0 (0) | 1 |
| | | | | | (0.0%) | | |
| 14453 | A-V | − | + | Reported | 0.0% | 0 (0) | 6 |
| | | | | | (0.0%) | | |
| 14459 | A-V | + | + | Cfrm | 0.0% | 3 (0) | 37 |
| | | | | | (0.0%) | | |
| 14482 | M-I | + | + | Cfrm | 0.0% | 2 (0) | 13 |
| | | | | | (0.0%) | | |
| 14482 | M-I | + | + | Cfrm | 0.0% | 0 (0) | 6 |
| | | | | | (0.0%) | | |
| 14484 | M-V | + | + | Cfrm | 0.1% | 57 (0) | 170 |
| | | | | | (0.0%) | | |
| 14487 | M-V | − | + | Cfrm | 0.0% | 0 (0) | 25 |
| | | | | | (0.0%) | | |
| 14495 | L-S | − | + | Cfrm | 0.0% | 2 (0) | 8 |
| | | | | | (0.0%) | | |
| 14498 | Y-C | + | + | Reported | 0.0% | 0 (0) | 4 |
| | | | | | (0.0%) | | |
| 14502 | I-V | + | − | Reported - | 0.4% | 186 (0) | 7 |
| | | | | possibly | (0.0%) | | |
| | | | | synergistic | | | |
| 14536 | frameshift | . | . | Reported | 0.0% | 0 (0) | 1 |
| | | | | | (0.0%) | | |
| 14568 | G-S | + | − | Cfrm | 0.0% | 6 (0) | 10 |
| | | | | | (0.0%) | | |
| 14577 | I-V | − | + | Reported | 0.8% | 411 (0) | 1 |
| | | | | | (0.0%) | | |
| 14582 | V-A | + | − | Reported: | 0.5% | 252 (0) | 1 |
| | | | | individually | (0.0%) | | |
| | | | | neutral | | | |
| | | | | variants | | | |
| | | | | causing | | | |
| | | | | LHON in | | | |
| | | | | combination | | | |
| 14596 | I-M | + | − | Reported | 0.0% | 0 (0) | 5 |
| | | | | | (0.0%) | | |
| 14600 | P-L | + | + | Reported | 0.0% | 0 (0) | 3 |
| | | | | | (0.0%) | | |
| 14668 | M-M | + | − | Reported | 4.1% | 2059 (0) | 1 |
| | | | | | (0.0%) | | |
| 14787 | frameshift | − | + | Reported | 0.0% | 0 (0) | 1 |
| | | | | | (0.0%) | | |
| 14831 | A-T | + | − | Reported | 0.2% | 104 (0) | 2 |
| | | | | | (0.0%) | | |
| 14841 | N-S | − | + | Reported | 0.0% | 21 (0) | 1 |
| | | | | | (0.0%) | | |
| 14846 | G-S | − | + | Reported | 0.0% | 0 (0) | 9 |
| | | | | | (0.0%) | | |
| 14849 | S-P | − | + | Cfrm | 0.0% | 0 (0) | 3 |
| | | | | | (0.0%) | | |
| 14864 | C-R | − | + | Cfrm | 0.0% | 2 (0) | 1 |
| | | | | | (0.0%) | | |
| 14894 | F-L | . | . | Reported | 0.0% | 8 (0) | 1 |
| | | | | | (0.0%) | | |
| 15024 | C-Y | + | − | Reported | 0.1% | 32 (0) | 1 |
| | | | | | (0.0%) | | |
| 15043 | G-G | + | − | Reported | 23.6% | 11837 (0) | 2 |
| | | | | | (0.0%) | | |
| 15059 | G-Ter | − | + | Reported | 0.0% | 0 (0) | 7 |
| | | | | | (0.0%) | | |
| 15077 | E-K | + | − | Reported | 0.2% | 102 (0) | 2 |
| | | | | | (0.0%) | | |
| 15084 | W-Ter | − | + | Reported | 0.0% | 0 (0) | 2 |
| | | | | | (0.0%) | | |
| 15092 | G-S | − | + | Reported | 0.0% | 0 (0) | 1 |
| | | | | | (0.0%) | | |
| 15150 | W-Ter | − | + | Reported | 0.0% | 0 (0) | 1 |
| | | | | | (0.0%) | | |
| 15153 | G-D | − | + | Reported | 0.0% | 6 (0) | 1 |
| | | | | | (0.0%) | | |
| 15158 | M-V | − | + | Reported | 0.0% | 0 (0) | 1 |
| | | | | | (0.0%) | | |
| 15168 | W-Ter | − | + | Reported | 0.0% | 0 (0) | 2 |
| | | | | | (0.0%) | | |
| 15170 | G-Ter | − | + | Reported | 0.0% | 0 (0) | 1 |
| | | | | | (0.0%) | | |
| 15197 | S-P | − | + | Reported | 0.0% | 0 (0) | 2 |
| | | | | | (0.0%) | | |
| 15209 | Y-H | + | − | Reported | 0.0% | 4 (0) | 1 |
| | | | | | (0.0%) | | |
| 15234 | W-Ter | . | . | Reported | 0.0% | 0 (0) | 1 |
| | | | | | (0.0%) | | |
| 15237 | I-T | + | − | Reported | 0.0% | 6 (0) | 1 |
| | | | | | (0.0%) | | |
| 15242 | G-Ter | − | + | Reported | 0.0% | 0 (0) | 2 |
| | | | | | (0.0%) | | |
| 15243 | G-E | − | + | Reported | 0.0% | 0 (0) | 2 |
| | | | | | (0.0%) | | |
| 15256 | V-V | + | − | Reported | 0.0% | 4 (0) | 1 |
| | | | | | (0.0%) | | |
| 15257 | D-N | + | − | Conflicting | 1.5% | 763 (0) | 45 |
| | | | | reports | (0.0%) | | |
| 15287 | F-L | − | + | Reported; | 0.2% | 80 (0) | 1 |
| | | | | hg I6a & | (0.0%) | | |
| | | | | H10c marker | | | |
| 15395 | K-E | + | − | Reported | 0.0% | 2 (0) | 1 |
| | | | | | (0.0%) | | |
| 15453 | L-P | + | − | Reported | 0.0% | 10 (0) | 1 |
| | | | | | (0.0%) | | |
| 15497 | G-S | + | − | Reported | 0.4% | 217 (0) | 5 |
| | | | | | (0.0%) | | |
| 15498 | GDPDNYTL- | − | + | Reported | 0.0% | 0 (0) | 2 |
| | del | | | | (0.0%) | | |
| 15498 | G-D | − | + | Reported | 0.0% | 13 (0) | 7 |
| | | | | | (0.0%) | | |
| 15579 | Y-C | − | + | Cfrm | 0.0% | 0 (0) | 4 |
| | | | | | (0.0%) | | |
| 15615 | G-D | − | + | Reported | 0.0% | 0 (0) | 3 |
| | | | | | (0.0%) | | |
| 15620 | L-I | − | + | Reported | 0.0% | 0 (0) | 1 |
| | | | | | (0.0%) | | |
| 15635 | S-P | + | − | Reported | 0.0% | 2 (0) | 1 |
| | | | | | (0.0%) | | |
| 15649 | ILAMIP- | − | + | Reported | 0.0% | 0 (0) | 1 |
| | del | | | | (0.0%) | | |
| 15662 | I-V | + | + | Reported | 0.4% | 188 (0) | 1 |
| | | | | | (0.0%) | | |
| 15674 | S-P | + | − | Reported | 0.3% | 146 (0) | 2 |
| | | | | | (0.0%) | | |
| 15693 | M-T | + | − | Reported | 1.2% | 589 (0) | 1 |
| | | | | | (0.0%) | | |
| 15699 | R-P | − | + | Reported | 0.0% | 0 (0) | 2 |
| | | | | | (0.0%) | | |
| 15723 | W-Ter | − | + | Reported | 0.0% | 0 (0) | 1 |
| | | | | | (0.0%) | | |
| 15761 | G-Ter | | + | Reported | 0.0% | 0 (0) | 1 |
| | | | | | (0.0%) | | |
| 15762 | G-E | − | + | Reported | 0.0% | 0 (0) | 1 |
| | | | | | (0.0%) | | |
| 15773 | V-M | + | − | Possibly | 0.1% | 59 (0) | 1 |
| | | | | synergistic | (0.0%) | | |
| 15784 | P-P | + | − | Reported | 3.5% | 1756 (0) | 3 |
| | | | | | (0.0%) | | |
| 15800 | Q-Ter | − | + | Reported | 0.0% | 0 (0) | 2 |
| | | | | | (0.0%) | | |
| 15804 | V-A | + | − | Reported | 0.1% | 27 (0) | 1 |
| | | | | | (0.0%) | | |
| 15812 | V-M | + | − | Reported/ | 0.9% | 466 (0) | 20 |
| | | | | Secondary | (0.0%) | | |
| 16081 | noncoding | − | + | Reported | 0.0% | 1 (31) | 1 |
| | | | | | (0.0%) | | |
| 16093 | noncoding | − | + | Reported | 5.7% | 2869 (4721) | 2 |
| | | | | | (6.4%) | | |
| 16129 | noncoding | − | + | Reported | 13.2% | 6605 (11486) | 1 |
| | | | | | (15.7%) | | |
| 16176 | noncoding | − | + | Reported | 0.6% | 303 (557) | 1 |
| | | | | | (0.8%) | | |
| 16183 | noncoding | . | . | Reported | 13.6% | 6812 (11124) | 1 |
| | | | | | (15.2%) | | |
| 16189 | noncoding | + | − | Reported | 25.9% | 12979 (19118) | 34 |
| | | | | | (26.1%) | | |
| 16192 | noncoding | . | . | Reported | 4.2% | 2099 (3183) | 1 |
| | | | | | (4.3%) | | |
| 16217 | noncoding | + | − | Reported | 7.3% | 3659 (4750) | 1 |
| | | | | | (6.5%) | | |
| 16270 | noncoding | . | . | Reported | 4.6% | 2317 (2348) | 1 |
| | | | | | (3.2%) | | |
| 16300 | noncoding | + | − | Reported | 0.5% | 261 (494) | 2 |
| | | | | | (0.7%) | | |
| 16318 | noncoding | . | . | Reported | 0.2% | 94 (109) | 1 |
| | | | | | (0.1%) | | |
| 16390 | noncoding | + | − | Reported | 5.9% | 2947 (4457) | 3 |
| | | | | | (6.1%) | | |
| 16519 | noncoding | + | − | Reported | 36.9% | 18531 (0) | 4 |
| | | | | | (0.0%) | | |

Column Heading Key:
A: Position;
B: Locus;
C: Disease;
D: Allele;
E: RNA;
F: Homoplasmy;
G: Heteroplasmy;
H: Status;
I: Mito Tip;
J: GB Freq FL (CR);
K: GB Seqs FL (CR);
L: Reference

TABLE 7
“Top 19” Primary Leber's Hereditary Optic Neuropathy (LHON) mutations, the first 3 mutations listed
(in boldface) represent approximately 95% of all cases. The remaining mutations are listed in nucleotide order.

| | | | AA | % | % | | Penetrance ^{c} | Penetrance ^{c} | % | |
|---|---|---|---|---|---|---|---|---|---|---|
| Mutation | NT Δ | AA Δ | Cons ^{a} | Patients | Controls | Het. ^{b} | % Relatives | % Males | Recovery ^{d} | Refs. |
| m.11778G > A | G-A | R340H | 100% | 69 | 0 | +/− | 33-60 | 82 | 4 | (27) |
| ND4 | | | | | | | | | | |
| m.3460G > A | G-A | A52T | 91% | 13 | 0 | +/− | 14-75 | 40-80 | 22 | (10, 16) |
| ND1 | | | | | | | | | | |
| m.14484T > C | T-C | M64V | 31% | 14 | 0 | +/− | 27-80 | 68 | 37-65 | (2, 13, 18) |
| ND6 | | | | | | | | | | |
| m.3376G > A | G-A | E24K | 98% | Rare | 0 | +/+ | NA | NA | NA | (33, 34, 35) |
| ND1 | | | | | | | | | | |
| m.3635G > A | G-A | S110N | 93% | Rare | 0 | +/− | 29 (range | 54 (range | Low | (3) |
| ND1 | | | | | | | 11-64) | 25-100) | | |
| m.3697G > A | G-A | G131S | 100% | Rare | 0 | +/+ | NA | NA | NA | (32) |
| ND1 | | | | | | | | | | |
| m.3700G > A | G-A | A112T | 93% | Rare | 0 | − | NA | NA | UN | (1a, 7) |
| ND1 | | | | | | | | | | |
| m.3733G > A | G-A | E143K | 100% | Rare | 0 | +/− | 24-30 | 36-44 | Yes | (1a, 26) |
| ND1 | | | | | | | | | | |
| m.4171C > A | C-A | L289M | 93% | Rare | 0 | +/− | 46 | 47 | Yes | (20) |
| ND1 | | | | | | | | | | |
| m.10197G > A | G-A | A47T | 96% | Rare | 4/42616 | +/+ | NA | NA | NA | (36) |
| ND3 | | | | | | | | | | |
| m.10663T > C | T-C | V65A | 89% | Rare | 0 | +/− | 56 | 60 | UN | (1a, 1b) |
| ND4L | | | | | | | | | | |
| m.13051G > A | G-A | G239S | 98% | Rare | 0 | − | 56 | 63 | UN | (5b, 14) |
| ND5 | | | | | | | | | | |
| m.13094T > C | T-C | V253A | 100% | Rare | 0 | + | NA | NA | Yes | (5c, 23b) |
| ND5 | | | | | | | | | | |
| m.14459G > A | G-A | A72V | 89% | Rare | 0 | + | NA | NA | Low | (8, 19, 24) |
| ND6 | | | | | | | | | | |
| m.14482C > A | C-A | M64I | 31% | Rare | 0 | +/− | NA | 89 | Yes | (1a, 25) |
| ND6 | | | | | | | | | | |
| m.14482C > G | C-G | M64I | 31% | Rare | 0 | − | NA | NA | UN | (11) |
| ND6 | | | | | | | | | | |
| m.14495A > G | A-G | L60S | 100% | Rare | 0 | + | NA | NA | Low | (4) |
| ND6 | | | | | | | | | | |
| m.14502T > C | T-C | I58V | 78% | Rare | 0 | − | 14502:10% 14502 + | 14502:11% 14502 + | UN | (1a, 30, 31) |
| ND6 | | | | | | | 11778:37% | 11778:47% | | |
| m.14568C > T | C-T | G36S | 87% | Rare | 0 | − | NA | NA | UN | (6, 28) |
| ND6 | | | | | | | | | | |

^{a} Conservation calculated using Mitomaster with the species set shown here
^{b} Het. = Heteroplasmy; + = detected, − = not detected.
^{c} NA = not applicable; UN = unknown; penetrance values are rough estimates.
^{d} Low = anecdotal low degree of vision recovery; Yes = anecdotal moderate to high degree of vision recovery; UN = unknown; NA = not applicable

TABLE 8
Other candidate LHON mutations found as single family or singleton cases.

| | | | AA | # | # | | | |
|---|---|---|---|---|---|---|---|---|
| Mutation | NT Δ | AA Δ | Cons ^{a} | Patients | Controls | Het. ^{b} | Recovery ^{d} | References |
| m.3472T > C ND1 | T-C | F56L | 96% | 1 case | 3 | − | UN | (22b) |
| m.4025C > T ND1 | C-T | T240M | 33% | 1 family; 3 cases | 0 | − | UN | (15) |
| m.4160T > C ND1 | T-C | L285P | 100% | 1 family; 9 cases | 1 | − | UN | (13) |
| m.4640C > A ND2 | C-A | I57M | 27% | 1 family; 4 cases | 0 | − | UN | (3) |
| m.5244G > A ND2 | G-A | G259S | 100% | 1 case | 0 | + | UN | (1b) |
| m.9101T > C ATP6 | T-C | I192T | 13% | 1 case | 0 | − | UN | (21) |
| m.9804G > A CO3 | G-A | A200T | 93% | Multiple unrelated | 0 | − | UN | (14, 17) |
| | | | | singleton cases | | | | |
| m.10237T > C ND3 | T-C | I60T | 100% | 1 family; 2 cases | 0 | − | UN | (9) |
| m.11253T > C ND4 | T-C | I165T | 42% | 1 case | 0 | − | Yes | (22) |
| m.11696G > A (ND4) & | G-A | V312I | 7% | 1 family; 11 cases | 0 | + | UN | (5) |
| m.14596A > T (ND6) | A-T | I26M | 84% | | | | | |
| m.12811T > C ND5 | T-C | Y159H | 56% | 1 family; 2 cases | 0 | − | UN | (15) |
| m.12848C > T ND5 | C-T | A171V | 98% | 1 case | 0 | + | UN | (23) |
| m.13637A > G ND5 | A-G | Q434R | 62% | 1 family; 3 cases | 0 | − | UN | (15) |
| m.13730G > A ND5 | G-A | G465E | 100% | 1 case | 0 | + | Yes | (12) |
| m.14279G > A ND6 | G-A | S132L | 47% | 1 family; 2 cases | 0 | − | UN | (29) |
| m.14325T > C ND6 | T-C | N117D | 18% | 1 case | 0 | − | UN | (14) |
| m.14498T > C ND6 | T-C | Y59C | 98% | 1 case | 0 | +/− | UN | (28) |
| m.14831G > A CytB | G-A | A29T | 42% | 1 case | 0 | − | UN | (7) |

^{a} Conservation calculated using Mitomaster with the species set shown here
^{b} Het. = Heteroplasmy; + = detected, − = not detected.
c. NA= not applicable; UN= unknown; penetrance values are rough estimates.
^{d} Low = anecdotal low degree of vision recovery; Yes = anecdotal moderate to high degree of vision recovery; UN = unknown; NA = not applicable

Other databases and/or tools that can be used to identify and/or characterize a mtDNA mutation in a mtDNA sequence can include PhyloTree (phylotree.org), Haplogrep (haplogrep.i-med.ac.at), MSeqDR (mseqdr.org/MITO/genes), AmtDB (amtdb.org), HmtDB (hmtdb.uniba.it), PON tRNA (structure.bmc.lu.se/PON-mt-tRNA/), Mitlmpact (mitimpact.css-mendel.it), HvrBase++ (hvrbase.cibiv.univie.ac.at), GiiB-JST mtSNP (mtsnp.tmig.or.jp/mtsnp/index_e.shtml), HmtVar (hmtvar.uniba.it), mt-DNA Server (mtdna-server.uibk.ac.at/index.html), EMPOP CR (empop.online), Mitominer (mitominer.mrc-mbu.cam.ac.uk/release-4.0/begin.do), POLG Pathogenicity Server (mitomap.org/polg/), MitoWheel (mitomap.org/MITOMAP), POLG @NIEHS (tools.niehs.nih.gov//polg/), MitoBreak (mitobreak.portugene.com/cgi-bin/Mitobreak_home.cgi), MitoAge (mitoage.info), Mamit-tRNA/mitotRNAdb (mttrna.bioinf.uni-leipzig.de/mtDataOutput/), MitoFit (mitofit.org/index.php/MitoFit), Misynpat (misynpat.org/misynpat/).

### Other Disease Applications

As noted above, mitochondrial diseases and disorders can also be a contributing factor to a wide range of other human diseases. Accordingly, treatment of other human diseases involving a mitochondrial disease or disorder component are further contemplated either alone or in combination with known therapies for those diseases. Exemplary applications in the context of other human diseases are further discussed below. In some embodiments the human disease includes cell or cells that exhibit a disease state. Exemplary disease states are shown in Table 9.

TABLE 9
DISEASE STATES

| | |
|---|---|
| Disease States | The disease state is an infection (e.g., a fungal infection, a bacterial infection, a parasite |
| | infection, or a viral infection), an organ disease, a blood disease, an immune system |
| | disease, a cancer, a brain and nervous system disease, an endocrine disease, a pregnancy |
| | or childbirth-related disease, an inherited disease, or an environmentally-acquired disease. |
| Viral Infections | Viral infections and diseases caused by a double-stranded RNA virus, a positive sense |
| | RNA virus, a negative sense RNA virus, a retrovirus, or a combination thereof, or the viral |
| | infection is caused by a Coronaviridae virus, a Picornaviridae virus, a Caliciviridae virus, |
| | a Flaviviridae virus, a Togaviridae virus, a Bornaviridae, a Filoviridae, a Paramyxoviridae, |
| | a Pneumoviridae, a Rhabdoviridae, an Arenaviridae, a Bunyaviridae, an |
| | Orthomyxoviridae, or a Deltavirus, or the viral infection is caused by Coronavirus, SARS, |
| | Poliovirus, Rhinovirus, Hepatitis A, Norwalk virus, Yellow fever virus, West Nile virus, |
| | Hepatitis C virus, Dengue fever virus, Zika virus, Rubella virus, Ross River virus, Sindbis |
| | virus, Chikungunya virus, Borna disease virus, Ebola virus, Marburg virus, Measles virus, |
| | Mumps virus, Nipah virus, Hendra virus, Newcastle disease virus, Human respiratory |
| | syncytial virus, Rabies virus, Lassa virus, Hantavirus, Crimean-Congo hemorrhagic fever |
| | virus, Influenza, or Hepatitis D virus. |
| Plant Viruses | Disease caused from plant viruses selected from the group comprising Tobacco mosaic |
| | virus (TMV), Tomato spotted wilt virus (TSWV), Cucumber mosaic virus (CMV), Potato |
| | virus Y (PVY), the RT virus Cauliflower mosaic virus (CaMV), Plum pox virus (PPV), |
| | Brome mosaic virus (BMV), Potato virus X (PVX), Citrus tristeza virus (CTV), Barley |
| | yellow dwarf virus (BYDV), Potato leafroll virus (PLRV), Tomato bushy stunt virus |
| | (TBSV), rice tungro spherical virus (RTSV), rice yellow mottle virus (RYMV), rice hoja |
| | blanca virus (RHBV), maize rayado fino virus (MRFV), maize dwarf mosaic virus |
| | (MDMV), sugarcane mosaic virus (SCMV), Sweet potato feathery mottle virus (SPFMV), |
| | sweet potato sunken vein closterovirus (SPSVV), Grapevine fanleaf virus (GFLV), |
| | Grapevine virus A (GVA), Grapevine virus B (GVB), Grapevine fleck virus (GFkV), |
| | Grapevine leafroll-associated virus-1, -2, and -3, (GLRaV-1, -2, and -3), Arabis mosaic |
| | virus (ArMV), or Rupestris stem pitting-associated virus (RSPaV). |
| DNA Viruses | Diseases caused from DNA viruses from the Family Myoviridae, Podoviridae, |
| | Siphoviridae, Alloherpesviridae, Herpesviridae (including human herpes virus, and |
| | Varicella Zozter virus), Malocoherpesviridae, Lipothrixviridae, R Rudiviridae, |
| | Adenoviridae, Ampullaviridae, Ascoviridae, Asfarviridae (including African swine fever |
| | virus), Baculoviridae, Cicaudaviridae, Clavaviridae, Corticoviridae, Fuselloviridae, |
| | Globuloviridae, Guttaviridae, Hytrosaviridae, Iridoviridae, Maseilleviridae, Mimiviridae, |
| | Nudiviridae, Nimaviridae, Pandoraviridae, Papillomaviridae, Phycodnaviridae, |
| | Plasmaviridae, Polydnaviruses, Polyomaviridae (including Simian virus 40, JC virus, BK |
| | virus), Poxviridae (including Cowpox and smallpox), Sphaerolipoviridae, Tectiviridae, |
| | Turriviridae, Dinodnavirus, Salterprovirus, Rhizidovirus, among others. |
| Retroviruses | Diseases caused by retroviruses that include one or more of, or any combination of, viruses |
| | of the Genus Alpharetrovirus, Betaretrovirus, Gammaretrovirus, Deltaretrovirus, |
| | Epsilonretrovirus, Lentivirus, Spumavirus, or the Family Metaviridae, Pseudoviridae, and |
| | Retroviridae (including HIV), Hepadnaviridae (including Hepatitis B virus), and |
| | Caulimoviridae (including Cauliflower mosaic virus). |
| Pathogenic Bacteria | Diseases caused from pathogenic bacteria, including, but not limited to, Acinetobacter |
| | baumanii, Actinobacillus sp., Actinomycetes, Actinomyces sp. (such as Actinomyces |
| | israelii and Actinomyces naeslundii), Aeromonas sp. (such as Aeromonas hydrophila, |
| | Aeromonas veronii biovar sobria (Aeromonas sobria), and Aeromonas caviae), Anaplasma |
| | phagocytophilum, Anaplasma marginal,e Alcaligenes xylosoxidans, Acinetobacter |
| | baumanii, Actinobacillus actinomycetemcomitans, Bacillus sp. (such as Bacillus anthracis, |
| | Bacillus cereus, Bacillus subtilis, Bacillus thuringiensis, and Bacillus stearothermophilus), |
| | Bacteroides sp. (such as Bacteroides fragilis), Bartonella sp. (such as Bartonella |
| | bacilliformis and Bartonella henselae, Bifidobacterium sp., Bordetella sp. ( such as |
| | Bordetella pertussis, Bordetella parapertussis, and Bordetella bronchiseptica), Borrelia |
| | sp. (such as Borrelia recurrentis, and Borrelia burgdorferi), Brucella sp. (such as Brucella |
| | abortus, Brucella canis, Brucella melintensis and Brucella suis), Burkholderia sp. (such as |
| | Burkholderia pseudomallei and Burkholderia cepacia), Campylobacter sp. (such as |
| | Campylobacter jejuni, Campylobacter coli, Campylobacter lari and Campylobacter fetus), |
| | Capnocytophaga sp., Cardiobacterium hominis, Chlamydia trachomatis, Chlamydophila |
| | pneumoniae, Chlamydophila psittaci, Citrobacter sp. Coxiella burnetii, Corynebacterium |
| | sp. (such as, Corynebacterium diphtheriae, Corynebacterium jeikeum and |
| | Corynebacterium), Clostridium sp. (such as Clostridium perfringens, Clostridium difficile, |
| | Clostridium botulinum and Clostridium tetani), Eikenella corrodens, Enterobacter sp. |
| | (such as Enterobacter aerogenes, Enterobacter agglomerans, Enterobacter cloacae and |
| | Escherichia coli, including opportunistic Escherichia coli, such as enterotoxigenic E. coli, |
| | enteroinvasive E. coli, enteropathogenic E. coli, enterohemorrhagic E. coli, |
| | enteroaggregative E. coli and uropathogenic E. coli) Enterococcus sp. (such as |
| | Enterococcus faecalis and Enterococcus faecium) Ehrlichia sp. (such as Ehrlichia |
| | chafeensia and Ehrlichia canis), Erysipelothrix rhusiopathiae, Eubacterium sp., |
| | Francisella tularensis, Fusobacterium nucleatum, Gardnerella vaginalis, Gemella |
| | morbillorum, Haemophilus sp. (such as Haemophilus influenzae, Haemophilus ducreyi, |
| | Haemophilus aegyptius, Haemophilus parainfluenzae, Haemophilus haemolyticus and |
| | Haemophilus parahaemolyticus, Helicobacter sp. (such as Helicobacter pylori, |
| | Helicobacter cinaedi and Helicobacter fennelliae), Kingella kingii, Klebsiella sp. ( such as |
| | Klebsiella pneumoniae, Klebsiella granulomatis and Klebsiella oxytoca), Lactobacillus |
| | sp., Listeria monocytogenes, Leptospira interrogans, Legionella pneumophila, Leptospira |
| | interrogans, Peptostreptococcus sp., Mannheimia hemolytica, Moraxella catarrhalis, |
| | Morganella sp., Mobiluncus sp., Micrococcus sp., Mycobacterium sp. (such as |
| | Mycobacterium leprae, Mycobacterium tuberculosis, Mycobacterium paratuberculosis, |
| | Mycobacterium intracellulare, Mycobacterium avium, Mycobacterium bovis, and |
| | Mycobacterium marinum), Mycoplasm sp. (such as Mycoplasma pneumoniae, |
| | Mycoplasma hominis, and Mycoplasma genitalium), Nocardia sp. (such as Nocardia |
| | asteroides, Nocardia cyriacigeorgica and Nocardia brasiliensis), Neisseria sp. (such as |
| | Neisseria gonorrhoeae and Neisseria meningitidis), Pasteurella multocida, Plesiomonas |
| | shigelloides. Prevotella sp., Porphyromonas sp., Prevotella melaninogenica, Proteus sp. |
| | (such as Proteus vulgaris and Proteus mirabilis), Providencia sp. (such as Providencia |
| | alcalifaciens, Providencia rettgeri and Providencia stuartii), Pseudomonas aeruginosa, |
| | Propionibacterium acnes, Rhodococcus equi, Rickettsia sp. (such as Rickettsia rickettsii, |
| | Rickettsia akari and Rickettsia prowazekii, Orientia tsutsugamushi (formerly: Rickettsia |
| | tsutsugamushi) and Rickettsia typhi), Rhodococcus sp., Serratia marcescens, |
| | Stenotrophomonas maltophilia, Salmonella sp. (such as Salmonella enterica, Salmonella |
| | typhi, Salmonella paratyphi, Salmonella enteritidis, Salmonella cholerasuis and |
| | Salmonella typhimurium), Serratia sp. (such as Serratia marcesans and Serratia |
| | liquifaciens), Shigella sp. (such as Shigella dysenteriae, Shigella flexneri, Shigella boydii |
| | and Shigella sonnei), Staphylococcus sp. (such as Staphylococcus aureus, Staphylococcus |
| | epidermidis, Staphylococcus hemolyticus, Staphylococcus saprophyticus), Streptococcus |
| | sp. (such as Streptococcus pneumoniae (for example chloramphenicol-resistant serotype 4 |
| | Streptococcus pneumoniae, spectinomycin-resistant serotype 6B Streptococcus |
| | pneumoniae, streptomycin-resistant serotype 9V Streptococcus pneumoniae, |
| | erythromycin-resistant serotype 14 Streptococcus pneumoniae, optochin-resistant |
| | serotype 14 Streptococcus pneumoniae, rifampicin-resistant serotype 18C Streptococcus |
| | pneumoniae, tetracycline-resistant serotype 19F Streptococcus pneumoniae, penicillin- |
| | resistant serotype 19F Streptococcus pneumoniae, and trimethoprim-resistant serotype |
| | 23F Streptococcus pneumoniae, chloramphenicol-resistant serotype 4 Streptococcus |
| | pneumoniae, spectinomycin-resistant serotype 6B Streptococcus pneumoniae, |
| | streptomycin-resistant serotype 9V Streptococcus pneumoniae, optochin-resistant |
| | serotype 14 Streptococcus pneumoniae, rifampicin-resistant serotype 18C Streptococcus |
| | pneumoniae, penicillin-resistant serotype 19F Streptococcus pneumoniae, or |
| | trimethoprim-resistant serotype 23F Streptococcus pneumoniae), Streptococcus |
| | agalactiae, Streptococcus mutans, Streptococcus pyogenes, Group A streptococci, |
| | Streptococcus pyogenes, Group B streptococci, Streptococcus agalactiae, Group C |
| | streptococci, Streptococcus anginosus, Streptococcus equismilis, Group D streptococci, |
| | Streptococcus bovis, Group F streptococci, and Streptococcus anginosus Group G |
| | streptococci), Spirillum minus, Streptobacillus moniliformi, Treponema sp. (such as |
| | Treponema carateum, Treponema petenue, Treponema pallidum and Treponema |
| | endemicum, Tropheryma whippelii, Ureaplasma urealyticum, Veillonella sp., Vibrio sp. |
| | (such as Vibrio cholerae, Vibrio parahemolyticus, Vibrio vulnificus, Vibrio |
| | parahaemolyticus, Vibrio vulnificus, Vibrio alginolyticus, Vibrio mimicus, Vibrio hollisae, |
| | Vibrio fluvialis, Vibrio metchnikovii, Vibrio damsela and Vibrio furnisii), Yersinia sp. |
| | (such as Yersinia enterocolitica, Yersinia pestis, and Yersinia pseudotuberculosis) and |
| | Xanthomonas maltophilia among others. |
| Fungal Microbes | Diseases caused by Aspergillus, Blastomyces, Candidiasis, Coccidiodomycosis, |
| | Cryptococcus neoformans, Cryptococcus gatti, Histoplasma, Mucroymcosis, |
| | Pneumocystis, Sporothrix, fungal eye infections, ringworm, Exserohilum, and |
| | Cladosporium. |
| Fungal Yeasts and | Diseases caused by Aspergillus species, a Geotrichum species, a Saccharomyces species, |
| Molds | a Hansenula species, a Candida species, a Kluyveromyces species, a Debaryomyces |
| | species, a Pichia species, or combination thereof. Example molds include, but are not |
| | limited to, a Penicillium species, a Cladosporium species, a Byssochlamys species, or a |
| | combination thereof. |
| Infectious Disease | Acne- Proprionibacterium acnes |
| Names and Their | Acute bacterial rhinosinusitis- most common = Streptococcus pneumoniae (G+ |
| Etiologies | coccus) and Haemophilus influenzae (G− pleomorphic rod) |
| (A) | Acute hemorrhagic conjunctivitis (*) - Coxsackie A-24 virus (Picornavirus: |
| | Enterovirus), Enterovirus 70 (Picornavirus: Enterovirus) |
| | Acute hemorrhagic cystitis (*) - Adenovirus 11 and 21 (Adenovirus) |
| | Acute rhinosinusitis- respiratory viruses usually |
| | Acquired Immunodeficiency Sydrome (AIDS) - Human Immunodeficiency |
| | Virus (HIV-1 and HIV-2) (retrovirus) |
| | Acrodermatitis chronica atrophicans (ACA)- late skin manifestation of latent |
| | Lyme disease- Borrelia burgdorferi (Spirochetes) |
| | Adult T-cell Leukemia-Lymphoma (ATLL) - Human T-cell Leukemia viruses I |
| | or II (retrovirus) |
| | African Sleeping Sickness - Trypanosomiasis - African = Trypanosoma brucei |
| | rhodesiense, Trypanosoma brucei gambiense (tsetse fly-borne) |
| | AIDS- Human immunodeficiency virus (HIV) |
| | Alveolar hydatid - Echinococcus multilocularis (larval cestode infection) |
| | Amebiasis - Entamoeba histolytica (protozoan parasite) |
| | Amebic meningoencephalitis- Naegleria fowleri, Acanthamoeba species, and |
| | Balamuthia mandrillaris (protozoan) |
| | Anthrax - Black Bane- Malignant pustule- Wool sorter's disease- Tanner's |
| | disease- Bacillus anthracis (G+ rod: sporulating: aerobic) |
| | Ascariasis - Roundworm infections - Ascaris lumbricoides (intestinal nematode) |
| | Aseptic meningitis (*)- Coxsackie B virus, Echovirus, Mumps virus, Coxsackie |
| | A virus, Polio virus, (5 most common) then Human Herpesvirus 1, Arboviruses, |
| | Lymphocytic choriomeningitis viruses (Arenavirus), Encephalomycarditis |
| | viruses, Louping Ill virus, Pseudolymphocytic meningitis virus, Hepatitis |
| | viruses, Adenoviruses, Rhinoviruses. |
| | Athlete's foot - Tinea pedis - Trichophyton spp., and Epidermophyton floccosum |
| | (fungi) |
| | Australian tick typhus- Australian Spotted Fever- Queensland Tick Typhus- |
| | Rickettsia australis, (G−; intracellular bacteria) |
| | Avian Influenza- Bird Flu- Influenza virus A H5N1 |
| (B) | Babesiosis - Babesia microti (protozoan parasite; transmitted by deer tick) |
| | Bacillary angiomatosis - Bartonella henselae (pleomorphic G−) |
| | Bacterial meningitis- Streptococcus agalactiae, Escherichia coli, Streptococcus |
| | pneumoniae, Neisseria meningitidis, Listeria monocytogenes, Gram negative |
| | rod-shaped bacteria |
| | Bacterial vaginosis- Gardnerella vaginalis, Mycoplasma hominis and various |
| | anaerobic bacteria including Mobiluncus sp., and Prevotella sp. |
| | Balanitis- Candida albicans (yeast)- most common. |
| | Balantidiasis- Balantidium coli (flagellated protozoan) |
| | Bang's disease - Brucellosis - Brucella sp. (G− coccobacillus; zoonoses) |
| | Bartonellosis - Verruga peruana- Carrion's disease - Oroya fever - Bartonella |
| | bacilliformis (weak G− polymorphic) sandfly bites at elevations of 600 to 2800 |
| | meter in Peru, Ecuador and Colombia. |
| | Bay sore - Chiclero's ulcer - Leishmania leishmania mexicana (protozoan |
| | parasite) sandfly |
| | Baylisascaris infection - Racoon roundworm infection- Baylisascaris procyonis |
| | Beaver fever - giardiasis - Giardia lamblia |
| | Beef tapeworm - Taenia saginata |
| | Bejel - endemic syphilis - Treponema pallidum var. endemicum |
| | Biphasic meningoencephalitis- Central European tick-borne encephalitis- |
| | Czechoslovak tick-borne encephalitis- Diphasic milk fever- Tick-borne |
| | encephalitis- Viral meningoencephalitis- Tick-borne encephalitis virus- |
| | Flaviviridae |
| | Bird Flu- Avian Influenza- Influenza virus A H5N1 |
| | Black Bane- Anthrax- Malignant pustule- Wool sorter's disease- Tanner's |
| | disease- Bacillus anthracis (G+ rod: sporulating: aerobic) |
| | ″Black death″ (plague) - Yersinia pestis (G− rod: facultative-straight: zoonoses) |
| | Black piedra- Piedraia hortai (fungal infection of hair shaft) |
| | Blackwater Fever- Malaria- Plasmodium falciparum (sporozoan parasite) |
| | Blastomycosis- Chicago disease- Gilchrist's disease- North American |
| | blastomycosis- Blastomyces dermatitidis (dimorphic fungus) |
| | Blennorrhea of the newborn- Chlamydia trachomatis |
| | Blepharitis- infestation of the eyelash follicle by a mite. This results in an |
| | allergic reaction which leads to an inflammatory reaction and secondary |
| | infection with Staphylococcus aureus or Staphylococcus epidermidis. |
| | Boils - Staphylococcus aureus (G+ coccus) |
| | Bornholm disease (pleurodynia) - Coxsackie B (Picornavirus: Enterovirus) |
| | Borrelia miyamotoi Disease- Borrelia miyamotoi (G− bacterium; spirochete) |
| | Botulism - Clostridium botulinum (G+ rod: sporulating: anaerobic) |
| | Boutonneuse fever- Fievre boutonneuse- Tick typhus- Rickettsia conori (G− |
| | intracellular; tick-borne) |
| | Brazilian purpuric fever - Haemophilus aegyptius (G− rod: facultative-straight: |
| | respiratory pathogens) |
| | Break Bone fever- dandy fever- Dengue virus (Flaviviridae) |
| | Brill-Zinsser disease - recrudescent typhus - Rickettsia prowazekii (G− |
| | intracellular; flea-borne) |
| | Bronchitis- Respiratory syncytial virus (Paramyxovirus), Parainfluenza virus |
| | (Paramyxovirus), Influenza virus |
| | Bronchiolitis (*) - Respiratory syncytial virus (Paramyxovirus), Parainfluenza |
| | virus (Paramyxovirus) |
| | Brucellosis - Brucella sp. (G− coccobacillus; zoonoses) |
| | Bubonic plague- Yersinia pestis |
| | Bullous impetigo- Staphylococcus aureus |
| | Buruli ulcers- Mycoburuli ulcers- Mycobacterium ulcerans |
| | Busse-Buschke disease- Cryptococcosis- Torulosis- European blastomycosis- |
| | Cryptococcus neoformans (encapsulated yeast) |
| (C) | California group encephalitis - California encephalitis virus, La Crosse virus, |
| | Jamestown Canyon, Snowshoe hare virus (Bunyavirus) mosquitoes |
| | Candidiasis- Candidosis- Moniliasis- infection of the mucous membranes |
| | (mouth, esophagus, vagina) caused by the yeast Candida albicans. |
| | Candidosis- Candidiasis- Moniliasis- infection of the mucous membranes |
| | (mouth, esophagus, vagina) caused by the yeast Candida albicans. |
| | Canefield fever- canicola fever- 7-day fever- Weil's disease - leptospirosis - |
| | nanukayami fever- Leptospira interrogans (spiral shaped bacteria) |
| | Canicola fever- 7-day fever- Weil's disease - leptospirosis - canefield fever- |
| | nanukayami fever- Leptospira interrogans (spiral shaped bacteria) |
| | Capillariasis - Capillaria philippinensis (intestinal nematode) |
| | Carate - Mal del pinto - Pinta - Treponema pallidum var. carateum |
| | Carbuncle - Staphylococcus aureus (G+ coccus) |
| | Carrion's disease - Bartonellosis - Oroya fever - Bartonella bacilliformis (weak |
| | G− polymorphic) sandfly bites at elevations of 600 to 2800 meter in Peru, |
| | Ecuador and Colombia. |
| | Cat Scratch fever - Cat Scratch Disease- Bartonella henselae (pleomorphic G−) |
| | Cave disease- Darling's Disease- spelunker's disease- Histoplasmosis- |
| | Histoplasma capsulatum (dimorphic fungus) |
| | Central Asian hemorrhagic fever- Congo-Crimean hemorrhagic fever- Crimean- |
| | Congo hemorrhagic fever- Congo fever- Crimean-Congo hemorrhagic fever |
| | virus- Bunyavirus- Nairovirus |
| | Central European tick-borne encephalitis- Diphasic milk fever- Biphasic |
| | meningoencephalitis, Czechoslovak tick-borne encephalitis, Tick-borne |
| | encephalitis, Viral meningoencephalitis, Tick-borne encephalitis virus- |
| | Flaviviridae |
| | Cervical cancer - human papilloma virus (Papovavirus) |
| | Chancroid - Haemophilus ducreyi (G− rod: facultative-straight: respiratory |
| | pathogens) |
| | Chicago disease- Blastomycosis- Gilchrist's disease- North American |
| | blastomycosis- Blastomyces dermatitidis (dimorphic fungus) |
| | Chikungunya fever- Chikungunya virus- Togaviridae- Alphavirus |
| | Chagas disease - Trypanosomiasis - American = Trypanosoma cruzi (Triatomine |
| | bugs = kissing bug or assassin bugs) |
| | Chickenpox - Varicella-Zoster virus (VZV or Human herpes 3 virus) |
| | Chiclero's ulcer - Bay sore - Leishmania leishmania mexicana (protozoan |
| | parasite) sandfly |
| | Chlamydia - Chlamydiae trachomatis (Obligate intracellular) |
| | Chlamydial infection- Chlamydiae trachomatis (Obligate intracellular) |
| | Cholera - Vibrio cholerae (G− rods: facultative-curved: enteric pathogens) |
| | Chromoblastomycosis - Fonsecaea pedrosoi (fungus) |
| | Clap - Gonorrhea - Neisseria gonorrhoeae (G− cocci) |
| | Clonorchiasis - Liver fluke infection - Clonorchis sinensis (liver flukes) |
| | Coccidioidomycosis- San Joaquin Valley fever, desert rheumatism, Posada- |
| | Wernicke disease- Coccidioides immitis (dimorphic fungus). |
| | Coenurosis - Taenia spp. (larval cestode infection) |
| | Colorado tick fever - Colorado tick fever virus (Reovirus) |
| | Congo fever- Congo-Crimean hemorrhagic fever- Crimean-Congo hemorrhagic |
| | fever- Crimean-Congo hemorrhagic fever virus- Central Asian hemorrhagic |
| | fever- Bunyavirus- Nairovirus |
| | Congo hemorrhagic fever virus- Congo-Crimean hemorrhagic fever-Crimean- |
| | Congo fever- Crimean-Central Asian hemorrhagic fever- Bunyavirus- |
| | Nairovirus |
| | Congo-Crimean hemorrhagic fever- Crimean-Congo hemorrhagic fever- Congo |
| | fever- Crimean-Congo hemorrhagic fever virus- Central Asian hemorrhagic |
| | fever- Bunyavirus- Nairovirus |
| | Condyloma accuminata - Warts - Papilloma virus |
| | Condyloma lata - Treponema pallidum subsp. pallidum (spirochete) secondary |
| | syphilis |
| | Conjunctivitis (*) - Haemophilus aegyptius (G− rod: facultative-straight: |
| | respiratory pathogens), Chlamydiae trachomatis (Obligate intracellular) |
| | Cowpox - vaccinia virus (Poxvirus) |
| | Crabs - Pediculosis - lice |
| | Creutzfeldt-Jakob disease - prion (a protein) |
| | Crimean-Congo hemorrhagic fever- Congo fever- Congo-Crimean hemorrhagic |
| | fever- Crimean-Congo hemorrhagic fever virus- Central Asian hemorrhagic |
| | fever- Bunyavirus- Nairovirus |
| | Croup, infectious - parainfluenza viruses 1-3 (Paramyxovirus) |
| | Cryptococcosis- Busse-Buschke disease- Torulosis- European blastomycosis- |
| | Cryptococcus neoformans (encapsulated yeast) |
| | Cutaneous Larval Migrans - Ancylostoma braziliense (filariform larvae; |
| | parasite) and many other parasitic worms normally found in animals. |
| | Cyclosporiasis- Cyclospora cayetanensis |
| | Cysticercosis - Taenia solium (larval form of the cestode) |
| | Cystic hydatid - Echinococcus granulosus (larval cestode infection) |
| | Cystitis(*) - most common = Escherichia coli, others include Klebsiella sp, |
| | Enterobacter sp., Serratia sp., Proteus sp., Providencia sp., Morganella sp., |
| | Pseudomonas aeruginosa, (the previous organisms are G− rods), Staphylococcus |
| | saprophyticus, Enterococcus sp., Staphylococcus aureus, Staphylococcus |
| | epidermidis, Streptococcus agalactiae, (G+ cocci), and Candida albicans (yeast) |
| | Czechoslovak tick-borne encephalitis,- Central European tick-borne |
| | encephalitis- Diphasic milk fever- Biphasic meningoencephalitis, Tick-borne |
| | encephalitis, Viral meningoencephalitis, Tick-borne encephalitis virus- |
| | Flaviviridae |
| (D) | Dacryocytitis- Staphylococcus aureus, Staphylococcus epidermidis, |
| | Streptococcus pneumoniae |
| | Dandy fever- Break Bone fever- Dengue virus (Flaviviridae) |
| | Darling's Disease- cave disease- spelunker's disease- Histoplasmosis- |
| | Histoplasma capsulatum (dimorphic fungus) |
| | Deer fly fever, tularemia, lemming fever, rabbit fever, O'Hara disease, Francis |
| | disease, Francisella tularensis (G− rods: facultative-straight: zoonoses) |
| | Dengue - Break Bone fever- dengue fever - dengue virus (Flavivirus) |
| | Desert rheumatism- Coccidioidomycosis- San Joaquin Valley fever- Posada- |
| | Wernicke disease- Coccidioides immitis (dimorphic fungus). |
| | ″Devil's grip″ (pleurodynia) - Coxsackie B (Picornavirus: Enterovirus) |
| | Diphasic milk fever- Biphasic meningoencephalitis, Central European tick- |
| | borne encephalitis, Czechoslovak tick-borne encephalitis, Tick-borne |
| | encephalitis, Viral meningoencephalitis, Tick-borne encephalitis virus- |
| | Flaviviridae |
| | Diphtheria - Corynebacterium diphtheriae (G+ rod: non-sporulating: non- |
| | filamentous) |
| | Disseminated Intravascular Coagulation(*) - most commonly Escherichia coli |
| | (G− rod) |
| | Dwarf tapeworm - Hymenolepis nana (intestinal cestode) |
| | Dog tapeworm - Diphylidium caninum (intestinal cestode) |
| | Donovanosis - Granuloma inguinale- Klebsiella granulomatis (G− rod; Donovan |
| | bodies) |
| | Dracontiasis - Guinea Worm - Dirofilaria medinensis (parasitic worm) |
| | Dracunculosis- Dracunculus medinensis (parasite; nematode; ″Little dragon of |
| | Medina″) |
| | Duke's disease- viral rash- Coxsackievirus or Echovirus |
| | Dum Dum Disease - Kala Azar - Visceral Leishmaniasis - Leishmania |
| | leishmania donovani, L. leishmania infantum, L. leishmania chagasi (protozoan |
| | parasite) sandfly |
| | Durand-Nicholas-Favre disease - Lymphogranuloma venereum (LGV) - |
| | Chlamydia trachomatis (intracellular G− bacteria; the L serotypes) |
| (E) | Eastern equine encephalitis - EEE virus (Togavirus) |
| | Ebola hemorrhagic fever - Ebola virus (Filovirus) |
| | Ectothrix - fungal infection of the hair shaft - Microsporum, Trichophyton, and |
| | Epidermophyton (fungi) |
| | Ehrlichiosis - Ehrlichia sp. (G− intracellular bacteria) transmitted by ticks |
| | Epidemic typhus- Rickettsia prowazekii, (G− intracellular; spread by lice) |
| | Encephalitis- Mumpsvirus, Human Herpesvirus 1 (Herpes Simplex 1 Virus), |
| | Any of 350 different arboviruses, Enteroviruses (polio, Coxsackie, |
| | ECHO), Adenovirus, Human Immunodeficiency Virus |
| | Endemic Relapsing fever- Borrelia sp. |
| | Endemic syphilis -Bejel - Treponema pallidum var. endemicum |
| | Endophthalmitis- Staphylococcus aureus, Staphylococcus epidermidis, Bacillus |
| | cereus, Streptococcus pneumoniae, Streptococcus pyogenes. |
| | Endothrix - fungal infection of the hair shaft - Microsporum, Trichophyton, and |
| | Epidermophyton (fungi) |
| | Enterobiasis - Pinworm infection - Enterobius vermicularis (intestinal |
| | nematode) |
| | Epidemic Relapsing fever- Borrelia recurrentis |
| | Epiglottitis (*)- Haemophilus influenzae (G− rod: facultative-straight: respiratory |
| | pathogens |
| | Erysipeloid - Erysipelothricosis - Erysipelothrix rhusiopathiae (G+ rod) |
| | Erysipelis- Streptococcus pyogenes |
| | Erythema chronicum migrans - seen in Lyme disease |
| | Erythema marginatum - seen in rheumatic fever |
| | Erythema multiforme - seen in coccidioidomycosis (Coccidioides immitis) |
| | Erythema nodosum - seen in coccidioidomycosis (Coccidioides immitis) |
| | Erythema nodosum leprosum - Mycobacterium leprae |
| | Erythema infectiosum - (Slapped cheek syndrome; fifth disease) Parvovirus B19 |
| | (Parvovirus) |
| | Erythrasma - Corynebacterium minutissimum |
| | Espundia - Leishmania viannia braziliensis (protozoan parasite) sandfly |
| | Eumycotic mycetoma- Madura foot- Pseudallescheria boydii, Madurella |
| | grisea, Madurella mycetomatis (fungi) |
| | European blastomycosis- Torulosis- Busse-Buschke disease- Cryptococcosis- |
| | Cryptococcus neoformans (encapsulated yeast) |
| | Eyeworm - Loiasis - Loa loa (parasitic worm) |
| | Exanthem subitum - Roseola infantum - Sixth disease - Zahorsky's disease- |
| | ″Sudden Rash″, Rose rash of infants, 3-day fever- Human Herpes virus 6 (HHV- |
| | 6) |
| (F) | Far Eastern tick-borne encephalitis- Spring-summer encephalitis- Russian |
| | spring-summer encephalitis- Taiga encephalitis- Russian spring-summer |
| | encephalitis virus- Flaviviridae |
| | Fascioliasis - Liver fluke infection - Fasciola hepatica (liver flukes) |
| | Fievre boutonneuse- Tick typhus- Rickettsia conori |
| | ″Fifth″ disease (erythema infectiosum) - Parvovirus B19 (Parvovirus) |
| | Filatow-Dukes' Disease- Scalded Skin Syndrome- Ritter's Disease- |
| | Staphylococcus aureus- (exfoliative toxin producing strains) |
| | Fish tapeworm - Diphyllobothrium latum |
| | Fitz-Hugh-Curtis syndrome - Perihepatitis - Neisseria gonorrhoeae (G− cocci) |
| | Five-day fever, Trench fever, Shinbone fever, Wolhynia fever, Quintana fever, |
| | His-Werner disease- Bartonella quintana (G− rod) |
| | Flinders Island Spotted Fever- Rickettsia honei |
| | Flu- Influenza - Influenza viruses A, B, and C (Orthomyxovirus) |
| | Four Corners Disease - Human Pulmonary Syndrome (HPS) - Sin Nombre Virus |
| | (Hantaan virus group; Bunyavirus) |
| | 14-day measles- Rubeola-measles- Morbilli- Hard measles- Rubeola virus |
| | Frambesia - Yaws -Treponema pallidum var. pertenue |
| | Francis disease, O'Hara disease, deer fly fever, lemming fever, tularemia, rabbit |
| | fever, Francisella tularensis (G− rods: facultative-straight: zoonoses) |
| | Furunculosis = boil- furuncle- Staphylococcus aureus (G+ coccus) |
| | Folliculitis - Staphylococcus aureus (G+ coccus) |
| (G) | Gas gangrene - Clostridium perfringens (G+ rod: sporulating: anaerobic) |
| | Gastroenteritis - Norwalk virus (Calicivirus), rotavirus (Reovirus) |
| | Genital Herpes- Herpes Simplex Virus-2 (Human Herpes Virus-2) occasionally |
| | HSV-1 (HHV-1) |
| | Genital Warts- Human Papilloma virus (various serotypes) |
| | German measles- Rubella- 3-day measles- Rubella virus |
| | Gerstmann-Straussler-Scheinker (GSS) - - prion (a protein) |
| | Giardiasis - Giardia lamblia |
| | Gilchrist's disease- Chicago disease- Blastomycosis- North American |
| | blastomycosis- Blastomyces dermatitidis (dimorphic fungus) |
| | Gingivostomatitis - HSV-1 (Herpesvirus) |
| | Gingivitis- various anaerobic bacteria in the mouth |
| | Glanders - Burkholderia mallei (used to be named Pseudomonas mallei; G− rod) |
| | Gnathostomiasis- Gnathostoma spinigerum (third stage larvae of a nematode |
| | (parasitic worm)) |
| | Gonorrhea - Neisseria gonorrhoeae (G− cocci) |
| | Granuloma inguinale - Donovanosis- Klebsiella granulomatis (G− rod) |
| | Guinea Worm - Dracontiasis - Dirofilaria medinensis (parasitic worm) |
| (H) | Hamburger disease- Hemolytic Uremic Syndrome- Escherichia coli O157 H7 |
| | strain. |
| | Hand-foot-mouth disease - Coxsackie A-16 virus (Picornavirus: Enterovirus) |
| | Hansen's disease - leprosy- Mycobacterium leprae (Acid-fast positive) |
| | Hantaan-Korean hemorrhagic fever - Hantavirus (Bunyavirus) |
| | Hantavirus Pulmonary Syndrome (HPS) - Hantavirus (Bunyavirus) |
| | Hard chancre - syphilis - Treponema pallidum subsp. pallidum |
| | Hard measles- Rubeola- measles- 14-day measles - Morbilli- Rubeola virus |
| | Haverhill fever - Rat bite fever - Streptobacillus moniliformis (G−; rod) |
| | Heartland fever - Heartland virus (phlebovirus)- transmitted by lone star tick- |
| | only two reported cases in Northwest Missouri |
| | Helicobacterosis - duodenal ulcers - Helicobacter pylori (G− curved rod) |
| | Hemolytic Uremic Syndrome- Hamburger disease- Escherichia coli O157 H7 |
| | strain. |
| | Hepatitis A - hepatitis A virus (Picornavirus: Enterovirus) |
| | Hepatitis B - hepatitis B virus (Hepadnavirus) |
| | Hepatitis C - hepatitis C virus (Flavivirus) |
| | Hepatitis D - hepatitis D virus (Deltavirus) |
| | Hepatitis E - hepatitis E virus (Calicivirus) |
| | Herpangina (*) - Coxsackie A (Picornavirus: Enterovirus), Enterovirus 7 |
| | (Picornavirus: Enterovirus) |
| | Herpes, genital - HSV-2 (Herpesvirus) |
| | Herpes labialis - HSV-1 (Herpesvirus) |
| | Herpes, neonatal - HSV-2 (Herpesvirus) |
| | Hidradenitis - Staphylococcus aureus (G+ coccus) |
| | HIV - human immunodeficiency virus (Retrovirus) |
| | Histoplasmosis - Histoplasma capsulatum (dimorphic fungus) |
| | His-Werner disease, Quintana fever, 5-day fever, Trench fever, Shinbone fever, |
| | Wolhynia fever- Bartonella quintana (G− rod) |
| | Hookworm infections - Ancylostoma duodenale, Necator americanus (intestinal |
| | nematode) |
| | Hordeola- Stye- Staphylococcus aureus |
| | HTLV- associated myelopathy (HAM) - Human T-cell Leukemia viruses I or II |
| | (retrovirus) |
| | Human Pulmonary Syndrome (HPS) - Four Corners Disease - Sin Nombre Virus |
| | (Hantaan virus group; Bunyavirus) |
| | Human monocytic ehrlichiosis - Ehrlichia chaffeensis. (G− intracellular bacteria) |
| | transmitted by ticks |
| | Human granulocytic ehrlichiosis - Ehrlichia equi. (G− intracellular bacteria) |
| | transmitted by ticks |
| | Hydatid cyst - Echinococcus granulosus, Echinococcus multilocularis, |
| | Echinococcus vogeli (larval cestode infection) |
| | Hydrophobia - Rabies - Rabies virus (Rhabdovirus) |
| (I) | Impetigo- Streptococcus pyogenes, Staphylococcus aureus |
| | Inclusion conjunctivitis - Swimming Pool conjunctivitis- Pannus - Chlamydia |
| | trachomatis (G− intracellular) eye infection |
| | Infantile diarrhea- Escherichia coli (ETEC- enterotoxigenic E. coli) |
| | Infectious Mononucleosis - Epstein-Barr virus (Herpesvirus; HHV-4) |
| | Infectious myocarditis (*) - Coxsackie B1-B5 (Picornavirus: Enterovirus) |
| | Infectious pericarditis (*)- Coxsackie B1-B5 (Picornavirus: Enterovirus) |
| | Influenza- Flu - Influenza viruses A, B, and C (Orthomyxovirus) |
| | Israeli spotted fever - unnamed Rickettsia (G− intracellular; tick-borne) |
| | Isosporiasis- Isospora belli (protozoan) |
| (J) | Japanese B encephalitis virus - JEE virus (Flavivirus) |
| | Jock itch - Tinea cruris - Microsporum, Trichophyton, and Epidermophyton |
| | (fungi) |
| | Jorge Lobo disease - lobomycosis, Lobo's mycosis, Keloidal blastomycosis - |
| | Paracoccidioides loboi (Fungus) |
| | Jungle yellow fever, Yellow fever, Sylvatic yellow fever, Urban yellow fever, |
| | Vomito negro, Yellow Jack, Yellow fever virus- Flaviviridae, Flavivirus |
| | Junin Argentinian hemorrhagic fever - Juninvirus (Arenavirus) |
| (K) | Kala Azar - Visceral Leishmaniasis - Leishmania leishmania donovani, L. |
| | leishmania infantum, L. leishmania chagasi (protozoan parasite) sandfly |
| | Keratoconjunctivitis (*) - Viral conjunctivitis- Adenovirus (Adenovirus), HSV-1 |
| | (Herpesvirus) |
| | Kaposi's sarcoma - Human Herpes Virus 8 (Herpesvirus) or Kaposi's Sarcoma- |
| | associated Herpes Virus (KSHV) |
| | Kuru - prion (a protein) |
| | Kyasanur forest disease - KFD virus (flavivirus) tick-borne |
| (L) | LaCrosse encephalitis - LaCross virus (Bunyavirus) |
| | Lassa hemorrhagic fever - Lassavirus (Arenavirus) |
| | Legionnaire's pneumonia - Legionella pneumophila (G− rod: facultative-straight: |
| | respiratory pathogens) |
| | Lemming fever- tularemia, rabbit fever, deer fly fever, O'Hara disease, Francis |
| | disease, Francisella tularensis (G− rods: facultative-straight: zoonoses) |
| | Leprosy (Hansen's disease) - Mycobacterium leprae (Acid-fast positive) |
| | Leptospirosis -Weil's disease- canicola fever- canefield fever- nanukayami |
| | fever- 7-day fever- Leptospira interrogans (spiral shaped bacteria) |
| | Lemierre's Syndrome- Fusobacterium necrophorum (G− rod; anaerobe) |
| | Listerosis - Listeria monocytogenes (G+ rod) |
| | Liver fluke infection - Clonorchis sinensis, Opisthorchis viverrini, O. felineus, |
| | Fasciola hepatica (liver flukes) |
| | Lockjaw - Tetanus - Clostridium tetani (G+ rod; anaerobe) |
| | Loiasis - Eyeworm - Loa loa (parasitic worm) |
| | Louping Ill - Flavivirus (arbovirus) ticks |
| | Ludwig's angina- usually a polymicrobial infection (cellulitis of the floor of the |
| | mouth with spread to the submental, sublingual and submandibular spaces). |
| | Bacteria from mouth. |
| | Lung fluke infection - Paragonimus westermani |
| | Lyme disease - Borrelia burgdorferi (Spirochetes) |
| | Lyme-like illness- Masters disease- Southern tick associated rash illness |
| | (STARI)- Borrelia lonestari (possible etiology) |
| | Lymphogranuloma venereum (LGV) - Chlamydia trachomatis (intracellular G− |
| | bacteria; the L serotypes) |
| (M) | Machupo Bolivian hemorrhagic fever - Machupovirus (Arenavirus) |
| | Madura foot- Eumycotic mycetoma- Pseudallescheria boydii, Madurella |
| | grisea, Madurella mycetomatis (fungi) |
| | Malaria - Plasmodium sp. (protozoan parasite) |
| | Mal del pinto - Pinta - Treponema pallidum var. carateum |
| | Malignant pustule- Black Bane- Anthrax- Wool sorter's disease- Tanner's |
| | disease- Bacillus anthracis (G+ rod: sporulating: aerobic) |
| | Malta fever - Brucellosis- Brucella sp. (G− rods: facultative-straight: zoonoses) |
| | Marburg hemorrhagic fever - Marburg virus (Filovirus) |
| | Masters disease- Southern tick associated rash illness (STARI)- Lyme-like |
| | illness- Borrelia lonestari (possible etiology) |
| | Measles - Morbilli- Hard measles- Rubeola- measles- 14-day measles- rubeola |
| | virus (Paramyxovirus) |
| | Mediterannean spotted fever- Rickettsia coronii, (G−; intracellular bacteria) |
| | Melioidosis - Whitmore's disease- Burkholderia pseudomallei (used to be called |
| | Pseudomonas pseudomallei; G− rod: aerobic) |
| | MERS ( Middle East Respiratory Syndrome)- Coronavirus called MERS-COV |
| | Meningitis, aseptic (*) - Coxsackie A and B (Picornavirus: Enterovirus), |
| | Echovirus (Picornavirus: Enterovirus), lymphocytic choriomeningitis virus |
| | (Arenavirus), HSV-2 (Herpesvirus), Mycobacterium tuberculosis (Acid-fast) |
| | Meningitis, bacterial (*) - Neisseria meningitidis (G− cocci), Haemophilus |
| | influenzae (G− rod: facultative-straight: respiratory pathogens), Listeria |
| | monocytogenes (G+ rod: non-sporulating: non-filamentous), Streptococcus |
| | pneumoniae (G+ cocci), Group B streptococcus (G+ cocci) |
| | Milker's nodule - Parapoxvirus |
| | Middle East Respiratory Syndrome (MERS)- Coronavirus called MERS-COV |
| | Molluscum contagiosum - Molluscipoxvirus (Poxvirus) |
| | Moniliasis- candidiasis- infection of the mucous membranes caused by the yeast |
| | Candida albicans. |
| | Monkeypox- Monkeypox virus- Poxviridae- Chordopoxvirus |
| | Mononucleosis - Epstein-Barr virus (Herpesvirus; HHV-4) |
| | Mononucleosis-like syndrome (*) - Cytomegalovirus (CMV; Herpesvirus; |
| | HHV-5) |
| | Montezuma's Revenge- Traveler's diarrhea - Any number of bacteria |
| | (Escherichia coli, Salmonella, Shigella, Yersinia, Vibrio, etc.), viruses |
| | (Rotaviruses, Norwalk-like agents), or parasites (Giardia, Entamoeba, |
| | Cryptosporidium) that cause diarrhea. |
| | Morbilli- Hard measles- Rubeola- measles- 14-day measles - Rubeola virus |
| | Mucormycosis- Zygomycosis- Rhizopus arrhizus (fungus) |
| | Multiple Organ Dysfunction Syndrome or MODS (*)- if infectious see Septic |
| | Shock for common causes. |
| | Mumps - mumps virus (Paramyxovirus) |
| | Murine typhus - Rickettsia typhi (G− intracellular; rodents and fleas) |
| | Murray Valley encephalitis - Flavivirus (arbovirus) mosquito |
| | Mycoburuli ulcers- Buruli ulcers- Mycobacterium ulcerans |
| | Mycotic vulvovaginitis- Candida albicans (yeast) |
| | Myositis- Streptococcus pyogenes, Staphylococcus aureus |
| (N) | Nanukayami fever- leptospirosis -Weil's disease- canicola fever- canefield |
| | fever-7-day fever- Leptospira interrogans (spiral shaped bacteria) |
| | Negishi - Flavivirus (arbovirus) vector unknown |
| | Necrotizing fasciitis- Type 1 = Streptococcus pyogenes: Type 2 = Staphylococcus |
| | aureus |
| | New world spotted fever, Rocky Mountain spotted fever, Sao Paulo fever - |
| | Rickettsia rickettsii (Obligate intracellular) |
| | Nocardiosis - Nocardia (G+: non-sporulating: filamentous) |
| | Nongonococcal urethritis(*) - Chlamydia trachomatis (G−; intracellular |
| | bacteria), Mycoplasma genitalium (bacterium without a cell wall), Ureaplasma |
| | urealyticum (bacterium without a cell wall), Gardnerella vaginalis (G variable |
| | rod), Trichomonas vaginalis (protozoan parasite), and Herpes Simplex virus |
| | (herpes virus) |
| | North American blastomycosis- Gilchrist's disease- Chicago disease- |
| | Blastomycosis- Blastomyces dermatitidis (dimorphic fungus) |
| | North Asian tick typhus - Rickettsia sibirica (G− intracellular; tick-borne) |
| | Norwegian itch - Scabies - Sarcoptes scabiei (parasitic mite) |
| (O) | O'Hara disease, deer fly fever, tularemia, lemming fever, rabbit fever, Francis |
| | disease, Francisella tularensis (G− rods: facultative-straight: zoonoses) |
| | Omsk hemorrhagic fever - OHF virus (Flavivirus; tick borne) |
| | Onchoceriasis - River Blindness - Onchocerca volvulus (parasitic worm) |
| | Onychomycosis- Tinea unguium - Ringworm of the nails- Trichophyton sp., and |
| | Epidermophyton floccosum (fungi) |
| | Opisthorchiasis - Liver fluke infection - Opisthorchis viverrini, O. felineus (liver |
| | flukes) |
| | Opthalmia neonatorium - Gonorrhea - Neisseria gonorrhoeae (G− cocci) |
| | Ornithosis - Parrot fever - Psittacosis - Chlamydia psittaci (G− intracellular) |
| | Oral hairy leukoplakia - Epstein Barr Virus (Human Herpes virus 4) |
| | Oriental Spotted Fever - Rickettsia japonica (G− intracellular; tick-borne) |
| | Oriental Sore - Leishmania leishmania major and L. leishmania tropica |
| | (protozoan parasite) sandfly |
| | Orf - Orfvirus (Poxvirus) |
| | Oroya fever - Carrion disease - Bartonellosis - Bartonella bacilliformis (weak |
| | G− polymorphic) sandfly bites at elevations of 600 to 2800 meter in Peru, |
| | Ecuador and Colombia. |
| | Otitis media- Streptococcus pneumoniae, Haemophilus influenzae, Moraxella |
| | catarrhalis, various viruses. |
| | Otitis externa (*) - Pseudomonas aeruginosa (G− rod: aerobic) |
| (P) | Parotitis - Mumps - Mumps virus (paramyxovirus) |
| | Paronychia - Candida albicans (yeast), Herpes Simplex virus (herpes virus) |
| | Parrot fever - Ornithosis- Psittacosis - Chlamydia psittaci (G− intracellular) |
| | Pannus - Chlamydia trachomatis (G− intracellular) eye infection |
| | Paragonimiasis - Lung fluke infection - Paragonimus westermani |
| | Paracoccidioidomycosis - Paracoccidioides brasiliensis (dimorphic fungi) |
| | PCP pneumonia- Pneumonia caused by Pneumocystis carinii |
| | Pediculosis - lice |
| | Peliosis hepatica - Bartonella henselae (pleomorphic G−) |
| | Pelvic Inflammatory Disease (PID) - two most common = Neiserria |
| | gonorrhoeae (G− coccus), Chlamydia trachomatis, then Anaerobic bacteria (ex. |
| | Bacteroides), Facultative Gram negative rods (ex. E. coli), Mycoplasma |
| | hominis, Actinomyces israelii (IUD recipients: G+ rod) |
| | Pertussis - Whooping cough- Bordetella pertussis (G− rods: facultative-straight: |
| | respiratory pathogens) |
| | Pharyngoconjunctival fever (*) - Adenovirus 1-3 and 5 (Adenovirus) |
| | Phaeohyphomycosis(*) - over 75 different species of fungi, most common = |
| | Phaeoaellomyces werneckii and P. hortae |
| | Piedra- Black Piedra = Piedraia hortai, White Piedra = Trichosporon beigelii |
| | Pigbel- beta-toxin of Clostridium perfringens type C |
| | ″Pink eye″ conjunctivitis (*) - Haemophilus aegyptius (G− rod: facultative- |
| | straight: respiratory pathogens) and/or Moraxella lacunata (G− diplococcus) |
| | Pinta - Treponema pallidum var. carateum |
| | Pinworm infection - Enterobiasis - Enterobius vermicularis (intestinal |
| | nematode) |
| | Pitted Keratolysis - Micrococcus sedentarius (G+ coccus) |
| | Pityriasis versicolor- Tinea versicolor- Malassezia furfur (fungus) |
| | Plague - Yersinia pestis (G− rod: facultative-straight: zoonoses) |
| | Pleurodynia - Coxsackie B (Picornavirus: Enterovirus) |
| | Pneumonia, viral (*) - respiratory syncytial virus (Paramyxovirus), CMV |
| | (Herpesvirus) |
| | Pneumocystosis - Pneumocystis carinii (protozoan parasite) |
| | Polio or Poliomyelitis - Polioviruses types I, II, and III (picornavirus) |
| | Polycystic hydatid - Echinococcus vogeli (larval cestode infection) |
| | Pontiac fever - Legionella pneumophila (G− rod: facultative-straight: respiratory |
| | pathogens) |
| | Pork tapeworm - Taenia solium |
| | Posada-Wernicke disease- Desert rheumatism- Coccidioidomycosis- San |
| | Joaquin Valley fever- Coccidioides immitis (dimorphic fungus) |
| | Postanginal septicemia- Lemierre's Syndrome- Fusobacterium necrophorum (G− |
| | rod; anaerobe) |
| | Powassan - Flavivirus (arbovirus) ticks |
| | Progressive multifocal leukencephalopathy - JC virus (Papovavirus) |
| | Progressive Rubella Panencephalitis - Rubella virus (togavirus) |
| | Prostatitis, bacterial(*) - most common = Escherichia coli, Klebsiella sp., |
| | Proteus sp., Pseudomonas sp., Enterobacter sp., Serratia sp., (G− rods), |
| | Enterococcus feacalis (G+ coccus) |
| | Pseudomembranous colitis - Clostridium difficile (G+ rod: sporulating: |
| | anaerobic) |
| | Psittacosis - Chlamydia psittaci (G− intracellular) |
| | Puerperal fever- Streptococcus pyogenes |
| | Pyelonephritis(*) - similar to cystitis |
| | Pylephlebitis - Bateroides fragilis (G− anaerobic rod), Peptostreptococcus spp |
| | (G+ anaerobic cocci), Clostridium spp.(G+ anaerobic rods), and several of the |
| | Enterobacteriaceae (G− rods; ferment glucose) |
| (Q) | Q fever - Coxiella burnetti (Obligate intracellular: Rickettsia) |
| | Australian tick typhus- Australian Spotted Fever- Queensland Tick Typhus- |
| | Rickettsia australis, (G−; intracellular bacteria) |
| | Quinsy- Peritonsillar abscess- a complication of untreated Strep. throat |
| | (Streptococcus pyogenes) |
| | Quintana fever, 5-day fever, Trench fever, Shinbone fever, Wolhynia fever, |
| | His-Werner disease- Bartonella quintana (G− rod) |
| (R) | Rabies - rabies virus (Rhabdovirus) |
| | Rabbit fever- deer fly fever, tularemia, lemming fever, O'Hara disease, Francis |
| | disease, Francisella tularensis (G− rods: facultative-straight: zoonoses) |
| | Racoon roundworm infection- Baylisascaris infection - Baylisascaris procyonis |
| | Rat bite fever - Streptobacillus moniliformis (G−; rod) |
| | Rat tapeworm - Hymenolepis diminuta |
| | Reiter Syndrome (*)- resulting from a nongonococcal sexually transmitted |
| | disease due usually to Chlamydia trachomatis or from an infectious diarrhea |
| | (Shigella, Salmonella, Yersinia). Persons with an HLA-B27 major |
| | histocompatibility complex are more likely to get this disease. |
| | Relapsing fever- Borrelia recurrentis |
| | Relapsing fever-like disease- Borrelia miyamotoi |
| | Rheumatic fever - Streptococcus pyogenes (nonsuppurative complication of |
| | Strep throat) |
| | Rhodotorulosis - Rhodotorula spp. (fungus) |
| | Rickettsialpox - Rickettsia akari (G−; intracellular) from mite bites |
| | Rift Valley Fever- Rift valley fever virus- Bunyavirus- Phlebovirus |
| | Ringworm - Microsporum, Trichophyton, and Epidermophyton (fungi) |
| | River Blindness - Onchoceriasis - Onchocerca volvulus (parasitic worm) |
| | Ritter's Disease- Filatow-Dukes' Disease, Scalded Skin Syndrome- |
| | Staphylococcus aureus- (exfoliative toxin producing strains) |
| | Rocky Mountain spotted fever, New world spotted fever, Sao Paulo fever - |
| | Rickettsia rickettsii (Obligate intracellular) |
| | Rose Handler's disease - Sporotrichosis - Sporothrix schenckii (dimorphic fungi) |
| | Rose rash of infants- Sixth disease - Zahorsky's disease - Roseola infantum - |
| | Exanthem subitum - ″Sudden Rash″- 3-day fever- Human Herpes virus 6 |
| | (HHV-6) |
| | Roseola - Roseola infantum - Sixth disease - Zahorsky's disease - Exanthem |
| | subitum - Human Herpes virus 6 (HHV-6) |
| | Roundworm infections - Ascariasis - Ascaris lumbricoides (intestinal nematode) |
| | Rotavirus infections - Rotavirus (reovirus) |
| | Rubella - German measles- 3-day measles- rubella virus (Togavirus) |
| | Rubeola-measles- 14-day measles- Hard measles- Morbilli- Rubeola virus |
| | Russian spring-summer encephalitis- Far Eastern tick-borne encephalitis- |
| | Spring-summer encephalitis- Taiga encephalitis- Russian spring-summer |
| | encephalitis virus- Flaviviridae |
| (S) | Salmonellosis - Salmonella spp. (G− rod) |
| | San Joaquin Valley fever- Posada-Wernicke disease- Desert rheumatism- |
| | Coccidioidomycosis- Coccidioides immitis (dimorphic fungus). |
| | Sao Paulo Encephalitis - Flavivirus (arbovirus) |
| | Sao Paulo fever, New world spotted fever, Rocky Mountain spotted fever- |
| | Rickettsia rickettsii (Obligate intracellular) |
| | SARS- Severe Acute Respiratory Syndrome- SARS-associated coronavirus or |
| | SARS-COV |
| | Scabies - Norwegian itch - Sarcoptes scabiei (parasitic mite) |
| | Scarlet fever - Scarlatina- Streptococcus group A (Streptococcus pyogenes) |
| | Scarlatina- Scarlet fever - Streptococcus group A (Streptococcus pyogenes) |
| | Scalded Skin Syndrome- Ritter's Disease- Filatow-Dukes' Disease- |
| | Staphylococcus aureus- (exfoliative toxin producing strains) |
| | Schistosomiasis - Schistosoma mansoni, S. japonicum, and S. haematobium |
| | (protozoan parasites; blood flukes) |
| | Scrub typhus - Rickettsia tsutsugamushi (G− intracellular; chigger bite) |
| | Sennetsu fever - Ehrlichiosis - Ehrlichia sp. (G− intracellular bacteria) |
| | transmitted by ticks |
| | Sepsis- See Septic Shock below. |
| | Septic Shock(*) - Most are due to bacterial infections. 50% due to Gram |
| | negative bacteria; 50% due to Gram positive bacteria. It depends on the location |
| | of the site of the initial infection. Most common sites of infection leading to |
| | sepsis are lungs, abdomen, and urinary tract (ex. urinary tract think Escherichia |
| | coli; community acquired pneumonia think Streptococcus pneumoniae). |
| | 7-day fever- Weil's disease - leptospirosis - canicola fever- canefield fever- |
| | nanukayami fever- Leptospira interrogans (spiral shaped bacteria) |
| | Severe Acute Respiratory Syndrome- SARS-coronavirus or SARS-COV |
| | Shigellosis - Shigella sp. (G− rod) |
| | Shingles (zoster) - varicella zoster virus (Herpesvirus) |
| | Shipping fever - Pasteurella multocida (G− rods: facultative-straight: zoonoses) |
| | Siberian tick typhus- Rickettsia sibirica, (G−; intracellular bacteria) |
| | Sinusitis(*) - most common causes overall are respiratory viruses; most |
| | common bacterial causes = Streptococcus pneumoniae (G+ coccus) and |
| | Haemophilus influenzae (G− pleomorphic rod) (renamed and now called acute |
| | rhinosinusitis or acute bacterial rhinosinusitis) |
| | Sixth disease - Zahorsky's disease - Roseola infantum - Exanthem subitum - |
| | ″Sudden Rash″- 3-day fever- Rose rash of infants- Human Herpes virus 6 |
| | (HHV-6) and HHV-7 (occasionally) |
| | ″Slapped cheek″ disease (erythema infectiosum; Fifth disease) - Parvovirus B19 |
| | (Parvovirus) |
| | Sleeping sickness- viral encephalitis - Mumps virus, Human Herpes virus 1, any |
| | of 350 different Arboviruses, Poxvirus, Enteroviruses (polio, Coxsackie, |
| | ECHO), Adenoviruses, Human Immunodeficiency Virus (retrovirus) |
| | Smallpox - variola virus (Poxvirus) - no naturally acquired cases since October |
| | 1977; Somalia |
| | Snail Fever- Schistosoma (protozoan parasite) |
| | Soft chancre - Chancroid - Haemophilus ducreyi (G− rod: facultative-straight: |
| | respiratory pathogens) |
| | Southern tick associated rash illness (STARI)- Lyme-like illness- Masters |
| | disease- Borrelia lonestari (possible etiology) |
| | Sparganosis - Spirometra sp.(cestode larvae infection) |
| | Spelunker's disease- Cave disease- Darling's Disease- Histoplasmosis- |
| | Histoplasma capsulatum (dimorphic fungus) |
| | Spotted fever- same as meningitis (bacterial) |
| | Sporadic typhus- Rickettsia prowazekii, (G−, intracellular bacterium; spread by |
| | fleas) |
| | Sporotrichosis - Sporothrix schenckii (dimorphic fungi) |
| | Spring-summer encephalitis- Far Eastern tick-borne encephalitis- Russian |
| | spring-summer encephalitis- Taiga encephalitis- Russian spring-summer |
| | encephalitis virus- Flaviviridae |
| | St. Louis encephalitis - SLE virus (Flavivirus) |
| | Strep. throat- Streptococcus pyogenes (G+ coccus). |
| | Stye- Hordeola- Staphylococcus aureus |
| | Strongyloiciasis - Threadworm - Strongyloides stercoralis (intestinal nematode) |
| | Subacute Sclerosing Panencephalitis (SSPE) - Measles virus |
| | Sudden Acute Respiratory Syndrome- SARS-COV- Coronavirus |
| | ″Sudden Rash″- 3-day fever- Exanthem subitum - Roseola infantum - Sixth |
| | disease - Zahorsky's disease- Rose rash of infants- Human Herpes virus 6 |
| | (HHV-6) |
| | Swimmer's ear- Otitis externa- Pseudomonas aeruginosa (common in diabetic |
| | patients) |
| | Swimmer's Itch - Schistosoma avium (bird schistosomes) (protozoan parasite) |
| | Swimming Pool conjunctivitis- Inclusion conjunctivitis - Pannus - Chlamydia |
| | trachomatis (G− intracellular) eye infection |
| | Swine flu- Influenza virus H1N1 |
| | Syphilis - Treponema pallidum subsp. pallidum (Spirochetes; bacteria) |
| | Systemic Inflammatory Response Syndrome or SIRS (*)- if infectious see |
| | Septic Shock for common causes. |
| | Sylvatic yellow fever, Yellow Jack, Jungle yellow fever, Yellow fever, Urban |
| | yellow fever, Vomito negro, Yellow fever virus- Flaviviridae, Flavivirus |
| (T) | Tabes dorsalis - tertiary syphilis - Treponema pallidum subsp. pallidum |
| | (Spirochetes) |
| | Taeniasis - see Tapeworm infections with Taenia species. |
| | Taiga encephalitis- Russian spring-summer encephalitis- Far Eastern tick-borne |
| | encephalitis- Spring-summer encephalitis- Russian spring-summer encephalitis |
| | virus- Flaviviridae |
| | Tanner's disease - Wool sorters' disease- Malignant pustule- Black Bane- |
| | Bacillus anthracis (G+ rod: sporulating: aerobic) |
| | Tapeworm infections - Taenia solium (pork tapeworm), Taenia saginata (beef |
| | tapeworm), Diphyllobothrium latum (fish tapeworm), Hymenolepis nana (dwarf |
| | tapeworm), Hymenolepis diminuta (rat tapeworm), Diphylidium caninum (dog |
| | tapeworm) (intestinal cestodes) |
| | TB- Tuberculosis - Mycobacterium tuberculosis (Acid-fast bacterium) |
| | Temporal lobe encephalitis (*) - HSV-1 (Herpesvirus) |
| | Tetanus - Clostridium tetani (G+ rod: sporulating: anaerobic) |
| | Threadworm infections - Strongyloiciasis - Strongyloides stercoralis (intestinal |
| | nematode) |
| | 3-day fever- Exanthem subitum - Roseola infantum - Sixth disease - Zahorsky's |
| | disease- ″Sudden Rash″, Rose rash of infants- Human Herpes virus 6 (HHV-6) |
| | 3-day measles- German measles- Rubella- Rubella virus |
| | Thrush - Candida albicans (yeast) |
| | Tick-borne encephalitis- Biphasic meningoencephalitis, Central European tick- |
| | borne encephalitis, Czechoslovak tick-borne encephalitis, Diphasic milk fever, |
| | Viral meningoencephalitis, Tick-borne encephalitis virus- Flaviviridae |
| | Tick typhus- Fievre boutonneuse- Rickettsia conori |
| | Tinea barbae - Trichophyton verrucosum, T. mentagrophytes, T. rubrum, T. |
| | megninii (fungi) |
| | Tinea capitis - Ringworm of the head- Microsporum sp., Trichophyton |
| | sp. (fungi) |
| | Tinea corporis - Ringworm of the body- Microsporum, Trichophyton, and |
| | Epidermophyton floccosum (fungi) |
| | Tinea manuum - Ringworm of the hand- Trichophyton sp., and Epidermophyton |
| | floccosum (fungi) |
| | Tinea cruris - Ringworm of the groin- Candida albicans (yeast), Trichophyton |
| | sp., and Epidermophyton floccosum (fungi) |
| | Tinea nigra- Exophiala werneckii |
| | Tinea pedis - Ringworm of the feet- Trichophyton sp., and Epidermophyton |
| | floccosum(fungi) |
| | Tinea unguium - Onychomycosis- Ringworm of the nails- Trichophyton sp., and |
| | Epidermophyton floccosum (fungi) |
| | Tinea versicolor- Pityriasis versicolor- Malassezia furfur (fungus) |
| | Torulopsosis - Torulopsis glabrata and T. candida (fungus) |
| | Torulosis- Busse-Buschke disease- Cryptococcosis- European blastomycosis- |
| | Cryptococcus neoformans (encapsulated yeast) |
| | Toxic Shock Syndrome - Staphylococcus aureus (G+ cocci; producing TSST) |
| | and Streptococcus pyogenes (G+ cocci) |
| | Toxoplasmosis - Toxoplasma gondii (protozoan parasite) |
| | Traveler's diarrhea - Any number of bacteria (Escherichia coli (most common), |
| | Salmonella, Shigella, Yersinia, Vibrio, etc.), viruses (Rotaviruses, Norwalk-like |
| | agents), or parasites (Giardia, Entamoeba, Cryptosporidium) that cause |
| | diarrhea. |
| | Trench fever, 5-day fever, Shinbone fever, Wolhynia fever, Quintana fever, His- |
| | Werner disease- Bartonella quintana (G− rod) |
| | Trench mouth or Vincent's disease- Various anaerobic bacteria in the mouth |
| | Trichinellosis- Trichinella spiralis (nematode parasite) |
| | Trichomoniasis - Vaginitis - Trichomonas vaginalis (protozoan parasite) |
| | Trichomycosis axillaris - Corynebacterium tenuis (G+ rod) |
| | Trichuriasis - Whipworm infection - Trichuris trichiura (intestinal nematode) |
| | Tropical Spastic Paraparesis (TSP) - Human T-cell Leukemia viruses I or II |
| | (retrovirus) |
| | Trypanosomiasis - African = Trypanosoma brucei rhodesiense, Trypanosoma |
| | brucei gambiense (tsetse fly-borne), American = Trypanosoma cruzi (Triatomine |
| | bugs = kissing bug or assassin bugs) |
| | Tuberculosis - TB- Mycobacterium tuberculosis (Acid-fast bacterium) |
| | Tularemia- lemming fever, rabbit fever, deer fly fever, O'Hara disease, Francis |
| | disease, Francisella tularensis (G− rods: facultative-straight: zoonoses) |
| | Typhoid fever - Salmonella typhi (G− rod: facultative-straight: enteric |
| | pathogens) |
| | Typhus fever - Rickettsia prowazekii (G− intracellular; louse-borne), Rickettsia |
| | typhi (G− intracellular; flea-borne) |
| (U) | Ulcus molle - Soft chancre - Chancroid - Haemophilus ducreyi (G− rod: |
| | facultative-straight: respiratory pathogens) |
| | Undulant fever - Brucella sp. (G− coccobacillus: zoonoses) |
| | Urban yellow fever, Sylvatic yellow fever, Yellow Jack, Jungle yellow fever, |
| | Yellow fever, Vomito negro, Yellow fever virus- Flaviviridae, Flavivirus |
| | Urethritis - Herpes Simplex virus, Chlamydia trachomatis, Ureaplasma |
| | urealyticum, Neisseria gonorrhoeae |
| (V) | Vaginosis, bacterial - Peptostreptococccus sp., Bacteriodes sp., Gardnerella |
| | vaginalis, Mobiluncus sp., Mycoplasma sp. (clue cells) |
| | Vaginitis - Candida albicans (yeast; Mycotic vulvovaginitis), Trichomonas |
| | vaginalis (protozoan parasite; Trichomoniasis) |
| | Varicella -chickenpox - Varicella-Zoster virus (VZV or Human herpes 3 virus) |
| | Venezuelan Equine encephalitis - Togaviridae, Alphavirus |
| | Verruga peruana- Carrion's disease - Bartonellosis - Oroya fever - Bartonella |
| | bacilliformis (weak G− polymorphic) sandfly bites at elevations of 600 to 2800 |
| | meter in Peru, Ecuador and Colombia. |
| | Vincent's disease or Trench mouth- Various anaerobic bacteria in the mouth |
| | Viral conjunctivitis (*) - Keratoconjunctivitis - Adenovirus (Adenovirus), HSV- |
| | 1 (Herpesvirus) |
| | Viral meningoencephalitis- Czechoslovak tick-borne encephalitis, Central |
| | European tick-borne encephalitis, Diphasic milk fever, Biphasic |
| | meningoencephalitis, Tick-borne encephalitis, Tick-borne encephalitis virus- |
| | Flaviviridae |
| | Viral rash- Duke's disease- Coxsackievirus or Echovirus |
| | Visceral Larval Migrans - Toxocara canis (parasitic nematode) |
| | Vomito negro, Urban yellow fever, Sylvatic yellow fever, Yellow Jack, Jungle |
| | yellow fever, Yellow fever, Yellow fever virus- Flaviviridae, Flavivirus |
| | Vulvovaginitis - Candida albicans (yeast), Trichomonas vaginalis (protozoan |
| | parasite), and the causes of bacterial vaginosis. |
| (W) | Warts - Papilloma viruses |
| | Waterhouse-Friderichsen syndrome - Neisseria meningitidis (G− cocci) |
| | Weil's disease - Leptospirosis - canicola fever- canefield fever- nanukayami |
| | fever- 7-day fever- Leptospira interrogans (spiral shaped bacteria) |
| | West Nile Fever- West Nile virus- Flavivirus Japanese Encephalitis Antigenic |
| | Complex |
| | Western equine encephalitis - WEE virus, Togaviridae, Alphavirus |
| | Whipple's disease - Tropheryma whippelii (G+ rod a actinomycete) |
| | Whipworm infection - Trichuriasis - Trichuris trichiura |
| | White Piedra- Trichosporon beigelii |
| | Whitmore's disease- Melioidosis - Burkholderia pseudomallei (used to be called |
| | Pseudomonas pseudomallei; G− rod: aerobic) |
| | Whitlow - paronchyia - Herpes simplex virus (herpesvirus) |
| | Whooping cough - Pertussis- Bordetella pertussis (G− small rod) |
| | Winter diarrhea - Rotavirus infections - Rotavirus (reovirus) |
| | Wolhynia fever, His-Werner disease, Quintana fever, 5-day fever, Trench fever, |
| | Shinbone fever- Bartonella quintana (G− rod) |
| | Wool sorters' disease - Anthrax- Tanner's disease- Malignant pustule- Black |
| | Bane- Bacillus anthracis (G+ rod: sporulating: aerobic) |
| (XYZ) | Yaws -Treponema pallidum var. pertenue (spirochete) |
| | Yellow fever, Jungle yellow fever, Sylvatic yellow fever, Urban yellow fever, |
| | Vomito negro, Yellow Jack, Yellow fever virus- Flaviviridae, Flavivirus |
| | Yellow Jack, Jungle yellow fever, Yellow fever, Sylvatic yellow fever, Urban |
| | yellow fever, Vomito negro, Yellow fever virus- Flaviviridae, Flavivirus |
| | Yersinosis - Yersinia enterocolitica |
| | Zahorsky's disease - Roseola infantum - Exanthem subitum - Sixth disease - |
| | Human Herpes virus 6 (HHV-6) |
| | Zika virus disease- Zika virus |
| | Zoster - shingles- Varicella-Zoster virus (VZV or Human herpes 3 virus) |
| | Zygomycosis- Mucormycosis- Rhizopus arrhizus (fungus) |
| Autoimmune | Examples of autoimmune diseases or disorders: acute disseminated encephalomyelitis |
| Diseases | (ADEM); Addison's disease; ankylosing spondylitis; antiphospholipid antibody |
| | syndrome (APS); aplastic anemia; autoimmune gastritis; autoimmune hepatitis; |
| | autoimmune thrombocytopenia; Behcet's disease; coeliac disease; dermatomyositis; |
| | diabetes mellitus type I; Goodpasture's syndrome; Graves' disease; Guillain-Barre |
| | syndrome (GBS); Hashimoto's disease; idiopathic thrombocytopenia purpura; |
| | inflammatory bowel disease (IBD) including Crohn's disease and ulcerative colitis; |
| | mixed connective tissue disease; multiple sclerosis (MS); myasthenia gravis; opsoclonus |
| | myoclonus syndrome (OMS); optic neuritis; Ord's thyroiditis; pemphigus; pernicious |
| | anaemia; polyarteritis nodosa; polymyositis; primary biliary cirrhosis; primary |
| | myoxedema; psoriasis; rheumatic fever; rheumatoid arthritis; Reiter's syndrome; |
| | scleroderma; Sjogren's syndrome; systemic lupus erythematosus; Takayasu's arteritis; |
| | temporal arteritis; vitiligo; warm autoimmune hemolytic anemia; or Wegener's |
| | granulomatosis. The MS may be any clinical variety or origin, and not limited to |
| | mammals. Non-limiting examples may include Experimental autoimmune |
| | encephalomyelitis (EAE), clinically isolated syndrome (CIS), Relapsing-remitting MS |
| | (RRMS), Secondary progressive MS (SPMS), or Primary progressive MS (PPMS). |
| | Examples of inflammatory diseases or disorders: asthma, allergy, allergic rhinitis, allergic |
| | airway inflammation, atopic dermatitis (AD), chronic obstructive pulmonary disease |
| | (COPD), inflammatory bowel disease (IBD), Irritable bowel syndrome (IBS), multiple |
| | sclerosis, arthritis, psoriasis, eosinophilic esophagitis, eosinophilic pneumonia, |
| | eosinophilic psoriasis, hypereosinophilic syndrome, graft-versus-host disease, uveitis, |
| | cardiovascular disease, pain, multiple sclerosis, lupus, vasculitis, chronic idiopathic |
| | urticaria and Eosinophilic Granulomatosis with Polyangiitis (Churg-Strauss Syndrome). |
| | The asthma may be allergic asthma, non-allergic asthma, severe refractory asthma, |
| | asthma exacerbations, viral-induced asthma or viral-induced asthma exacerbations, |
| | steroid resistant asthma, steroid sensitive asthma, eosinophilic asthma or non- |
| | eosinophilic asthma and other related disorders characterized by airway inflammation or |
| | airway hyperresponsiveness (AHR). The COPD may be a disease or disorder associated |
| | in part with, or caused by, cigarette smoke, air pollution, occupational chemicals, allergy |
| | or airway hyperresponsiveness. The allergy may be associated with foods, pollen, mold, |
| | dust mites, animals, or animal dander. The IBD may be ulcerative colitis (UC), Crohn's |
| | Disease, collagenous colitis, lymphocytic colitis, ischemic colitis, diversion colitis, |
| | Behcet's syndrome, infective colitis, indeterminate colitis, and other disorders |
| | characterized by inflammation of the mucosal layer of the large intestine or colon. The |
| | arthritis may be selected from the group consisting of osteoarthritis, rheumatoid arthritis |
| | and psoriatic arthritis. |
| Cancer | Examples of cancer include but are not limited to glioblastoma, melanoma, |
| | non-small cell lung cancer, head-and-neck cancer, prostate cancer, colon cancer, breast |
| | cancer, bladder cancer, ovarian cancer, cervical cancer, endometrial cancer, renal cancer |
| | and pancreatic cancer. |

### Treating Diseases of the Circulatory System

In one embodiment, the composition, system, and/or component thereof described herein can be used to treat and/or prevent a circulatory system disease. In one embodiment, the plasma exosomes of Wahlgren et al. (Nucleic Acids Research, 2012, Vol. 40, No. 17 e130) can be used to deliver the composition, system, and/or component thereof described herein to the blood. In one embodiment, the circulatory system disease can be treated by using a lentivirus to deliver the composition, system, and/or component thereof described herein to modify hematopoietic stem cells (HSCs) in vivo or ex vivo (see e.g. Drakopoulou, “Review Article, The Ongoing Challenge of Hematopoietic Stem Cell-Based Gene Therapy for β-Thalassemia,” Stem Cells International, Volume 2011, Article ID 987980, 10 pages, doi:10.4061/2011/987980, which can be adapted for use with the composition, system, and/or component thereof described herein in view of the description herein). In one embodiment, the circulatory system disorder can be treated by correcting HSCs as to the disease using a composition, system, or a component thereof described herein, wherein the composition, system, and/or component thereof optionally includes a suitable HDR repair template (see e.g. Cavazzana, “Outcomes of Gene Therapy for β-Thalassemia Major via Transplantation of Autologous Hematopoietic Stem Cells Transduced Ex Vivo with a Lentiviral βA-T87Q-Globin Vector.”; Cavazzana-Calvo, “Transfusion independence and HMGA2 activation after gene therapy of human β-thalassaemia,” Nature 467, 318-322 (16 Sep. 2010) doi:10.1038/nature09328; Nienhuis, “Development of Gene Therapy for Thalassemia, Cold Spring Harbor Perspectives in Medicine,” doi: 10.1101/cshperspect.a011833 (2012), LentiGlobin BB305, a lentiviral vector containing an engineered β-globin gene (PA-T87Q); and Xie et al., “Seamless gene correction of β-thalassaemia mutations in patient-specific iPSCs using CRISPR/Cas9 and piggyback” Genome Research gr.173427.114 (2014) www.genome.org/cgi/doi/10.1101/gr.173427.114 (Cold Spring Harbor Laboratory Press; Watts, “Hematopoietic Stem Cell Expansion and Gene Therapy” Cytotherapy 13(10):1164-1171. doi:10.3109/14653249.2011.620748 (2011), which can be adapted for use with the composition, system, and/or component thereof described herein in view of the description herein). In one embodiment, iPSCs can be modified using a composition, system, described herein to correct a disease polynucleotide associated with a circulatory disease. In this regard, the teachings of Xu et al. (Sci Rep. 2015 Jul. 9; 5:12065. doi: 10.1038/srep12065) and Song et al. (Stem Cells Dev. 2015 May 1; 24(9):1053-65. doi: 10.1089/scd.2014.0347. Epub 2015 Feb. 5) with respect to modifying iPSCs can be adapted for use in view of the description herein with the composition, system, and/or component thereof described herein.

The term “Hematopoietic Stem Cell” or “HSC” refers broadly those cells considered to be an HSC, e.g., blood cells that give rise to all the other blood cells and are derived from mesoderm; located in the red bone marrow, which is contained in the core of most bones. HSCs of the disclosure include cells having a phenotype of hematopoietic stem cells, identified by small size, lack of lineage (lin) markers, and markers that belong to the cluster of differentiation series, like: CD34, CD38, CD90, CD133, CD105, CD45, and also c-kit,—the receptor for stem cell factor. Hematopoietic stem cells are negative for the markers that are used for detection of lineage commitment, and are, thus, called Lin−; and, during their purification by FACS, a number of up to 14 different mature blood-lineage markers, e.g., CD13 & CD33 for myeloid, CD71 for erythroid, CD19 for B cells, CD61 for megakaryocytic, etc. for humans; and, B220 (murine CD45) for B cells, Mac-1 (CD11b/CD18) for monocytes, Gr-1 for Granulocytes, Ter119 for erythroid cells, Il7Ra, CD3, CD4, CD5, CD8 for T cells, etc. Mouse HSC markers: CD34lo/−, SCA-1+, Thy1.1+/lo, CD38+, C-kit+, lin-, and Human HSC markers: CD34+, CD59+, Thy1/CD90+, CD38lo/−, C-kit/CD117+, and lin−. HSCs are identified by markers. Hence in embodiments discussed herein, the HSCs can be CD34+ cells. HSCs can also be hematopoietic stem cells that are CD34−/CD38−. Stem cells that may lack c-kit on the cell surface that are considered in the art as HSCs are within the ambit of the disclosure, as well as CD133+ cells likewise considered HSCs in the art.

In one embodiment, the treatment or prevention for treating a circulatory system or blood disease can include modifying a human cord blood cell with any modification described herein. In one embodiment, the treatment or prevention for treating a circulatory system or blood disease can include modifying a granulocyte colony-stimulating factor-mobilized peripheral blood cell (mPB) with any modification described herein. In one embodiment, the human cord blood cell or mPB can be CD34+. In one embodiment, the cord blood cell(s) or mPB cell(s) modified can be autologous. In one embodiment, the cord blood cell(s) or mPB cell(s) can be allogenic. In addition to the modification of the disease gene(s), allogenic cells can be further modified using the composition, system, described herein to reduce the immunogenicity of the cells when delivered to the recipient. Such techniques are described elsewhere herein and e.g., Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20 (2010) 857-862, which can be adapted for use with the composition, system, herein. The modified cord blood cell(s) or mPB cell(s) can be optionally expanded in vitro. The modified cord blood cell(s) or mPB cell(s) can be derived to a subject in need thereof using any suitable delivery technique.

The compositions may be engineered to target genetic locus or loci in HSCs. In one embodiment, the METTL17 polypeptide(s) can be codon-optimized for a eukaryotic cell and especially a mammalian cell, e.g., a human cell, for instance, HSC, or iPSC and nucleic acid component targeting a locus or loci in HSC, such as circulatory disease, can be prepared. These may be delivered via particles. The particles may be formed by the METTL17 polypeptide and the nucleic acid component being admixed. The nucleic acid component and METTL17 polypeptide mixture can be, for example, admixed with a mixture comprising or consisting essentially of or consisting of surfactant, phospholipid, biodegradable polymer, lipoprotein and alcohol, whereby particles containing the nucleic acid component and METTL17 polypeptide may be formed. The disclosure comprehends so making particles and particles from such a method as well as uses thereof. Particles suitable delivery of the composition in the context of blood or circulatory system or HSC delivery to the blood or circulatory system are described in greater detail elsewhere herein.

In one embodiment, after ex vivo modification the HSCs or iPCS can be expanded prior to administration to the subject. Expansion of HSCs can be via any suitable method such as that described by, Lee, “Improved ex vivo expansion of adult hematopoietic stem cells by overcoming CUL4-mediated degradation of HOXB4.” Blood. 2013 May 16; 121(20):4082-9. doi: 10.1182/blood-2012-09-455204. Epub 2013 Mar. 21.

In one embodiment, the HSCs or iPSCs modified can be autologous. In one embodiment, the HSCs or iPSCs can be allogenic. In addition to the modification of the disease gene(s), allogenic cells can be further modified using the composition, system, described herein to reduce the immunogenicity of the cells when delivered to the recipient. Such techniques are described elsewhere herein and e.g., Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20 (2010) 857-862, which can be adapted for use with the composition, system, herein.

### Treating Neurological Diseases

In one embodiment, the compositions, systems, described herein can be used to treat diseases of the brain and CNS. Delivery options for the brain include encapsulation of METTL17 polypeptide and nucleic acid component molecule in the form of either DNA or RNA into liposomes and conjugating to molecular Trojan horses for trans-blood brain barrier (BBB) delivery. Molecular Trojan horses have been shown to be effective for delivery of B-gal expression vectors into the brain of non-human primates. The same approach can be used to delivery vectors containing METTL17 polypeptide and nucleic acid component molecule. For instance, Xia C F and Boado R J, Pardridge W M (“Antibody-mediated targeting of siRNA via the human insulin receptor using avidin-biotin technology.” Mol Pharm. 2009 May-Jun. 6(3):747-51. doi: 10.1021/mp800194) describes how delivery of short interfering RNA (siRNA) to cells in culture, and in vivo, is possible with combined use of a receptor-specific monoclonal antibody (mAb) and avidin-biotin technology. The authors also report that because the bond between the targeting mAb and the siRNA is stable with avidin-biotin technology, and RNAi effects at distant sites such as brain are observed in vivo following an intravenous administration of the targeted siRNA, the teachings of which can be adapted for use with the compositions, systems, herein. In other embodiments, an artificial virus can be generated for CNS and/or brain delivery. See e.g., Zhang et al. (Mol Ther. 2003 Jan. 7(1):11-8.)), the teachings of which can be adapted for use with the compositions, systems, herein.

### Treating Hearing Diseases

In one embodiment the composition and system described herein can be used to treat a hearing disease or hearing loss in one or both ears. Deafness is often caused by lost or damaged hair cells that cannot relay signals to auditory neurons. In such cases, cochlear implants may be used to respond to sound and transmit electrical signals to the nerve cells. But these neurons often degenerate and retract from the cochlea as fewer growth factors are released by impaired hair cells.

In one embodiment, the composition, system, or modified cells can be delivered to one or both ears for treating or preventing hearing disease or loss by any suitable method or technique. Suitable methods and techniques include, but are not limited to, those set forth in US Patent Publication No. 20120328580 describes injection of a pharmaceutical composition into the ear (e.g., auricular administration), such as into the luminae of the cochlea (e.g., the Scala media, Sc vestibulae, and Sc tympani), e.g., using a syringe, e.g., a single-dose syringe. For example, one or more of the compounds described herein can be administered by intratympanic injection (e.g., into the middle ear), and/or injections into the outer, middle, and/or inner ear; administration in situ, via a catheter or pump (see e.g. McKenna et al., (U.S. Patent Publication No. 2006/0030837) and Jacobsen et al., (U.S. Pat. No. 7,206,639); administration in combination with a mechanical device such as a cochlear implant or a hearing aid, which is worn in the outer ear (see e.g., U.S. Patent Publication No. 2007/0093878, which provides an exemplary cochlear implant suitable for delivery of the compositions, systems, described herein to the ear). Such methods are routinely used in the art, for example, for the administration of steroids and antibiotics into human ears. Injection can be, for example, through the round window of the ear or through the cochlear capsule. Other inner ear administration methods are known in the art (see, e.g., Salt and Plontke, Drug Discovery Today, 10:1299-1306, 2005). In one embodiment, a catheter or pump can be positioned, e.g., in the ear (e.g., the outer, middle, and/or inner ear) of a patient during a surgical procedure. In one embodiment, a catheter or pump can be positioned, e.g., in the ear (e.g., the outer, middle, and/or inner ear) of a patient without the need for a surgical procedure.

In general, the cell therapy methods described in US Patent Publication No. 20120328580 can be used to promote complete or partial differentiation of a cell to or towards a mature cell type of the inner ear (e.g., a hair cell) in vitro. Cells resulting from such methods can then be transplanted or implanted into a patient in need of such treatment. The cell culture methods required to practice these methods, including methods for identifying and selecting suitable cell types, methods for promoting complete or partial differentiation of selected cells, methods for identifying complete or partially differentiated cell types, and methods for implanting complete or partially differentiated cells are described below.

Cells suitable for use in the present disclosure include, but are not limited to, cells that are capable of differentiating completely or partially into a mature cell of the inner ear, e.g., a hair cell (e.g., an inner and/or outer hair cell), when contacted, e.g., in vitro, with one or more of the compounds described herein. Exemplary cells that are capable of differentiating into a hair cell include, but are not limited to stem cells (e.g., inner ear stem cells, adult stem cells, bone marrow derived stem cells, embryonic stem cells, mesenchymal stem cells, skin stem cells, iPS cells, and fat derived stem cells), progenitor cells (e.g., inner ear progenitor cells), support cells (e.g., Deiters' cells, pillar cells, inner phalangeal cells, tectal cells and Hensen's cells), and/or germ cells. The use of stem cells for the replacement of inner ear sensory cells is described in Li et al., (U.S. Patent Publication No. 2005/0287127) and Li et al., (U.S. patent application Ser. No. 11/953,797). The use of bone marrow derived stem cells for the replacement of inner ear sensory cells is described in Edge et al., PCT/US2007/084654. iPS cells are described, e.g., at Takahashi et al., Cell, Volume 131, Issue 5, Pages 861-872 (2007); Takahashi and Yamanaka, Cell 126, 663-76 (2006); Okita et al., Nature 448, 260-262 (2007); Yu, J. et al., Science 318(5858):1917-1920 (2007); Nakagawa et al., Nat. Biotechnol. 26:101-106 (2008); and Zaehres and Scholer, Cell 131(5):834-835 (2007). Such suitable cells can be identified by analyzing (e.g., qualitatively or quantitatively) the presence of one or more tissue specific genes. For example, gene expression can be detected by detecting the protein product of one or more tissue-specific genes. Protein detection techniques involve staining proteins (e.g., using cell extracts or whole cells) using antibodies against the appropriate antigen. In this case, the appropriate antigen is the protein product of the tissue-specific gene expression. Although, in principle, a first antibody (i.e., the antibody that binds the antigen) can be labeled, it is more common (and improves the visualization) to use a second antibody directed against the first (e.g., an anti-IgG). This second antibody is conjugated either with fluorochromes, or appropriate enzymes for colorimetric reactions, or gold beads (for electron microscopy), or with the biotin-avidin system, so that the location of the primary antibody, and thus the antigen, can be recognized.

The composition and system may be delivered to the ear by direct application of pharmaceutical composition to the outer ear, with compositions modified from US Patent Publication No. 20110142917. In one embodiment the pharmaceutical composition is applied to the ear canal. Delivery to the ear may also be referred to as aural or otic delivery.

In one embodiment, the compositions, systems, or components thereof and/or vectors or vector systems can be delivered to ear via a transfection to the inner ear through the intact round window by a novel proteidic delivery technology which may be applied to the METTL17 system of the present disclosure (see, e.g., Qi et al., Gene Therapy (2013), 1-9). About 40 μl of 10 mM RNA may be contemplated as the dosage for administration to the ear.

According to Rejali et al. (Hear Res. 2007 June; 228(1-2):180-7), cochlear implant function can be improved by good preservation of the spiral ganglion neurons, which are the target of electrical stimulation by the implant and brain derived neurotrophic factor (BDNF) has previously been shown to enhance spiral ganglion survival in experimentally deafened ears. Rejali et al. tested a modified design of the cochlear implant electrode that includes a coating of fibroblast cells transduced by a viral vector with a BDNF gene insert. To accomplish this type of ex vivo gene transfer, Rejali et al. transduced guinea pig fibroblasts with an adenovirus with a BDNF gene cassette insert, and determined that these cells secreted BDNF and then attached BDNF-secreting cells to the cochlear implant electrode via an agarose gel, and implanted the electrode in the scala tympani. Rejali et al. determined that the BDNF expressing electrodes were able to preserve significantly more spiral ganglion neurons in the basal turns of the cochlea after 48 days of implantation when compared to control electrodes and demonstrated the feasibility of combining cochlear implant therapy with ex vivo gene transfer for enhancing spiral ganglion neuron survival. Such a system may be applied to the METTL17 overexpression system of the present disclosure for delivery to the ear.

In one embodiment, the system set forth in Mukherjea et al. (Antioxidants & Redox Signaling, Volume 13, Number 5, 2010) can be adapted for transtympanic administration of the composition, system, or component thereof to the ear. In one embodiment, a dosage of about 2 mg to about 4 mg of METTL17 polypeptide for administration to a human.

In one embodiment, the system set forth in [Jung et al. (Molecular Therapy, vol. 21 no. 4, 834-841 Apr. 2013) can be adapted for vestibular epithelial delivery of the composition, system, or component thereof to the ear. In one embodiment, a dosage of about 1 to about 30 mg of METTL17 polypeptide for administration to a human.

### Treating Diseases in Non-Dividing Cells

In one embodiment, the gene or transcript to be corrected is in a non-dividing cell. Exemplary non-dividing cells are muscle cells or neurons. Non-dividing (especially non-dividing, fully differentiated) cell types present issues for gene targeting or genome engineering, for example because homologous recombination (HR) is generally suppressed in the G1 cell-cycle phase. However, while studying the mechanisms by which cells control normal DNA repair systems, Durocher discovered a previously unknown switch that keeps HR “off” in non-dividing cells and devised a strategy to toggle this switch back on. Orthwein et al. (Daniel Durocher's lab at the Mount Sinai Hospital in Ottawa, Canada) recently reported (Nature 16142, published online 9 Dec. 2015) have shown that the suppression of HR can be lifted and gene targeting successfully concluded in both kidney (293T) and osteosarcoma (U20S) cells. Tumor suppressors, BRCA1, PALB2 and BRAC2 are known to promote DNA DSB repair by HR. They found that formation of a complex of BRCA1 with PALB2-BRAC2 is governed by a ubiquitin site on PALB2, such that action on the site by an E3 ubiquitin ligase. This E3 ubiquitin ligase is composed of KEAP1 (a PALB2-interacting protein) in complex with cullin-3 (CUL3)-RBX1. PALB2 ubiquitylation suppresses its interaction with BRCA1 and is counteracted by the deubiquitylase USP11, which is itself under cell cycle control. Restoration of the BRCA1-PALB2 interaction combined with the activation of DNA-end resection is sufficient to induce homologous recombination in G1, as measured by a number of methods including a Cas polypeptide nuclease-based gene-targeting assay directed at USP11 or KEAP1 (expressed from a pX459 vector). However, when the BRCA1-PALB2 interaction was restored in resection-competent G1 cells using either KEAP1 depletion or expression of the PALB2-KR mutant, a robust increase in gene-targeting events was detected. These teachings can be adapted for and/or applied to the compositions, systems, described herein.

Thus, reactivation of HR in cells, especially non-dividing, fully differentiated cell types is preferred. In one embodiment, promotion of the BRCA1-PALB2 interaction is preferred. In one embodiment, the target cell is a non-dividing cell. In one embodiment, the target cell is a neuron or muscle cell. In one embodiment, the target cell is targeted in vivo. In one embodiment, the cell is in G1 and HR is suppressed. In one embodiment, use of KEAP1 depletion, for example inhibition of expression of KEAP1 activity, is preferred. KEAP1 depletion may be achieved through siRNA, for example as shown in Orthwein et al. Alternatively, expression of the PALB2-KR mutant (lacking all eight Lys residues in the BRCA1-interaction domain is preferred, either in combination with KEAP1 depletion or alone. PALB2-KR interacts with BRCA1 irrespective of cell cycle position. Thus, promotion or restoration of the BRCA1-PALB2 interaction, especially in G1 cells, is preferred. In one embodiment, especially where the target cells are non-dividing, or where removal and return (ex vivo gene targeting) is problematic, for example neuron or muscle cells. KEAP1 siRNA is available from ThermoFischer. In one embodiment, a BRCA1-PALB2 complex may be delivered to the G1 cell. In one embodiment, PALB2 deubiquitylation may be promoted for example by increased expression of the deubiquitylase USP 11, so it is envisaged that a construct may be provided to promote or up-regulate expression or activity of the deubiquitylase USP11.

### Treating Diseases of the Eye

In one embodiment, the disease to be treated is a disease that affects the eyes. Thus, in one embodiment, the composition, system, or component thereof described herein is delivered to one or both eyes.

The composition, system, can be used to correct ocular defects that arise from several genetic mutations further described in Genetic Diseases of the Eye, Second Edition, edited by Elias I. Traboulsi, Oxford University Press, 2012.

In one embodiment, the condition to be treated or targeted is an eye disorder. In one embodiment, the eye disorder may include glaucoma. In one embodiment, the eye disorder includes a retinal degenerative disease. In one embodiment, the retinal degenerative disease is selected from Stargardt disease, Bardet-Biedl Syndrome, Best disease, Blue Cone Monochromacy, Choroidermia, Cone-rod dystrophy, Congenital Stationary Night Blindness, Enhanced S-Cone Syndrome, Juvenile X-Linked Retinoschisis, Leber Congenital Amaurosis, Malattia Leventinesse, Norrie Disease or X-linked Familial Exudative Vitreoretinopathy, Pattern Dystrophy, Sorsby Dystrophy, Usher Syndrome, Retinitis Pigmentosa, Achromatopsia or Macular dystrophies or degeneration, Retinitis Pigmentosa, Achromatopsia, and age related macular degeneration. In one embodiment, the retinal degenerative disease is Leber Congenital Amaurosis (LCA) or Retinitis Pigmentosa. Other exemplary eye diseases are described in greater detail elsewhere herein.

In one embodiment, the composition, system, is delivered to the eye, optionally via intravitreal injection or subretinal injection. Intraocular injections may be performed with the aid of an operating microscope. For subretinal and intravitreal injections, eyes may be prolapsed by gentle digital pressure and fundi visualized using a contact lens system consisting of a drop of a coupling medium solution on the cornea covered with a glass microscope slide coverslip. For subretinal injections, the tip of a 10-mm 34-gauge needle, mounted on a 5-μl Hamilton syringe may be advanced under direct visualization through the superior equatorial sclera tangentially towards the posterior pole until the aperture of the needle was visible in the subretinal space. Then, 2 μl of vector suspension may be injected to produce a superior bullous retinal detachment, thus confirming subretinal vector administration. This approach creates a self-sealing sclerotomy allowing the vector suspension to be retained in the subretinal space until it is absorbed by the RPE, usually within 48 h of the procedure. This procedure may be repeated in the inferior hemisphere to produce an inferior retinal detachment. This technique results in the exposure of approximately 70% of neurosensory retina and RPE to the vector suspension. For intravitreal injections, the needle tip may be advanced through the sclera 1 mm posterior to the corneoscleral limbus and 2 μl of vector suspension injected into the vitreous cavity. For intracameral injections, the needle tip may be advanced through a corneoscleral limbal paracentesis, directed towards the central cornea, and 2 μl of vector suspension may be injected. For intracameral injections, the needle tip may be advanced through a corneoscleral limbal paracentesis, directed towards the central cornea, and 2 μl of vector suspension may be injected. These vectors may be injected at titers of either 1.0-1.4×10¹⁰ or 1.0-1.4×10⁹ transducing units (TU)/ml.

In one embodiment, lentiviral vectors may be used for administration to the eye. In one embodiment, the lentiviral vector is an equine infectious anemia virus (EIAV) vector. Exemplary EIAV vectors for eye delivery are described in Balagaan, J Gene Med 2006; 8: 275-285, Published online 21 Nov. 2005 in Wiley InterScience (www.interscience.wiley.com). DOI: 10.1002/jgm.845; Binley et al., HUMAN GENE THERAPY 23:980-991 (September 2012), which can be adapted for use with the composition, system, described herein. In one embodiment, the dosage can be 1.1×105 transducing units per eye (TU/eye) in a total volume of 100 μl.

Other viral vectors can also be used for delivery to the eye, such as AAV vectors, such as those described in Campochiaro et al., Human Gene Therapy 17:167-176 (February 2006), Millington-Ward et al. (Molecular Therapy, vol. 19 no. 4, 642-649 April 2011; Dalkara et al. (Sci Transl Med 5, 189ra76 (2013)), which can be adapted for use with the composition, system, described herein. In one embodiment, the dose can range from about 106 to 109.5 particle units. In the context of the Millington-Ward AAV vectors, a dose of about 2×10¹¹ to about 6×10¹³ virus particles can be administered. In the context of Dalkara vectors, a dose of about 1×10¹⁵ to about 1×10¹⁶ vg/ml administered to a human.

In one embodiment, the sd-rxRNA® system of RXi Pharmaceuticals may be used/and or adapted for delivering composition, system, to the eye. In this system, a single intravitreal administration of 3 g of sd-rxRNA results in sequence-specific reduction of PPIB mRNA levels for 14 days. The sd-rxRNA® system may be applied to the METTL17 system of the present disclosure, contemplating a dose of about 3 to 20 mg of composition administered to a human.

In other embodiments, the methods of US Patent Publication No. 20130183282, which is directed to methods of cleaving a target sequence from the human rhodopsin gene, may also be modified to the METTL17 system of the present disclosure.

In other embodiments, the methods of US Patent Publication No. 20130202678 for treating retinopathies and sight-threatening ophthalmologic disorders relating to delivering of the Puf-A gene (which is expressed in retinal ganglion and pigmented cells of eye tissues and displays a unique anti-apoptotic activity) to the sub-retinal or intravitreal space in the eye may be used or adapted. In particular, desirable targets are zgc:193933, prdm1a, spata2, tex10, rbb4, ddx3, zp2.2, Blimp-1 and HtrA2, all of which may be targeted by the composition, system, of the present disclosure.

Wu (Cell Stem Cell, 13:659-62, 2013) designed a guide RNA that led Cas9 to a single base pair mutation that causes cataracts in mice, where it induced DNA cleavage. Then using either the other wild-type allele or oligos given to the zygotes repair mechanisms corrected the sequence of the broken allele and corrected the cataract-causing genetic defect in mutant mouse. This approach can be adapted to and/or applied to the METTL17 compositions, systems, described herein.

US Patent Publication No. 20120159653, describes use of zinc finger nucleases to genetically modify cells, animals and proteins associated with macular degeneration (MD), the teachings of which can be applied to and/or adapted for the METTL17 compositions, systems, described herein.

One aspect of US Patent Publication No. 20120159653 relates to editing of any chromosomal sequences that encode proteins associated with MD which may be applied to the METTL17 system of the present disclosure.

### Treating Muscle Diseases and Cardiovascular Diseases

In one embodiment, the composition, system can be used to treat and/or prevent a muscle disease and associated circulatory or cardiovascular disease or disorder. The present disclosure also contemplates delivering the composition, system, described herein, e.g., METTL17 effector protein systems, to the heart. For the heart, a myocardium tropic adeno-associated virus (AAVM) is preferred, in particular AAVM41 which showed preferential gene transfer in the heart (see, e.g., Lin-Yanga et al., PNAS, Mar. 10, 2009, vol. 106, no. 10). Administration may be systemic or local. A dosage of about 1-10×10¹⁴ vector genomes is contemplated for systemic administration. See also, e.g., Eulalio et al. (2012) Nature 492: 376 and Somasuntharam et al. (2013) Biomaterials 34: 7790, the teachings of which can be adapted for and/or applied to the compositions, systems, described herein.

For example, US Patent Publication No. 20110023139, the teachings of which can be adapted for and/or applied to the compositions, systems, described herein describes use of zinc finger nucleases to genetically modify cells, animals and proteins associated with cardiovascular disease. Cardiovascular diseases generally include high blood pressure, heart attacks, heart failure, and stroke and TIA. Any chromosomal sequence involved in cardiovascular disease or the protein encoded by any chromosomal sequence involved in cardiovascular disease may be utilized in the methods described in this disclosure. The cardiovascular-related proteins are typically selected based on an experimental association of the cardiovascular-related protein to the development of cardiovascular disease. For example, the production rate or circulating concentration of a cardiovascular-related protein may be elevated or depressed in a population having a cardiovascular disorder relative to a population lacking the cardiovascular disorder. Differences in protein levels may be assessed using proteomic techniques including, but not limited to, Western blot, immunohistochemical staining, enzyme linked immunosorbent assay (ELISA), and mass spectrometry. Alternatively, the cardiovascular-related proteins may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR).

The compositions, systems, herein can be used for treating diseases of the muscular system. The present disclosure also contemplates delivering the composition, system, described herein, effector protein systems, to muscle(s).

In one embodiment, the muscle disease to be treated is a muscle dystrophy such as DMD. In one embodiment, the composition, system, such as a system capable of RNA modification, described herein can be used to achieve exon skipping to achieve correction of the diseased gene. As used herein, the term “exon skipping” refers to the modification of pre-mRNA splicing by the targeting of splice donor and/or acceptor sites within a pre-mRNA with one or more complementary antisense oligonucleotide(s) (AONs). By blocking access of a spliceosome to one or more splice donor or acceptor site, an AON may prevent a splicing reaction thereby causing the deletion of one or more exons from a fully-processed mRNA. Exon skipping may be achieved in the nucleus during the maturation process of pre-mRNAs. In some examples, exon skipping may include the masking of key sequences involved in the splicing of targeted exons by using a composition, system, described herein capable of RNA modification. In one embodiment, exon skipping can be achieved in dystrophin mRNA. In one embodiment, the composition, system, can induce exon skipping at exon 1, 2, 3, 4, 5, 6, 7, 8, 10, 11, 12, 13, 14, 15, 16, 17, 18, 19, 20, 21, 22, 23, 24, 25, 26, 27, 28, 29, 30, 31, 32, 33, 34, 45, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or any combination thereof of the dystrophin mRNA. In one embodiment, the composition, system, can induce exon skipping at exon 43, 44, 50, 51, 52, 55, or any combination thereof of the dystrophin mRNA. Mutations in these exons, can also be corrected using non-exon skipping polynucleotide modification methods.

In one embodiment, for treatment of a muscle disease, the method of Bortolanza et al. (Molecular Therapy vol. 19 no. 11, 2055-264 November 2011) may be applied to an AAV expressing METTL17 polypeptide and injected into humans at a dosage of about 2×10¹⁵ or 2×10¹⁶ vg of vector. The teachings of Bortolanza et al., can be adapted for and/or applied to the compositions, systems, described herein.

In one embodiment, the method of Dumonceaux et al. (Molecular Therapy vol. 18 no. 5, 881-887 May 2010) may be applied to an AAV expressing METTL17 polypeptide and injected into humans, for example, at a dosage of about 10¹⁴ to about 10¹⁵ vg of vector. The teachings of Dumonceaux described herein can be adapted for and/or applied to the compositions, systems, described herein.

In one embodiment, the method of Kinouchi et al. (Gene Therapy (2008) 15, 1126-1130) may be applied to compositions described herein and injected into a human, for example, at a dosage of about 500 to 1000 ml of a 40 M solution into the muscle.

In one embodiment, the method of Hagstrom et al. (Molecular Therapy Vol. 10, No. 2, August 2004) can be adapted for and/or applied to the compositions, systems, herein and injected at a dose of about 15 to about 50 mg into the great saphenous vein of a human.

In one embodiment, the method comprises treating a sickle cell related disease, e.g., sickle cell trait, sickle cell disease such as sickle cell anemia, β-thalassaemia. For example, the method and system may be used to modify the genome of the sickle cell, e.g., by correcting one or more mutations of the β-globin gene. In the case of β-thalassaemia, sickle cell anemia can be corrected by modifying HSCs with the systems. The system allows the specific editing of the cell's genome by cutting its DNA and then letting it repair itself. The METTL17 polypeptide is inserted and directed by a nucleic acid component molecule to the mutated point and then it cuts the DNA at that point. Simultaneously, a healthy version of the sequence is inserted. This sequence is used by the cell's own repair system to fix the induced cut. In this way, the METTL17 polypeptide allows the correction of the mutation in the previously obtained stem cells. The methods and systems may be used to correct HSCs as to sickle cell anemia using a system that targets and corrects the mutation (e.g., with a suitable HDR template that delivers a coding sequence for β-globin, advantageously non-sickling β-globin); specifically, the nucleic acid component molecule can target mutation that give rise to sickle cell anemia, and the HDR can provide coding for proper expression of β-globin. A nucleic acid component molecule that targets the mutation-and-METTL17 polypeptide containing particle is contacted with HSCs carrying the mutation. The particle also can contain a suitable HDR template to correct the mutation for proper expression of 0-globin; or the HSC can be contacted with a second particle or a vector that contains or delivers the HDR template. The so contacted cells can be administered; and optionally treated/expanded; cf. Cartier. The HDR template can provide for the HSC to express an engineered β-globin gene (e.g., PA-T87Q), or β-globin.

### Treating Diseases of the Liver and Kidney

In one embodiment, the composition, system, or component thereof described herein can be used to treat a disease of the kidney or liver. Thus, in one embodiment, delivery of the composition or component thereof described herein is to the liver or kidney.

Delivery strategies to induce cellular uptake of the therapeutic nucleic acid include physical force or vector systems such as viral-, lipid- or complex-based delivery, or nanocarriers. From the initial applications with less possible clinical relevance, when nucleic acids were addressed to renal cells with hydrodynamic high-pressure injection systemically, a wide range of gene therapeutic viral and non-viral carriers have been applied already to target posttranscriptional events in different animal kidney disease models in vivo (Csaba Révész and Péter Hamar (2011). Delivery Methods to Target RNAs in the Kidney, Gene Therapy Applications, Prof. Chunsheng Kang (Ed.), ISBN: 978-953-307-541-9, InTech, Available from: www.intechopen.com/books/gene-therapy-applications/delivery-methods-to-target-rnas-inthe-kidney). Delivery methods to the kidney may include those in Yuan et al. (Am J Physiol Renal Physiol 295: F605-F617, 2008). The method of Yuang et al. may be applied to the composition of the present disclosure contemplating a 1-2 g subcutaneous injection of polypeptide nuclease conjugated with cholesterol to a human for delivery to the kidneys. In one embodiment, the method of Molitoris et al. (J Am Soc Nephrol 20: 1754-1764, 2009) can be adapted to the composition and a cumulative dose of 12-20 mg/kg to a human can be used for delivery to the proximal tubule cells of the kidneys. In one embodiment, the methods of Thompson et al. (Nucleic Acid Therapeutics, Volume 22, Number 4, 2012) can be adapted to the compositions and a dose of up to 25 mg/kg can be delivered via i.v. administration. In one embodiment, the method of Shimizu et al. (J Am Soc Nephrol 21: 622-633, 2010) can be adapted to the compositions and a dose of about of 10-20 mol compositions complexed with nanocarriers in about 1-2 liters of a physiologic fluid for i.p. administration can be used.

Other various delivery vehicles can be used to deliver the composition, system to the kidney such as viral, hydrodynamic, lipid, polymer nanoparticles, aptamers and various combinations thereof (see e.g., Larson et al., Surgery, (August 2007), Vol. 142, No. 2, pp. (262-269); Hamar et al., Proc Natl Acad Sci, (October 2004), Vol. 101, No. 41, pp. (14883-14888); Zheng et al., Am J Pathol, (October 2008), Vol. 173, No. 4, pp. (973-980); Feng et al., Transplantation, (May 2009), Vol. 87, No. 9, pp. (1283-1289); Q. Zhang et al., PloS ONE, (July 2010), Vol. 5, No. 7, e11709, pp. (1-13); Kushibikia et al., J Controlled Release, (July 2005), Vol. 105, No. 3, pp. (318-331); Wang et al., Gene Therapy, (July 2006), Vol. 13, No. 14, pp. (1097-1103); Kobayashi et al., Journal of Pharmacology and Experimental Therapeutics, (February 2004), Vol. 308, No. 2, pp. (688-693); Wolfrum et al., Nature Biotechnology, (September 2007), Vol. 25, No. 10, pp. (1149-1157); Molitoris et al., J Am Soc Nephrol, (August 2009), Vol. 20, No. 8 pp. (1754-1764); Mikhaylova et al., Cancer Gene Therapy, (March 2011), Vol. 16, No. 3, pp. (217-226); Y. Zhang et al., J Am Soc Nephrol, (April 2006), Vol. 17, No. 4, pp. (1090-1101); Singhal et al., Cancer Res, (May 2009), Vol. 69, No. 10, pp. (4244-4251); Malek et al., Toxicology and Applied Pharmacology, (April 2009), Vol. 236, No. 1, pp. (97-108); Shimizu et al., J Am Soc Nephrology, (April 2010), Vol. 21, No. 4, pp. (622-633); Jiang et al., Molecular Pharmaceutics, (May-June 2009), Vol. 6, No. 3, pp. (727-737); Cao et al, J Controlled Release, (June 2010), Vol. 144, No. 2, pp. (203-212); Ninichuk et al., Am J Pathol, (March 2008), Vol. 172, No. 3, pp. (628-637); Purschke et al., Proc Natl Acad Sci, (March 2006), Vol. 103, No. 13, pp. (5173-5178).

In one embodiment, delivery is to liver cells. In one embodiment, the liver cell is a hepatocyte. Delivery of the composition and system herein may be via viral vectors, especially AAV (and in particular AAV2/6) vectors. These can be administered by intravenous injection. A preferred target for the liver, whether in vitro or in vivo, is the albumin gene. This is a so-called “safe harbor” as albumin is expressed at very high levels and so some reduction in the production of albumin following successful gene editing is tolerated. It is also preferred as the high levels of expression seen from the albumin promoter/enhancer allows for useful levels of correct or transgene production (from the inserted recombination template) to be achieved even if only a small fraction of hepatocytes are edited. See sites identified by Wechsler et al. (reported at the 57th Annual Meeting and Exposition of the American Society of Hematology—abstract available online at ash.confex.com/ash/2015/webprogram/Paper86495.html and presented on 6 Dec. 2015) which can be adapted for use with the compositions, systems, herein.

Exemplary liver and kidney diseases that can be treated and/or prevented are described elsewhere herein.

### Treating Epithelial and Lung Diseases

In one embodiment, the disease treated or prevented by the composition and system described herein can be a lung or epithelial disease. The compositions and systems described herein can be used for treating epithelial and/or lung diseases. The present disclosure also contemplates delivering the composition, system, described herein, to one or both lungs.

In one embodiment, as viral vector can be used to deliver the composition, system, or component thereof to the lungs. In one embodiment, the AAV is an AAV-1, AAV-2, AAV-5, AAV-6, and/or AAV-9 for delivery to the lungs. (see, e.g., Li et al., Molecular Therapy, vol. 17 no. 12, 2067-277 December 2009). In one embodiment, the MOI can vary from 1×10³ to 4×10⁵ vector genomes/cell. In one embodiment, the delivery vector can be an RSV vector as in Zamora et al. (Am J Respir Crit Care Med Vol 183. pp 531-538, 2011. The method of Zamora et al. may be applied to the METTL17 overexpression system of the present disclosure and an aerosolized composition, for example with a dosage of 0.6 mg/kg, may be contemplated for the present disclosure.

Subjects treated for a lung disease may for example receive pharmaceutically effective amount of aerosolized AAV vector system per lung endobronchially delivered while spontaneously breathing. As such, aerosolized delivery is preferred for AAV delivery in general. An adenovirus or an AAV particle may be used for delivery. Suitable gene constructs, each operably linked to one or more regulatory sequences, may be cloned into the delivery vector. In this instance, the following constructs are provided as examples: Cbh or EF1a promoter for METTL17, U6 or H1 promoter for nucleic acid component molecule. A preferred arrangement is to use a CFTRdelta508 targeting nucleic acid component molecule, a repair template for deltaF508 mutation and a codon optimized composition, with optionally one or more nuclear localization signal or sequence(s) (NLS(s)), e.g., two (2) NLSs.

### Treating Diseases of the Skin

The compositions and systems described herein can be used for the treatment of skin diseases. The present disclosure also contemplates delivering the composition and system, described herein, to the skin.

In one embodiment, delivery to the skin (intradermal delivery) of the composition, system, or component thereof can be via one or more microneedles or microneedle containing device. For example, in one embodiment, the device and methods of Hickerson et al. (Molecular Therapy-Nucleic Acids (2013) 2, e129) can be used and/or adapted to deliver the composition, system, described herein, for example, at a dosage of up to 300 μl of 0.1 mg/ml compositions to the skin.

In one embodiment, the methods and techniques of Leachman et al. (Molecular Therapy, vol. 18 no. 2, 442-446 February 2010) can be used and/or adapted for delivery of a compositions described herein to the skin.

In one embodiment, the methods and techniques of Zheng et al. (PNAS, Jul. 24, 2012, vol. 109, no. 30, 11975-11980) can be used and/or adapted for nanoparticle delivery of a compositions described herein to the skin. In one embodiment, as dosage of about 25 nM applied in a single application can achieve gene knockdown in the skin.

### Methods for Treating Cancer/Enhancing Adoptive Cell Therapies

The compositions, systems, described herein can be used for the treatment of cancer. The present disclosure also contemplates delivering the composition, system, described herein, to a cancer cell. Also, as is described elsewhere herein the compositions, systems, can be used to modify an immune cell, such as a CAR or CAR T cell, which can then in turn be used to treat and/or prevent cancer. This is also described in International Patent Publication No. WO 2015/161276, the disclosure of which is hereby incorporated by reference and described herein below.

In some embodiments, the treatment or prevention of for cancer can also include modification or targeting of one or more genes. In one embodiment, target genes for cancer treatment and prevention can also include those described in International Patent Publication No. WO 2015/048577 the disclosure of which is hereby incorporated by reference and can be adapted for and/or applied to the composition, system, described herein.

### Methods for Improving Gamete Function and Fertilization

The respiration demands on spermatozoa and oocytes in the immediate events leading up to fertilization, during, and through early embryonic development are dependent at least in part on adequate cellular respiration. For example, sperm cells have increased demand to propel themselves through the female reproductive track to reach the oocyte for fertilization. The oocyte must undergo many cellular processes upon contact with a sperm to facilitate entry of the sperm into the oocyte and zygote formation. In some embodiments, the cells contain one or more embodiments of compositions of the present disclosure that do not modify the genome. In some of these embodiments the cells are spermatids, oogonia, oocytes, or spermatozoa. In some embodiments, non-human animal spermatids, oogonia, oocytes, or spermatozoa comprise one or more compositions of the present disclosure. Such cells can have improved respiration and thus the inventive compositions herein can be useful for improving fertilization during natural or in vitro fertilization. In some embodiments, the compositions delivered to spermatozoa are mRNA or protein compositions as spermatozoa generally do not carry out transcription. In some embodiments, the inventive compositions can be included in a formulation adapted for culturing, storing, extending, diluting, or otherwise containing spermatozoa or oocytes. Such formulations, in some embodiments, can also include one or more spermatozoa or oocytes.

In some embodiments, a method of increasing fertilization comprises delivering to a spermatid, spermatozoa, oogonia, or oocyte, a composition of the present disclosure described herein, a polynucleotide of the present disclosure described herein, a delivery system of the present disclosure described herein, or any combination thereof, wherein the composition increases the respiration of the spermatid, spermatozoa, oogonia, or oocyte, or any combination thereof, and wherein the composition does not modify the genome of a human spermatid, spermatozoa, oogonia, or oocyte. In some embodiments, the method further comprises in vitro fertilization.

In some embodiments, the mitochondria disease is a disease caused by a nuclear DNA mutation that results in a dysfunctional mitochondria.

In some embodiments, the mitochondria disease is characterized by a decrease in the number of mitochondria.

In some embodiments, the mitochondria disease is the result of the normal aging.

In some embodiments, the mitochondria disease is an age-related disease.

In some embodiments, the mitochondria disease is a brain or nervous system disease. In some embodiments, mitochondria disease is a cognitive disease or dementia. Exemplary brain and nervous system diseases are described elsewhere herein.

In some embodiments, the mitochondria disease is a muscle disease, such as a muscular dystrophy. Exemplary muscle diseases are described elsewhere herein.

### Non-Mitochondrial Disease Applications and Enhanced Anti-Aging Interventions

In some embodiments, the compositions of the present disclosure can increase the lifespan, increase longevity and/or slow the aging of a subject or cell(s) thereof. Without being bound by theory, improving mitochondrial function can inter alia reduce cellular damage and free radicals thus slowing the aging process and reduce apoptosis.

In certain embodiments, the composition is co-administered with another therapeutic or supplement meant to counter age-related deficiencies and/or increase lifespan, such as nicotinamide adenine dinucleotide (NAD) or NAD precursor (e.g., nicotinamide mononucleotide (NMN), a telomerase activating compound, antioxidant, MOTS-c analogs, a rapalog, a senolytic agent, a therapy capable of clearing senescent cells (e.g., a gene therapy, antibodies targeting senescent cells, immunosenescence drug (e.g., mTORC1 inhibitor) etc.), a SAMolytic agent, agents optimized to control protein homeostasis, agents capable of reducing oxidative stress (e.g., Jivenon), 17-α-estradiol, 17-β-estradiol, acarbose, an autophagy promoting agent (e.g., Metformin, Rapamycin, and Resveratrol) any combination thereof and/or the like. In some embodiments, the compositions of the present disclosure are administered along with a calorie restricted diet.

See also, Magalhaes et al., Trends Biotechnol. 35(1):1062-1073) (2017) Ashok K. Shetty, Maheedhar Kodali, Raghavendra Upadhya, Leelavathi N. Madhu. Emerging Anti-Aging Strategies—Scientific Basis and Efficacy. Aging and disease. 2018, 9(6): 1165-1184 doi: 10.14336/AD.2018.1026; and Berja et al., Front. Cell Dev. Biol., 6 Sep. 2019|doi: 10.3389/fcell.2019.00183, the teachings of which can be adapted for use with the present disclosure.

### Methods of Delivery

The present disclosure also provides delivery systems for introducing components of the systems and compositions herein to cells, tissues, organs, or organisms. A delivery system may comprise one or more delivery vehicles and/or cargos. Exemplary delivery systems and methods include those described in paragraphs [00117] to [00278] of Feng Zhang et al., (WO 2016/106236A1), and pages 1241-1251 and Table 1 of Lino C A et al., Delivering CRISPR: a review of the challenges and approaches, DRUG DELIVERY, 2018, VOL. 25, NO. 1, 1234-1257, which are incorporated by reference herein in their entireties and can be adapted for use with the METTL17 proteins disclosed herein.

In one embodiment, the delivery systems may be used to introduce the components of the systems and compositions to plant cells. For example, the components may be delivered to plant using electroporation, microinjection, aerosol beam injection of plant cell protoplasts, biolistic methods, DNA particle bombardment, and/or Agrobacterium-mediated transformation. Examples of methods and delivery systems for plants include those described in Fu et al., Transgenic Res. 2000 Feb. 9(1):11-9; Klein R M, et al., Biotechnology. 1992; 24:384-6; Casas A M et al., Proc Natl Acad Sci USA. 1993 Dec. 1; 90(23): 11212-11216; and U.S. Pat. No. 5,563,055, Davey M R et al., Plant Mol Biol. 1989 Sep. 13(3):273-85, which are incorporated by reference herein in their entireties.

The example delivery compositions, systems, and methods described herein related to composition or METTL17 polypeptide also apply to functional domains and other components (e.g., other proteins and polynucleotides related to the METTL17 polypeptide, such as reverse transcriptase, nucleotide deaminase, retrotransposon, donor polynucleotide, etc.). In a preferred embodiment, the composition comprises delivery of the polypeptides via mRNA.

mRNA Delivery

In one embodiment, the METTL17 polynucleotide is delivered as an mRNA encoding the METTL17 polypeptide. The in vivo translation efficiency of mRNA molecules may be further increased by RNA engineering. To achieve effective translation, mRNA requires five structural elements, including the 5′ cap, 3′ poly(A) tail, protein-coding sequence and 5′ and 3′ untranslated regions (UTRs) with sequence engineering of one or more of these elements may be utilized to improve translation in vivo.

In some embodiments, the isolated mRNA is not self-replicating.

In some embodiment, the isolated mRNA comprises and/or encodes one or more 5′ terminal cap (or cap structure), 3′ terminal cap, 5′ untranslated region, 3′ untranslated region, a tailing region, or any combination thereof.

In some embodiments, the capping region of the isolated mRNA region may be from 1 to 10, e.g., 2-9, 3-8, 4-7, 1-5, 5-10, or at least 2, or 10 or fewer nucleotides in length. In some embodiments, the cap is absent.

In an exemplary embodiment, mRNA can be synthesized in vitro and transferred directly into target cells, and may be further modified. For example, the mRNA may comprise a 5′ end of endogenous mRNAs modified with a 7-methylguanosine cap structure, with polyadenylated 3′ end, which may facilitate protein production. Modification of pyrimidine residues may also be performed to enhance transgene expression from delivered mRNAs, as it may lower stimulation of the innate immune system of host cells. In an example embodiment, the mRNA comprises an anti-reverse cap analog and a 120-nt poly(A) tail, and optionally may comprise cytosine and uridine residues replaced with 5-methylcytosine and pseudouridine. See, U.S. Patent Publication 2019/0151474, incorporated herein by reference.

In some embodiments, a 5′-cap structure is cap0, cap1, ARCA, inosine, N1-methyl-guanosine, 2′-fluoro-guanosine, 7-deaza-guanosine, 8-oxo-guanosine, 2-amino-guanosine, LNA-guanosine, or 2-azido-guanosine.

In some embodiments, the 5′ terminal cap is 7mG(5′)ppp(5′)NlmpNp, m7GpppG cap, N7-methylguanine. In some embodiments, the 3′ terminal cap is a 3′-O-methyl-m7GpppG.

In some embodiments, the 3′-UTR is an alpha-globin 3′-UTR. In some embodiments, the 5′-UTR comprises a Kozak sequence.

In some embodiments, the tailing sequence may range from absent to 500 nucleotides in length (e.g., at least 60, 70, 80, 90, 120, 140, 160, 180, 200, 250, 300, 350, 400, 450, or 500 nucleotides). In some embodiments, the tailing region is or includes a polyA tail. Where the tailing region is a polyA tail, the length may be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional. In some embodiments, the poly-A tail is at least 160 nucleotides in length.

In some embodiments, the mRNA polynucleotide includes a stabilization element. In some embodiments, the stabilization element is a histone stem-loop. In some embodiments, the stabilization element is a nucleic acid sequence having increased GC content relative to wild type sequence.

In an embodiment, it is desirable to reduce the immunogenic sequence motifs of the mRNA for delivery. Exemplary techniques are known in the art, see, e.g., International Patent Publication WO 2020/033720, discussing exemplary immunogenic sequence motifs for removal, including those that can bind human TLR8), incorporated herein by reference.

The isolated mRNA(s) can be made in part or using only in vitro transcription. Methods of making polynucleotides by in vitro transcription are known in the art and are described in U.S. Provisional Patent Application Nos. 61/618,862, 61/681,645, 61/737,130, 61/618,866, 61/681,647, 61/737,134, 61/618,868, 61/681,648, 61/737,135, 61/618,873, 61/681,650, 61/737,147, 61/618,878, 61/681,654, 61/737,152, 61/618,885, 61/681,658, 61/737,155, 61/618,896, 61/668,157, 61/681,661, 61/737,160, 61/618,911, 61/681,667, 61/737,168, 61/618,922, 61/681,675, 61/737,174, 61/618,935, 61/681,687, 61/737,184, 61/618,945, 61/681,696, 61/737,191, 61/618,953, 61/681,704 61/737,203, International Publication Nos WO 2013/151666, WO 2013/151668, WO 2013/151663, WO 2013/151669, WO 2013/151670, WO 2013/151664, WO 2013/151665, WO 2013/151736, WO 2013/151672, WO 2013/151671 WO 2013/151667, and WO 2020/205793A1; the contents of each of which are herein incorporated by reference in their entireties. Cell-free production methods of making ribonucleic acid, including large scale syntheses are described, for example in U.S. Pat. No. 10,954,541, incorporated herein by reference in its entirety.

Targeted delivery of mRNA and endosomal escape are generally requirements of effective mRNA use. Lipids, including lipid nanoparticles, lipid-like materials, polymers are particularly preferred delivery vehicles, as detailed elsewhere herein.

The delivery systems may comprise one or more cargos. The cargos may comprise one or more components of the systems and compositions herein. A cargo may comprise one or more of the following: i) a plasmid encoding one or more proteins components in the compositions and systems such as the METTL17 polypeptide and/or functional domains; ii) a plasmid encoding one or more nucleic acid components, iii) mRNA of one or more one or more proteins components in the compositions and systems such as the METTL17 polypeptide and/or functional domains; iv) one or more nucleic acid component molecules; v) one or more proteins components in the compositions and systems such as the METTL17 polypeptide and/or functional domains; vi) any combination thereof. The one or more protein components may include the nuclei acid-guided nuclease (e.g., Cas), reverse transcriptase, nucleotide deaminase, retrotransposon protein, other functional domain, or any combination thereof.

In some examples, a cargo may comprise a plasmid encoding one or more proteins components in the compositions and systems such as the METTL17 polypeptide and/or functional domains and one or more (e.g., a plurality of) nucleic acid component molecules. In some cases, the plasmid may also encode a recombination template (e.g., for HDR). In one embodiment, a cargo may comprise mRNA encoding one or more protein components and one or more nucleic acid component molecules.

In some examples, a cargo may comprise one or more protein components and one or more nucleic acid component molecules, e.g., in the form of ribonucleoprotein complexes (RNP). The ribonucleoprotein complexes may be delivered by methods and systems herein. In some cases, the ribonucleoprotein may be delivered by way of a polypeptide-based shuttle agent. In one example, the ribonucleoprotein may be delivered using synthetic peptides comprising an endosome leakage domain (ELD) operably linked to a cell penetrating domain (CPD), to a histidine-rich domain and a CPD, e.g., as describe in WO 2016/161516. RNP may also be used for delivering the compositions and systems to plant cells, e.g., as described in Wu J W, et al., Nat Biotechnol. 2015 November; 33(11):1162-4.

### Physical Delivery

In one embodiment, the cargos may be introduced to cells by physical delivery methods. Examples of physical methods include microinjection, electroporation, and hydrodynamic delivery. Both nucleic acid and proteins may be delivered using such methods. For example, one or more protein components may be prepared in vitro, isolated, (refolded, purified if needed), and introduced to cells.

### Microinjection

Microinjection of the cargo directly to cells can achieve high efficiency, e.g., above 90% or about 100%. In one embodiment, microinjection may be performed using a microscope and a needle (e.g., with 0.5-5.0 m in diameter) to pierce a cell membrane and deliver the cargo directly to a target site within the cell. Microinjection may be used for in vitro and ex vivo delivery.

Plasmids comprising coding sequences for one or more protein components and/or nucleic acid components, mRNAs, and/or nucleic acid component molecules, may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery nucleic acid component directly to the nucleus and mRNA to the cytoplasm, e.g., facilitating translation and shuttling of one or more protein components to the nucleus.

Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transiently up- or down-regulate a specific gene within the genome of a cell, e.g., using METTL17.

### Electroporation

In one embodiment, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The cargos, e.g., nucleic acids, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses, non-viral vehicles, and other delivery reagents described herein.

The delivery vehicles in accordance with the present disclosure may have a greatest dimension (e.g., diameter) of less than 100 microns (μm). In one embodiment, the delivery vehicles have a greatest dimension of less than 10 μm. In one embodiment, the delivery vehicles may have a greatest dimension of less than 2000 nanometers (nm). In one embodiment, the delivery vehicles may have a greatest dimension of less than 1000 nanometers (nm). In one embodiment, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In one embodiment, the delivery vehicles may have a greatest dimension ranging between 25 nm and 200 nm.

In one embodiment, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles). Nanoparticles may also be used to deliver the compositions and systems to plant cells, e.g., as described in International Patent Publication No. WO 2008/042156, US Publication Application No. US 2013/0185823, and International Patent Publication No WO 2015/089419.

### Vectors

The systems, compositions, and/or delivery systems may comprise one or more vectors. The present disclosure also includes vector systems. A vector system may comprise one or more vectors. In one embodiment, a vector refers to a nucleic acid molecule capable of transporting another nucleic acid to which it has been linked. Vectors include nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. A vector may be a plasmid, e.g., a circular double stranded DNA loop into which additional DNA segments can be inserted, such as by standard molecular cloning techniques. Certain vectors may be capable of autonomous replication in a host cell into which they are introduced (e.g., bacterial vectors having a bacterial origin of replication and episomal mammalian vectors). Some vectors (e.g., non-episomal mammalian vectors) are integrated into the genome of a host cell upon introduction into the host cell, and thereby are replicated along with the host genome. In certain examples, vectors may be expression vectors, e.g., capable of directing the expression of genes to which they are operatively-linked. In some cases, the expression vectors may be for expression in eukaryotic cells. Common expression vectors of utility in recombinant DNA techniques are often in the form of plasmids.

Examples of vectors include pGEX, pMAL, pRIT5, E. coli expression vectors (e.g., pTrc, pET lid, yeast expression vectors (e.g., pYepSec1, pMFa, pJRY88, pYES2, and picZ, Baculovirus vectors (e.g., for expression in insect cells such as SF9 cells) (e.g., pAc series and the pVL series), mammalian expression vectors (e.g., pCDM8 and pMT2PC.

A vector may comprise i) one or more protein components encoding sequence(s), and/or ii) a single, or at least 2, at least 3, at least 4, at least 5, at least 6, at least 7, at least 8, at least 9, at least 10, at least 12, at least 14, at least 16, at least 32, at least 48, at least 50 nucleic acid component molecule(s) encoding sequences. In a single vector there can be a promoter for each RNA coding sequence. Alternatively or additionally, in a single vector, there may be a promoter controlling (e.g., driving transcription and/or expression) multiple RNA encoding sequences.

Furthermore, that compositions or systems may be delivered via a vector, e.g., a separate vector or the same vector that is encoding the complex. When provided by a separate vector, the RNA that targets METTL17 polypeptide expression can be administered sequentially or simultaneously. When administered sequentially, the RNA that targets METTL17 polypeptide expression is to be delivered after the RNA that is intended for e.g., gene editing or gene engineering. This period may be a period of minutes (e.g., 5 minutes, 10 minutes, 20 minutes, 30 minutes, 45 minutes, 60 minutes). This period may be a period of hours (e.g., 2 hours, 4 hours, 6 hours, 8 hours, 12 hours, 24 hours). This period may be a period of days (e.g., 2 days, 3 days, 4 days, 7 days). This period may be a period of weeks (e.g., 2 weeks, 3 weeks, 4 weeks). This period may be a period of months (e.g., 2 months, 4 months, 8 months, 12 months). This period may be a period of years (2 years, 3 years, 4 years). In this fashion, the METTL17 polypeptide associates with a first nucleic acid component molecule capable of hybridizing to a first target, such as a genomic locus or loci of interest and undertakes the function(s) desired of the system (e.g., gene engineering); and subsequently the METTL17 polypeptide may then associate with the second nucleic acid component molecule capable of hybridizing to the sequence comprising at least part of the METTL17 polypeptide. Where the nucleic acid component molecule targets the sequences encoding expression of the METTL17 polypeptide, the enzyme becomes impeded and the system becomes self-inactivating. In the same manner, RNA that targets METTL17 polypeptide expression applied via, for example liposome, lipofection, particles, microvesicles as explained herein, may be administered sequentially or simultaneously. Similarly, self-inactivation may be used for inactivation of one or more nucleic acid component molecule used to target one or more targets.

### Viral Vectors

The cargos may be delivered by viruses. In one embodiment, viral vectors are used. A viral vector may comprise virally-derived DNA or RNA sequences for packaging into a virus (e.g., retroviruses, replication defective retroviruses, adenoviruses, replication defective adenoviruses, and adeno-associated viruses). Viral vectors also include polynucleotides carried by a virus for transfection into a host cell. Viruses and viral vectors may be used for in vitro, ex vivo, and/or in vivo deliveries.

### Adeno Associated Virus (AAV)

The systems and compositions herein may be delivered by adeno associated virus (AAV). AAV vectors may be used for such delivery. AAV, of the Dependovirus genus and Parvoviridae family, is a single stranded DNA virus. In one embodiment, AAV may provide a persistent source of the provided DNA, as AAV delivered genomic material can exist indefinitely in cells, e.g., either as exogenous DNA or, with some modification, be directly integrated into the host DNA. In one embodiment, AAV do not cause or relate with any diseases in humans. The virus itself is able to efficiently infect cells while provoking little to no innate or adaptive immune response or associated toxicity.

Examples of AAV that can be used herein include AAV-1, AAV-2, AAV-3, AAV-4, AAV-5, AAV-6, AAV-8, and AAV-9. The type of AAV may be selected with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. AAV-2-based vectors were originally proposed for CFTR delivery to CF airways, other serotypes such as AAV-1, AAV-5, AAV-6, and AAV-9 exhibit improved gene transfer efficiency in a variety of models of the lung epithelium. Examples of cell types targeted by AAV are described in Grimm, D. et al, J. Virol. 82: 5887-5911 (2008)), and shown as follows in Table 10.

TABLE 10

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

The AAV particles may be created in HEK 293 T cells. Once particles with specific tropism have been created, they are used to infect the target cell line much in the same way that native viral particles do. This may allow for persistent presence of the components in the infected cell type, and what makes this version of delivery particularly suited to cases where long-term expression is desirable. Examples of doses and formulations for AAV that can be used include those describe in U.S. Pat. Nos. 8,454,972 and 8,404,658.

Various strategies may be used for delivery the systems and compositions herein with AAVs. In some examples, coding sequences of METTL17 polypeptide and nucleic acid component may be packaged directly onto one DNA plasmid vector and delivered via one AAV particle. In some examples, AAVs may be used to deliver nucleic acid components into cells that have been previously engineered to express METTL17 polypeptide. In some examples, coding sequences of METTL17 polypeptide and nucleic acid component may be made into two separate AAV particles, which are used for co-transfection of target cells. In some examples, markers, tags, and other sequences may be packaged in the same AAV particles as coding sequences of METTL17 polypeptide and/or nucleic acid components.

### Lentiviruses

The systems and compositions herein may be delivered by lentiviruses. Lentiviral vectors may be used for such delivery. Lentiviruses are complex retroviruses that have the ability to infect and express their genes in both mitotic and post-mitotic cells.

Examples of lentiviruses include human immunodeficiency virus (HIV), which may use its envelope glycoproteins of other viruses to target a broad range of cell types; minimal non-primate lentiviral vectors based on the equine infectious anemia virus (EIAV), which may be used for ocular therapies. In one embodiment, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) may be used/and or adapted to the METTL17 overexpression system herein.

Lentiviruses may be pseudo-typed with other viral proteins, such as the G protein of vesicular stomatitis virus. In doing so, the cellular tropism of the lentiviruses can be altered to be as broad or narrow as desired. In some cases, to improve safety, second- and third-generation lentiviral systems may split essential genes across three plasmids, which may reduce the likelihood of accidental reconstitution of viable viral particles within cells.

In some examples, leveraging the integration ability, lentiviruses may be used to create libraries of cells comprising various genetic modifications, e.g., for screening and/or studying genes and signaling pathways.

### Adenoviruses

The systems and compositions herein may be delivered by adenoviruses. Adenoviral vectors may be used for such delivery. Adenoviruses include nonenveloped viruses with an icosahedral nucleocapsid containing a double stranded DNA genome. Adenoviruses may infect dividing and non-dividing cells. In one embodiment, adenoviruses do not integrate into the genome of host cells, which may be used for limiting off-target effects of systems in gene editing applications.

### Viral Vehicles for Delivery to Plants

The systems and compositions may be delivered to plant cells using viral vehicles. In particular embodiments, the compositions and systems may be introduced in the plant cells using a plant viral vector (e.g., as described in Scholthof et al. 1996, Annu Rev Phytopathol. 1996; 34:299-323). Such viral vector may be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector may be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses may be non-integrative vectors.

### Non-Viral Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, gold nanoparticles, streptolysin O, multifunctional envelope-type nanodevices (MENDs), lipid-coated mesoporous silica particles, and other inorganic nanoparticles. Targeted delivery of RNA and endosomal escape are generally requirements of effective RNA use. Lipids, including lipid nanoparticles, lipid-like materials, polymers are particularly preferred delivery vehicles for RNA, as detailed further below.

### Nanoparticles

Delivery vehicles for use with the present compositions may comprise nanoparticles including lipid nanoparticles. Other particle systems, including polymer based materials such as calcium phosphate-silicate nanoparticle, a calcium phosphate nanoparticle, a silica nanoparticle, and poly(amido-amine), poly-beta amino-esters (PBAEs), and polyethylenimine (PEI) can be used. See, e.g., Trepotec et al. Mol. Therapy 27:4 Apr. 2019. In an embodiment, the exemplary nanoparticle comprises modified dendrimers comprising cores, one or more of homogeneous or heterogeneous intermediate and terminal layers for the enclosure and delivery of nucleic acid, e.g., mRNA. Modified dendrimers can be preferably comprise one or more polyester dendrimers, for example, comprising a core branching into one or more generations of polyester units, with polyester attached at surface via amine linkers (e.g., polyamine) to hydrophobic units (e.g., fatty acid derivative), including polyamidoamine (PAMAM) dendrimers, polypropylene imine (PPI) dendrimers, or polyethylene imine (PEI) dendrimers. The plurality of intermediate layers may comprise both at least one layer modified for endosomal escape and a polyfluorocarbon. Exemplary molecules and methods of making can be found in WO 2020/132196, and WO 2021/207020, incorporated herein by reference. Formulas IB, II and III of International Patent Publication WO 2021/207020 are specifically incorporated herein by reference as exemplary nanoparticle delivery vehicles for the delivery of nucleic acids.

### Lipid Particles

The delivery vehicles may comprise lipid particles, e.g., lipid nanoparticles (LNPs) and liposomes. Lipidic aminoglycosides and derivatives thereof are known in the art for delivery of RNA, including dioleylamine-A-succinyl-neomycin (“DOSN”), dioleylamine-A-succinyl-paromomycin (“DOSP”), NeoCHol. NeoSucChol, ParomoChol. ParomoCapSucDOLA, ParamoLysSucDOLA, NeoDiSucDODA, NeodiLysSucDOLA, and [ParomoLys]2-Glu-Lys-[SucDOLA]2 as detailed in International Patent Publicaiton WO 2008/040792, incorporated herein by reference.

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of METTL17 polypeptide and/or nucleic acid component) and/or RNA molecules (e.g., mRNA of METTL17 polypeptide, nucleic acid component molecules). In certain cases, LNPs may be use for delivering RNP complexes of METTL17 polypeptide/nucleic acid component.

Cationic lipids form complexes with mRNA to form a lipoplex which is then endocytosed by cells. In an example embodiment, the LNP comprises a cationic lipid, a helper lipid, cholesterol, and polyethylene glycol (PEG). In an example embodiment, the LNP can comprise paromomycin-based cationic lipids, with either an amide or a phosphoramide linker, and on the other hand two imidazole-based neutral lipids, having as well either an amide or a phosphoramide function as linker. In an embodiment, assemblies can be obtained when the cationic and helper lipids comprise different linkers. See, Colombani, et al., Self-assembling complexes between binary mixtures of lipids with different linkers and nucleic acids promote universal mRNA, DNA and siRNA delivery. J. Control Release. (2017) doi: 10.1016/j.jconrel.2017.01.041

In an embodiment, the nanoparticles can be developed according to selective organ targeting (SORT) wherein multiple classes of lipid nanoparticles are systematically engineered to exclusively edit extrahepatic tissues via addition of a supplemental SORT molecule. See, e.g., Cheng et al., Nature Nanotechnology 15, 313-320 2020). The approach has been shown with dendrimer lipid nanoparticles (DLNPs), stable nucleic acid lipid particles (SNALPs), and lipid-like nanoparticles (LLNPs), including with use of ionizable cationic lipids (5A2-SC8, C12-200, or DLin-MC3-DMA)36,48,49, zwitterionic lipids (DOPE or DSPC), cholesterol, DMG-PEG, and permanently cationic lipids (DOTAP, DDAB or EPC). Wei et al., Systemic nanoparticle delivery of CRISPR-Cas9 ribonucleproteins for effective tissue specific genome editing, Nature Comm. (2020) 11:3232, doi:10.1038/s4146020170293, incorporated herein by reference.

In one embodiment, the composition comprises a plurality of lipid nanoparticles comprising a cationic lipid, a neutral lipid, a cholesterol, a PEG lipid, or a combination thereof, wherein the plurality of lipid nanoparticles optionally has a mean particle size of between 80 nm and 160 nm; and wherein the lipid nanoparticles comprise one or more polynucleotides encoding at least one polypeptide of the present disclosure, e.g., METTL17 polypeptide.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-2200, December 2011).

Further cationic lipids may comprise di-O-octadecenyl-3-trimethylammonium-propane, (DOTMA), 1,2-dioleoyl-sn-glycero-3-phosphoethanolamine (DOPE), 1,2-dioleoyl-3-trimethylammonium-propane (DOTAP), a biodegradable analogue of DOTMA, alone or in combination with further materials such as, for example cholesterol. Such Cationic lipid LNPs can be delivered as, for example, nanoemulsions and may further incorporate carbonate apatite (increase interaction between particles and cell membranes), or with conjugation with fibronectin, accelerating endocytosis. Other quaternary ammonium lipids, such as Dimethyldioctadecylammonium bromide (DDAB) are also 2,3-dioleyloxy-N-[2-(sperminecarboxamido) ethyl]-N,N-dimethyl-1-propanaminium trifluoroacetate (DOSPA) are also contemplated for use in delivery.

Lipid nanoparticles for mRNA delivery can comprise 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H—cyclopenta[a]phenanthren-3-yl)oxy)carbonyl)amino)-N,N-bis(2-hydroxyethyl)-N-methylethan-1-aminium bromide (BHEM—Cholesterol). See, Zhang, Y. et al. In situ repurposing of dendritic cells with CRISPR/Cas9-based nanomedicine to induce transplant tolerance. Biomaterials 217, 119302 (2019), incorporated herein by reference.

In some embodiments, the lipid nanoparticle comprises a molar ratio of 20-60% ionizable cationic lipid, 5-25% non-cationic lipid, 25-55% sterol, and 0.5-15% PEG-modified lipid.

In some embodiments, the lipid nanoparticle is any nanoparticle described in U.S. Pat. No. 10,442,756, and/or comprises any compound described in U.S. Pat. No. 10,442,756, including but not limited to a nanoparticle according to any one of Formulas (IA) or (II) described therein.

In some embodiments, the lipid nanoparticle is any nanoparticle described in e.g., U.S. Pat. No. 10,266,485, and/or comprises any compound described in U.S. Pat. No. 10,266,485, including but not limited to a nanoparticle according to Formula (II) described therein.

In some embodiments, the lipid nanoparticle is a nanoparticle described in U.S. Pat. No. 9,868,692, and/or comprises a compound described in e.g., U.S. Pat. No. 9,868,692, including but not limited to a nanoparticle according to Formula (I), (IA), (II), (IIa), (IIb), (IIc), (IId), (IIe),

In some embodiments, a lipid nanoparticle comprises compounds of Formula (I) and/or Formula (II) as described in U.S. patent Ser. No. 10/272,150.

In some embodiments, the mRNA is formulated in a lipid nanoparticle that comprises a compound selected from Compounds 3, 18, 20, 25, 26, 29, 30, 60, 108-112 and 122 of U.S. Pat. No. 10,272,150.

In some embodiments, at least 80% (e.g., 85%, 90%, 95%, 98%, 99%) of the uracil in the open reading frame have a chemical modification, optionally wherein the vaccine is formulated in a lipid nanoparticle (e.g., a lipid nanoparticle comprises a cationic lipid, a PEG-modified lipid, a sterol and a non-cationic lipid).

In some embodiments, the lipid nanoparticle has a mean diameter of 50-200 nm.

In some embodiments, a lipid nanoparticle comprises Compounds 3, 18, 20, 25, 26, 29, 30, 60, 108-112, or 122 as set forth in U.S. patent Ser. No. 10/272,150.

In some embodiments, the lipid nanoparticle has a polydispersity value of less than 0.4 (e.g., less than 0.3, 0.2 or 0.1).

In some embodiments, a plurality of lipid nanoparticles, such as when contained in a formulation, has a mean PDI of between 0.02 and 0.2. In some embodiments, a plurality of lipid nanoparticles, such as when contained in a formulation comprising one or more polynucleotide(s), has a mean lipid to polynucleotide ratio (wt/wt) of between 10 and 20.

In some embodiments, the lipid nanoparticle has a net neutral charge at a neutral pH value.

### Liposomes

In one embodiment, a lipid particle may be liposome. Liposomes are spherical vesicle structures composed of a uni- or multilamellar lipid bilayer surrounding internal aqueous compartments and a relatively impermeable outer lipophilic phospholipid bilayer. In one embodiment, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In one embodiment, the liposome comprises a transport polymer, which may optionally be branched, comprising at least 10 amino acids and a ratio of histidine to non-histidine amino acids greater than 1.5 and less than 10. The branched transport polymer can comprise one or more backbones, one or more terminal branches, and optionally, one or more non-terminal branches. See, U.S. Pat. No. 7,070,807, incorporated herein by reference in its entirety. In one embodiment, the transport polymer is a Histidine-Lysine co-polymer (HKP) used to package and deliver mRNA and other cargos. See, U.S. Pat. Nos. 7,163,695, and 7,772,201, incorporated herein by reference in their entireties.

### Stable Nucleic-Acid-Lipid Particles (SNALPs)

In one embodiment, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMA).

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

### Lipoplexes/Polyplexes

In one embodiment, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2 (e.g., forming DNA/Ca²⁺ microcomplexes), polyethenimine (PEI) (e.g., branched PEI), and poly(L-lysine) (PLL). Core-shell structured lipoplyplex delivery platforms can also be used and are one preferred delivery for mRNA, particularly because the core-shell structured particle can protein and gradually release mRNA upon degradation of the polymers. See, U.S. Patent Publication 2018/0360756, incorporated herein by reference.

### Cell Penetrating Peptides

In one embodiment, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin 33 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the METTL17 polypeptide directly, which is then complexed with the nucleic acid component and delivered to cells. In some examples, separate delivery of CPP—METTL17 and CPP-nucleic acid component to multiple cells may be performed. CPP may also be used to delivery RNPs.

CPPs may be used to deliver the compositions and systems to plants. In some examples, CPPs may be used to deliver the components to plant protoplasts, which are then regenerated to plant cells and further to plants.

### DNA Nanoclews

In one embodiment, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. DNA nanoclew may have a palindromic sequences to be partially complementary to the nucleic acid component molecule within the METTL17 polypeptide:nucleic acid component ribonucleoprotein complex. A DNA nanoclew may be coated, e.g., coated with PEI to induce endosomal escape.

### Gold Nanoparticles

In one embodiment, the delivery vehicles comprise gold nanoparticles (also referred to AuNPs or colloidal gold). Gold nanoparticles may form complex with cargos, e.g., METTL17 polypeptide:nucleic acid component RNP. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901.

iTOP

In one embodiment, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D′Astolfo D S, Pagliero R J, Pras A, et al. (2015). Cell 161:674-690.

### Polymer-Based Particles

In one embodiment, the delivery vehicles may comprise polymer-based particles (e.g., nanoparticles). In one embodiment, the polymer-based particles may mimic a viral mechanism of membrane fusion. The polymer-based particles may be a synthetic copy of Influenza virus machinery and form transfection complexes with various types of nucleic acids (siRNA, miRNA, plasmid DNA or nucleic acid component, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In one embodiment, the polymer-based particles may comprise alkylated and carboxyalkylated branched polyethylenimine. In some examples, the polymer-based particles are VIROMER, e.g., VIROMER RNAi, VIROMER RED, VIROMER mRNA. Example methods of delivering the systems and compositions herein include those described in Bawage S S et al., Synthetic mRNA expressed Cas13a mitigates RNA virus infections, biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data, doi:10.13140/RG.2.2.23912.16642.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6:e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In one embodiment, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; El-Andaloussi S, et al., Nat Protoc. 2012 Dec. 7(12):2112-26; Uno Y, et al., Hum Gene Ther. 2011 Jun. 22(6):711-9; Zou W, et al., Hum Gene Ther. 2011 Apr. 22(4):465-75. Exemplary exosomes can be generated from 293F cells, with mRNA-loaded exosomes driving higher mRNA expression than mRNA loaded LNPs in some instances. See, e.g., J. Biol. Chem. (2021) 297(5) 101266.

In some examples, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427 h.

### Retrovirus Like Delivery Systems

The delivery vehicle may comprise a retro-virus like protein, such as PEG10, which is capable of incorporating a cargo into a virus-like particle. As such systems can be re-programmed to package specific cargos, polynucleotides encoding components of the METTL17 systems disclosed herein may be further modified with a recognition sequence that leads to selective packaging of the METTL17 components into such retro-virus like VLPs. Said VLPs may be further modified with fusogenic proteins that impart tissue or cell specificity. Example systems are disclosed in Segel et al. Mammalian retrovirus-like protein PEG10 packages its own mRNA and can be pseudotyped for mRNA delivery. 373 Science, 882-889 (2021), which is incorporated herein by reference. The harnessing of natural proteins that formvirus-like particles and can deliver mRNA cargo, or Selective Endogenous eNcapsidation for cellular Delivery (SEND), may reduce immunogenic response compared to other delivery approaches.

### Methods of Screening

Described in certain example embodiments are methods of identifying, from one or more candidate (or test) agents, capable of increasing expression or activity a METTL17 encoding polynucleotide or polypeptide. In some embodiments activity of METTL17 comprises Fe—S cluster binding Screening can be low or high-throughput. In some embodiments, screening of test agents involves testing a combinatorial library containing a large number of potential METTL17 modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks. A chemical library can contain nucleic acids (e.g., guide RNAs, RNAi molecules, or functional RNAs), peptides and polypeptides, small molecules, and/or the like. Exemplary chemical screening libraries are generally known and can be applied in a method of the present disclosure. See e.g., CORE library and EXPRESS-Pick library from Chem Bridge. Others will be readily known to one of ordinary skill in the art. In some embodiments, the screen is a gRNA screen.

A further aspect of the disclosure relates to a method for identifying an agent capable of modulating, e.g., increasing, expression and/or activity of a METTL17 encoding polynucleotide and/or polypeptide as disclosed herein, comprising: a) applying a candidate agent to the cell or cell population; b) modulation of one or more phenotypic aspects of the cell or cell population by the candidate agent, thereby identifying the agent. The phenotypic aspects of the cell or cell population that is modulated may be e.g., an increase in expression and/or activity of a METTL17 encoding polynucleotide or polypeptide, a gene signature or biological program specific to an increase in mitochondria function and/or activity, particularly respiration, and/or modulation of mitochondria function and/or activity, particularly respiration. In some embodiments activity of METTL17 comprises Fe—S cluster binding. In certain embodiments, steps can include administering candidate modulating agents to cells, detecting identified cell (sub)populations for changes in signatures, or identifying relative changes in cell (sub) populations which may comprise detecting relative abundance of particular gene signatures.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of an immune cell or immune cell population may be modulated without substantially altering other (unintended, undesired) phenotypic aspect(s).

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein. Agents can include complexes or components of complexes, such as CRISPR-Cas systems or CRISPR-based systems such as any of those described elsewhere herein.

Screening can be done in vitro, ex vivo, in situ, or in vivo.

Further embodiments are illustrated in the following Examples which are given for illustrative purposes only and are not intended to limit the scope of the disclosure.

### EXAMPLES

### Example 1—Testing METTL17 Expression Effects in Models of Mitochondrial Disease

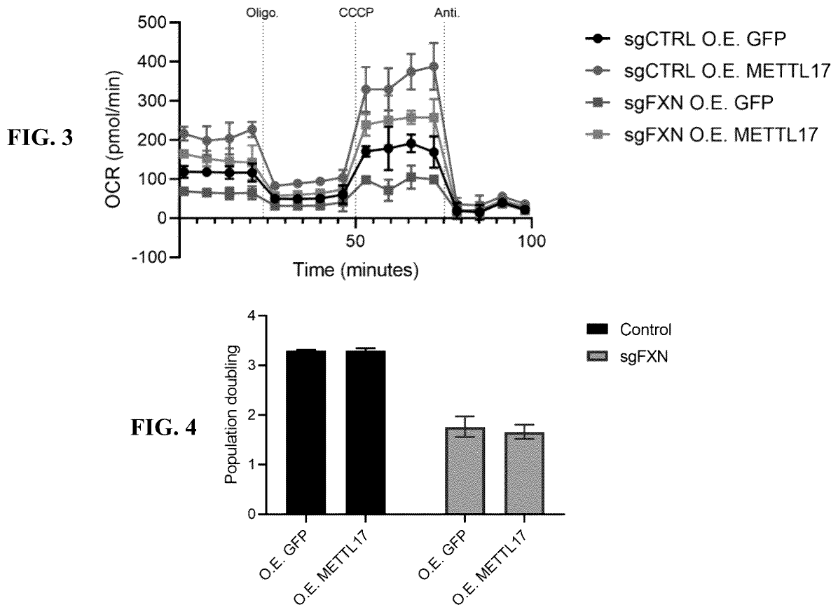

Applicants have identified a factor, METTL17, that appears to be sufficient for boosting mitochondrial respiratory chain activity and can rescue some cellular models of mitochondrial disease. Using proteomics, Applicants found that this factor is depleted in cellular models of Friedreich's ataxia, the most common Mendelian mitochondrial disease due to a recessive loss of a gene called frataxin (FXN). Consistent with the METTL17's known role in intra-mitochondrial protein translation, FXN null cells exhibit blunted intra-mitochondrial translation and the synthesis of the mitochondrial encoded respiratory chain proteins. Applicants observed that if Applicants overexpress METTL17, it is properly targeted to the mitochondria (FIG. 1). METTL17 overexpression serves to boost intra-mitochondrial protein translation in FXN null cells, but what is surprising is that it also boosted in control cells, indicating it can be a limiting factor. Moreover, this boost in translation upon METTL17 overexpression in Friedreich's ataxia cells is sufficient to restore their respiratory capacity, as attested to by their ability to survive on the non-fermentable carbon source galactose (FIG. 2). Moreover, METTL17 overexpression boosts both basal and maximal respiration in control and FXN null cells (FIG. 3). A key insight from Applicant's work is that METTL17 is limiting for the expression of the mtDNA encoded subunits of the mitochondrial respiratory chain. However, METTL17 overexpression has no effect, either beneficial or detrimental, to growth in both control and Friedreich's ataxia cells (FIG. 4). Thus, METTL17 overexpression can boost mitochondrial bioenergetics.

Applicants point out that this is a rather unique feature of METTL17. There are many factors, which when depleted, will lead to a loss of mitochondrial respiratory chain activity. However, there are far fewer that Applicants are aware of that are naturally limiting, i.e., their overexpression will boost mitochondrial respiration even in control cells. This is a unique property and holds the potential to be applicable to a myriad of mitochondrial diseases independent of the mutation.

Applicants envision overexpression of METTL17, either as a gene replacement or through another intervention that will boost its expression, as a treatment for diseases and disorders linked to deficiency of the mitochondrial respiratory chain, ranging from monogenic mitochondrial diseases due to mutations in the mtDNA or in the nuclear genome to age-associated diseases. Boosting METTL17 activity would serve to enhance mitochondrial translation, compensating for the deficits in the respiratory chain in these diseases, hence providing a bioenergetic rescue. In the case of Friedreich's ataxia, Applicants know that METTL17 levels are down and hence this represents a potential targeted rescue of the downstream mitochondrial consequence of this disease. But in other diseases of the respiratory chain overexpression of METTL17 is expected to confer a bioenergetic rescue potentially agnostic of the genetic cause. Overexpression of METTL17 in CAR-T cells is predicted to yield a bioenergetic boost that could be useful in cancer therapy. Applicants are currently testing overexpression of METTL17 in other cellular models of mitochondrial disease.

### Example 2—METTL17 is an Fe—S Cluster Checkpoint for Mitochondrial Translation

Friedreich's ataxia (FA) is a progressive neurological disorder impacting 1 in 50,000 people (Keita et al., 2022; Koeppen, 2011; Pandolfo, 2012). While the primary feature of FA is ataxia, this disease is in fact multisystemic. Patients can also develop diabetes, scoliosis, hearing and vision loss, as well as cardiomyopathy, the latter being a leading cause of premature mortality at a median age of 37.5 years (Harding, 1981; Tsou et al., 2011). FA is caused by a recessive depletion of a nuclear-encoded mitochondrial protein frataxin (FXN) (Campuzano et al., 1996), that functions as an allosteric activator of iron-sulfur (Fe—S) cluster biosynthesis (Maio et al., 2020; Parent et al., 2015; Patra and Barondeau, 2019; Srour et al., 2020). Tissue and cell samples from FA patients contain 5-30% residual FXN levels (Campuzano et al., 1997; Deutsch et al., 2010). This depletion is most often due to the expansion of a naturally occurring GAA track found within the first intron of the gene (Campuzano et al., 1996; Durr et al., 1996; Filla et al., 1996; Reetz et al., 2015) which triggers loss of FXN expression (Greene et al., 2007; Groh et al., 2014; Soragni et al., 2008). FA is the most common monogenic mitochondrial disease and the most common inherited ataxia. Yet there still is a lack of approved therapies for FA, and the standard of care focuses on symptomatic management.

Fe—S clusters are ancient and universal redox cofactors (Beinert et al., 1997; Boyd et al., 2014; Braymer et al., 2021; Tsaousis, 2019). In humans, there are ˜60 known Fe—S cluster binding apoproteins (Andreini et al., 2016; Lill and Freibert, 2020) that operate throughout the mitochondrion, cytosol and nucleus. For all these subcellular compartments, cluster biosynthesis is initiated in the mitochondria where FXN accelerates Fe—S cluster formation (Parent et al., 2015; Patra and Barondeau, 2019; Srour et al., 2020). These versatile cofactors can take part in a variety of functions; while the most widely appreciated one is electron transfer, they also participate in enzyme catalysis, sulfur mobilization, and redox sensing (Braymer et al., 2021; Lill and Freibert, 2020; Maio and Rouault, 2020). In addition, there is evidence that in some cases Fe—S clusters contribute to the structural stability of the apoproteins into which they are integrated. Fe—S cluster binding apoproteins function in diverse cellular processes such as DNA replication and repair, nucleotide biosynthesis and energy metabolism (Maio and Rouault, 2020; Rouault, 2015). Indeed, in FA it has been well documented in patient samples as well as several disease models that there is a deficit in mitochondrial oxidative phosphorylation (OXPHOS) leading to bioenergetic defects (Gonzalez-Cabo et al., 2005; Lin et al., 2017; Lodi et al., 1999; Puccio et al., 2001; Rotig et al., 1997). It has been suggested that one reason for the neuronal and cardiac deficits observed in FA might be the high-energy demand of these tissues (Burk, 2017; Cooper and Schapira, 2003; Lynch et al., 2012).

To systematically understand the cellular consequences of FXN loss, Applicants performed both proteomic and genetic interaction mapping on the background of FXN deficiency. Unexpectedly, Applicants find that a consequence of FXN loss is a significant impairment in mitochondrial protein translation. Applicants determined that METTL17—a conserved mitoribosome assembly factor (Shi et al., 2019)—harbors a previously unrecognized Fe—S cluster that stabilizes its binding to the mitoribosomal small subunit. As a result, in FXN deficient cells, METTL17 activity is diminished. The data suggest that METTL17 loss is a mitochondrial repercussion of FXN deficiency and contributes to an intra-organelle translational defect.

### Methods

### Cell Lines

K562 (female), HEK293T (female) and A549 (male) cells were obtained from the ATCC and maintained in DMEM (GIBCO) with 25 mM glucose, 10% fetal bovine serum (FBS, Invitrogen), 4 mM Glutamine, 1 mM sodium pyruvate, 50 μg/mL uridine, and 100 U/mL penicillin/streptomycin under 5% CO2 at 37° C. When necessary, K562 cells were selected with 2 μg/ml puromycin (GIBCO) or 500ug/ml Geneticin (GIBCO), HEK293T and A549 cells were selected with 1 μg/ml puromycin (GIBCO). Cell lines were authenticated by STR profiling (ATCC). Cells were tested to ensure absence of mycoplasma by PCR-based assay once every 3 months. For experiments involving 1% oxygen, cells were placed in 37° C. incubators, attached to a nitrogen supply which pulsed N2 and maintained in 1% 02 and 5% CO2.

### Plasmids

Individual sgRNAs were cloned into pLentiCRISPRv2 (Addgene 52961) (Sanjana et al., 2014). For genetic interaction assays, cells were infected with pRDA_186 plasmid (Addgene 133458), a gift from John Doench (Broad Institute), bearing guides against a control locus or FXN. C-terminally FLAG tagged, codon optimized cDNAs were cloned in pLYS6, bearing a Neomycin selection cassette, using the NheI and EcoRI sites. All plasmids were verified by sequencing. pMD2.G (Addgene 12259) and psPAX2 (Addgene 12260) were used for lentiviral packaging.

### Lentivirus Production

2.5×106 HEK293T cells were seeded in 5 ml in a T25 cm² flask (one flask per lentivirus). The following day the cells were transfected with 1 ml of transfection mixture per well. The transfection mixture contained 25 μl Lipofectamine 2000 (Thermo Fisher Scientific), 3.75pg psPAX2, 2.5 μg pMD2.G, 5 μg of the lentiviral vector of interest and Opti-MEM medium (GIBCO) up to 1 ml. The mixture was incubated at room temperature for 20 min before adding it to cells. 6 h following transfection, the media was replaced with fresh DMEM. Two days after transfection, media was collected, filtered through a 0.45 um filter and stored at −80 degrees C.

### Proteomics

sgCtrl and sgFXN K562 cells were grown in duplicate flasks for 10 days. Quantitative proteomics was performed at the Thermo Fisher Scientific Center for Multiplexed Proteomics (Harvard).

### Sample Preparation for Mass Spectrometry

Samples were prepared essentially as previously described (Li et al., 2021; Navarrete-Perea et al., 2018). Following lysis, protein precipitation, reduction/alkylation and digestion, peptides were quantified by micro-BCA assay and 100 μg of peptide per sample were labeled with TMT reagents (Thermo-Fisher) for 2 hrs at room temperature. Labeling reactions were quenched with 0.5% hydroxylamine and acidified with TFA. Acidified peptides were combined and desalted by Sep-Pak (Waters).

### Basic pH Reversed-Phase Separation (BPRP)

TMT labeled peptides were solubilized in 5% ACN/10 mM ammonium bicarbonate, pH 8.0 and separated by an Agilent 300 Extend C18 column (3.5 μm particles, 4.6 mm ID and 250 mm in length). An Agilent 1260 binary pump coupled with a photodiode array (PDA) detector (Thermo Scientific) was used to separate the peptides. A 45 minute linear gradient from 10% to 40% acetonitrile in 10 mM ammonium bicarbonate pH 8.0 (flow rate of 0.6 mL/min) separated the peptide mixtures into a total of 96 fractions (36 seconds). A total of 96 Fractions were consolidated into 24 samples in a checkerboard fashion, acidified with 20 μL of 10% formic acid and vacuum dried to completion. Each sample was desalted via Stage Tips and re-dissolved in 5% FA/5% ACN for LC-MS3 analysis.

### Liquid Chromatography Separation and Tandem Mass Spectrometry (LC-MS3)

Proteome data were collected on an Orbitrap Eclipse mass spectrometer (ThermoFisher Scientific) coupled to a Proxeon EASY-nLC 1200 LC pump (ThermoFisher Scientific). Fractionated peptides were separated using a 180 min gradient at 500 nL/min on a 35 cm column (i.d. 100 m, Accucore, 2.6 m, 150 Å) packed in-house. MS1 data were collected in the Orbitrap (120,000 resolution; maximum injection time 50 ms; AGC 4×10⁵). Top 10 precursors with charge states between 2 and 5 were required for MS2 analysis, and a 90 s dynamic exclusion window was used. MS2 scans were performed in the ion trap with CID fragmentation (isolation window 0.5 Da; Rapid; NCE 35%; maximum injection time 35 ms; AGC 1.5×10⁴). An on-line real-time search algorithm (Orbiter) was used to trigger MS3 scans for quantification (Schweppe et al., 2020). MS3 scans were collected in the Orbitrap using a resolution of 50,000, NCE of 55%, maximum injection time of 150 ms, and AGC of 1.5×10⁵. The close out was set at two peptides per protein per fraction (Schweppe et al., 2020).

### Data Analysis

Raw files were converted to mzXML, and monoisotopic peaks were re-assigned using Monocle (Rad et al., 2021). Searches were performed using SEQUEST (Eng et al., 1994) against a human database downloaded from Uniprot in 2014. Applicants used a 50 ppm precursor ion tolerance and 0.9 Da product ion tolerance for MS2 scans collected in the ion. TMT on lysine residues and peptide N-termini (+229.1629 Da) and carbamidomethylation of cysteine residues (+57.0215 Da) were set as static modifications, while oxidation of methionine residues (+15.9949 Da) was set as a variable modification.

Each run was filtered separately to 1% False Discovery Rate (FDR) on the peptide-spectrum match (PSM) level. Then proteins were filtered to the target 1% FDR level across the entire combined data set. For reporter ion quantification, a 0.003 Da window around the theoretical m/z of each reporter ion was scanned, and the most intense m/z was used. Reporter ion intensities were adjusted to correct for isotopic impurities of the different TMT reagents according to manufacturer specifications. Proteins were filtered to include only those with a summed signal-to-noise (SN)≥100 across all TMT channels. For each protein, the filtered peptide TMT SN values were summed to generate protein quantification values. To control for different total protein loading within a TMT experiment, the summed protein quantities of each channel were adjusted to be equal within the experiment.

### Mito Translation Assay

2.5×10⁶ cells expressing the corresponding sgRNAs were washed in PBS and incubated for 30 min in 1 mL of labeling medium (10% dFBS, 1 mM sodium pyruvate, and 50 g/mL uridine in DMEM without methionine/cysteine; Life Technologies). Emetine (Sigma) was added to a final concentration of 200 g/mL, and cells were incubated for 5 min before addition of 200 μCi 35S-labeled methionine/cysteine mixture (PerkinElmer) and incubation for 1 hr at 37° C. Cells were recovered and washed twice in PBS before lysis in RIPA buffer (50 mM Tris-HCl pH 7.5, 150 mM NaCl, 1.0% NP-40, 0.5% sodium deoxycholate, 0.1% SDS, 1× protease and phosphatase inhibitor (Cell Signaling), and 250 units/ml benzonase nuclease (Sigma)). 40 g of total proteins were loaded on a 10%-20% SDS-PAGE (Life Technologies) and transferred to a nitrocellulose membrane, 0.45 μm (BioRad). Total protein in each lane was assessed by ponceau S (ThermoFisher) staining and recorded before autoradiography. In all experiments, a replicate of the control lane was treated with chloramphenicol (50 g/mL) to ensure the mitochondrial origin of the 35S signal. The name associated with each band is proposed based on their relative abundance and molecular weight and is informed by prior studies (Fernindez-Silva et al., 2007).

### Genetic Interaction Screening

### Screening

K562 cells were infected with pRDA_186 lentiviral vectors, which express sgRNA against a control locus or FXN, blasticidin resistance from the PGK promoter, and a 2A site-expressing EGFP. Cells were sorted for low GFP expression and expanded.

For the screen, the all-in-one Brunello barcoded library was utilized. This library contains 77,441 sgRNA; an average of 4 guides per gene and 1000 non-targeting control guides. Infections were performed in distinct duplicate at a predetermined MOI of ˜0.5 in 12-well plates with 5 μg/mL polybrene supplementation. Cells were infected for 2 h under centrifugation at 1000×g, incubated for 22 h under standard culturing conditions and pooled 24 h post-centrifugation. Infections were performed with 6.5×107 cells per replicate, in order to achieve a representation of at least 300 cells per sgRNA following puromycin selection. 48 hours after infection, cells were selected with puromycin for 2 days to remove uninfected cells. Cells were passaged in fresh media every 2-3 days. Cells were harvested 11 days after initiation of treatment.

For all screens, genomic DNA (gDNA) was isolated using the XL Maxi NucleoSpin Blood kit (Macherey-Nagel) according to the manufacturer's protocol. PCR and sequencing were performed as previously described (Doench et al., 2016; Piccioni et al., 2018). Samples were sequenced on a MiSeq (Illumina).

### Analysis

Analysis of genetic interactors was performed as previously described (To et al. 2019). Briefly, raw sgRNA read counts were normalized to reads per million and then log 2 transformed using the following formula:

Log₂(Reads from an individual sgRNA/Total reads in the sample×10⁶+1)

Log₂ fold-change of each sgRNA was determined relative to guide abundance in the lentiviral library. The replicates were paired throughout the course of the screens.

For each gene and for each replicate, the mean log 2 fold-change in the abundance of all 4 sgRNAs was calculated. Log₂ fold-changes were averaged by taking the mean across replicates. For each treatment, a null distribution was defined by the 3,726 genes with lowest expression (log 2 FPKM=−7) according to publicly available K562 RNA-seq dataset (sample GSM854403 in GEO series GSE34740). To score each gene, its mean log 2 fold-change across replicates was Z-score transformed, using the statistics of the null distribution defined as above.

### Polyacrylamide Gel Electrophoresis and Protein Immunoblotting

2-5×10⁶ cells were harvested, washed in cold PBS and lysed for 10 min on ice in RIPA lysis buffer (50 mM Tris-HCl pH 7.5, 150 mM NaCl, 1.0% NP-40, 0.5% sodium deoxycholate, 0.1% SDS, 1x HALT protease phosphatase inhibitor (Thermo Fisher Scientific), and Pierce Universal Nuclease for Cell Lysis (Thermo Fisher Scientific). Lysates were further clarified by centrifugation for 10 min at 16000×g at 4 degrees C. Protein concentration was measured using Pierce 660 nm Protein Assay (Thermo Fisher Scientific). 30 g was loaded per well. Electrophoresis was carried out on Novex Tris-Glycine 4-20% or 10-20% gels (Life Technologies) before transfer on a nitrocellulose membrane, 0.45 μm (BioRad). Membranes were blocked for 60 mins with Odyssey Blocking Buffer (LI-COR Biosciences) at RT. Membranes were then incubated with primary antibody, diluted in 3% BSA, for 1 h at RT or overnight at 4 degrees C. Membranes were then washed at RT 5 times in TBST for 5 mins. The membrane was incubated with goat α-rabbit or α-mouse conjugated to IRDye800 or to IRDye680 (LI-COR Biosciences), diluted in 5% milk, for 1 h at RT. Membranes were washed 3 times in TBST for 5 mins and were scanned for infrared signal using the Odyssey Imaging System (LI-COR Biosciences). Band intensities were analyzed with Image Studio Lite (LI-COR Biosciences).

### Antibodies

Antibodies used are shown in Table 11.

TABLE 11
Antibodies used in Example 2

| Antigen | Cat. Number | Vendor |
|---|---|---|
| ACTIN | A3853 | Sigma |
| CDK5RAP1 | 14740-1-AP | Proteintech |
| DYKDDDDK | 2368 | Cell Signaling |
| (SEQ ID NO: 59) TAG | | |
| FLAG | F1804 | Sigma |
| FXN | ab175402 | Abcam |
| HSP60 | ab45134 | Abcam |
| METTL17 | HPA002955 | Atlas |
| OXPHOS | ab110411 | Abcam |
| POLD1 | 15646-1-AP | Prteintech |
| TUBULIN | MA5-16308 | Thermo Fisher |
| TUBULIN | 2128 | Cell signaling |

Cell Viability Assay in Galactose Vs. Glucose

To measure viability in galactose vs glucose, cells were washed in PBS, counted and an equal number of cells was seeded in culture media containing 25 mM glucose or 25 mM galactose with 10% dialyzed FBS. 24 h later, cells were collected and viable cells were determined using a Vi-Cell Counter (Beckman).

qPCR

2.5×10⁶ cells were collected per sample. RNA was extracted from total cells with an RNeasy plus kit (QIAGEN) and DNase-I digested before murine leukemia virus (MLV) reverse transcription using random primers (Promega). qPCR was performed using the TaqMan technology (Life Technologies), using probes Hs02596859_g1 (12S), Hs02596860_s1 (16S), Hs00224159_ml (METTL17) and Hs00427620_ml (TBP). All data were normalized to TBP.

Formaldehyde RNA Immunoprecipitation (fRIP) Assay to METTL17-12S Binding

fRIP assay was performed as previously described (Hendrickson et al, 2016) with some minor changes. Briefly, 5×10⁶ K562 cells were cross-linked with 0.1% formaldehyde (Polysciences, 04018) and slowly rotated for 10 min at room temperature. To quench this reaction, glycine was added to a final concentration of 125 mM and incubated for 5 min at room temperature with rotation. Cells were collected and spun down at 300 g for 5 min at 4° C. The supernatant was removed and the pellet was washed with 1 mL cold PBS. The supernatant was removed and the pellet was resuspended in 1 ml RIPA buffer (buffer (50 mM Tris-HCl pH 7.5, 150 mM NaCl, 1.0% NP-40, 0.5% sodium deoxycholate, 0.1% SDS, 1× protease and phosphatase inhibitor (Cell Signaling), and 250 units/ml benzonase nuclease (Sigma)). The cells were sonicated for 3 times each (10 sec per cycle, amplitude 7 m) with a sonifier (Branson). Samples were then further lysed on ice for 10 min. To remove cell debris, the extract was centrifuged at 12,000 g for 10 min at 4° C. 150 μL of the supernatant was removed and stored at −20° C. (input sample), and the remainder of the sample was used for precipitation. Anti-FLAG® M2 magnetic beads (Sigma) were equilibrated 3 times with 1 mL RIPA buffer. 20 μL of anti-FLAG beads were used for precipitation. The IP was performed overnight at 4° C. on a spinning wheel.

Beads were recovered after extensive washing in RIPA buffer. After the last washing step, the supernatant was removed, 100 μL of RNAse free water with RNAsin (40 U/mL) was added and samples incubated in a thermomixer (Eppendorf) at 300 rpm for at 55° C. for 1 h for decrosslinking. 1 ml of Trizol (Invitrogen) was then added to each sample, and RNA extraction was performed per the manufacturer's recommendations. RNA samples were subsequently quantified for 12S levels by qPCR, and the values were normalized to input 12S and FLAG tagged protein levels.

mtDNA Copy Number

1×10⁵ cells from each condition (n=3) were harvested and lysed in 100 μL mtDNA lysis buffer (25 mM NaOH, 0.2 mM EDTA) before incubation at 95° C. for 15 min. 100 μL of 40 mM Tris-HCl pH 7.5 was added to neutralize the reaction on ice. Samples were diluted 50x and the ratio between mitochondrial and nuclear DNA was determined using a custom Taqman based assay and qPCR using a CFX Opus 384 quantitative PCR machine (Biorad). Relative mtDNA abundance was determine using the ΔΔCt method.

### Mitostring

2×10⁶ cells were collected per sample. RNA was extracted from total cells with an RNeasy kit (QIAGEN). MitoString is a mitochondrial-specific version of NanoString and was performed as previously described (Wolf and Mootha, 2014). All counts were normalized to TUBB.

### Mitochondrial Isolation

5×10⁷ cells were harvested, washed in PBS, and washed once with 10 mL of MB buffer (210 mM mannitol, 70 mM sucrose, 10 mM HEPES-KOH at pH 7.4, 1 mM EDTA, protease/phosphatase inhibitor). Cells were resuspended in 1 mL of MB buffer supplemented with 1×HALT protease phosphatase inhibitor (Thermo Fisher Scientific) and transferred to 2 mL glass homogenizer (Kontes). Cells were broken with ˜35 strokes of a large pestle on ice. Sample volume was then increased to 6 mL with MB buffer. The mixture was centrifuged at 2,000×g for 5 min and the pellet (nuclei and intact cells) was discarded. The supernatant was centrifuged at 13,000×g for 10 min at 4° C. The mitochondrial pellets were washed with MB buffer once, and resuspended in RIPA lysis buffer.

### Oxygen Consumption

1.50×10⁵ K562 cells per well were plated on a Seahorse plate coated with Cell-Tak Cell and Tissue Adhesive (Corning Life Sciences) in XF DMEM (Agilent) containing 5.55 mM glucose, 4 mM Glutamine, 1 mM sodium pyruvate, and oxygen consumption was recorded using a Seahorse XF96 Analyzer (Seahorse Biosciences). Each measurement was performed over 6 min after a 3 min mix and a 3 min wait period. Basal measurements were collected 4 times, followed by 4 measurements after addition of oligomycin (final concentration 2 μM), followed by 4 measurements after addition of Bam15 (final concentration 3 μM), followed by 4 measurements after addition of Piericidin A+ Antimycin A (final concentration 0.5 μM).

### Proliferation Assays

Cell proliferation assays were performed 7-8 days following lentiviral infection. Cells were seeded at an initial density of 1×105 cells/mL and cultured for 3 days. Viable cell number was then determined using a Vi-Cell Counter (Beckman).

### Expression and Purification of METTL17-8XHIS

A human METTL17 construct was designed for expression in E. coli in which the N-terminal mitochondrial targeting sequence was removed and a C-terminal TEV site and 8×HIS-tag were introduced. The METTL17-8XHIS gene was codon optimized for E. coli and ligated into a pET-30a(+) expression vector. The protocol for overexpression and purification of METTL17-8XHIS was adapted from that used for the yeast homolog (Alam et al., 2021). METTL17-8XHIS was overexpressed in OverExpress C41(DE3) cells in LB supplemented with 50 M FeCl3 and 300 M L-cysteine at 37° C. Cells were grown until an OD600 of 0.8 and then expression was induced with 1 mM IPTG. Cells were pelleted after 4 hours, then frozen and stored in liquid nitrogen until purification. At the time of purification, pellets were thawed and resuspended in Buffer A containing 500 mM NaCl, 5 mM imidazole, 5% glycerol, 1 mM TCEP, 40 mM HEPES pH 7.4, and supplemented with lysonase, benzonase, DNase, ribonuclease A, Roche Complete protease, and PMSF. The cells were lysed using sonication and clarified through centrifugation at 40,000×g for one hour. METTL17-8XHIS was then purified over TALON cobalt resin. After washing the column with Buffer A containing 40 mM imidazole, the protein was washed with 30 CVs of a solution containing 2.5 M NaCl, 5% glycerol, 1 mM TCEP, 40 mM HEPES pH 7.4, and 0.1 mg/mL ribonuclease A and DNase I to remove nucleic acid contamination. The protein was eluted with Buffer A containing 300 mM imidazole and then run over a PD10 column into a buffer containing 500 mM NaCl, 5% glycerol, 1 mM TCEP, and 40 mM HEPES pH 7.4. Sizing of the protein was done over a Superdex 200 increase 10/300 GL column. The variant CYSMut-8×HIS containing the mutations C333S, C339S, C347S, and C404S was expressed and purified using the same method.

### UV/Vis Spectroscopy

UV/vis spectra were collected at room temperature with a Cary 50 UV/vis spectrophotometer using a 1 cm pathlength quartz cuvette. Aerobically purified METTL17-8XHIS or CYSMut-8×HIS was transferred into a Coy Labs glovebox (<5 ppm 02). The protein solution was equilibrated to the glovebox atmosphere for at least 30 min on ice before it was centrifuged for 3 min at 14,000×g and room temperature, then buffer-exchanged into an anaerobic buffer containing 25 mM HEPES, pH 7.5, 10% glycerol, 500 mM NaCl using a PD-10 column (GE Healthcare). Samples were diluted (if necessary) with the same buffer and transferred to a quartz cuvette.

### EPR Spectroscopy

Electron paramagnetic resonance (EPR) spectra were recorded on a Bruker EMX spectrometer at 9.37 GHz as frozen solutions. Samples were transferred to a quartz tube (made from clear fused quartz tubing of 3 mm I.D. & 4 mm O.D. from Wale Apparatus) in a Coy Labs glovebox (<5 ppm 02), capped with a rubber septum, and frozen in liquid N2 outside the glovebox. Each sample contained approximately 200 μL of 30 M aerobically purified METTL17-8XHIS with/without 2 mM Na₂S₂O₄ as a reductant or 0.5 mM indigodisulfonic acid as an oxidant.

### Fe Analysis

Protein concentrations were determined by BCA assay using bovine serum albumin (Thermo Scientific) as standards (Smith et al., 1985). Fe concentrations were determined by inductively coupled plasma mass spectrometry (ICP-MS). ICP-MS data were recorded on an Agilent 7900 ICP-MS instrument. Samples were prepared by first digesting the protein in 70% nitric acid (TraceMetal™ Grade, Fischer) at 60° C., and then diluting it with Milli-Q water such that the final concentration of nitric acid is 2%. Standards for Fe were prepared from a 100 ppm transition metal standard solution (Specpure, Thermo Fischer). All samples and standards contained 1 ppb Tb (final concentration) as an internal standard.

### Mitoribosome Purification for Structural Studies

Yeast mitochondria were harvested as previously described (Amunts et al., 2014). Four litres of S. cerevisiae were grown in YPG media (1% yeast extract, 2% peptone, 3% glycerol) until an optical density at 600 nm (OD600) of 1.8. The cells were then centrifuged at 4,500×g, for 9 min, the pellet washed with pre-cooled distilled water and further centrifuged for 15 min at 4,500×g. The pellet was resuspended in pre-warmed dithiothreitol (DTT) buffer (100 mM Tris-HCl pH 9.3, 10 mM DTT). The cells were pelleted by centrifugation at 3,500×g, for 10 min and resuspended in Zymolyase buffer (20 mM K₂HPO₄—HCl pH 7.4, 1.2 M sorbitol). 1 mg ZymolyaseF100T (MP Biomedicals, LLC) was added per gram wet weight and the solution was shaken slowly at 30° C. for 60 min. This was followed by centrifugation at 4,000×g, for 15 min. The pellet was further resuspended in Zymolyase buffer and centrifuged for a further 15 min at 4,000×g. The pellet was then resuspended in homogenization buffer (20 mM HEPES-KOH pH 7.45, 0.6 M sorbitol, 1 mM EDTA) and lysed with 15 strokes in a glass homogenizer. To separate the cell debris and nuclei from mitochondria the solution was centrifuged at 2000×g, for 20 min and the supernatant collected, followed by further centrifugation at 4500×g, for 20 min. Crude mitochondria were pelleted at centrifugation at of 13,000×g, for 25 min, and further purified on a sucrose gradient in SEM buffer (250 mM sucrose, 20 mM HEPES-KOH pH 7.5, 1 mM EDTA) by ultracentrifugation at 141,000×g for 1 hour. Mitochondrial samples were pooled and lysed in a buffer of 25 mM HEPES-KOH pH 7.5, 100 mM KCl, 25 mM Mg(OAc)₂, 1.7% Triton X-100, 2 mM DTT). Upon centrifugation at 30,000×g, for 20 min, the supernatant was loaded on a 1 M sucrose cushion in a buffer containing 20 mM HEPES-KOH pH 7.5, 100 mM KCl, 20 mM Mg(OAc)₂, 1% Triton X-100, 2 mM DTT. The pellet was resuspended in 10 mM Tris-HCl, pH 7.0, 60 mM KCl, 60 mM NH₄Cl, 10 mM Mg(OAc)₂, and applied on a 15-40% sucrose gradient prepared in the same buffer. The fractions containing the SSU were collected, sucrose removed and buffer exchanged to 20 mM HEPES-KOH pH 7.5, 50 mM KCl, 5.0 mM Mg(OAc)₂, 2.0 mM DTT and 0.05% n-dodecyl-β-D-maltoside by passing through a concentrator (Vivaspin) with a 30 kDa molecular weight cutoff. The final concentration of the mitoribosomes measured at an optical density at 280 nm (OD280) was 3.6.

Production of mtIF3

The sequence of mtIF3/AIM23 gene of yeast S. cerevisiae was inserted in pET30a vector between Nde I and Xho I restriction sites. The plasmid was used to transform E. coli Rosetta strain. The culture was inoculated by diluting 100× with LB and grown until OD600 reached the value of 0.8. Protein production was induced by 0.25 mM IPTG at 30° C. for 3 hours. Biomass was collected by centrifugation at 3500×g for 5 min and resuspended in native lysis buffer (25 mM NaHPO₄, 500 mM NaCl, 25 mM imidazol, pH 7.4). The suspension was sonicated 6×10 sec with 20 sec breaks in between. The debris pelleted by centrifugation at 30000×g for 20 min. The mtIF3 was then purified on HisTrap HP column by metallo-chelating chromatography. The second step of purification and buffer exchange was done on Superdex 75 10/300 GL column in 20 mM HEPES-KOH, 200 mM NaCl, pH 7.0. The fractions were analysed by SDS-PAGE, pooled and concentrated on Vivaspin20 concentrators to 85 μM. Purified mtIF3 was added to the SSU in 5× molar excess and incubated for 30 min.

### Cryo-EM Data Collection and Image Processing

For cryo-EM, 3 μL of −100 nM (A260 4.0 or 6.0) mitoribosome sample was applied onto a glow-discharged (20 mA for 30 sec) holey-carbon grid (Quantifoil R2/1 copper, mesh 300) coated with continuous carbon (of −3 nm thickness) and incubated for 30 sec in a controlled environment of 100% humidity and 4° C. temperature. The grids were blotted for 3.5 sec, followed by plunge-freezing in liquid ethane, using a Vitrobot MKIV (FEI/Thermofischer). The datasets were collected on FEI Titan Krios (FEI/Thermofischer) transmission electron microscope operated at 300 keV with a slit width of 20 eV on a GIF quantum energy filter (Gatan). A K2 Summit detector (Gatan) was used at a pixel size of 0.83 Å (magnification of 165,000×) with a dose of 32 electrons/Å² fractionated over 20 frames. A defocus range of 1.2 to 2.8 m was applied.

Beam-induced motion correction was performed for all data sets using RELION 3.0 (Zivanov et al., 2018). Motion-corrected micrographs were used for contrast transfer function (CTF) estimation with GCTF (Zhang, 2016). Particles were picked by Gautomatch (mrc-lmb.cam.ac.uk/kzhang) with reference-free, followed by reference-aided particle picking procedures. Reference-free 2D classification was carried out to sort useful particles from falsely picked objects, which were then subjected to 3D refinement, followed by a 3D classification with local-angular search. UCFS Chimera (Pettersen et al., 2004) was used to visualize and interpret the maps. 3D classes corresponding to unaligned or low-quality particles were removed. Well-resolved classes were pooled and subjected to 3D refinement and CTF refinement (beam-tilt, per-particle defocus, per-micrograph astigmatism) by RELION 3.1 (Zivanov et al., 2020), followed by Bayesian polishing. Particles were separated into multi optics groups based on acquisition areas and date of data collection. Second round of 3D refinement and CTF refinement (beam-tilt, trefoil and fourth-order aberrations, magnification anisotropy, per-particle defocus, per-micrograph astigmatism) were performed, followed by 3D refinement.

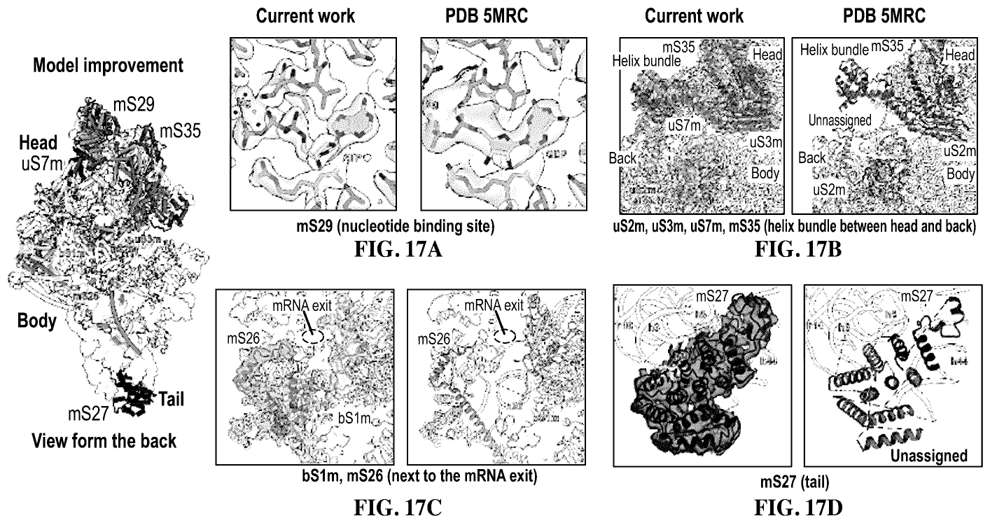

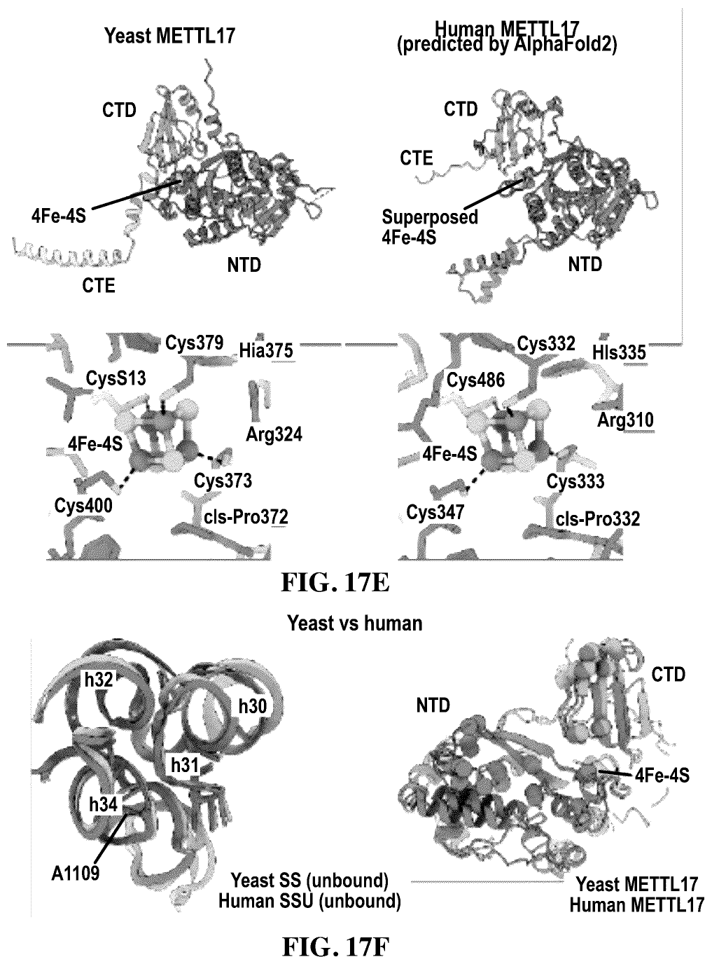

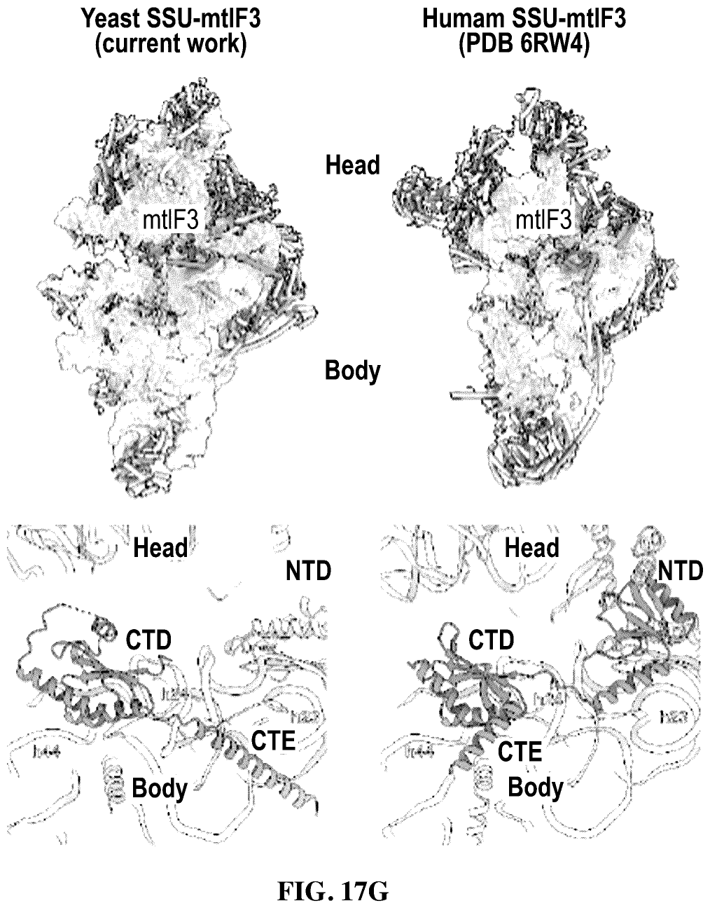

To classify the factor binding states, a non-align focus 3D classifications with particle-signal subtraction using the mask covering the factor binding were done with RELION 3.1. The particles of each state were pooled, subtracted signal was reverted and 3D refinement was done with the corresponding solvent mask. To improve the local resolution, the several local masks were prepared and used for local-masked 3D refinements. Nominal resolutions are based on gold-standard, applying the 0.143 criterion on the Fourier Shell Correlation (FSC) between reconstructed half-maps. Maps were subjected to B-factor sharpening and local-resolution filtering by RELION 3.1, superposed to the overall map and combined for the model refinement. Cryo-EM results are shown at least in FIG. 17A-17G.

### Model Building Refinement, and Analysis

The starting models for SSU was PDB ID 5MRC (Desai et al., 2017). These SSU model was rigid body fitted into the maps, followed by manual revision. Initial model of mtIF3 was generated by SWISS-MODEL (Waterhouse et al., 2018), while that of METTL17 was built manually. Ligands and specific extensions/insertions were built manually based on the density. Coot 0.9 with Ramachandran and torsion restraints (Emsley et al., 2010) was used for manual fitting and revision of the model. Geometrical restraints of modified residues and ligands were calculated by Grade Web Server (grade.globalphasing.org). Final models were subjected to refinement of energy minimization and ADP estimation by Phenix.real_space_refine (Liebschner et al., 2019) with rotamer restraints without Ramachandran restrains, against the composed maps with B-factor sharpening and local-resolution filtering. Reference restrains was also applied for non-modified protein residues, using the input models from Coot as the reference. Metal-coordination restrains were generated by ReadySet in the PHENIX suite and used for the refinement with some modifications. Refined models were validated with MolProbity (Williams et al., 2018) and EMRinger (Barad et al., 2015) in the PHENIX suite. UCSF ChimeraX 0.91 (Goddard et al., 2018) was used to make figures.

### Results

### Loss of FXN Leads to a Widespread Depletion of Fe—S Cluster Containing Proteins

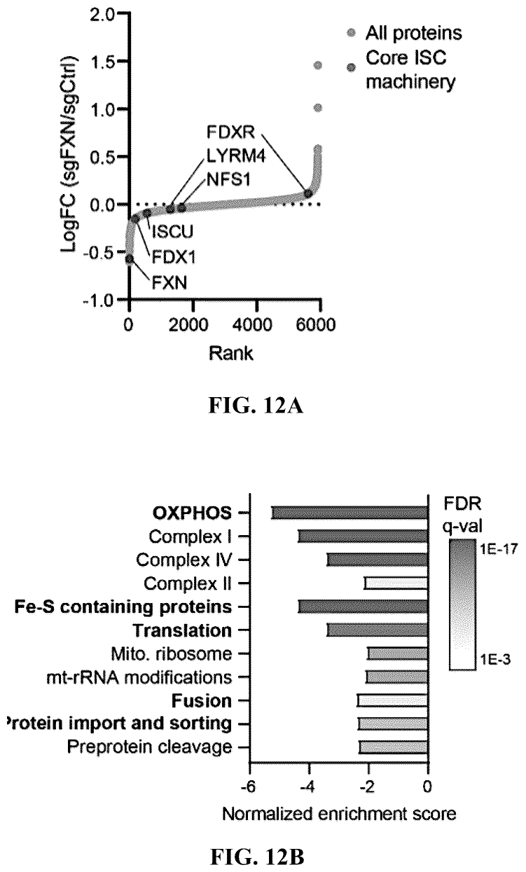

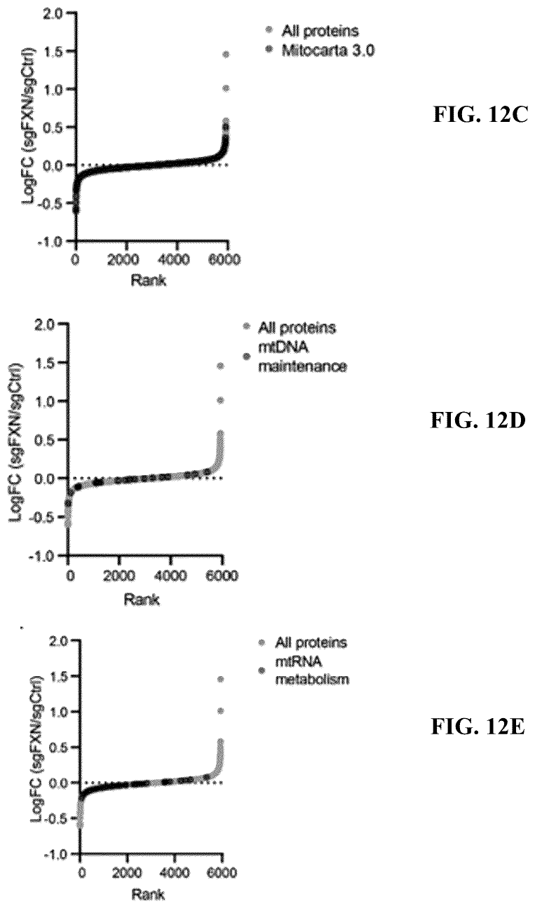

Applicants sought to determine the cellular consequences of loss of FXN, an allosteric activator of Fe—S cluster biosynthesis found in the mitochondria. Applicants performed quantitative, whole-proteome profiling of human K562 cells following CRISPR-based disruption of FXN versus OR11A1, a non-expressed gene that serves as an editing control (FIG. 5A). In this analysis (Table 12), FXN itself scored as the 2nd most depleted protein (FIG. 5B), while its partner ISC machinery (ISCU, NFS1, LYRM4) remained unchanged (FIG. 12A). On the upgoing side, IRP2, an iron regulatory protein known to be stabilized in FXN deficiency, was among the top 100 upregulated proteins. In addition, one of the top upgoing proteins was an apolipoprotein B receptor, APOBR, consistent with recent work showing a striking change in cellular lipid metabolism following the loss of Fe—S cluster biosynthesis (Chen et al., 2016; Crooks et al., 2018; Turchi et al., 2020; Wang et al., 2022). What immediately caught Applicants' attention was that of the 34 known Fe—S cluster containing proteins detected in Applicants' proteomics analysis, 33 were downregulated (FIG. 5B). The only unaffected Fe—S cluster containing protein was NARF, a nuclear protein that is a component of a prelamin-A endoprotease complex (Barton and Worman, 1999). This analysis revealed that depletion of FXN leads to a near universal diminution in Fe—S cluster binding proteins. It is likely that this pervasive depletion takes place at the post-transcriptional level, as these proteins function in and are regulated by distinct pathways. The ubiquity of this depletion suggests that proper incorporation of an Fe—S cluster is essential for the structural integrity of virtually all human Fe—S cluster apoproteins.

TABLE 12
Data collection, processing, model refinement and validation statistics.

| | SSU-METTL17 | SSU-mtIF3 | Consensus SSU |
|---|---|---|---|
| Data collection and | | | |
| Processing | | | |
| Microscope | Titan Krios | Titan Krios | Titan Krios |
| Voltage (kV) | 300 | 300 | 300 |
| Camera | K2 Summit | K2 Summit | K2 Summit |
| Magnification | 165,000 | 165,000 | 165,000 |
| Pixel size at detector | 0.83 | 0.83 | 0.83 |
| (Å/pixel) | | | |
| Total electron | 32 | 32 | 32 |
| exposure (e−/Å2) | | | |
| Exposure rate | 5.6 | 5.6 | 5.6 |
| (e−/pixel/sec) | | | |
| Number of frames | 20 | 20 | 20 |
| Defocus range (μm) | −1.2 to −2.8 | −1.2 to −2.8 | −1.2 to −2.8 |
| Energy filter slit | 20 | 20 | 20 |
| width (V) | | | |
| Micrographs | 14,200 | 6,578 | 14,200 |
| collected (no.) | | | |
| Final particles (no.) | 283,744 | 53,922 | 778,978 |
| Point group | C₁ | C₁ | C₁ |
| Resolution | 2.59 | 2.83 | 2.32 |
| (global, Å) | | | |
| FSC 0.143 | | | |
| (masked) | | | |
| Map-sharpening B | −41 | −37 | −43 |
| factor (Å²) | | | |
| Model composition | | | |
| Atoms | 91,232 | 88,476 | 88,581 |
| RNA residues | 1,478 | 1,477 | 1,485 |
| Protein residues | 7386/1 | 7,056/1 | 7,038/1 |
| (non-modified/ | | | |
| modified) | | | |
| Metal ions | 63/8 | 58/8 | 103/21 |
| (Mg²⁺/K+) | | | |
| Ligands | 1/1 | 1/0 | 1/0 |
| (ATP/4Fe-4S) | | | |
| Waters | 0 | 3 | 3 |
| Model Refinement | | | |
| Model-Map CC | 0.73/0.79/ | 0.80/0.85/ | 0.80/0.84/ |
| (CCmask/CCbox/ | 0.71/0.75 | 0.78/0.82 | 0.77/0.81 |
| CCpeaks/CCvolume) | | | |
| Resolution (Å) by | 2.10/2.12 | 2.40/2.40 | 1.70/1.70 |
| model-to-map FSC, | | | |
| threshold 0.50 | | | |
| (masked/unmasked) | | | |
| Average B factor | 27/21 | 29/35 | 27/23 |
| (Å²) (RNA/protein) | | | |
| R.m.s. deviations, | 0.009/0.799 | 0.002/0.505 | 0.002/0.508 |
| bond lengths (Å)/ | | | |
| bond angles (°) | | | |
| Validation | | | |
| MolProbity score | 1.79 | 1.32 | 1.34 |
| CaBLAM outliers | 1.40 | 1.02 | 1.02 |
| (%) | | | |
| Clash score | 6.82 | 5.82 | 5.48 |
| Rotamer outliers (%) | 3.04 | 0.73 | 1.16 |
| C_{β} deviations | 0 | 0 | 0 |
| Ramachandran plot | 97.90/2.20/0.00 | 98.59/1.39/0.01 | 98.55/1.45/0.00 |
| (%) (Favored/ | | | |
| allowed/disallowed) | | | |

### FXAT Loss Results in a Decrease of OXPHOS Subunits as Well as Translational Regulator METTL17

Applicants next asked at an organelle-wide level what pathways are most altered following FXN depletion. Applicants systematically considered all 149 MitoPathways from the MitoCarta 3.0 inventory (Rath et al., 2021) and found that in addition to the Fe—S containing proteins, the levels of respiratory chain Complex I and Complex II were significantly reduced (FIG. 5C and FIG. 12B). Indeed, these are the respiratory chain complexes that contain Fe—S clusters, and moreover, biopsy material from FA models will often exhibit moderate defects in these two complexes (Puccio et al., 2001; Rotig et al., 1997). Of note, the analysis did not reveal a widespread decrease in all mitochondrial-localized proteins (FIG. 12C), indicating that the overall mitochondrial content is preserved despite loss of FXN.

Applicants were intrigued to see that cells edited for FXN also displayed reduced levels of the mitochondrial ribosome, specifically proteins that make up the small (SSU), but not large (LSU) subunit of the mitochondrial ribosome (FIG. 5D and FIG. 12B). The mitoribosome is essential for the intra-organellar translation of the 13 mtDNA encoded OXPHOS subunits in humans (Christian and Spremulli, 2012; Mai et al., 2017; Ott et al., 2016), without which mitochondrial respiration cannot occur. Further examination of other proteins involved in mtDNA expression, i.e., mtDNA maintenance and mtRNA metabolism, revealed no such similar reduction in FXN null cells (FIG. 12D-12E). Intriguingly, one of the top depleted proteins in the FXN-null cells was METTL17 (FIG. 5D), a seven-beta-strand methyltransferase which has recently been suggested to function as a SSU assembly factor (Shi et al., 2019) but has no known connections to Fe—S cluster biosynthesis. These findings indicated a profound, yet poorly fleshed out, connection between Fe—S cluster levels and mitochondrial translation which Applicants set out to further explore.

mtRNA Translation is Attenuated in the Absence of FXN

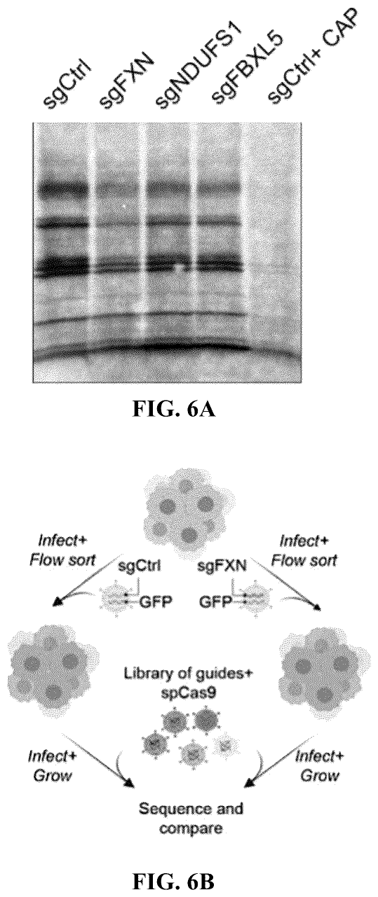

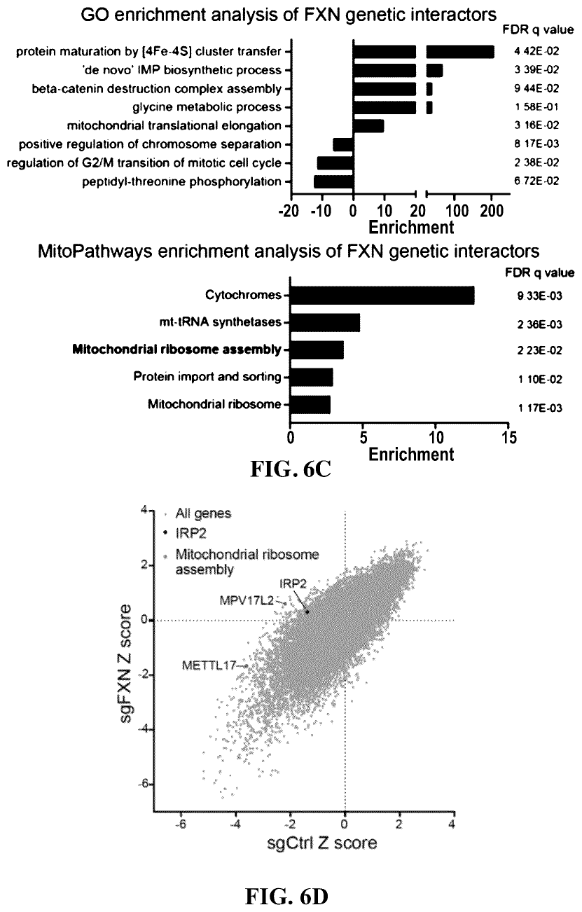

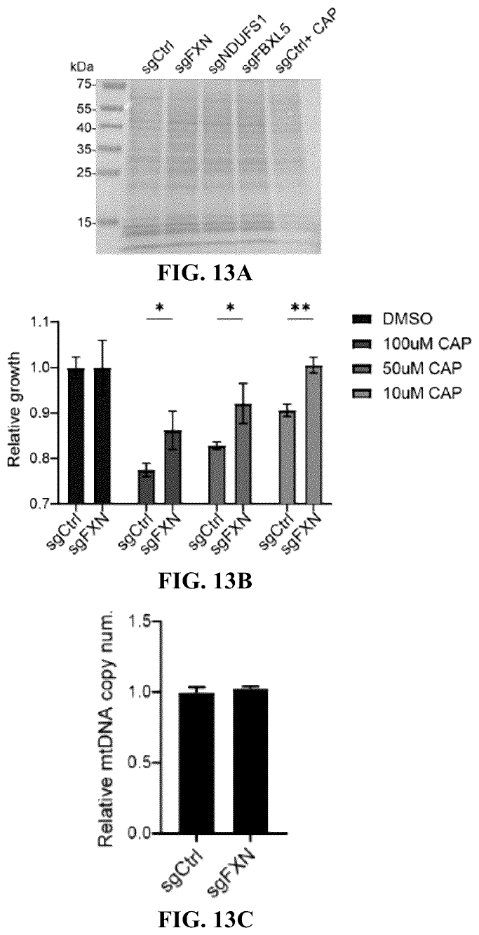

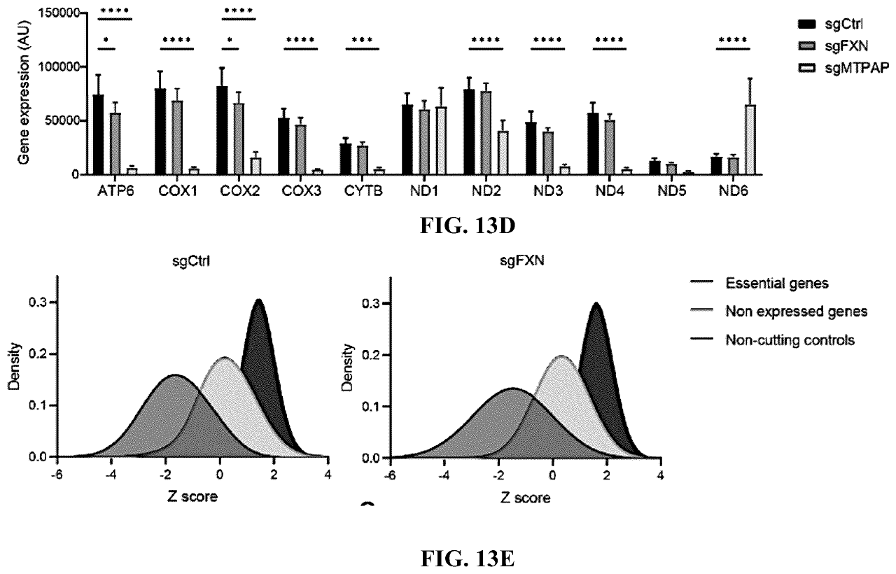

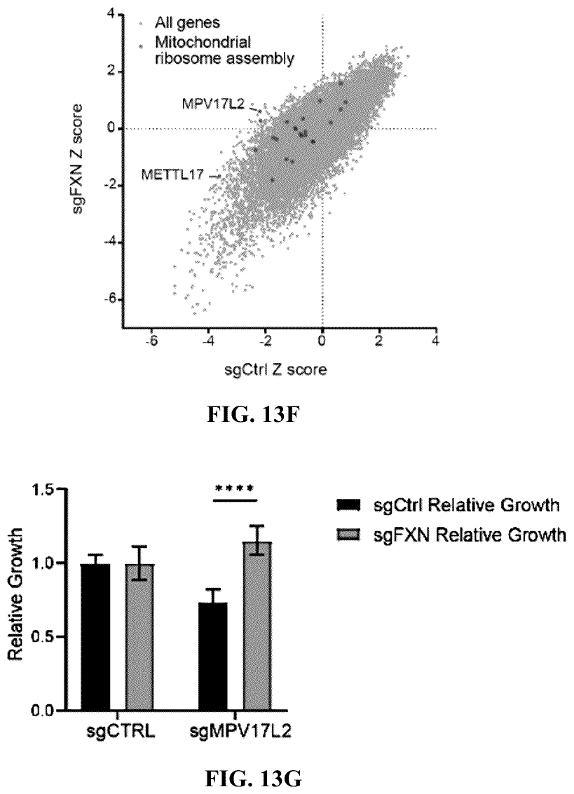

Applicants next directly tested whether FXN depletion results in a defect in intra-mitochondrial protein translation. To this end, Applicants assessed the rates of protein synthesis in the mitochondria in K562 cells edited for FXN, NDUFS1 or FBXL5 (FIG. 6A and FIG. 13A). The latter two genes would indicate whether any changes Applicants observe in FXN null cells are secondary to OXPHOS deficiency or activation of the iron-starvation response, respectively, two known consequences of FXN loss. While FXN deficient cells had dampened intra-mitochondrial protein translation, Applicants did not observe a comparable effect in NDUFS1 or FBXL5 deficient cells. Furthermore, FXN null cells were comparatively resistant to chloramphenicol (FIG. 13B), which directly inhibits mitochondrial translation. This translation defect was not a secondary consequence of a defect in mtDNA homeostasis or expression, as mtDNA copy number and mtRNA levels were intact upon loss of FXN (FIG. 13C-13D).

Applicants wondered which of the many genetic pathways downstream of FXN deficiency contribute to the reduced fitness observed in these cells. Applicants performed a genome-wide CRISPR screen (FIG. 6B) on a FXN null background to highlight pathways that become conditionally essential (synthetic sick) vs. redundant or epistatic (buffering) (DeWeirdt et al., 2020). The screen worked from a technical perspective (FIG. 13E), and Applicants recovered positive genes/pathways (e.g., IRP2; FIG. 6D) (Ast et al., 2019) and Fe—S cluster dependent pathways (e.g., de novo IMP biosynthesis caused by a loss of PPAT; FIG. 6C) in the screen. Intriguingly, enrichment analysis using either GO terms or MitoPathways (from MitoCarta 3.0) highlighted mitochondrial translation as one of the key cellular pathways that was buffered in FXN null cells (FIG. 6C). Specifically, MitoPathways enrichment highlighted that a key deficit in FXN null cells is mitochondrial ribosome assembly, with loss of both the mitoribosome assembly factors MPV17L2 and METTL17 scoring as significant buffering interactions in this screen (FIG. 6D and FIG. 13F-13G). This genetic interaction further highlighted METTL17 as worthy of additional investigation.

### METTLJ7 is Essential to Maintain Robust Mitochondrial Translation and is Post-Transcriptionally Depleted in the Absence of FXN

METTL17 is a highly conserved mitochondrial matrix protein that has recently been linked to mitochondrial translation (Arroyo et al., 2016). Specifically, METTL17 belongs to the methyltransferase-like family (Wong and Eirin-Lopez, 2021) and has been suggested to take part in the assembly of the mitoribosomal small subunit (Shi et al., 2019). However, with respect to the mitoribosome, its methylation target remains controversial (Chen et al., 2020; Itoh et al., 2022; Van Haute et al., 2019), and so the contribution of METTL17 to mitoribosome maturation remains unresolved.

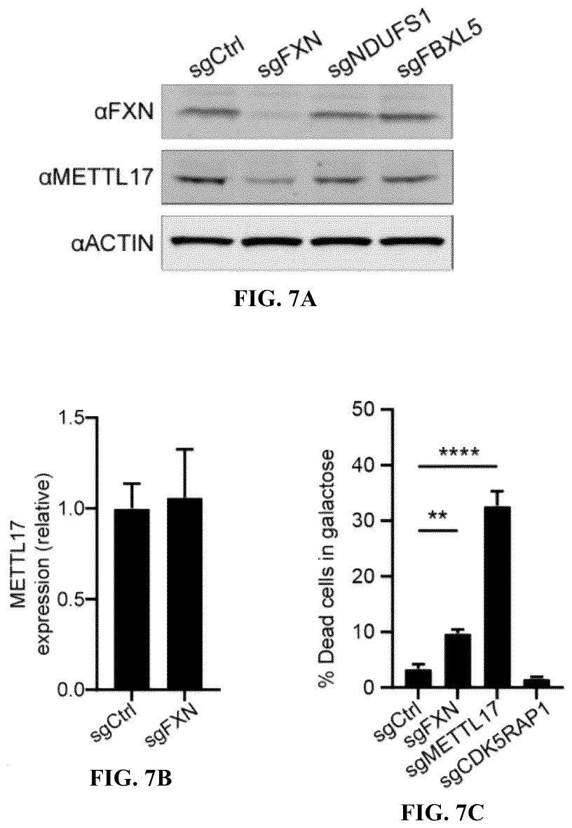

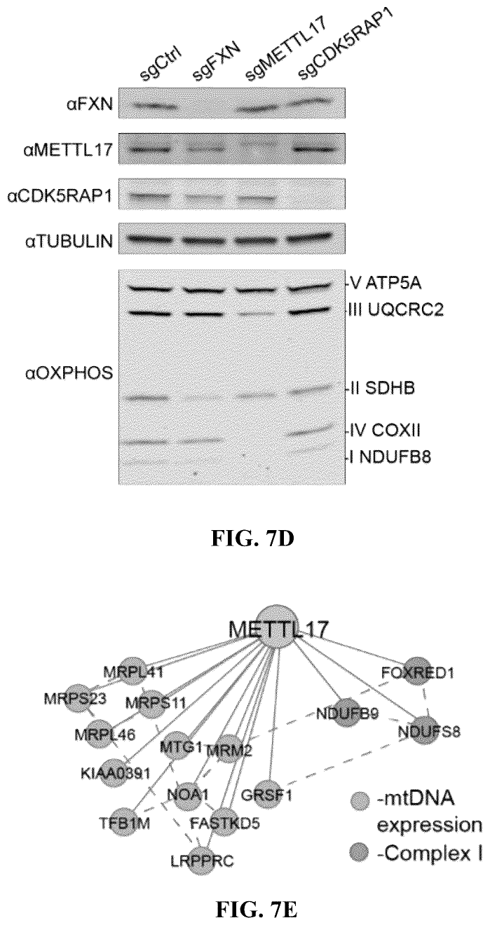

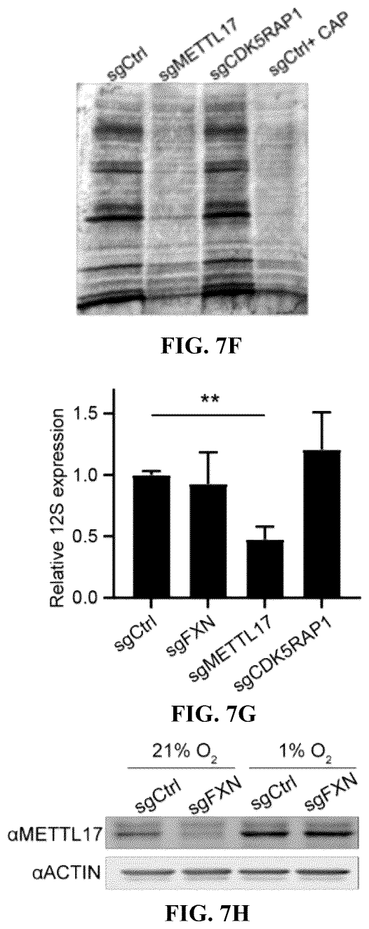

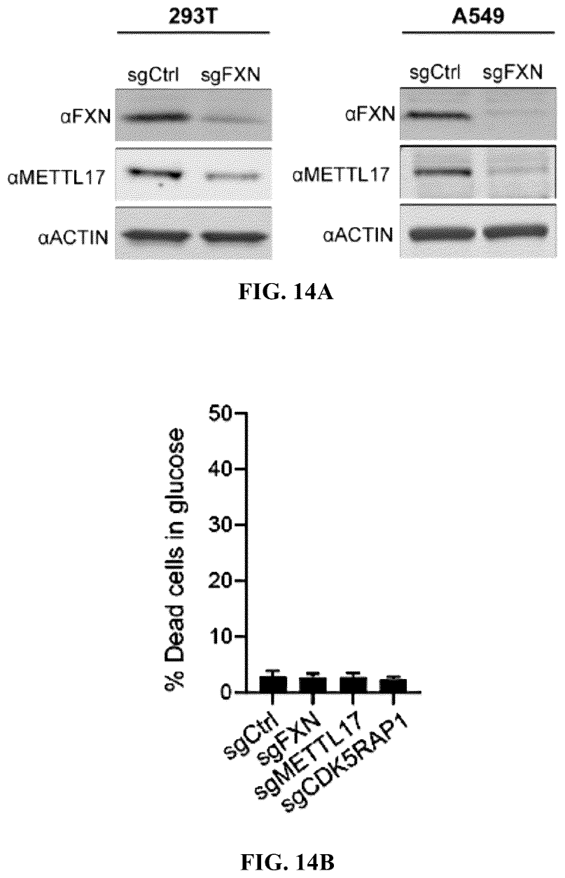

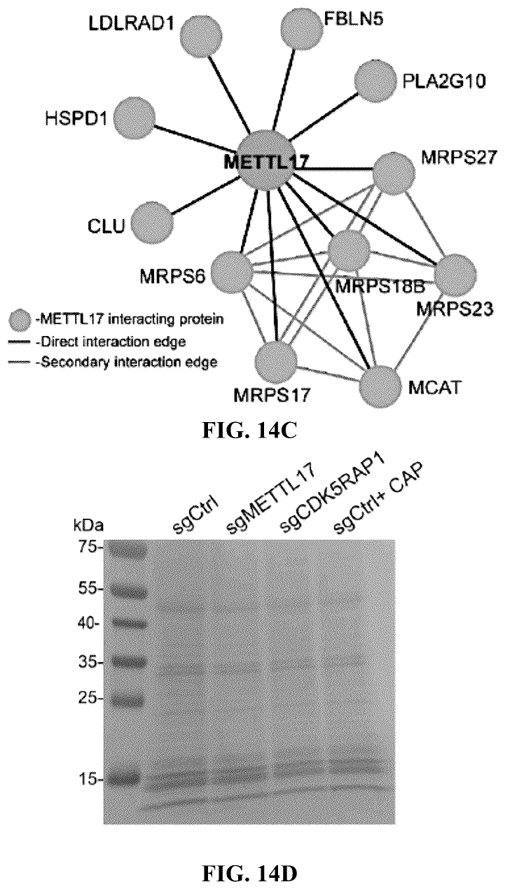

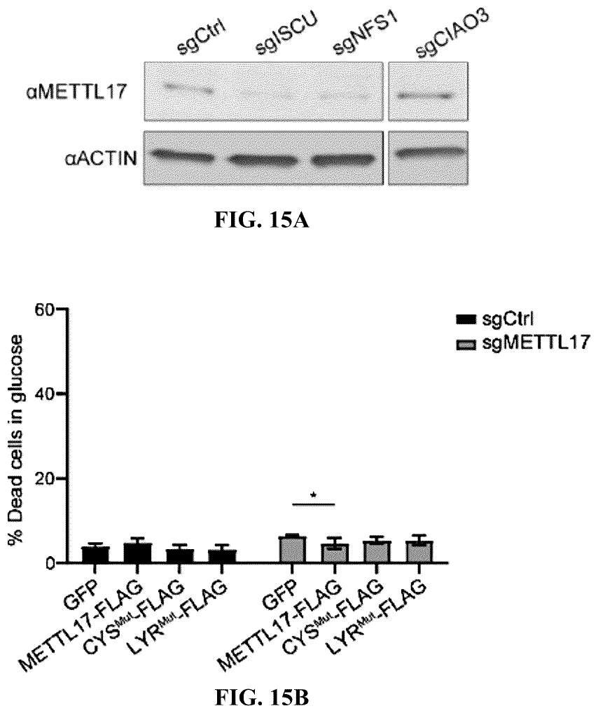

First, Applicants validated the proteomics result that METTL17 is indeed depleted in FXN edited cells (FIG. 7A, FIG. 14A). Moreover, this depletion could not be phenocopied upon loss of NDUFS1 or FBXL5, indicating that it was likely not due to a secondary consequence of OXPHOS deficiency or iron starvation signaling. Importantly, METTL17 mRNA levels were preserved in FXN null cells (FIG. 7B), indicating that METTL17 protein is lost post-transcriptionally level in the absence of FXN.

Next, Applicants sought to determine the extent to which METTL17 depletion attenuates mitochondrial translation and OXPHOS. As with FXN depleted cells, METTL17 null cells undergo extensive cell death when grown on galactose (FIG. 7C and FIG. 14B), a sugar source that forces cells to rely exclusively on OXPHOS for ATP production (Arroyo et al., 2016). No significant galactose-induced death was observed in the absence of CDK5RAP1, a bona-fide Fe—S cluster dependent tRNA methylthiotransferase dual localized to the mitochondria and nucleus (Reiter et al., 2012). Furthermore, both FXN and METTL17 null cells displayed combined respiratory chain deficiencies (FIG. 7D), in contrast to CDK5RAP1 depleted cells. METTL17 likely plays a pervasive and crucial role in the maturation of the SSU, consistent with its Cancer Dependency Map profile (FIG. 7E), as well as its extensive physical interaction with SSU subunits identified in BioPlex (FIG. 14C) (Huttlin et al., 2021). In line with these unbiased methods, Applicants observed near complete ablation of mitochondrial translation and depletion of the 12S (the SSU rRNA) in the absence of METTL17 (FIG. 7F-7G and FIG. 14D). Applicants note that METTL17 null cells appear to have an even more extreme phenotype than FXN edited cells in many of these assays. This may be due to the kinetics of METTL17 depletion in METTL17 versus FXN edited cells, the latter experiencing the METTL17 deficiency as secondary. Finally, Applicants tested whether hypoxia could restore METTL17 levels following loss of FXN, as Applicants have previously seen it buffering many FXN-associated phenotypes (Ast et al., 2019). Indeed, culturing FXN edited cells in 1% 02 was sufficient to revert METTL17 levels back to control conditions (FIG. 711), consistent with it being a downstream consequence of FXN deficiency. Collectively, these experiments confirm that METTL17 is indeed intimately linked to SSU functionality in a way that is dependent on intact FXN levels.

### METTL17 Contains a Putative Fe—S Cluster Binding Motif Essential for its Function

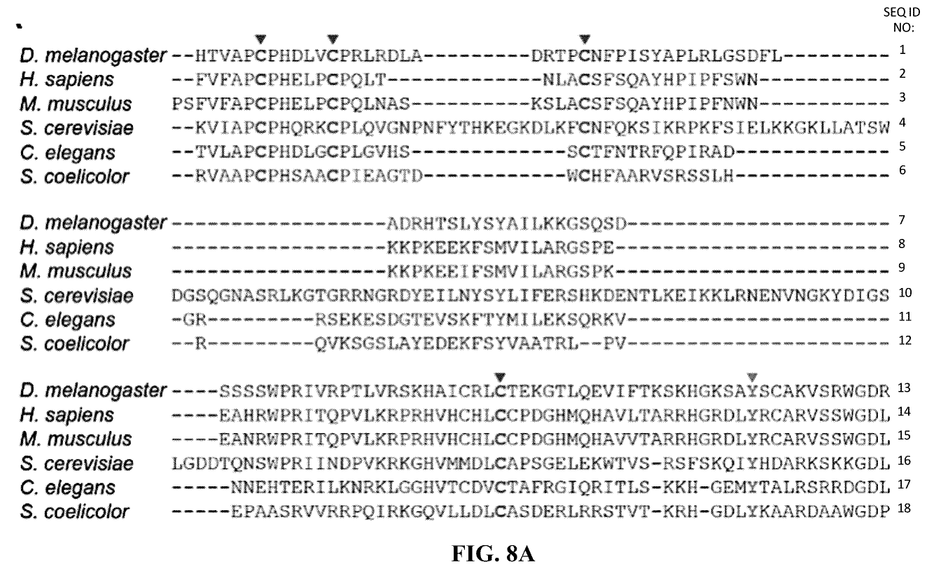

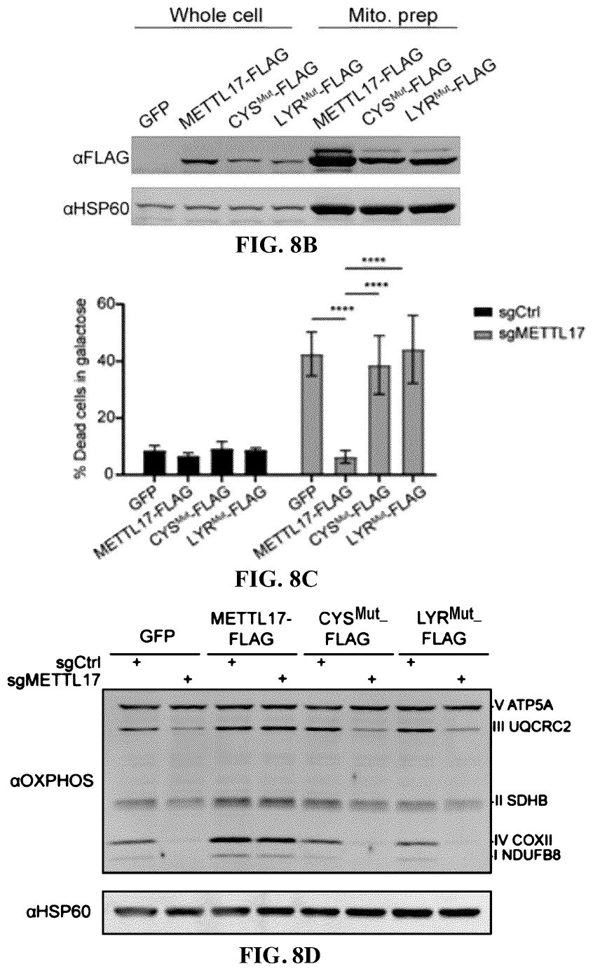

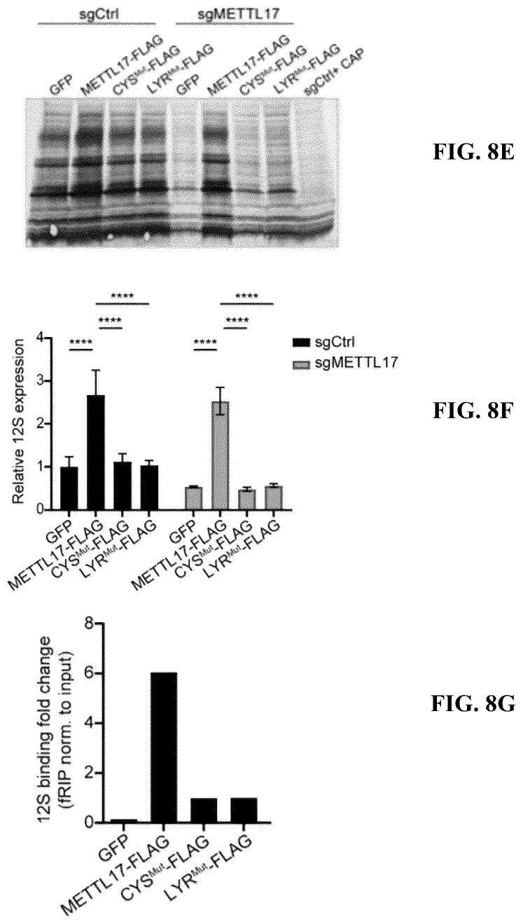

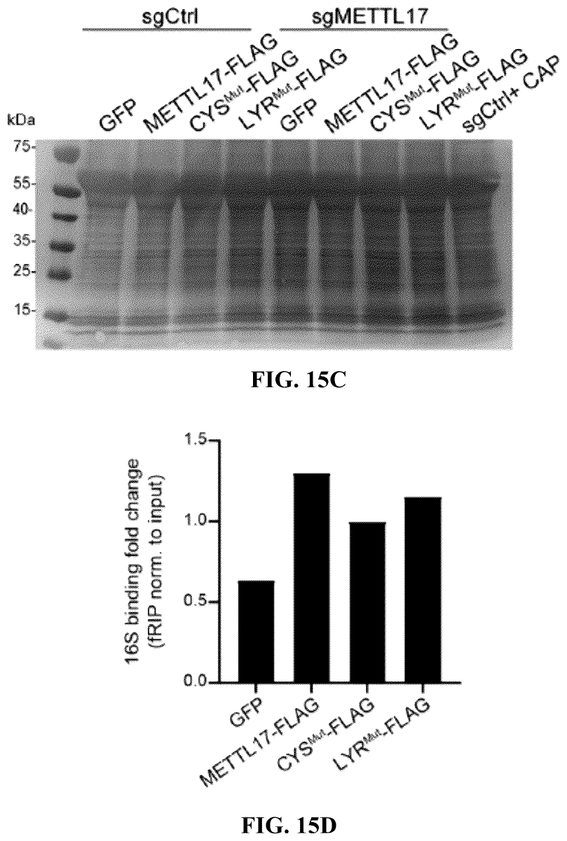

The striking loss of METTL17 protein levels in the absence of FXN resembles that of bona-fide Fe—S cluster containing proteins (FIG. 5B) and made Applicants wonder if perhaps METTL17 might also harbor a previously unappreciated Fe—S cluster. Multiple sequence alignment (FIG. 8A) of six METTL17 orthologs spanning bacteria, fungi, and humans revealed two striking features: (1) four conserved cysteine residues and (2) a tyrosine residue that forms part of an ‘LYR’ (leucine-tyrosine-arginine) motif in human METTL17. The former is likely a metal binding site, and indeed a recent cryogenic electron microscopy (cryo-EM) structure of the trypanosomal METTL17 homologue revealed coordination of Zn2+ through these residues (Saurer et al., 2019). It is known that Zn2+ can replace a labile Fe—S cluster in aerobically purified proteins (Imlay, 2006; Nicolet and Fontecilla-Camps, 2014; Wachnowsky and Cowan, 2017). The LYR motif (not to be confused for LYRM protein) is often found in Fe—S cluster apoproteins (Maio et al., 2014) which is proposed to engage the Fe—S cluster handoff machinery. The AlphaFold2.0 predicted structure of human METTL17 (Jumper et al., 2021; Varadi et al., 2022) shows that the conserved cysteines are spatially positioned so as to potentially form an Fe—S binding pocket while the LYR motif is surface accessible, further strengthening the likelihood that these motifs are functional. Moreover, METTL17 remains stable following disruption of the cytosolic Fe—S biosynthesis machinery (FIG. 15A), further hinting at a specific connection between loss of METTL17 and mitochondrial Fe—S cluster assembly. Collectively, these lines of evidence led Applicants to hypothesize that METTL17 is a mitochondrial Fe—S cluster binding apoprotein, and that perhaps this was previously missed given the labile nature of Fe—S clusters.

Applicants asked whether these candidate Fe—S binding motifs were indeed important for the activity of METTL17. To this end, Applicants generated three constructs for METTL17, a WT FLAG-tagged version as well as two mutants: CYSMut-FLAG in which the conserved cysteines were changed to serines, and LYRMut-FLAG in which the LYR residues were mutated to alanines. All three of these variant proteins were enriched in mitochondrial fractions (FIG. 8B), demonstrating their proper subcellular targeting, although the two mutants were less abundant than the WT (as is typical of Fe—S cluster apoproteins lacking their cofactor). Applicants next tested if these mutants could support mitochondrial translation in the absence of endogenous METTL17. METTL17-FLAG expression could rescue for loss of the endogenous gene as shown by reduced death in galactose and restored OXPHOS subunit levels, mitochondrial de-novo translation and 12S levels (FIG. 8C-8F and FIG. 15B-15C). However, the CYSMut-FLAG and LYRMut-FLAG mutants failed to rescue any of the defects assayed. This would indicate that both motifs are crucial for METTL17's role in supporting SSU activity. Of note, METTL17-FLAG expression served to boost mitochondrial translation and the steady state levels of multiple OXPHOS subunits (most notably in complexes I, II and IV) even above the GFP control, suggesting that it is a rate limiting factor in mitochondrial protein synthesis.

Applicants next wondered whether the cysteine binding pocket and LYR motif were also required for METTL17 engagement with the SSU. Applicants performed a formaldehyde RNA immunoprecipitation assay (D et al., 2016) to test for the interaction between the various constructs and the 12S (FIG. 8G). In this assay, cells are lightly crosslinked, lysed, the protein of interest is immunoprecipitated and bound RNA is quantified by qPCR. Applicants could observe that while METTL17-FLAG showed robust binding to the 12S as compared to a GFP control, the interaction between CYSMut-FLAG or LYRMut-FLAG and the 12S was diminished (even taking into account the lower basal expression levels for the constructs compared with 12S). In contrast, there was only marginal enrichment for 16S binding for these constructs when compared to the GFP control, and no notable difference between the WT and mutant forms of METTL17 (FIG. 15D). These findings suggest that apart from structural stability, the putative Fe—S cluster in METTL17 is also important for contributing (directly or indirectly) to SSU and 12S binding.

### Biophysical Studies Confirm METTL17 Harbors an Fe—S Cluster

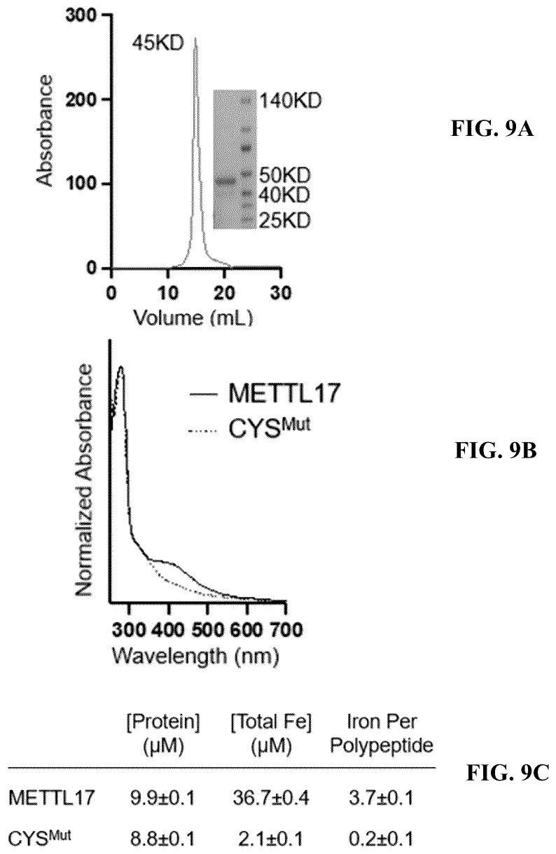

Applicants then set out to study and analyze the purified human METTL17 under conditions that would be optimal to preserve any Fe—S cluster present. Affinity chromatography of METTL17 gave a clean preparation of the protein that resulted in both a single band on an SDS-PAGE gel and a monodisperse analytical gel filtration peak corresponding to a molecular weight near 50 kDa (FIG. 9A). Purified METTL17 precipitates upon exposure to air as is common for air-sensitive Fe—S proteins (Wachnowsky and Cowan, 2017). Inductively coupled plasma mass spectrometry (ICP-MS) analysis revealed the presence of 3.7 Fe ions per polypeptide (FIG. 9B). In a variant in which the four cysteines predicted to coordinate the cluster (C333, C339, C347, and C404) were mutated to serines (‘CYSMut’), almost all the iron was lost (0.2 Fe ions per polypeptide). The UV-Vis spectrum of METTL17 contains a broad charge-transfer band around 400 nm, suggesting the presence of either an [Fe4S4]2+ cluster or an [Fe3S4]+ cluster (FIG. 9C) (Agar et al., 2000; Bruschi et al., 1976; Emptage et al., 1983; Khoroshilova et al., 1997); this band is absent in the spectrum of the CYSMut sample. No electron paramagnetic resonance (EPR) signal was observed for the METTL17 sample between 5 and 50 K (data not shown), and addition of the reducing agent dithionite or the oxidizing agent indigodisulfonic acid also did not produce an EPR signal. The lack of an EPR signal in the as-isolated and oxidized samples rules out the presence of an [Fe3S4]+ cluster, which has a characteristic EPR signal (Hagen, 1992). Taken together, the Fe quantitation, UV-Vis spectroscopy, and EPR spectroscopy results are most consistent with the presence of an [Fe4S4]2+ cluster coordinated by C333, C339, C347, and C404 on human METTL17.

### The [Fe4S4]2+ Cluster on METTLJ7 Stabilizes its Binding to the Mitoribosomal Small Subunit Prior to Translation Initiation

Next, Applicants sought to directly visualize the Fe—S cluster in METTL17 using cryo-EM, with a focus on capturing it on the mitoribosome. Applicants made initial attempts to study the human SSU-METTL17 complex, but METTL17 escaped detection as its binding is likely not stable enough. As the putative Fe—S cluster binding motif is conserved (FIG. 8A), Applicants used mitoribosomes from the yeast S. cerevisiae, which provide a suitable model for structural studies due to stabilising rRNA expansion segments and protein extensions (Amunts et al., 2014) that would be amenable for studying assembly intermediates. Although previous fungi mitoribosome structures have been reported (Desai et al., 2017; Itoh et al., 2020), they did not investigate SSU assembly.

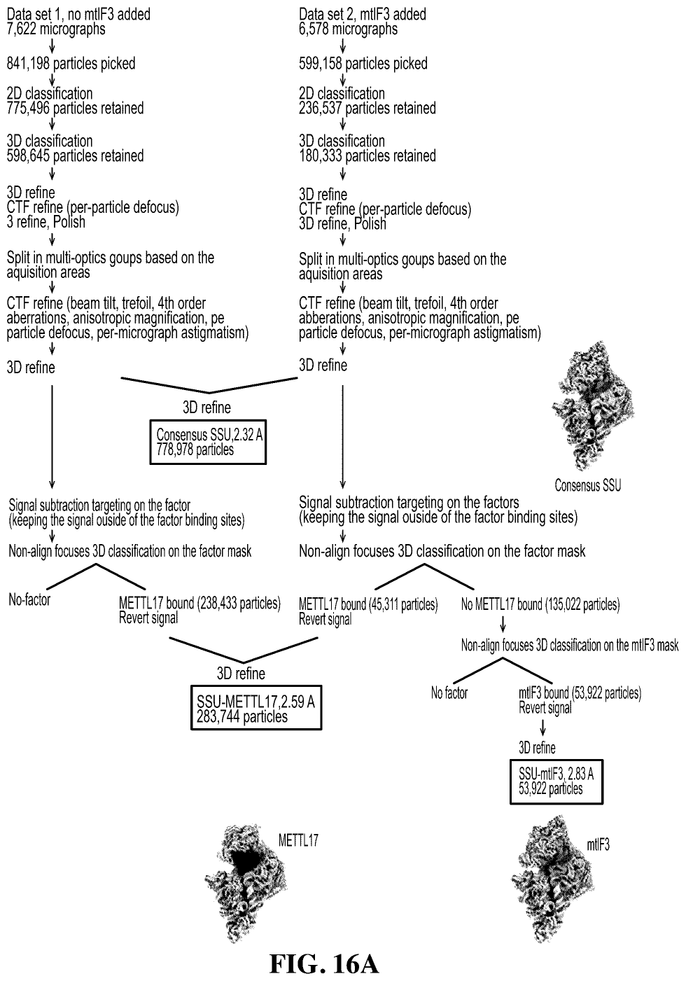

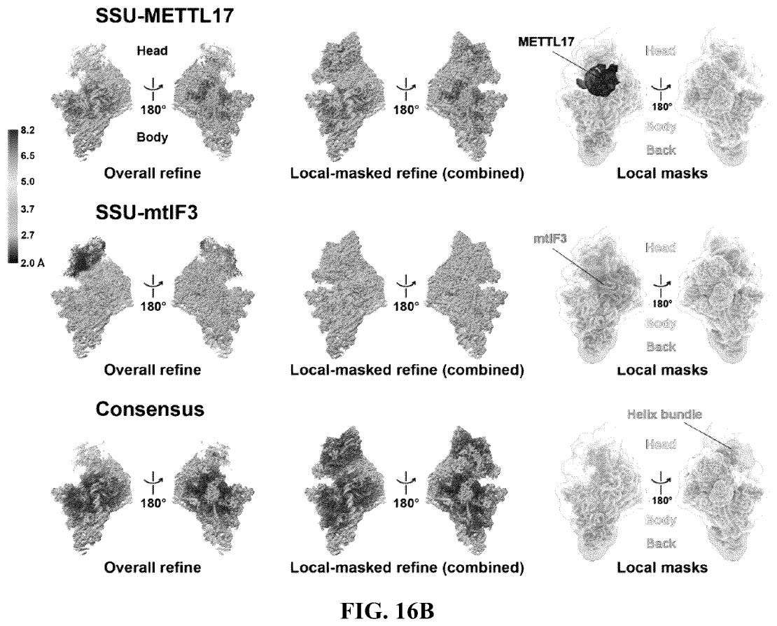

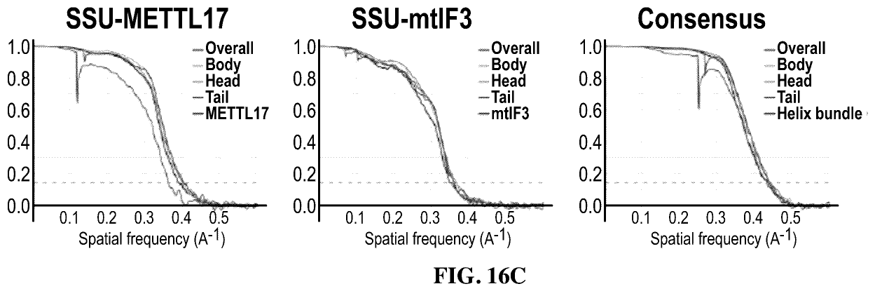

Reconstructions from the first cryo-EM dataset followed by 3D classification and refinement revealed two distinct classes, the empty and METTL17-bound states of the SSU (FIG. 16A-16C). In the second dataset, Applicants added a recombinant initiation factor mtIF3, and obtained three states: free SSU, METTL17-bound, mtIF3-bound. Applicants then grouped particles from the two datasets and resolved the structures of the SSU, SSU-METTL17, and SSU-mtIF3 at an overall resolution of 2.3 Å, 2.6 Å, and 2.8 Å, respectively (Table 12). Compared to the previous report of S. cerevisiae SSU at 3.5 Å resolution (Desai et al., 2017), the improved reconstruction allowed us to build a more accurate model. Specifically, Applicants modelled extensions of the mitoribosomal proteins bS1m, uS2m, uS3m and uS7m as well as corrected the assignment of a guanosine diphosphate (GDP) to adenosine triphosphate (ATP) in the nucleotide pocket of the protein mS29 (S6 Å-D).

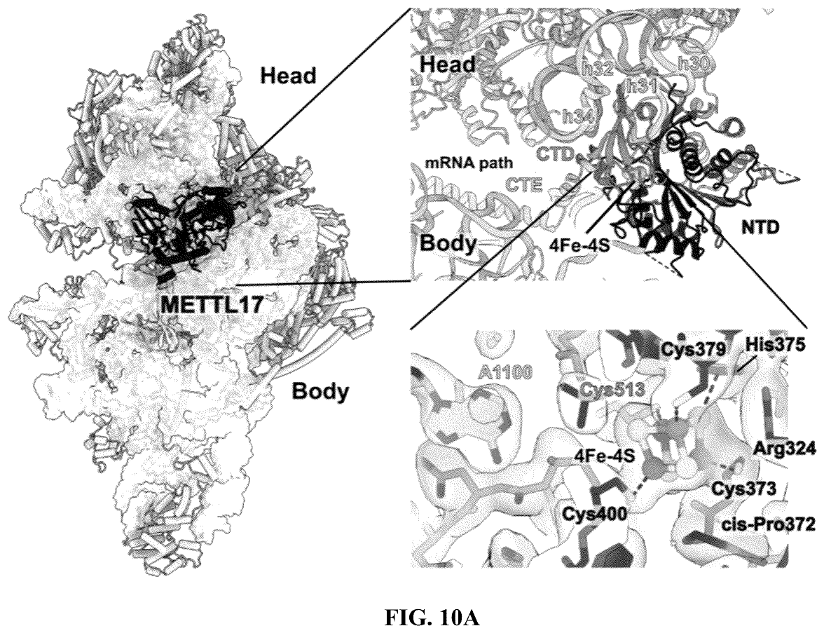

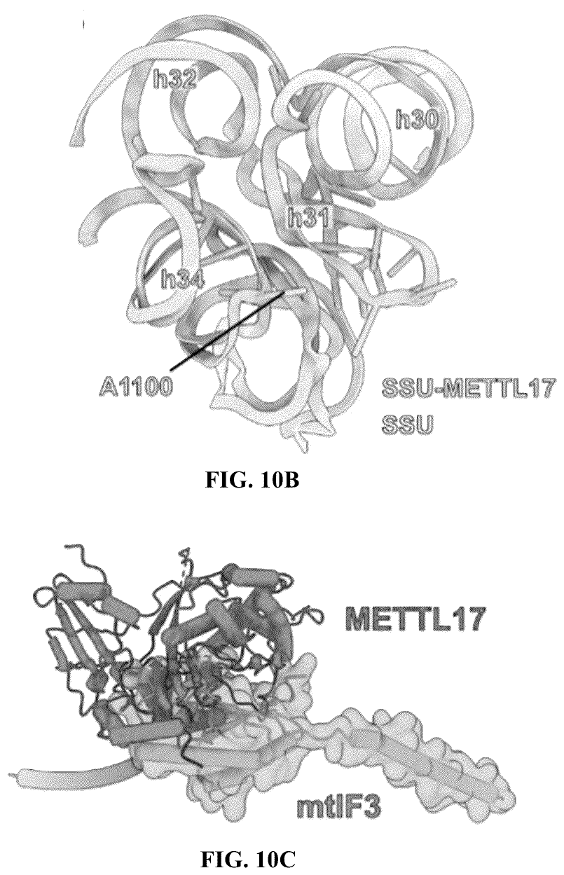

In the SSU-METTL17 complex, the 72-kDa factor is found in the cleft between the head and the body and its presence prevents a monosome formation (FIG. 10A). In contrast to previously characterized SSU assembly factors (Itoh et al., 2022), METTL17 binds exclusively to the rRNA of the head, and the binding involves both, the N-terminal domain (NTD) and the C-terminal domain (CTD). The NTD is a Rossmann-fold methyltransferase domain, characterized by a seven-stranded beta-sheet core sandwiched by six alpha-helices, while the CTD is a unique fold that consists of a four-stranded beta-sheet with an alpha-helix. The comparison with the non-bound state shows that the association of METTL17 withdraws the rRNA helix h31 causing rearrangement of h32 and h34, and the entire head is rotated by approximately 3 Å up to open the tRNA binding cleft. Thus, h31 is more exposed, and the rRNA nucleotide A1100 in h34 is flipped out (FIG. 10B). In addition, the mitochondrial C-terminal extension fills the mRNA channel, blocking its premature binding.

The SAM motif Gly-X-Gly-X-Gly of S. cerevisiae METTL17 is disrupted with an Ala at the last Gly position and a Tyr at the first X position, suggesting that the methylation is not a conserved function of this protein in the process of the mitoribosome assembly. In fact, the 15S SSU rRNA in yeast is not methylated (Klootwijk et al., 1975) and no electron density was observed for a SAM cofactor. Of note, a distance of 25 Å separates the metal binding site from the SAM binding motif in the cryo-EM structure of the trypanosomal METTL17 homologue (Saurer et al., 2019), arguing against METTL17 functioning as a radical SAM enzyme in this organism as well. Applicants identified an ordered density, adjacent to sulfhydryl groups of four cysteine residues Cys373, Cys379, Cys400, Cys513 (yeast numbering, equivalent to human Cys333, Cys339, Cys347, Cys404) (FIG. 10A). The density corresponds to eight atoms organized in a cube, which is consistent with [Fe4S4]2+ cluster, where the four iron atoms are bound by cysteines S atoms and bridge sulfur atoms. The unique feature of the embedded [Fe4S4]2+ cluster in Applicants' structure is that it brings together residues that are 140 amino acids apart, thus stabilising the N- and C-terminal domains of METTL17 when it's bound to the SSU. The resulting orientation of Cys400 and Cys513 forms a pocket, where rRNA base A1100 adapts, which contributes to a prominent binding of all the components together on the mitoribosome. Moreover, Applicants identified a cis-proline Pro372 of METTL17 that orients the Cys373 side chain towards the [Fe4S4]2+ cluster and further supports the binding. Furthermore, Applicants identified His375 and Arg324 within cluster interaction distance, suggesting a potential involvement in transfer and ligation of the [Fe4S4]2+ cluster. The AlphaFold2.0 predicted structure of the human METTL17 (Jumper et al., 2021; Varadi et al., 2022) is overall consistent with Applicants' model (FIG. 17E), and the superposition with the high resolution structure of the human SSU (Itoh et al., 2022) suggests conserved interacting interfaces (FIG. 17F).

To further clarify a putative role of METTL17, Applicants formed a preinitiation complex by purifying the SSU-METTL17 and incubating it with a recombinant mtIF3. A cryo-EM density map was then calculated, and Applicants identified a new class containing a subset of 53,922 particles that was processed with a signal subtraction, followed by 3D classification using the mask for the mtIF3-binding site (FIG. 16A-16C). The map features the complex SSU-mtIF3, where the initiation factor occupies a position on the SSU that is similar to the human counterpart (FIG. 17G) (Khawaja et al., 2020) and mutually exclusive with METTL17, as a superposition of the two states shows clashes between the factors (FIG. 10C). Thus, the binding of the regulatory factor METTL17 is stabilized by the [Fe4S4]2+ cluster and precedes the initiation of translation.

### Forced Expression of METTL17 Rescues Bioenergetic, but not Growth, Defects of FXN Null Cells

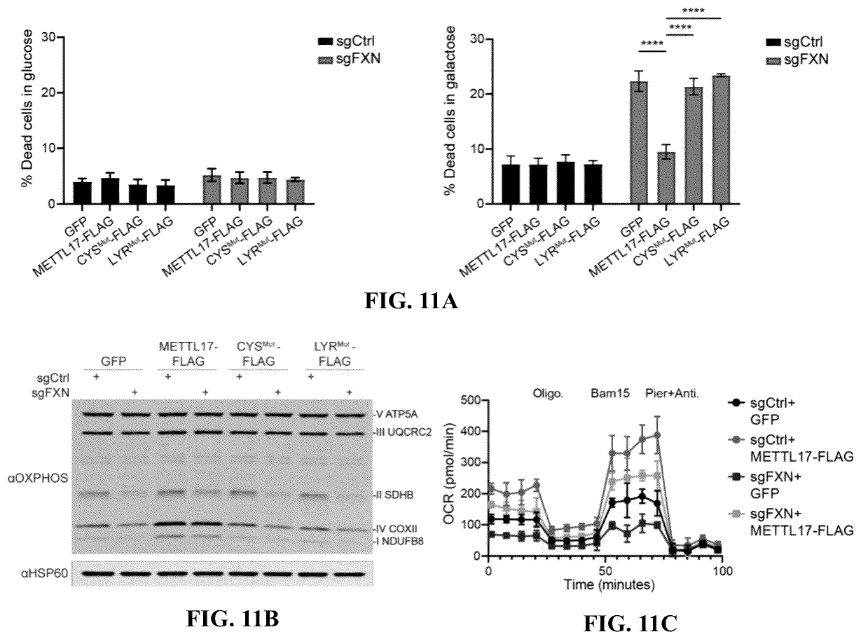

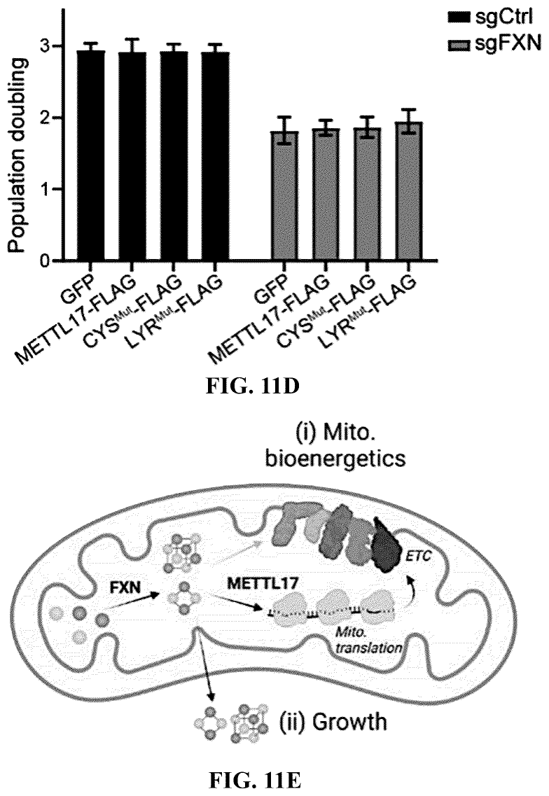

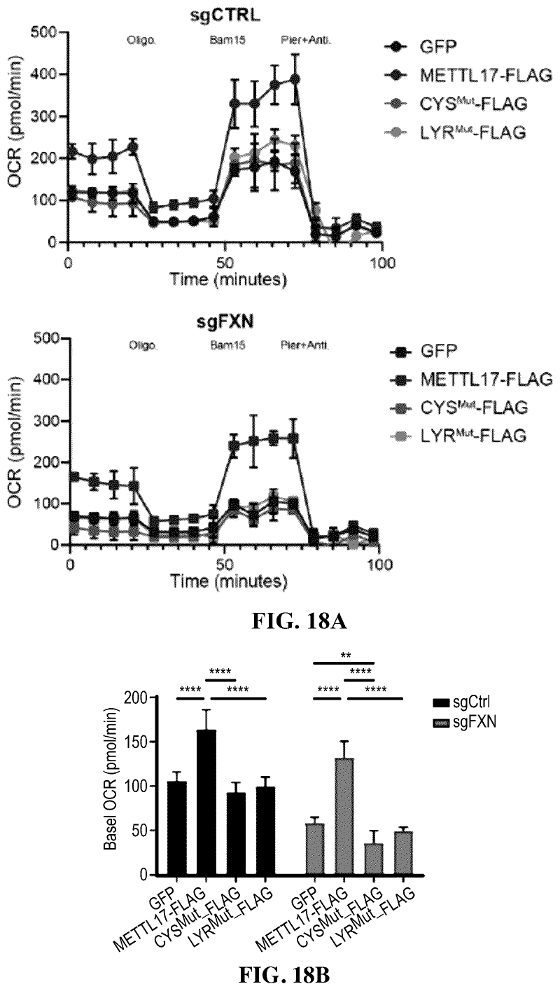

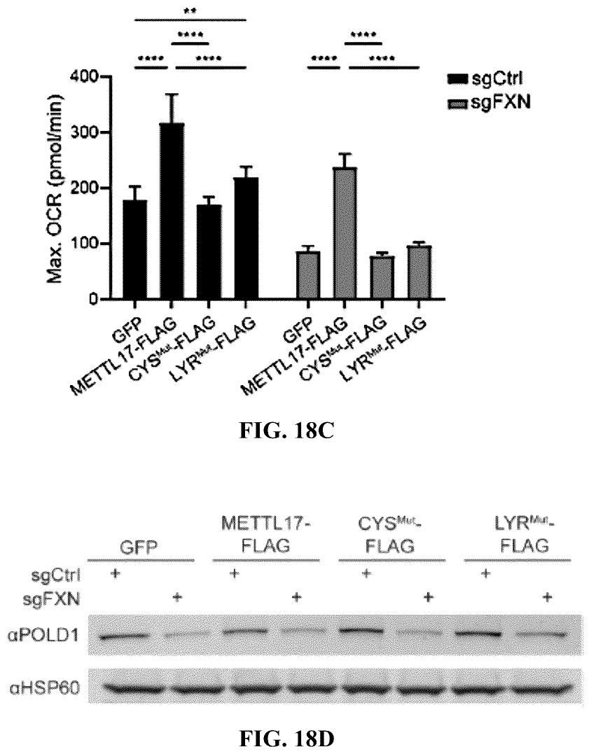

Applicants sought to determine which of the downstream phenotypic consequences of FXN deficiency could be prevented by forced expression of METTL17. Applicants tested the effects of overexpressing METTL17, or its mutants, in FXN null cells. Remarkably, Applicants observed that the galactose-induced death of FXN null cells could be reversed by the forced overexpression of METTL17-FLAG, but not CYSMut-FLAG or LYRMut-FLAG (FIG. 11A). In line with these findings, Applicants showed that the loss of various OXPHOS protein subunits and the lowered oxygen consumption of FXN null cells were restored to WT levels by overexpression of METTL17-FLAG (FIG. 11B-11C and FIG. 18A-18C). Thus, it seems that the bioenergetic defect of FXN depleted cells could largely be restored by re-expressing one mitochondrial Fe—S cluster containing protein: METTL17.

Next, Applicants wondered if other cellular defects outside of mitochondria and observed in FXN null cells could be rescued by METTL17 overexpression, as human Fe—S cluster biosynthesis is required for other essential processes such as nuclear DNA maintenance, repair and nucleotide synthesis. When Applicants tested the growth of FXN null cells upon METTL17 overexpression, however, Applicants saw no benefit (FIG. 11D). Moreover, the cytosolic Fe—S apoprotein POLD1 remained depleted in FXN null cells irrespective of METTL17 overexpression (FIG. 18D).

Together, these data demonstrate for the first time that the growth and bioenergetic defects observed in FXN null cells are separable, and that they can be uncoupled from each other through the expression of the Fe—S cluster containing protein METTL17. Fe—S clusters that are made in the mitochondria can in principle be directed to two distinct routes: one for local use within mitochondria supporting bioenergetic demand vs. one for export to the cytosol to support growth (FIG. 11E). METTL17 expression can evidently privilege Fe—S clusters for use within mitochondria.

### Discussion

Here Applicants have made the discovery that METTL17, a previously described putative SSU maturation factor, harbors a hitherto unrecognized [Fe₄S₄] cluster that is important for its function in protein translation. Applicants report for the first time that METTL17 deficiency lies downstream of FXN loss and appears to be a key effector of impaired mitochondrial protein translation. Re-expressing METTL17 is sufficient to rescue the bioenergetic, but not growth, phenotypes associated with FXN deficiency. These findings demonstrate that the Fe—S cluster supply within mitochondria can be uncoupled from that of the cytosol, and that factors such as METTL17 can privilege the availability of Fe—S clusters for specific routes.

Applicants' proteome-wide analysis indicates that nearly all the Fe—S cluster containing proteins Applicants could detect are de-stabilized when the Fe—S cluster supply is reduced. While it has long been known that one function of Fe—S clusters (in addition to their many roles in electron transfer, oxyanion binding, etc.) is protein stability (Lill and Freibert, 2020), these findings show that in fact this is a nearly universal role of the cluster. Previous proteomic profiling of FA patient cells has described deficits in some, but not all, Fe—S cluster containing proteins (Telot et al., 2018). However, it is likely that a near total depletion of FXN, such as is generated by CRISPR editing is not observed in patients.

These structural studies have revealed that the [Fe4S4]2+ cluster stabilizes METTL17 and assists in its coupling to the rRNA of the mitoribosomal small subunit during the assembly process. Throughout evolution, the presence of an [Fe₄S₄]²⁺ cluster on METTL17 is more conserved than that of SAM binding. When examining the METTL17 ortholog D. discoideum, a member of the Amoebozoa outgroup of metazoa and fungi, Applicants found that both the SAM motif and the four cysteine residues coordinating Fe—S cluster binding are present indicating that this is likely the more ancient form of the protein. The obtained and predicted structure of METTL17 argues against a radical SAM role for the Fe—S, given its distance to the SAM binding motif and the fact that the iron sulfur cluster is coordinated by four cysteines rather than three. The obtained structure of the SSU-METTL17 complex allows us to put METTL17 in the context of the dynamic SSU assembly, extending its part in the view of the mitoribosome formation. Comparison with structural studies of earlier assembly intermediate states (Itoh et al., 2022) suggests that METTL17 can be accommodated on the SSU in the presence of RBFA, and Applicants also show that METTL17 is replaced by the initiation factor mtIF3. Thereby, METTL17 facilitates a productive assembly pathway through acting on relatively late stages, which reflects the structural importance of the [Fe₄S₄]²⁺ bound METTL17 for the biological function of the mitoribosome biogenesis.

Applicants' data indicates that METTL17 operates as an Fe—S cluster “checkpoint” for protein translation. The mitochondrial respiratory chain is rich with Fe—S clusters, and Applicants' work shows that mitochondrial translation is dependent on METTL17 being present with an intact Fe—S cluster for proper ribosome assembly. Applicants have shown that METTL17 is very labile in the absence of Fe—S levels, and moreover, that METTL17 is limiting for mitochondrial protein translation. Hence, METTL17 can ensure that mitochondrial protein translation proceeds only when Fe—S cluster levels are replete. As solvent exposed Fe—S clusters are extremely labile to reactive nitrogen and oxygen species, METTL17 could serve as a “kill switch” for turning off the production of mitochondrial machinery in unfavorable environments, enabling sub-organelle regulation of mitochondrial protein translation to match local redox conditions (Allen, 2015).

These findings indicate that at least in cultured cells, FXN deficiency results in a previously under-appreciated deficit in mitochondrial protein translation, which appears to be largely downstream of METTL17. Although OXPHOS deficiency, specifically loss of CI and CII, have long been appreciated to be features of FA samples (Puccio et al., 2001; Rotig et al., 1997), to the best of Applicants' knowledge, no prior report has documented defects in mitochondrial protein translation. Future efforts should be aimed at determining whether patient derived specimens show any evidence of impaired mitochondrial translation. Recent work has identified Fe—S cluster proteins involved in mitochondrial translation embedded in tRNA maturation (Reiter et al., 2012; Wei et al., 2015) as well as in the SSU (Itoh et al., 2022). However, overexpression of METTL17 seems to be sufficient to restore the bioenergetic defects of FXN null cells, suggesting it may be limiting for mitochondrial protein translation.

A curious feature of METTL17 is that its expression uncouples the mitochondrial versus cytosolic routing of Fe—S clusters. In animal cells, de novo Fe—S cluster biosynthesis begins inside of mitochondria, and this supply is then conveyed within the organelle and also exported to the cytosol (Lill and Freibert, 2020; Maio and Rouault, 2020; Srour et al., 2020). Simply forcing the expression of METTL17 seems to privilege mitochondrial-generated Fe—S clusters for intra-mitochondrial use. It is interesting to speculate whether additional, as yet unidentified factors prioritize cytosolic routing of Fe—S clusters. The ability to uncouple these routes by expressing METTL17 could be useful for dissecting the pathogenesis of FA, since at present, Applicants do not know which is dominant in the etiopathogenesis: bioenergetic defect or growth defect. If the bioenergetic defects predominate in vivo, boosting METTL17 could hold therapeutic potential.

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

### REFERENCES RELATED TO EXAMPLE 2

Agar, J. N., Krebs, C., Frazzon, J., Huynh, B. H., Dean, D R., and Johnson, M. K. (2000). IscU as a scaffold for iron-sulfur cluster biosynthesis: sequential assembly of [2Fe-2S] and [4Fe-4S] clusters in IscU. Biochemistry 39, 7856-7862. 10.1021/bi00093In.
Alam, J., Rahman, F. T., Sah-Teli, S. K., Venkatesan, R., Koski, M. K., Autio, K. J., Hiltunen, J. K., and Kastaniotis, A. J. (2021). Expression and analysis of the SAM-dependent RNA methyltransferase Rsm22 from Saccharomyces cerevisiae. Acta Crystallogr D Struct Biol 77, 840-853. 10.1107/52059798321004149.
Allen, J. F. (2015). Why chloroplasts and mitochondria retain their own genomes and genetic systems: Colocation for redox regulation of gene expression. Proc Natl Acad Sci USA 112, 10231-10238. 10.1073/pnas.1500012112.
Amici, D. R., Jackson, J. M., Truica, M. I., Smith, R. S., Abdulkadir, S. A., and Mendillo, M. L. (2021). FIREWORKS: a bottom-up approach to integrative coessentiality network analysis. Life Sci Alliance 4.10.26508/lsa.202000882.
Amunts, A., Brown, A., Bai, X. C., Llacer, J. L., Hussain, T., Emsley, P., Long, F., Murshudov, G., Scheres, S. H. W., and Ramakrishnan, V. (2014). Structure of the yeast mitochondrial large ribosomal subunit. Science 343, 1485-1489. 10.1126/science.1249410.
Andreini, C., Banci, L., and Rosato, A. (2016). Exploiting Bacterial Operons To Illuminate Human Iron-Sulfur Proteins. J Proteome Res 15, 1308-1322. 10.1021/acs.jproteome.6b00045.
Arroyo, J. D., Jourdain, A. A., Calvo, S. E., Ballarano, C. A., Doench, J. G., Root, D. E., and Mootha, V. K. (2016). A Genome-wide CRISPR Death Screen Identifies Genes Essential for Oxidative Phosphorylation. Cell Metab 24, 875-885. 10.1016/j.cmet.2016.08.017.
Ast, T., Meisel, J. D., Patra, S., Wang, H., Grange, R. M. H., Kim, S. H., Calvo, S. E., Orefice, L. L., Nagashima, F., Ichinose, F., et al. (2019). Hypoxia Rescues Frataxin Loss by Restoring Iron Sulfur Cluster Biogenesis. Cell 177, 1507-1521 e1516. 10.1016/j.cell.2019.03.045.
Barad, B. A., Echols, N., Wang, R. Y., Cheng, Y., DiMaio, F., Adams, P. D., and Fraser, J. S. (2015). EMRinger: side chain-directed model and map validation for 3D cryo-electron microscopy. Nat Methods 12, 943-946. 10.1038/nmeth.3541.
Barton, R. M., and Worman, H. J. (1999). Prenylated prelamin A interacts with Narf, a novel nuclear protein. J Biol Chem 274, 30008-30018. 10.1074/jbc.274.42.30008.
Beinert, H., Holm, R. H., and Munck, E. (1997). Iron-sulfur clusters: nature's modular, multipurpose structures. Science 277, 653-659. 10.1126/science.277.5326.653.
Boyd, E. S., Schut, G. J., Shepard, E. M., Broderick, J. B., Adams, M. W., and Peters, J. W. (2014). 17 Origin and evolution of Fe—S proteins and enzymes. Iron-Sulfur Clusters in Chemistry and Biology, 445.
Braymer, J. J., Freibert, S. A., Rakwalska-Bange, M., and Lill, R. (2021). Mechanistic concepts of iron-sulfur protein biogenesis in Biology. Biochim Biophys Acta Mol Cell Res 1868, 118863. 10.1016/j.bbamcr.2020.118863.
Bruschi, M., Hatchikian, C., Le Gall, J., Moura, J. J., and Xavier, A. V. (1976). Purification, characterization and biological activity of three forms of ferredoxin from the sulfate-reducing bacterium Desulfovibrio gigas. Biochim Biophys Acta 449, 275-284. 10.1016/0005-2728(76)90139-0.
Burk, K. (2017). Friedreich Ataxia: current status and future prospects. Cerebellum Ataxias 4, 4. 10.1186/s40673-017-0062-x.
Campuzano, V., Montermini, L., Lutz, Y., Cova, L., Hindelang, C., Jiralerspong, S., Trottier, Y., Kish, S. J., Faucheux, B., Trouillas, P., et al. (1997). Frataxin is reduced in Friedreich ataxia patients and is associated with mitochondrial membranes. Hum Mol Genet 6, 1771-1780. 10.1093/hmg/6.11.1771.
Campuzano, V., Montermini, L., Molto, M. D., Pianese, L., Cossee, M., Cavalcanti, F., Monros, E., Rodius, F., Duclos, F., Monticelli, A., et al. (1996). Friedreich's ataxia: autosomal recessive disease caused by an intronic GAA triplet repeat expansion. Science 271, 1423-1427. 10.1126/science.271.5254.1423.
48
Chen, H., Shi, Z., Guo, J., Chang, K. J., Chen, Q., Yao, C. H., Haigis, M. C., and Shi, Y. (2020). The human mitochondrial 12S rRNA m(4)C methyltransferase METTL15 is required for mitochondrial function. J Biol Chem 295, 8505-8513. 10.1074/jbc.RA119.012127.
Chen, K., Lin, G., Haelterman, N. A., Ho, T. S., Li, T., Li, Z., Duraine, L., Graham, B. H., Jaiswal, M., Yamamoto, S., et al. (2016). Loss of Frataxin induces iron toxicity, sphingolipid synthesis, and Pdk1/Mef2 activation, leading to neurodegeneration. Elife 5. 10.7554/eLife.16043.
Christian, B. E., and Spremulli, L. L. (2012). Mechanism of protein biosynthesis in mammalian mitochondria. Biochim Biophys Acta 1819, 1035-1054. 10.1016/j.bbagrm.2011.11.009.
Cooper, J. M., and Schapira, A. H. (2003). Friedreich's Ataxia: disease mechanisms, antioxidant and Coenzyme Q10 therapy. Biofactors 18, 163-171. 10.1002/biof 0.5520180219.
Crooks, D. R., Maio, N., Lane, A. N., Jarnik, M., Higashi, R. M., Haller, R. G., Yang, Y., Fan, T. W., Linehan, W. M., and Rouault, T. A. (2018). Acute loss of iron-sulfur clusters results in metabolic reprogrammingand generation of lipid droplets in mammalian cells. J Biol Chem 293, 8297-8311.10.1074/jbc.RA118.001885.
D, G. H., Kelley, D. R., Tenen, D., Bernstein, B., and Rinn, J. L. (2016). Widespread RNA binding by chromatin-associated proteins. Genome Biol 17, 28. 10.1186/s13059-016-0878-3.
Desai, N., Brown, A., Amunts, A., and Ramakrishnan, V. (2017). The structure of the yeast mitochondrial ribosome. Science 355, 528-531. 10.1126/science.aa12415.
Deutsch, E. C., Santani, A. B., Perlman, S. L., Farmer, J. M., Stolle, C. A., Marusich,
M. F., and Lynch, D. R. (2010). A rapid, noninvasive immunoassay for frataxin: utility in assessment of Friedreich ataxia. Mol Genet Metab 101, 238-245. 10.1016/j.ymgme.2010.07.001.
DeWeirdt, P. C., Sangree, A. K., Hanna, R. E., Sanson, K. R., Hegde, M., Strand, C., Persky, N. S., and Doench, J. G. (2020). Genetic screens in isogenic mammalian cell lines without single cell cloning. Nat Commun 11, 752. 10.1038/s41467-020-14620-6.
Durr, A., Cossee, M., Agid, Y., Campuzano, V., Mignard, C., Penet, C., Mandel, J. L., Brice, A., and Koenig, M. (1996). Clinical and genetic abnormalities in patients with Friedreich's ataxia. N Engl J Med 335, 1169-1175. 10.1056/NEJM199610173351601.
Emptage, M. H., Dreyers, J. L., Kennedy, M. C., and Beinert, H. (1983). Optical and EPR characterization of different species of active and inactive aconitase. J Biol Chem 258, 11106-11111.
Emsley, P., Lohkamp, B., Scott, W. G., and Cowtan, K. (2010). Features and development of Coot. Acta Crystallogr D Biol Crystallogr 66, 486-501. 10.1107/S0907444910007493.
Eng, J. K., McCormack, A. L., and Yates, J. R. (1994). An approach to correlate tandem mass spectral data of peptides with amino acid sequences in a protein database. J Am Soc Mass Spectrom 5, 976-989. 10.1016/1044-0305(94)80016-2.
Filla, A., De Michele, G., Cavalcanti, F., Pianese, L., Monticelli, A., Campanella, G., and Cocozza, S. (1996). The relationship between trinucleotide (GAA) repeat length and clinical features in Friedreich ataxia. Am J Hum Genet 59, 554-560.
Goddard, T. D., Huang, C. C., Meng, E. C., Pettersen, E. F., Couch, G. S., Morris, J. H., and Ferrin, T. E. (2018). UCSF ChimeraX: Meeting modern challenges in visualization and analysis. Protein Sci 27, 14-25. 10.1002/pro.3235.
Gonzalez-Cabo, P., Vazquez-Manrique, R. P., Garcia-Gimeno, M. A., Sanz, P., and Palau, F. (2005). Frataxin interacts functionally with mitochondrial electron transport chain proteins. Hum Mol Genet 14, 2091-2098. 10.1093/hmg/ddi214.
Greene, E., Mahishi, L., Entezam, A., Kumari, D., and Usdin, K. (2007). Repeat-induced epigenetic changes in intron 1 of the frataxin gene and its consequences in Friedreich ataxia. Nucleic Acids Res 35, 3383-3390. 10.1093/nar/gkm271.
Groh, M., Lufino, M. M., Wade-Martins, R., and Gromak, N. (2014). R-loops associated with triplet repeat expansions promote gene silencing in Friedreich ataxia and fragile X syndrome. PLoS Genet 10, e1004318. 10.1371/journal.pgen.1004318.
Hagen, W. R. (1992). EPR spectroscopy of iron-sulfur proteins. In Advances in inorganic chemistry, (Elsevier), pp. 165-222.
49
Harding, A. E. (1981). Friedreich's ataxia: a clinical and genetic study of 90 families with an analysis of early diagnostic criteria and intrafamilial clustering of clinical features. Brain 104, 589-620. 10.1093/brain/104.3.589.
Huttlin, E. L., Bruckner, R. J., Navarrete-Perea, J., Cannon, J. R., Baltier, K., Gebreab, F., Gygi, M. P., Thornock, A., Zarraga, G., Tam, S., et al. (2021). Dual proteome-scale networks reveal cell-specific remodeling of the human interactome. Cell 184, 3022-3040 e3028. 10.1016/j.cell.2021.04.011.
Imlay, J. A. (2006). Iron-sulphur clusters and the problem with oxygen. Mol Microbiol 59, 1073-1082. 10.1111/j.1365-2958.2006.05028.x.
Itoh, Y., Khawaja, A., Laptev, I., Cipullo, M., Atanassov, I., Sergiev, P., Rorbach, J., and Amunts, A. (2022). Mechanism of mitoribosomal small subunit biogenesis and preinitiation. Nature 606, 603-608. 10.1038/s41586-022-04795-x.
Itoh, Y., Naschberger, A., Mortezaei, N., Herrmann, J. M., and Amunts, A. (2020). Analysis of translating mitoribosome reveals functional characteristics of translation in mitochondria of fungi. Nat Commun 11, 5187. 10.1038/s41467-020-18830-w.
Jumper, J., Evans, R., Pritzel, A., Green, T., Figurnov, M., Ronneberger, O., Tunyasuvunakool, K., Bates, R., Zidek, A., Potapenko, A., et al. (2021). Highly accurate protein structure prediction with AlphaFold. Nature 596, 583-589. 10.1038/s41586-021-03819-2.
Keita, M., McIntyre, K., Rodden, L. N., Schadt, K., and Lynch, D. R. (2022). Friedreich ataxia: clinical features and new developments. Neurodegener Dis Manag. 10.2217/nmt-2022-0011.
Khawaja, A., Itoh, Y., Remes, C., Spahr, H., Yukhnovets, O., Hofig, H., Amunts, A., and Rorbach, J. (2020). Distinct pre-initiation steps in human mitochondrial translation. Nat Commun 11, 2932. 10.1038/s41467-020-16503-2.
Khoroshilova, N., Popescu, C., Munck, E., Beinert, H., and Kiley, P. J. (1997). Iron-sulfur cluster disassembly in the FNR protein of Escherichia coli by 02: [4Fe-4S] to [2Fe-2S] conversion with loss of biological activity. Proc Natl Acad Sci USA 94, 6087-6092. 10.1073/pnas.94.12.6087.
Klootwijk, J., Klein, I., and Grivell, L. A. (1975). Minimal post-transcriptional modification of yeast mitochondrial ribosomal RNA. J Mol Biol 97, 337-350. 10.1016/s0022-2836(75)80044-1.
Koeppen, A. H. (2011). Friedreich's ataxia: pathology, pathogenesis, and molecular genetics. J Neurol Sci 303, 1-12. 10.1016/j.jns.2011.01.010.
Li, J., Cai, Z., Bomgarden, R. D., Pike, I., Kuhn, K., Rogers, J. C., Roberts, T. M., Gygi, S. P., and Paulo, J. A. (2021). TMTpro-18plex: The Expanded and Complete Set of TMTpro Reagents for Sample Multiplexing. J Proteome Res 20, 2964-2972. 10.1021/acs.jproteome.1c00168.
Liebschner, D., Afonine, P. V., Baker, M. L., Bunkoczi, G., Chen, V. B., Croll, T. I., Hintze, B., Hung, L. W., Jain, S., McCoy, A. J., et al. (2019). Macromolecular structure determination using X-rays, neutrons and electrons: recent developments in Phenix. Acta Crystallogr D Struct Biol 75, 861-877. 10.1107/S2059798319011471.
Lill, R., and Freibert, S. A. (2020). Mechanisms of Mitochondrial Iron-Sulfur Protein Biogenesis. Annu Rev Biochem 89, 471-499. 10.1146/annurev-biochem-013118-111540.
Lin, H., Magrane, J., Rattelle, A., Stepanova, A., Galkin, A., Clark, E. M., Dong, Y. N., Halawani, S. M., and Lynch, D. R. (2017). Early cerebellar deficits in mitochondrial biogenesis and respiratory chain complexes in the KIKO mouse model of Friedreich ataxia. Dis Model Mech 10, 1343-1352. 10.1242/dmm.030502.
Lodi, R., Cooper, J. M., Bradley, J. L., Manners, D., Styles, P., Taylor, D. J., and Schapira, A. H. (1999). Deficit of in vivo mitochondrial ATP production in patients with Friedreich ataxia. Proc Natl Acad Sci USA 96, 11492-11495. 10.1073/pnas.96.20.11492.
Lynch, D. R., Deutsch, E. C., Wilson, R. B., and Tennekoon, G. (2012). Unanswered questions in Friedreich ataxia. J Child Neurol 27, 1223-1229. 10.1177/0883073812453498.
Mai, N., Chrzanowska-Lightowlers, Z. M., and Lightowlers, R. N. (2017). The process of mammalian mitochondrial protein synthesis. Cell Tissue Res 367, 5-20. 10.1007/s00441-016-2456-0.
Maio, N., Jain, A., and Rouault, T. A. (2020). Mammalian iron-sulfur cluster biogenesis: Recent insights into the roles of frataxin, acyl carrier protein and ATPase-mediated transfer to recipient proteins. Curr Opin Chem Biol 55, 34-44. 10.1016/j.cbpa.2019.11.014.
Maio, N., and Rouault, T. A. (2020). Outlining the Complex Pathway of Mammalian Fe—S Cluster Biogenesis. Trends Biochem Sci 45, 411-426. 10.1016/j.tibs.2020.02.001.
Maio, N., Singh, A., Uhrigshardt, H., Saxena, N., Tong, W. H., and Rouault, T. A. (2014). Cochaperone binding to LYR motifs confers specificity of iron sulfur cluster delivery. Cell Metab 19, 445-457. 10.1016/j.cmet.2014.01.015.
Navarrete-Perea, J., Yu, Q., Gygi, S. P., and Paulo, J. A. (2018). Streamlined Tandem Mass Tag (SL-TMT) Protocol: An Efficient Strategy for Quantitative (Phospho)proteome Profiling Using Tandem Mass Tag-Synchronous Precursor Selection-MS3. J Proteome Res 17, 2226-2236. 10.1021/acs.jproteome.8b00217.
Nicolet, Y., and Fontecilla-Camps, J. C. (2014). 12 Iron-sulfur clusters and molecular oxygen: function, adaptation, degradation, and repair. Iron-Sulfur Clusters in Chemistry and Biology, 359.
Ott, M., Amunts, A., and Brown, A. (2016). Organization and Regulation of Mitochondrial Protein Synthesis. Annu Rev Biochem 85, 77-101. 10.1146/annurev-biochem-060815-014334.
Pandolfo, M. (2012). Friedreich ataxia. Handb Clin Neurol 103, 275-294. 10.1016/B978-O-444-51892-7.00017-6.
Parent, A., Elduque, X., Cornu, D., Belot, L., Le Caer, J. P., Grandas, A., Toledano, M. B., and D′Autreaux, B. (2015). Mammalian frataxin directly enhances sulfur transfer of NFS1 persulfide to both ISCU and free thiols. Nat Commun 6, 5686. 10.1038/ncomms6686.
Patra, S., and Barondeau, D. P. (2019). Mechanism of activation of the human cysteine desulfurase complex by frataxin. Proc Natl Acad Sci USA 116, 19421-19430. 10.1073/pnas.1909535116.
Pettersen, E. F., Goddard, T. D., Huang, C. C., Couch, G. S., Greenblatt, D. M., Meng, E. C., and Ferrin, T. E. (2004). UCSF Chimera—a visualization system for exploratory research and analysis. J Comput Chem 25, 1605-1612. 10.1002/jcc.20084.
Puccio, H., Simon, D., Cossee, M., Criqui-Filipe, P., Tiziano, F., Melki, J., Hindelang, C., Matyas, R., Rustin, P., and Koenig, M. (2001). Mouse models for Friedreich ataxia exhibit cardiomyopathy, sensory nerve defect and Fe—S enzyme deficiency followed by intramitochondrial iron deposits. Nat Genet 27, 181-186. 10.1038/84818.
Rad, R., Li, J., Mintseris, J., O'Connell, J., Gygi, S. P., and Schweppe, D. K. (2021). Improved Monoisotopic Mass Estimation for Deeper Proteome Coverage. J Proteome Res 20, 591-598. 10.1021/acs.jproteome.0c00563.
Rath, S., Sharma, R., Gupta, R., Ast, T., Chan, C., Durham, T. J., Goodman, R. P., Grabarek, Z., Haas, M. E., Hung, W. H. W., et al. (2021). MitoCarta3.0: an updated mitochondrial proteome now with sub-organelle localization and pathway annotations. Nucleic Acids Res 49, D1541-D1547. 10.1093/nar/gkaa1011.
Reetz, K., Dogan, I., Costa, A. S., Dafotakis, M., Fedosov, K., Giunti, P., Parkinson, M. H., Sweeney, M. G., Mariotti, C., Panzeri, M., et al. (2015). Biological and clinical characteristics of the European Friedreich's Ataxia Consortium for Translational Studies (EFACTS) cohort: a cross-sectional analysis of baseline data. Lancet Neurol 14, 174-182. 10.1016/S1474-4422(14)70321-7.
Reiter, V., Matschkal, D. M., Wagner, M., Globisch, D., Kneuttinger, A. C., Muller, M., and Carell, T. (2012). The CDK5 repressor CDK5RAP1 is a methylthiotransferase acting on nuclear and mitochondrial RNA. Nucleic Acids Res 40, 6235-6240. 10.1093/nar/gks240.
Rotig, A., de Lonlay, P., Chretien, D., Foury, F., Koenig, M., Sidi, D., Munnich, A., and Rustin, P. (1997). Aconitase and mitochondrial iron-sulphur protein deficiency in Friedreich ataxia. Nat Genet 17, 215-217.10.1038/ng1097-215.
Rouault, T. A. (2015). Iron-sulfur proteins hiding in plain sight. Nat Chem Biol 11, 442-445. 10.1038/nchembio.1843.
Saurer, M., Ramrath, D. J. F., Niemann, M., Calderaro, S., Prange, C., Mattei, S., Scaiola, A., Leitner, A., Bieri, P., Horn, E. K., et al. (2019). Mitoribosomal small subunit biogenesis in trypanosomes involves an extensive assembly machinery. Science 365, 1144-1149. 10.1126/science.aaw5570.
Schweppe, D. K., Eng, J. K., Yu, Q., Bailey, D., Rad, R., Navarrete-Perea, J., Huttlin, E. L., Erickson, B. K., Paulo, J. A., and Gygi, S. P. (2020). Full-Featured, Real-Time Database Searching Platform Enables Fast and Accurate Multiplexed Quantitative Proteomics. J Proteome Res 19, 2026-2034. 10.1021/acs.jproteome.9b00860.
Shi, Z., Xu, S., Xing, S., Yao, K., Zhang, L., Xue, L., Zhou, P., Wang, M., Yan, G., Yang, P., et al. (2019). METTL17, a regulator of mitochondrial ribosomal RNA modifications, is required for the translation of mitochondrial coding genes. FASEB J 33, 13040-13050. 10.1096/fj.201901331R.
Smith, P. K., Krohn, R. I., Hermanson, G. T., Mallia, A. K., Gartner, F. H., Provenzano, M. D., Fujimoto, E. K., Goeke, N. M., Olson, B. J., and Klenk, D. C. (1985). Measurement of protein using bicinchoninic acid. Anal Biochem 150, 76-85. 10.1016/0003-2697(85)90442-7.
Soragni, E., Herman, D., Dent, S. Y., Gottesfeld, J. M., Wells, R. D., and Napierala, M. (2008). Long intronic GAA*TTC repeats induce epigenetic changes and reporter gene silencing in a molecular model of Friedreich ataxia. Nucleic Acids Res 36, 6056-6065. 10.1093/nar/gkn604.
Srour, B., Gervason, S., Monfort, B., and D′Autreaux, B. (2020). Mechanism of Iron-Sulfur Cluster Assembly: In the Intimacy of Iron and Sulfur Encounter. Inorganics 8, 55.
Telot, L., Rousseau, E., Lesuisse, E., Garcia, C., Morlet, B., Leger, T., Camadro, J. M., and Serre, V. (2018). Quantitative proteomics in Friedreich's ataxia B-lymphocytes: A valuable approach to decipher the biochemical events responsible for pathogenesis. Biochim Biophys Acta Mol Basis Dis 1864, 997-1009. 10.1016/j.bbadis.2018.01.010.
Tsaousis, A. D. (2019). On the Origin of Iron/Sulfur Cluster Biosynthesis in Eukaryotes. Front Microbiol 10, 2478. 10.3389/fmicb.2019.02478.
Tsou, A. Y., Paulsen, E. K., Lagedrost, S. J., Perlman, S. L., Mathews, K. D., Wilmot, G. R., Ravina, B., Koeppen, A. H., and Lynch, D. R. (2011). Mortality in Friedreich ataxia. J Neurol Sci 307, 46-49. 10.1016/j.jns.2011.05.023.
Turchi, R., Tortolici, F., Guidobaldi, G., Iacovelli, F., Falconi, M., Rufini, S., Faraonio, R., Casagrande, V., Federici, M., De Angelis, L., et al. (2020). Frataxin deficiency induces lipid accumulation and affects thermogenesis in brown adipose tissue. Cell Death Dis 11, 51. 10.1038/s41419-020-2253-2.
Van Haute, L., Hendrick, A. G., D'Souza, A. R., Powell, C. A., Rebelo-Guiomar, P., Harbour, M. E., Ding, S., Fearnley, I. M., Andrews, B., and Minczuk, M. (2019). METTL15 introduces N4-methylcytidine into human mitochondrial 12S rRNA and is required for mitoribosome biogenesis. Nucleic Acids Res 47, 10267-10281. 10.1093/nar/gkz735.
Varadi, M., Anyango, S., Deshpande, M., Nair, S., Natassia, C., Yordanova, G., Yuan, D., Stroe, O., Wood, G., Laydon, A., et al. (2022). AlphaFold Protein Structure Database: massively expanding the structural coverage of protein-sequence space with high-accuracy models. Nucleic Acids Res 50, D439-D444. 10.1093/nar/gkab1061.
Wachnowsky, C., and Cowan, J. A. (2017). In Vitro Studies of Cellular Iron-Sulfur Cluster Biosynthesis, Trafficking, and Transport. Methods Enzymol 595, 55-82. 10.1016/bs.mie.2017.06.045.
Wang, D., Ho, E. S., Cotticelli, M. G., Xu, P., Napierala, J. S., Hauser, L. A., Napierala, M., Himes, B. E., Wilson, R. B., Lynch, D. R., and Mesaros, C. (2022). Skin fibroblast metabolomic profiling reveals that lipid dysfunction predicts the severity of Friedreich's ataxia. J Lipid Res 63, 100255. 10.1016/j.jlr.2022.100255.
Waterhouse, A., Bertoni, M., Bienert, S., Studer, G., Tauriello, G., Gumienny, R., Heer, F. T., de Beer, T. A. P., Rempfer, C., Bordoli, L., et al. (2018). SWISS-MODEL: homology modelling of protein structures and complexes. Nucleic Acids Res 46, W296-W303. 10.1093/nar/gky427.
Wei, F. Y., Zhou, B., Suzuki, T., Miyata, K., Ujihara, Y., Horiguchi, H., Takahashi, N., Xie, P., Michiue, H., Fujimura, A., et al. (2015). Cdk5rap1-mediated 2-methylthio modification of mitochondrial tRNAs governs protein translation and contributes to myopathy in mice and humans. Cell Metab 21, 428-442. 10.1016/j.cmet.2015.01.019.
Williams, C. J., Headd, J. J., Moriarty, N. W., Prisant, M. G., Videau, L. L., Deis, L. N., Verma, V., Keedy, D. A., Hintze, B. J., Chen, V. B., et al. (2018). MolProbity: More and better reference data for improvedall-atom structure validation. Protein Sci 27, 293-315. 10.1002/pro.3330.
Wong, J. M., and Eirin-Lopez, J. M. (2021). Evolution of Methyltransferase-Like (METTL) Proteins in Metazoa: A Complex Gene Family Involved in Epitranscriptomic Regulation and Other Epigenetic Processes. Mol Biol Evol 38, 5309-5327. 10.1093/molbev/msab267.
Zhang, K. (2016). Gctf: Real-time CTF determination and correction. J Struct Biol 193, 1-12. 10.1016/j.jsb.2015.11.003.
Zivanov, J., Nakane, T., Forsberg, B. O., Kimanius, D., Hagen, W. J., Lindahl, E., and Scheres, S. H. (2018). New tools for automated high-resolution cryo-EM structure determination in RELION-3. Elife 7. 10.7554/eLife.42166.
Zivanov, J., Nakane, T., and Scheres, S. H. W. (2020). Estimation of high-order aberrations and anisotropic magnification from cryo-EM data sets in RELION-3.1. IUCrJ 7, 253-267. 10.1107/S2052252520000081.

1-37. (canceled)

38. A method of enhancing intra-mitochondrial protein translation in a subject in need thereof, the method comprising:
administering a therapeutically effective amount of a composition comprising one or more agents effective to increase (i) methyltransferase like 17 (METTL17) gene expression, (ii) METTL17 protein expression and/or activity, or both (i) and (ii).

39. The method of claim 38, wherein the composition comprising one or more agents comprises DNA incorporated into a vector.

40. The method of claim 39, wherein the vector is a viral vector.

41. The method of claim 40, wherein the viral vector is selected from the group consisting of a lentiviral vector, an adenovirus vector, and an adeno-associated virus (AAV) vector.

42. The method of claim 39, wherein the vector is configured for stable integration of the DNA encoding METTL17 into a nuclear genome of target cells.

43. The method of claim 39, wherein the composition comprising one or more agents comprises an mRNA encoding METTL17.

44. The method of claim 43, wherein the mRNA is contained in a delivery vehicle.

45. The method of claim 43, wherein the delivery vehicle is selected from the group consisting of a viral capsid, a retroelement capsid, an engineered viral like particle (eVLP), and a nanoparticle.

46. The method of claim 38, wherein the composition comprising one or more agents is a gene editing system comprising a CRISPR-associated transposase (CAST) system comprising: i) a catalytically inactive Cas polypeptide and a transposase fused to or otherwise capable of associating with the Cas polypeptide; ii) a guide molecule capable of forming a complex with the Cas polypeptide and directing the complex to a target insertion site in the subject; and iii) a donor construct comprising the polynucleotide encoding METTL17, or a functional component thereof, and one or more transposase recognition sequences capable of facilitating recognition by the transposase, whereby the transposase facilitates insertion of the polynucleotide encoding METTL17 at the target insertion site in the subject.

47. The method of claim 38, wherein the composition comprising one or more agents comprises an engineered transcriptional activator system comprising a DNA-binding domain and a transcriptional activator configured to bind an enhancer of the METTL17 gene such that expression of METLL17 is increased in the subject.

48. The method of claim 47, wherein the DNA-binding domain is a catalytically inactive Cas polypeptide, and the composition further comprises a guide molecule capable of forming a complex with the Cas polypeptide and directing binding of the complex and an epigenetic modification domain to a target region of the genome of the subject such that the epigenetic modification domain modifies a chromosomal architecture such that METLL17 expression is increased in the subject.

49. The method of claim 48, wherein the epigenetic modification domain is a demethylation domain that demethylates one or more CpG islands responsible for silencing expression of METTL17.

50. The method of claim 38, wherein the subject in need thereof is affected by age-related mitochondrial dysfunction or decreased mitochondrial activity not associated with mitochondrial disease.

51. A method of treating a mitochondrial disease in a subject in need thereof, the method comprising:
administering a therapeutically effective amount of a composition comprising one or more agents effective to increase (i) methyltransferase like 17 (METTL17) gene expression, (ii) METTL17 protein expression and/or activity, or both (i) and (ii).

52. The method of claim 51, wherein the mitochondrial disease is caused by a mutation in either the mitochondrial DNA (mtDNA) or nuclear DNA (nucDNA) of the subject.

53. The method of claim 51, wherein the mitochondrial disease is a homoplasmic or a heteroplasmic mitochondrial DNA (mtDNA) disease.

54. The method of claim 52, wherein the mitochondrial disease is a monogenic mitochondrial disease.

55. The method of claim 51, wherein the mitochondrial disease is due to mutation of the frataxin (FXN) gene.

56. The method of claim 51, wherein the mitochondrial disease is Friedrich's ataxia.

57. A composition for enhancing intra-mitochondrial protein translation comprising one or more agents effective to increase (i) methyltransferase like 17 (METTL17) gene expression, (ii) METTL17 protein expression and/or activity, or both (i) and (ii).
